(19) United States
(12) Patent Application Publication
(10) Pub. No.: US 2024/0026407 A1
(43) Pub. Date: Jan. 25, 2024

(54) MODIFICATION OF SAPONINS

(71) Applicant: GLAXOSMITHKLINE BIOLOGICALS SA, Rixensart (BE)

(72) Inventors: Murray BROWN, Stevenage (GB); Edward CHAPMAN, Worthing (GB); Andrew COLLIS, Ulverston (GB); Douglas FUERST, Collegeville, PA (US); Joseph HOSFORD, Stevenage (GB); Christopher MACDERMAID, Collegeville, PA (US); James MORRISON, Collegeville, PA (US)

(73) Assignee: GLAXOSMITHKLINE BIOLOGICALS SA, Rixensart (BE)

(21) Appl. No.: 18/039,514

(22) PCT Filed: Dec. 8, 2021
(86) PCT No.: PCT/EP2021/084813

§ 371 Date: May 31, 2023

Related U.S. Application Data
(60) Provisional application No. 63/123,011, filed on Dec. 9, 2020.
(60) Provisional application No. 63/213,382, filed on Jun. 22, 2021.
(60) Provisional application No. 63/213,407, filed on Jun. 22, 2021.
(60) Provisional application No. 63/213,340, filed on Jun. 22, 2021.

(51) Int. Cl. C12P 19/14; C12N 9/42; C12N 9/24; A61K 39/39

(52) U.S. Cl. CPC C12P 19/14; C12N 9/2445; C12Y 302/01021; C12N 9/2402; C12Y 302/0104; A61K 39/39; A61K 2039/55577

(57) ABSTRACT

Methods for the enzymatic modification of saponins, products made thereby, uses of said products and also to other associated aspects. The saponins may be extracts of Quillaja species, such as extracts of Quillaja saponaria Molina.

### TECHNICAL FIELD

The present application generally relates to saponins, in particular methods for the enzymatic modification of saponins, products made thereby, uses of said products and also to other associated aspects. The present application further relates to glucosidases and rhamnosidases, in particular mutated glucosidases and rhamnosidases which may be of use in methods for the enzymatic modification of saponins. The saponins may be obtainable from Quillaja species, including extracts obtainable from Quillaja species, such as extracts of Quillaja saponaria Molina.

### BACKGROUND

Saponins are steroid or terpenoid glycosides. They have a broad range of uses from fire extinguisher foams to food additives and immunostimulants (Reichert, 2019).

Quillaja extract (E 999) is currently approved by the European Food Safety Authority under EU Regulation 1129/2011 as a food additive in flavoured drinks (14.1.4), cider and perry (14.2.3). Quillaja extract (E 999) is described as being obtained by aqueous extraction of the milled inner bark or wood of Quillaja saponaria, or other Quillaja species. It is also described as containing a number of triterpenoid saponins consisting of glycosides of quillaic acid. Sugars—including glucose, galactose, arabinose, xylose, and rhamnose—are also said to be present, along with tannin, calcium oxalate and other minor components. (EFSA Journal 2019 17(3):5622)

Saponins have been of interest as immunostimulants for many decades (see, for example, Hyslop, 1969). Quil A is a saponin fraction derived from an aqueous extract from the bark of Quillaja saponaria (Dalsgaard, 1974). Quil A itself contains a plurality of components with the four most predominant Quil A fractions purified by reverse phase chromatography, namely QS-7, QS-17, QS-18 and QS-21, all having immunostimulatory activity although varying in haemolytic activity and toxicity (Kensil, 1991; Kensil, 1995). The main saponin fraction, QS-18, was found to be highly toxic in mice but saponin fractions QS-7 and QS-21 were far less toxic. QS-21, being more abundant than QS-7, has been the most widely studied saponin adjuvant (Ragupathi, 2011).

Liquid chromatography/mass spectrometry analysis of Quillaja saponaria bark water/methanol extracts has revealed over 100 saponins, many of which have been assigned structures (Nyberg, 2000; Nyberg, 2003; Kite, 2004).

Quillaja brasiliensis (A St.-Hil & Tul) Mart. extracts have been described, with the identity of various components therein determined by mass spectrometry. Many saponin components in Quillaja brasiliensis extracts correspond to saponins found in Quillaja saponaria extracts (Wallace, 2017; Wallace, 2019) and Quillaja brasiliensis extracts have also been shown to have immunostimulant effects (Cibulski, 2018; Yendo, 2017).

The Adjuvant System 01 (AS01) is a liposome-based adjuvant which contains two immunostimulants, 3-O-desacyl-4′-monophosphoryl lipid A (3D-MPL) and QS-21 (Garcon, 2011; Didierlaurent, 2017). 3D-MPL is a non-toxic derivative of the lipopolysaccharide from Salmonella minnesota. AS01 is included in vaccines for malaria (RTS, S-Mosquirix™) and Herpes zoster (HZ/su—Shingrix™), and in multiple candidate vaccines. AS01 injection results in rapid and transient activation of innate immunity in animal models. Neutrophils and monocytes are rapidly recruited to the draining lymph node (dLN) upon immunization. Moreover, AS01 induces recruitment and activation of MHCII^{high} dendritic cells (DC), which are necessary for T cell activation (Didierlaurent, 2014). Some data are also available on the mechanism of action of the components of AS01. 3D-MPL signals via TLR4, stimulating NF-κB transcriptional activity and cytokine production and directly activates antigen-presenting cells (APCs) both in humans and in mice (De Becker, 2000; Ismaili, 2002; Martin, 2003; Mata-Haro, 2007). QS-21 promotes high antigen-specific antibody responses and CD8⁺ T-cell responses in mice (Kensil, 1998; Newman, 1992; Soltysik, 1995) and antigen-specific antibody responses in humans (Livingston, 1994). Because of its physical properties, it is thought that QS-21 might act as a danger signal in vivo (Lambrecht, 2009; Li, 2008). Although QS-21 has been shown to activate ASC-NLRP3 inflammasome and subsequent IL-1β/IL-18 release (Marty-Roix, 2016), the exact molecular pathways involved in the adjuvant effect of saponins have yet to be clearly defined.

Extracts of Quillaja saponaria are commercially available, including fractions thereof with differing degrees of purity such as Quil A, Fraction A, Fraction B, Fraction C, QS-7, QS-17, QS-18 and QS-21.

The enzymatic hydrolysis of Quil-A by Rapidase® Revelation Aroma has been described during the development of a quality control method to provide a degraded reference sample (Lecas, 2021).

Availability of saponins is constrained, particularly those obtained from rarer plants or where saponins of interest are present in relatively low amounts. Furthermore, separation of certain saponins from other components, particularly other saponin components which may have similar structures, can be burdensome. Consequently, there remains a need for new methods which may improve the yield of saponins of interest and/or facilitate removal of undesired saponin components.

Modestobacter marinus glucosidase (Uniparc reference UPI000260A2FA, Uniprot reference I4EYD5) is a naturally occurring glucosidase. There remains a need for further glucosidases which may have improved properties.

Kribbella flavida rhamnosidase (Uniparc reference UPI00019BDB13, Uniprot reference D2PMT) is a naturally occurring rhamnosidase. There remains a need for further rhamnosidases which may have improved properties.

### SUMMARY OF THE INVENTION

The present invention provides a method for making a product saponin, said method comprising the step of enzymatically converting a starting saponin to the product saponin. Suitably the method uses a polypeptide of the invention, such as an engineered glucosidase polypeptide or an engineered rhamnosidase polypeptide.

Also provided is a method for increasing the amount of a product saponin in a composition, said method comprising the step of enzymatically converting a starting saponin to the product saponin. Suitably the method uses a polypeptide of the invention, such as an engineered glucosidase polypeptide or an engineered rhamnosidase polypeptide.

Further provided is a method for reducing the amount of a starting saponin in a composition, said method comprising the step of enzymatically converting the starting saponin to a product saponin. Suitably the method uses a polypeptide of the invention, such as an engineered glucosidase polypeptide or an engineered rhamnosidase polypeptide.

The use of a glycosidase for enzymatically converting a starting saponin to a product saponin is also provided by the invention. Suitably the glycosidase is a polypeptide of the invention, such as an engineered glucosidase polypeptide or an engineered rhamnosidase polypeptide.

Additionally provided is a method for identifying a candidate enzyme having beta exo glucosidase activity, comprising selecting an enzyme comprising, such as consisting of: (i) an amino acid sequence according to SEQ ID No. 262, 208, 63, 229, 250, 5, 101, 207, 169, 247, 302, 324, 319, 9, 240, 325, 338, 850, 879, 868, 826, 804, 888, 881, 891, 816, 827, 857, 853, 842, 814, 886, 885, 838, 829, 808, 828, 870, 873, 844, 882, 874, 825, 824, 823, 810, 894, 849, 803, 890, 841, 832, 830, 845, 871, 837, 883 or 809 or functional variants thereof; or (ii) an amino acid sequence according to SEQ ID No. 262, 208, 63, 229, 250, 5, 101, 207, 169, 247, 302, 324, 319, 9, 240, 325, 338, 850, 879, 868, 826, 804, 888, 881, 891, 816, 827, 857, 853, 842, 814, 886, 885, 838, 829, 808, 828, 870, 873, 844, 882, 874, 825, 824, 823, 810, 894, 849, 803, 890, 841, 832, 830, 845, 871, 837, 883 or 809 or functional variants thereof

Also provided is a method for identifying a candidate enzyme having alpha exo rhamnosidase activity, comprising selecting an enzyme comprising, such as consisting of, an amino acid sequence according to SEQ ID No. 992, 1003, 1052, 1073, 1017, 1055, 1075, 1001, 1007, 1061, 1079, 1027, 1039, 1041, 989, 1053, 1018, 1066, 1082, 1076, 993, 1077, 1046, 1015, 1063, 1054, 1074, 1067 or 1033, or functional variants thereof.

Also provided are engineered glucosidase and rhamnosidase polypeptides as further detailed below (referred to as polypeptides of the invention).

The present invention provides an engineered glucosidase polypeptide comprising, such as consisting of, an amino acid sequence that is at least 80%, 85%, 90%, 95%, 96%, 97%, 98%, or 99% identical to the amino acid sequence of SEQ ID No. 262, or a functional fragment thereof, wherein the engineered glucosidase polypeptide includes at least one residue substitution from:
F44Y;
V60L;
G117A;
F170N;
V263G or V263L;
N351H or N351Q;
A355H, A355I, A355L, A355M, A355R, A355T or A355W;
A356P;
R357A, R357C, R357K, R357M or R357Q;
G362C;
T365A, T365N or T365S;
L367C;
V394R;
V395Y;
Q396E, Q396G, Q396N, Q396P, Q396R, Q396S or Q396Y;
F430W;
R435F;
V438T;
V440F;
F442M or F442Q;
G444T;
A473F or A473R;
L474C, L474I or L474V;
I475F;
L492C, L492G, L492H, L492I, L492N, L492Q, L492V, L492W or L492Y;
Q493F or Q493H;
P494H or P494I;
S495I, S495K or S495Q;
G496P or G496W;
D498A, D498E, D498F, D498I, D498K, D498L, D498N, D498P, D498R, D498S, D498T or D498V;
A502R;
M504G or M504R;
L507A or L507R;
T508M;
L529M;
F535P;
A536D or A536E;
A537R;
F541A, F541I, F541L, F541M or F541V;
L542I;
Q543G or Q543L;
E547L; and
Y585W.

The present invention provides an engineered rhamnosidase polypeptide comprising, such as consisting of, an amino acid sequence that is at least 80%, 85%, 90%, 95%, 96%, 97%, 98%, or 99% identical to the amino acid sequence of SEQ ID No. 1017, or a functional fragment thereof, wherein the engineered rhamnosidase polypeptide includes at least one residue substitution from:
(i) A56C
(ii) A143P
(iii) Q181H, Q181R or Q181S
(iv) L214M
(v) G215S
(vi) F216M
(vii) G218D or G218N
(viii) K219G
(ix) A238M
(x) T252Y
(xi) T311W
(xii) V326C
(xiii) G357C
(xiv) S369C, S369I, S369K or S369M
(xv) I487M, I487Q or I487V
(xvi) K492N
(xvii) V499T
(xviii) G508S
(xix) R543C
(xx) L557Y
(xxi) G634A
(xxii) S635N
(xxiii) A690C and
(xxiv) Q921H.

The invention also provides a saponin prepared by the methods herein, a saponin containing composition comprising a product saponin prepared by the methods herein, adjuvant compositions comprising said saponins or saponin containing compositions, and adjuvant compositions prepared using said saponins or saponin containing compositions. Use of a saponin or saponin containing composition of the invention in the manufacture of an adjuvant composition is also provided.

Further provided are immunogenic compositions comprising a saponin or saponin containing composition according to the invention and an antigen or a polynucleotide encoding an antigen. Kits comprising (i) a saponin or saponin containing composition according to the invention and (ii) an antigen or a polynucleotide encoding an antigen are also provided.

### BRIEF DESCRIPTION OF THE FIGURES

FIG. 1 HPLC chromatogram of a crude aqueous Quillaja saponaria bark extract

FIG. 2 HPLC-UV chromatogram of a crude aqueous Quillaja saponaria bark extract

FIG. 3 UPLC-UV chromatogram of a crude aqueous Quillaja saponaria bark extract

FIG. 4 UPLC-UV chromatogram of a polystyrene purified Quillaja saponaria QS-21 saponin extract with low content of 2018 component

FIG. 5 UPLC-UV/MS chromatogram of a Quillaja saponaria QS-21 purified saponin extract with low content of 2018 component

FIG. 6 UPLC-UV/MS chromatogram detail of a Quillaja saponaria QS-21 purified saponin extract with low content of 2018 component

FIG. 7A-7B Extracted mass chromatograms for 1988 (FIG. 7A) and 2002 (FIG. 7B) molecular weight ions of a Quillaja saponaria QS-21 purified saponin extract with low content of 2018 component

FIG. 8 Combined centroid spectrum of Quillaja saponaria QS-21 purified saponin extract with low content of 2018 component

FIG. 9 UPLC-UV chromatogram of Quillaja saponaria QS-21 purified saponin extract with low 2018 component

FIG. 10 LCMS/MS chromatogram for QS-18 2150 (Panel A) and QS-21 1988 (Panel B) component content in QS-21 standard in Example 4-2

FIG. 11 LCMS/MS chromatogram for QS-18 2150 (Panel A) and QS-21 1988 (Panel B) component content following negative control treatment in Example 4-2

FIG. 12 LCMS/MS chromatogram for QS-18 2150 (Panel A) and QS-21 1988 (Panel B) component content following glucosidase SEQ ID No. 262 treatment in Example 4-2

FIG. 13 UV HPLC chromatogram following glucosidase SEQ ID No. 262 treatment (upper trace) and negative control treatment (lower trace) of Crude Bark Extract (CBE) in Example 4-4

FIG. 14 LCMS/MS chromatogram for QS-17 2296 (Panel A) and QS-18 2150 (Panel B) component content following negative control treatment in Example 6-1

FIG. 15 LCMS/MS chromatogram for desglucosyl-QS-17 2134 (Panel A) and QS-21 1988 (Panel B) component content following negative control treatment in Example 6-1

FIG. 16 LCMS/MS chromatogram for QS-17 2310 (Panel A) and QS-18 2164 (Panel B) component content following negative control treatment in Example 6-1

FIG. 17 LCMS/MS chromatogram for QS-17 2296 (Panel A) and QS-18 2150 (Panel B) component content following rhamnosidase SEQ ID No. 1017 treatment in Example 6-1

FIG. 18 LCMS/MS chromatogram for desglucosyl-QS-17 2134 (Panel A) and QS-21 1988 (Panel B) component content following rhamnosidase SEQ ID No. 1017 treatment in Example 6-1

FIG. 19 LCMS/MS chromatogram for QS-17 2310 (Panel A) and QS-18 2164 (Panel B) component content following rhamnosidase SEQ ID No. 1017 treatment in Example 6-1

FIG. 20 UV HPLC chromatogram following rhamnosidase SEQ ID No. 1017 treatment (upper trace) and negative control treatment (lower trace) of Treated Bark Extract (TBE) in Example 6-2

FIG. 21 UV HPLC chromatogram following rhamnosidase SEQ ID No. 1017 treatment (upper trace) and negative control treatment (lower trace) of CBE in Example 6-3

FIG. 22 LCMS/MS chromatogram for QS-21 1988 component content at TO (Panel A) and at 24 hrs (Panel B) following dual enzyme treatment of CBE in Example 7

FIG. 23A-B Illustrative UV HPLC chromatogram following glucosidase enzyme treatment of CBE (FIG. 23a) and negative control treatment of CBE (FIG. 23b) in Example 8

FIG. 24 Illustrative UV HPLC chromatogram following rhamnosidase enzyme treatment of CBE (upper trace) and negative control treatment of CBE (lower trace) in Example 9

FIG. 25 HPLC-UV chromatogram of untreated and enzyme treated CBE at 1 L scale from Example 11

FIG. 26 UPLC-UV chromatogram following purification of untreated and enzyme treated CBE at 1 L scale from Example 11 (full acquisition)

FIG. 27 UPLC-UV chromatogram following purification of untreated and enzyme treated CBE at 1 L scale from Example 11 (zoom)

### BRIEF DESCRIPTION OF THE SEQUENCE IDENTIFIERS

Seq ID No. 1: Amino acid sequence for Cyberlindnera fabianii glucosidase, Uniparc reference UPI00049B1A8C, Uniprot reference A0A061B3J2.
Seq ID No. 2: Amino acid sequence for Flavobacterium gilvum glucosidase, Uniparc reference UPI0004E3EF7B, Uniprot reference A0A085EII0.
Seq ID No. 3: Amino acid sequence for Algibacter lectus glucosidase, Uniparc reference UPI00050EE490, Uniprot reference A0A090X649.
Seq ID No. 4: Amino acid sequence for Microbacterium azadirachtae glucosidase, Uniparc reference UPI0005ECB51E, Uniprot reference A0A0F0LB94.
Seq ID No. 5: Amino acid sequence for Actinobacteria bacterium glucosidase, Uniparc reference UPI0006588DAD, Uniprot reference A0A0J0UT37.
Seq ID No. 6: Amino acid sequence for Chloroflexi bacterium glucosidase, Uniparc reference UPI0007968552, Uniprot reference A0A136KWB3.
Seq ID No. 7: Amino acid sequence for Komagataeibacter rhaeticus glucosidase, Uniparc reference UPI0002080410, Uniprot reference A0A181C809.
Seq ID No. 8: Amino acid sequence for Bacteroides sp. glucosidase, Uniparc reference UPI0008211BFC, Uniprot reference A0A1C5WEL8.
Seq ID No. 9: Amino acid sequence for Streptomyces rubrolavendulae glucosidase, Uniparc reference UPI00085A2BD0, Uniprot reference A0A1D8FZW3.
Seq ID No. 10: Amino acid sequence for Clostridium roseum glucosidase, Uniparc reference UPI00098C60F6, Uniprot reference A0A1S8KYM5.
Seq ID No. 11: Amino acid sequence for uncultured bacterium glucosidase, Uniparc reference UPI0009CE0D4C, Uniprot reference A0A1V5M6V6.
Seq ID No. 12: Amino acid sequence for Firmicutes bacterium glucosidase, Uniparc reference UPI0009D4127D, Uniprot reference A0A1V6AN95.
Seq ID No. 13: Amino acid sequence for Anthracocystis flocculosa glucosidase, Uniparc reference UPI00045601AB, Uniprot reference A0A061H1Z3.
Seq ID No. 14: Amino acid sequence for Bifidobacterium boum glucosidase, Uniparc reference UPI0004FF77C7, Uniprot reference A0A086ZKU2.
Seq ID No. 15: Amino acid sequence for Jejuia pallidilutea glucosidase, Uniparc reference UPI00051EDBDE, Uniprot reference A0A098LTR2.
Seq ID No. 16: Amino acid sequence for Ceratocystis fimbriata glucosidase, Uniparc reference UPI00062105AB, Uniprot reference A0A0F8B2B0.
Seq ID No. 17: Amino acid sequence for Actinobacteria bacterium glucosidase, Uniparc reference UPI0006583AB1, Uniprot reference A0A0J0UVW7.
Seq ID No. 18: Amino acid sequence for Rhodococcus sp. glucosidase, Uniparc reference UPI0007AABFAD, Uniprot reference A0A143QAX3.
Seq ID No. 19: Amino acid sequence for Valsa mali glucosidase, Uniparc reference UPI0007F2D02D, Uniprot reference A0A194VF47.
Seq ID No. 20: Amino acid sequence for uncultured Bacteroides sp. glucosidase, Uniparc reference UPI000821004D, Uniprot reference A0A1C5WSI4.
Seq ID No. 21: Amino acid sequence for Eisenbergiella tayi glucosidase, Uniparc reference UPI00084089B2, Uniprot reference A0A1E3ALT2.
Seq ID No. 22: Amino acid sequence for Streptomyces sp. glucosidase, Uniparc reference UPI000978E914, Uniprot reference A0A1V2MY14.
Seq ID No. 23: Amino acid sequence for Firmicutes bacterium glucosidase, Uniparc reference UPI0009CBF21C, Uniprot reference A0A1V5MH90.
Seq ID No. 24: Amino acid sequence for Tenericutes bacterium glucosidase, Uniparc reference UPI0009D5B1F0, Uniprot reference A0A1V6BAK3.
Seq ID No. 25: Amino acid sequence for Gluconobacter oxydans glucosidase, Uniparc reference UPI0004A87350, Uniprot reference A0A067Z479.
Seq ID No. 26: Amino acid sequence for Bifidobacterium catenulatum glucosidase, Uniparc reference UPI00050787A2, Uniprot reference A0A087B8Q8.
Seq ID No. 27: Amino acid sequence for Bionectria ochroleuca glucosidase, Uniparc reference UPI0005965863, Uniprot reference A0A0B7K538.
Seq ID No. 28: Amino acid sequence for Parcubacteria sp. glucosidase, Uniparc reference UPI0006377CA4, Uniprot reference A0A0G0GD78.
Seq ID No. 29: Amino acid sequence for Microbacterium ketosireducens glucosidase, Uniparc reference UPI0006228575, Uniprot reference A0A0M2H276.
Seq ID No. 30: Amino acid sequence for Roseburia faecis glucosidase, Uniparc reference UPI0006C454B4, Uniprot reference A0A173R3W4.
Seq ID No. 31: Amino acid sequence for Kwoniella dejecticola glucosidase, Uniparc reference UPI0007F1D695, Uniprot reference A0A1A6A050.
Seq ID No. 32: Amino acid sequence for uncultured Clostridium sp. glucosidase, Uniparc reference UPI0008232A70, Uniprot reference A0A1C6C862.
Seq ID No. 33: Amino acid sequence for Cyberlindnera jadinii glucosidase, Uniparc reference UPI000866CB30, Uniprot reference A0A1E4S2F8.
Seq ID No. 34: Amino acid sequence for Bacteroidetes bacterium glucosidase, Uniparc reference UPI0009D3483D, Uniprot reference A0A1V5G4W6.
Seq ID No. 35: Amino acid sequence for Verrucomicrobia bacterium glucosidase, Uniparc reference UPI0009CC9AEA, Uniprot reference A0A1V5Q4R4.
Seq ID No. 36: Amino acid sequence for Bacteroidetes bacterium glucosidase, Uniparc reference UPI0009C6DE73, Uniprot reference A0A1V6BV25.
Seq ID No. 37: Amino acid sequence for Lichtheimia ramosa glucosidase, Uniparc reference UPI0004E051A9, Uniprot reference A0A077WUK7.
Seq ID No. 38: Amino acid sequence for Bifidobacterium mongoliense glucosidase, Uniparc reference UPI0005060F52, Uniprot reference A0A087BWT7.
Seq ID No. 39: Amino acid sequence for Vibrio ishigakensis glucosidase, Uniparc reference UPI0005910ED9, Uniprot reference A0A0B8NZY1.
Seq ID No. 40: Amino acid sequence for Phaeomoniella chlamydospora glucosidase, Uniparc reference UPI00063B706F, Uniprot reference A0A0G2HEV5.
Seq ID No. 41: Amino acid sequence for Ardenticatena maritima glucosidase, Uniparc reference UPI0006C04F59, Uniprot reference A0A0M8K5H7.
Seq ID No. 42: Amino acid sequence for Coprococcus comes glucosidase, Uniparc reference UPI000197E031, Uniprot reference A0A173WPC4.
Seq ID No. 43: Amino acid sequence for Nocardioides dokdonensis glucosidase, Uniparc reference UPI0007DDBAB3, Uniprot reference A0A1A9GNJ0.
Seq ID No. 44: Amino acid sequence for uncultured Clostridium sp. glucosidase, Uniparc reference UPI000822F7EB, Uniprot reference A0A1C6EBC1.
Seq ID No. 45: Amino acid sequence for Acetobacterium wieringae glucosidase, Uniparc reference UPI000878EB40, Uniprot reference A0A1F2PFB4.
Seq ID No. 46: Amino acid sequence for Tenericutes bacterium glucosidase, Uniparc reference UPI0009D0A2C1, Uniprot reference A0A1V5HNJ3.
Seq ID No. 47: Amino acid sequence for Tenericutes bacterium glucosidase, Uniparc reference UPI0009CB8A73, Uniprot reference A0A1V5UFI1.
Seq ID No. 48: Amino acid sequence for uncultured bacterium glucosidase, Uniparc reference UPI0009CC90AF, Uniprot reference A0A1V6CDT2.
Seq ID No. 49: Amino acid sequence for Parabacteroides distasonis glucosidase, Uniparc reference UPI0004D8E473, Uniprot reference A0A078SYD0.
Seq ID No. 50: Amino acid sequence for Bifidobacterium psychraerophilum glucosidase, Uniparc reference UPI0005006C6A, Uniprot reference A0A087CJB0.
Seq ID No. 51: Amino acid sequence for Hebeloma cylindrosporum glucosidase, Uniparc reference UPI00059A3BA8, Uniprot reference A0A0C2YJF2.
Seq ID No. 52: Amino acid sequence for Brenneria goodwinii glucosidase, Uniparc reference UPI0006579FEA, Uniprot reference A0A0G4JRR8.
Seq ID No. 53: Amino acid sequence for Aspergillus calidoustus glucosidase, Uniparc reference UPI00073C9E60, Uniprot reference A0A0U5GN20.
Seq ID No. 54: Amino acid sequence for Bacteroides finegoldii glucosidase, Uniparc reference UPI0006C6E0C3, Uniprot reference A0A174BEZ2.
Seq ID No. 55: Amino acid sequence for Altererythrobacter dongtanensis glucosidase, Uniparc reference UPI0008153D6D, Uniprot reference A0A1B2A943.
Seq ID No. 56: Amino acid sequence for uncultured Anaerotruncus sp. glucosidase, Uniparc reference UPI0008206E7B, Uniprot reference A0A1C6FWD9.
Seq ID No. 57: Amino acid sequence for Candidatus firestone glucosidase, Uniparc reference UPI0008AC0E17, Uniprot reference A0A1F5UJR6.
Seq ID No. 58: Amino acid sequence for Tenericutes bacterium glucosidase, Uniparc reference UPI0009C6C8E4, Uniprot reference A0A1V5HS22.
Seq ID No. 59: Amino acid sequence for Lentisphaerae bacterium glucosidase, Uniparc reference UPI0009CEE1AB, Uniprot reference A0A1V5VBL2.
Seq ID No. 60: Amino acid sequence for Planctomycetes bacterium glucosidase, Uniparc reference UPI0009CAAB0A, Uniprot reference A0A1V6FZ47.
Seq ID No. 61: Amino acid sequence for Pseudallescheria apiosperma glucosidase, Uniparc reference UPI0004DD62AC, Uniprot reference A0A084G332.
Seq ID No. 62: Amino acid sequence for Nonlabens sediminis glucosidase, Uniparc reference UPI000507F00A, Uniprot reference A0A090Q4N8.
Seq ID No. 63: Amino acid sequence for Gynuella sunshinyii glucosidase, Uniparc reference UPI0005CC42CA, Uniprot reference A0A0C5VDU3.
Seq ID No. 64: Amino acid sequence for Verticillium longisporum glucosidase, Uniparc reference UPI00063E4005, Uniprot reference A0A0G4N9Q7.
Seq ID No. 65: Amino acid sequence for Cellulomonas sp. glucosidase, Uniparc reference UPI00073C6CD3, Uniprot reference A0A0V8TAB5.
Seq ID No. 66: Amino acid sequence for Hungatella hathewayi glucosidase, Uniparc reference UPI0006C024BB, Uniprot reference A0A174FBX7.
Seq ID No. 67: Amino acid sequence for Mesorhizobium sp. glucosidase, Uniparc reference UPI000688E74D, Uniprot reference A0A1C2DG64.
Seq ID No. 68: Amino acid sequence for Clostridium sp. glucosidase, Uniparc reference UPI000822FAB7, Uniprot reference A0A1C6GRT5.
Seq ID No. 69: Amino acid sequence for Chlamydiales bacterium glucosidase, Uniparc reference UPI0009284E74, Uniprot reference A0A1M3CSY6.
Seq ID No. 70: Amino acid sequence for Spirochaetes bacterium glucosidase, Uniparc reference UPI0009CB461D, Uniprot reference A0A1V5HUX3.
Seq ID No. 71: Amino acid sequence for bacterium glucosidase, Uniparc reference UPI0009C92D6A, Uniprot reference A0A1V5VHB9.
Seq ID No. 72: Amino acid sequence for Thermotogae bacterium glucosidase, Uniparc reference UPI0009D12B86, Uniprot reference A0A1V6H2W5.
Seq ID No. 73: Amino acid sequence for Pseudallescheria apiosperma glucosidase, Uniparc reference UPI0004DCBED3, Uniprot reference A0A084GGE2.
Seq ID No. 74: Amino acid sequence for Algibacter lectus glucosidase, Uniparc reference UPI00050E0BE3, Uniprot reference A0A090VF17.
Seq ID No. 75: Amino acid sequence for Paxillus involutus glucosidase, Uniparc reference UPI0005B075C8, Uniprot reference A0A0C9TWP5.
Seq ID No. 76: Amino acid sequence for Verticillium longisporum glucosidase, Uniparc reference UPI00063DF006, Uniprot reference A0A0G4NA55.
Seq ID No. 77: Amino acid sequence for Mucilaginibacter gotjawali glucosidase, Uniparc reference UPI00076F8EA4, Uniprot reference A0A110B1H1.
Seq ID No. 78: Amino acid sequence for Bacteroides uniformis glucosidase, Uniparc reference UPI0006C0FFEF, Uniprot reference A0A174IWW4.
Seq ID No. 79: Amino acid sequence for Coprococcus sp. glucosidase, Uniparc reference UPI0008222B77, Uniprot reference A0A1C5W0N6.
Seq ID No. 80: Amino acid sequence for Blautia sp. glucosidase, Uniparc reference UPI0006C36823, Uniprot reference A0A1C6K2X5.
Seq ID No. 81: Amino acid sequence for Cellulomonas sp. glucosidase, Uniparc reference UPI00092B3CD4, Uniprot reference A0A1M3ELH4.
Seq ID No. 82: Amino acid sequence for bacterium glucosidase, Uniparc reference UPI0009C85428, Uniprot reference A0A1V5J984.
Seq ID No. 83: Amino acid sequence for Spirochaetes bacterium glucosidase, Uniparc reference UPI0009CA1D5C, Uniprot reference A0A1V5WHQ7.
Seq ID No. 84: Amino acid sequence for Tenericutes bacterium glucosidase, Uniparc reference UPI0009C7041F, Uniprot reference A0A1V6IJ57.
Seq ID No. 85: Amino acid sequence for Flavobacterium gilvum glucosidase, Uniparc reference UPI0004E2A41F, Uniprot reference A0A085EG29.
Seq ID No. 86: Amino acid sequence for Algibacter lectus glucosidase, Uniparc reference UPI0005102470, Uniprot reference A0A090WWZ1.
Seq ID No. 87: Amino acid sequence for Hydnomerulius pinastri glucosidase, Uniparc reference UPI0005B0F0AE, Uniprot reference A0A0C9WDY0.
Seq ID No. 88: Amino acid sequence for Nocardia farcinica glucosidase, Uniparc reference UPI00065C2666, Uniprot reference A0A0H5NWN2.
Seq ID No. 89: Amino acid sequence for Bacteroides cellulosilyticus glucosidase, Uniparc reference UPI000760375B, Uniprot reference A0A125MG18.
Seq ID No. 90: Amino acid sequence for Fonsecaea erecta glucosidase, Uniparc reference UPI0007DF4250, Uniprot reference A0A178ZBN2.
Seq ID No. 91: Amino acid sequence for Bacteroides sp. glucosidase, Uniparc reference UPI0008209852, Uniprot reference A0A1C5W9N9.
Seq ID No. 92: Amino acid sequence for Tannerella forsythia glucosidase, Uniparc reference UPI00086C34C1, Uniprot reference A0A1D3UGH8.
Seq ID No. 93: Amino acid sequence for Microbacterium esteraromaticum glucosidase, Uniparc reference UPI00097E83BB, Uniprot reference A0A1R4KI92.
Seq ID No. 94: Amino acid sequence for bacterium glucosidase, Uniparc reference UPI0009D255E5, Uniprot reference A0A1V5LJK9.
Seq ID No. 95: Amino acid sequence for Candidatus hydrogenedentes glucosidase, Uniparc reference UPI0009C5A3CF, Uniprot reference A0A1V5Z2L2.
Seq ID No. 96: Amino acid sequence for Bacteroidetes bacterium glucosidase, Uniparc reference UPI0009C55799, Uniprot reference A0A1V6J4J8.
Seq ID No. 97: Amino acid sequence for Penicillium solitum glucosidase, Uniparc reference UPI0009D4067F, Uniprot reference A0A1V6RQ41.
Seq ID No. 98: Amino acid sequence for Weissella soli glucosidase, Uniparc reference UPI0008737AA2, Uniprot reference A0A288Q8I2.
Seq ID No. 99: Amino acid sequence for Acetatifactor muris glucosidase, Uniparc reference UPI000CAC57D4, Uniprot reference A0A2K4ZN91.
Seq ID No. 100: Amino acid sequence for Corynespora cassiicola glucosidase, Uniparc reference UPI000D237A4A, Uniprot reference A0A2T2NYD4.
Seq ID No. 101: Amino acid sequence for Meira miltonrushii glucosidase, Uniparc reference UPI000D77C91D, Uniprot reference A0A316V6M3.
Seq ID No. 102: Amino acid sequence for Bacteroides fragilis glucosidase, Uniparc reference UPI00004E1F76, Uniprot reference A0A380YVC7.
Seq ID No. 103: Amino acid sequence for Malassezia restricta glucosidase, Uniparc reference UPI000F0C30E8, Uniprot reference A0A3G2SB79.
Seq ID No. 104: Amino acid sequence for Fusarium euwallaceae glucosidase, Uniparc reference UPI000FFFEFB2, Uniprot reference A0A430LYA2.
Seq ID No. 105: Amino acid sequence for Psathyrella aberdarensis glucosidase, Uniparc reference UPI0010251887, Uniprot reference A0A4Q2E070.
Seq ID No. 106: Amino acid sequence for Aeromonas hydrophila glucosidase, Uniparc reference UPI0000E69509, Uniprot reference A0KLP6.
Seq ID No. 107: Amino acid sequence for Saccharopolyspora erythraea glucosidase, Uniparc reference UPI00000B86CB, Uniprot reference A4F7P9.
Seq ID No. 108: Amino acid sequence for Streptomyces sviceus glucosidase, Uniparc reference UPI000180240E, Uniprot reference B5I181.
Seq ID No. 109: Amino acid sequence for Naematelia encephala glucosidase, Uniparc reference UPI000A250F78, Uniprot reference A0A1Y2AWB7.
Seq ID No. 110: Amino acid sequence for Hartmannibacter diazotrophicus glucosidase, Uniparc reference UPI000C0221F1, Uniprot reference A0A2C9D6I2.
Seq ID No. 111: Amino acid sequence for Pontimonas salivibrio glucosidase, Uniparc reference UPI000CEB5AB1, Uniprot reference A0A2L2BPE2.
Seq ID No. 112: Amino acid sequence for Cadophora sp. glucosidase, Uniparc reference UPI000D5B9C38, Uniprot reference A0A2V1CH24.
Seq ID No. 113: Amino acid sequence for Meira miltonrushii glucosidase, Uniparc reference UPI000D779558, Uniprot reference A0A316V8S0.
Seq ID No. 114: Amino acid sequence for Monilinia fructigena glucosidase, Uniparc reference UPI000DC42E3D, Uniprot reference A0A395IJW4.
Seq ID No. 115: Amino acid sequence for Hortaea werneckii glucosidase, Uniparc reference UPI000F3E476C, Uniprot reference A0A3M6XGS0.
Seq ID No. 116: Amino acid sequence for Streptomyces netropsis glucosidase, Uniparc reference UPI00101460D7, Uniprot reference A0A445N7U0.
Seq ID No. 117: Amino acid sequence for Aureobasidium pullulans glucosidase, Uniparc reference UPI001139C6A8, Uniprot reference A0A4S9IFI0.
Seq ID No. 118: Amino acid sequence for Aspergillus clavatus glucosidase, Uniparc reference UPI0000EA5CFF, Uniprot reference A1CTN9.
Seq ID No. 119: Amino acid sequence for Clavibacter michiganensis glucosidase, Uniparc reference UPI0001523037, Uniprot reference A5CT94.
Seq ID No. 120: Amino acid sequence for Penicillium rubens glucosidase, Uniparc reference UPI0001831CF5, Uniprot reference B6H7R5.
Seq ID No. 121: Amino acid sequence for Lachnoclostridium sp. glucosidase, Uniparc reference UPI000B365547, Uniprot reference A0A1Y4NTL9.
Seq ID No. 122: Amino acid sequence for Rhodobacteraceae bacterium glucosidase, Uniparc reference UPI000C09BF88, Uniprot reference A0A2D5IXB9.
Seq ID No. 123: Amino acid sequence for Bacteroides fragilis glucosidase, Uniparc reference UPI0004B5EEF2, Uniprot reference A0A2M9UUC4.
Seq ID No. 124: Amino acid sequence for Aspergillus indologenus glucosidase, Uniparc reference UPI000D7FE1ED, Uniprot reference A0A2V5IY78.
Seq ID No. 125: Amino acid sequence for Acaromyces ingoldii glucosidase, Uniparc reference UPI000D802B25, Uniprot reference A0A316YR39.
Seq ID No. 126: Amino acid sequence for Monilinia fructigena glucosidase, Uniparc reference UPI000DC60823, Uniprot reference A0A395J1U5.
Seq ID No. 127: Amino acid sequence for Paenibacillus xylanexedens glucosidase, Uniparc reference UPI000F52D773, Uniprot reference A0A3N6CA02.
Seq ID No. 128: Amino acid sequence for Actinomyces howellii glucosidase, Uniparc reference UPI000F6DAAAE, Uniprot reference A0A448HIG0.
Seq ID No. 129: Amino acid sequence for Friedmanniomyces endolithicus glucosidase, Uniparc reference UPI00113D19DF, Uniprot reference A0A4V5N9I4.
Seq ID No. 130: Amino acid sequence for Neosartorya fischeri glucosidase, Uniparc reference UPI0000EA8672, Uniprot reference A1DNS0.
Seq ID No. 131: Amino acid sequence for Pseudomonas aeruginosa glucosidase, Uniparc reference UPI0000D7314B, Uniprot reference A6V4K6.
Seq ID No. 132: Amino acid sequence for Talaromyces stipitatus glucosidase, Uniparc reference UPI00018E7266, Uniprot reference B8MF24.
Seq ID No. 133: Amino acid sequence for Aquimixticola soesokkakensis glucosidase, Uniparc reference UPI000A1A5FD7, Uniprot reference A0A1Y5RVF8.
Seq ID No. 134: Amino acid sequence for Rhodobacterales bacterium glucosidase, Uniparc reference UPI000C98D37C, Uniprot reference A0A2D9YGV1.
Seq ID No. 135: Amino acid sequence for Methylorubrum extorquens glucosidase, Uniparc reference UPI0006F9793E, Uniprot reference A0A2N9AS40.
Seq ID No. 136: Amino acid sequence for Clostridium perfringens glucosidase, Uniparc reference UPI00000CF7C8, Uniprot reference A0A2X2YBP0.
Seq ID No. 137: Amino acid sequence for Acholeplasmatales bacterium glucosidase, Uniparc reference UPI0008AEBEA3, Uniprot reference A0A348NID6.
Seq ID No. 138: Amino acid sequence for Bacteroidetes bacterium glucosidase, Uniparc reference UPI000EC3C979, Uniprot reference A0A3B8VHE8.
Seq ID No. 139: Amino acid sequence for Clostridium carnis glucosidase, Uniparc reference UPI000F637E38, Uniprot reference A0A3P6K8E8.
Seq ID No. 140: Amino acid sequence for Mycolicibacterium flavescens glucosidase, Uniparc reference UPI000B93B5C9, Uniprot reference A0A448HNB5.
Seq ID No. 141: Amino acid sequence for Streptococcus gallolyticus glucosidase, Uniparc reference UPI000F6EFA71, Uniprot reference A0A4V6LJ94.
Seq ID No. 142: Amino acid sequence for Yersinia enterocolitica glucosidase, Uniparc reference UPI0000EB54CC, Uniprot reference A1JNB7.
Seq ID No. 143: Amino acid sequence for Anaeromyxobacter sp. glucosidase, Uniparc reference UPI0000ED8A80, Uniprot reference A7HFG4.
Seq ID No. 144: Amino acid sequence for Talaromyces stipitatus glucosidase, Uniparc reference UPI00018E7D70, Uniprot reference B8MK55.
Seq ID No. 145: Amino acid sequence for Hortaea werneckii glucosidase, Uniparc reference UPI000A2E3FAA, Uniprot reference A0A1Z5SL14.
Seq ID No. 146: Amino acid sequence for Micavibrio sp. glucosidase, Uniparc reference UPI000C529025, Uniprot reference A0A2E2Q8X2.
Seq ID No. 147: Amino acid sequence for Acidobacteriia bacterium glucosidase, Uniparc reference UPI000CE6B996, Uniprot reference A0A2N9MBS0.
Seq ID No. 148: Amino acid sequence for Corynebacterium jeikeium glucosidase, Uniparc reference UPI000DA3A972, Uniprot reference A0A2X4T570.
Seq ID No. 149: Amino acid sequence for Clostridiaceae bacterium glucosidase, Uniparc reference UPI000E8D37A1, Uniprot reference A0A353PZH8.
Seq ID No. 150: Amino acid sequence for Anaerolineaceae bacterium glucosidase, Uniparc reference UPI000748C096, Uniprot reference A0A3B9PA35.
Seq ID No. 151: Amino acid sequence for Gymnopilus dilepis glucosidase, Uniparc reference UPI000FF41956, Uniprot reference A0A409WSY0.
Seq ID No. 152: Amino acid sequence for Kocuria rosea glucosidase, Uniparc reference UPI000F7105D4, Uniprot reference A0A448R8N0.
Seq ID No. 153: Amino acid sequence for Teredinibacter sp. glucosidase, Uniparc reference UPI0011696FAB, Uniprot reference A0A509DWZ3.
Seq ID No. 154: Amino acid sequence for Aspergillus niger glucosidase, Uniparc reference UPI0000EFB564, Uniprot reference A2QS42.
Seq ID No. 155: Amino acid sequence for Laccaria bicolor glucosidase, Uniparc reference UPI000164423D, Uniprot reference B0D734.
Seq ID No. 156: Amino acid sequence for Podosphaera parvula glucosidase, Uniparc reference UPI00017357F6, Uniprot reference B9XH33.
Seq ID No. 157: Amino acid sequence for Megamonas hypermegale glucosidase, Uniparc reference UPI00042469F6, Uniprot reference A0A239TGH2.
Seq ID No. 158: Amino acid sequence for Armillaria gallica glucosidase, Uniparc reference UPI000BC209C3, Uniprot reference A0A2H3E300.
Seq ID No. 159: Amino acid sequence for Micromonospora sp. glucosidase, Uniparc reference UPI000D2EAE87, Uniprot reference A0A2P8AV03.
Seq ID No. 160: Amino acid sequence for Klebsiella oxytoca glucosidase, Uniparc reference UPI000DA286EE, Uniprot reference A0A2X5CJC5.
Seq ID No. 161: Amino acid sequence for Candidatus ozemobacter glucosidase, Uniparc reference UPI000DFAEF6C, Uniprot reference A0A367ZIH8.
Seq ID No. 162: Amino acid sequence for Coleophoma crateriformis glucosidase, Uniparc reference UPI000E399EE8, Uniprot reference A0A3D8R2C2.
Seq ID No. 163: Amino acid sequence for Apiotrichum porosum glucosidase, Uniparc reference UPI000FBC01E3, Uniprot reference A0A427XHS2.
Seq ID No. 164: Amino acid sequence for Acholeplasma hippikon glucosidase, Uniparc reference UPI00068E4E50, Uniprot reference A0A449BJ27.
Seq ID No. 165: Amino acid sequence for Streptomyces spectabilis glucosidase, Uniparc reference UPI001185F074, Uniprot reference A0A516RGT1.
Seq ID No. 166: Amino acid sequence for Aspergillus niger glucosidase, Uniparc reference UPI0000EFCED2, Uniprot reference A2R8G2.
Seq ID No. 167: Amino acid sequence for Xanthomonas campestris glucosidase, Uniparc reference UPI00000D8BFA, Uniprot reference B0RYA0.
Seq ID No. 168: Amino acid sequence for Lactobacillus paracasei glucosidase, Uniparc reference UPI00019C9CD7, Uniprot reference C2FDL2.
Seq ID No. 169: Amino acid sequence for Bifiguratus adelaidae glucosidase, Uniparc reference UPI000BC490A3, Uniprot reference A0A261XUH4.
Seq ID No. 170: Amino acid sequence for bacterium glucosidase, Uniparc reference UPI000CC31AE2, Uniprot reference A0A2H5YYA1.
Seq ID No. 171: Amino acid sequence for Actinomadura parvosata glucosidase, Uniparc reference UPI000D26C9C6, Uniprot reference A0A2P9IY35.
Seq ID No. 172: Amino acid sequence for Melissococcus plutonius glucosidase, Uniparc reference UPI00024F22B8, Uniprot reference A0A2Z5Y4P3.
Seq ID No. 173: Amino acid sequence for Enterococcus durans glucosidase, Uniparc reference UPI000E020871, Uniprot reference A0A377KJS3.
Seq ID No. 174: Amino acid sequence for Malassezia restricta glucosidase, Uniparc reference UPI000DD17A7D, Uniprot reference A0A3G2S2J6.
Seq ID No. 175: Amino acid sequence for Apiotrichum porosum glucosidase, Uniparc reference UPI000FA2DE87, Uniprot reference A0A427XZQ0.
Seq ID No. 176: Amino acid sequence for Streptomonospora sp. glucosidase, Uniparc reference UPI0010355193, Uniprot reference A0A4P6PWP5.
Seq ID No. 177: Amino acid sequence for Lactobacillus gasseri glucosidase, Uniparc reference UPI00119640A1, Uniprot reference A0A558LH47.
Seq ID No. 178: Amino acid sequence for Aspergillus niger glucosidase, Uniparc reference UPI0000EFD0AA, Uniprot reference A2RAJ1.
Seq ID No. 179: Amino acid sequence for Neosartorya fumigata glucosidase, Uniparc reference UPI000170BF91, Uniprot reference B0XXG1.
Seq ID No. 180: Amino acid sequence for Eubacterium eligens glucosidase, Uniparc reference UPI0001A5B234, Uniprot reference C4Z6T5.
Seq ID No. 181: Amino acid sequence for Bifiguratus adelaidae glucosidase, Uniparc reference UPI000BC64A75, Uniprot reference A0A261XVM8.
Seq ID No. 182: Amino acid sequence for bacterium glucosidase, Uniparc reference UPI000CAA854E, Uniprot reference A0A2H6EX57.
Seq ID No. 183: Amino acid sequence for Corynespora cassiicola glucosidase, Uniparc reference UPI000D24EC2C, Uniprot reference A0A2T2N5H9.
Seq ID No. 184: Amino acid sequence for Pseudomicrostroma glucosiphilum glucosidase, Uniparc reference UPI000D77A0B7, Uniprot reference A0A316UK83.
Seq ID No. 185: Amino acid sequence for Staphylococcus saprophyticus glucosidase, Uniparc reference UPI0002DAAFCC, Uniprot reference A0A380HD31.
Seq ID No. 186: Amino acid sequence for Malassezia restricta glucosidase, Uniparc reference UPI000F0C6B8E, Uniprot reference A0A3G2S932.
Seq ID No. 187: Amino acid sequence for Saitozyma podzolica glucosidase, Uniparc reference UPI000FBA70E2, Uniprot reference A0A427YCL1.
Seq ID No. 188: Amino acid sequence for Tremella mesenterica glucosidase, Uniparc reference UPI00102822B4, Uniprot reference A0A4Q1BRC6.
Seq ID No. 189: Amino acid sequence for Arthrobacter sp. glucosidase, Uniparc reference UPI0000527506, Uniprot reference A0JZ86.
Seq ID No. 190: Amino acid sequence for Scheffersomyces stipitis glucosidase, Uniparc reference UPI000157388C, Uniprot reference A3LRB0.
Seq ID No. 191: Amino acid sequence for Leptothrix cholodnii glucosidase, Uniparc reference UPI0001712E02, Uniprot reference B1XZK8.
Seq ID No. 192: Amino acid sequence for Thauera sp. glucosidase, Uniparc reference UPI000166883C, Uniprot reference C4ZLL7.
Seq ID No. 193: Amino acid sequence for Kosmotoga olearia glucosidase, Uniparc reference UPI00018494AB, Uniprot reference C5CDW5.
Seq ID No. 194: Amino acid sequence for Roseburia intestinalis glucosidase, Uniparc reference UPI0001CD671F, Uniprot reference D4L3Y2.
Seq ID No. 195: Amino acid sequence for Streptococcus equinus glucosidase, Uniparc reference UPI0001E0DC00, Uniprot reference E0PDF8.
Seq ID No. 196: Amino acid sequence for Streptococcus cristatus glucosidase, Uniparc reference UPI0001F80C4B, Uniprot reference E8JUK5.
Seq ID No. 197: Amino acid sequence for Cellulosilyticum lentocellum glucosidase, Uniparc reference UPI0001D2DBBC, Uniprot reference F2JLH3.
Seq ID No. 198: Amino acid sequence for Streptococcus gallolyticus glucosidase, Uniparc reference UPI0001C48657, Uniprot reference F5WYI5.
Seq ID No. 199: Amino acid sequence for Ketogulonicigenium vulgare glucosidase, Uniparc reference UPI00021D443A, Uniprot reference F9Y8Z7.
Seq ID No. 200: Amino acid sequence for Spathaspora passalidarum glucosidase, Uniparc reference UPI000228255D, Uniprot reference G3AGX1.
Seq ID No. 201: Amino acid sequence for Niastella koreensis glucosidase, Uniparc reference UPI00023F6F5F, Uniprot reference G8T9J3.
Seq ID No. 202: Amino acid sequence for Cellvibrio sp. glucosidase, Uniparc reference UPI000260108C, Uniprot reference I3IDC0.
Seq ID No. 203: Amino acid sequence for Flavobacterium sp. glucosidase, Uniparc reference UPI000272D1E0, Uniprot reference J1ACA0.
Seq ID No. 204: Amino acid sequence for Macrophomina phaseolina glucosidase, Uniparc reference UPI00028E7FE1, Uniprot reference K2S5D3.
Seq ID No. 205: Amino acid sequence for Kosmotoga olearia glucosidase, Uniparc reference UPI00018483A2, Uniprot reference C5CHI5.
Seq ID No. 206: Amino acid sequence for Blautia obeum glucosidase, Uniparc reference UPI0001CD5918, Uniprot reference D4LRF6.
Seq ID No. 207: Amino acid sequence for Bifidobacterium dentium glucosidase, Uniparc reference UPI0001E18CDA, Uniprot reference E0Q541.
Seq ID No. 208: Amino acid sequence for Anaerolinea thermophila glucosidase, Uniparc reference UPI0001F55F8F, Uniprot reference E8N5R8.
Seq ID No. 209: Amino acid sequence for Coriobacterium glomerans glucosidase, Uniparc reference UPI0002050DA2, Uniprot reference F2N7E4.
Seq ID No. 210: Amino acid sequence for Microlunatus phosphovorus glucosidase, Uniparc reference UPI000210C886, Uniprot reference F5XJQ3.
Seq ID No. 211: Amino acid sequence for Streptomyces sp. glucosidase, Uniparc reference UPI0001C18877, Uniprot reference G0Q1U8.
Seq ID No. 212: Amino acid sequence for Spathaspora passalidarum glucosidase, Uniparc reference UPI0002282B61, Uniprot reference G3AIV6.
Seq ID No. 213: Amino acid sequence for Glarea lozoyensis glucosidase, Uniparc reference UPI0002402F4A, Uniprot reference H0ET34.
Seq ID No. 214: Amino acid sequence for Glaciozyma antarctica glucosidase, Uniparc reference UPI0002633B36, Uniprot reference I3UJK0.
Seq ID No. 215: Amino acid sequence for Arthrobacter sp. glucosidase, Uniparc reference UPI00027DFD35, Uniprot reference J7LN00.
Seq ID No. 216: Amino acid sequence for Fusarium pseudograminearum glucosidase, Uniparc reference UPI00028D698C, Uniprot reference K3VMA9.
Seq ID No. 217: Amino acid sequence for Hypocrea rufa glucosidase, Uniparc reference UPI000006AA61, Uniprot reference C6GGC9.
Seq ID No. 218: Amino acid sequence for Ruminococcus torques glucosidase, Uniparc reference UPI0001CDA964, Uniprot reference D4M6W6.
Seq ID No. 219: Amino acid sequence for Bifidobacterium dentium glucosidase, Uniparc reference UPI0001E17370, Uniprot reference E0Q9Z7.
Seq ID No. 220: Amino acid sequence for Bacteroides salanitronis glucosidase, Uniparc reference UPI0001FC71F6, Uniprot reference F0R2D7.
Seq ID No. 221: Amino acid sequence for Bacteroides coprosuis glucosidase, Uniparc reference UPI00020E6D19, Uniprot reference F3ZQ40.
Seq ID No. 222: Amino acid sequence for Marinomonas posidonica glucosidase, Uniparc reference UPI00020D4CC6, Uniprot reference F6CWF6.
Seq ID No. 223: Amino acid sequence for Chaetomium thermophilum glucosidase, Uniparc reference UPI000227E8ED, Uniprot reference G0SE64.
Seq ID No. 224: Amino acid sequence for Tetragenococcus halophilus glucosidase, Uniparc reference UPI00022B9A15, Uniprot reference G4L5K1.
Seq ID No. 225: Amino acid sequence for Paenibacillus sp. glucosidase, Uniparc reference UPI00024F0867, Uniprot reference H6CCZ8.
Seq ID No. 226: Amino acid sequence for Turneriella parva glucosidase, Uniparc reference UPI000265AA4B, Uniprot reference I4B8U7.
Seq ID No. 227: Amino acid sequence for Arthrobacter sp. glucosidase, Uniparc reference UPI00027DFDB2, Uniprot reference J7LQK9.
Seq ID No. 228: Amino acid sequence for Agaricus bisporus glucosidase, Uniparc reference UPI00029074B1, Uniprot reference K5W7V1.
Seq ID No. 229: Amino acid sequence for Nectria haematococca glucosidase, Uniparc reference UPI0001B67634, Uniprot reference C7YIP3.
Seq ID No. 230: Amino acid sequence for Bacteroides xylanisolvens glucosidase, Uniparc reference UPI0001A25287, Uniprot reference D4VSZ0.
Seq ID No. 231: Amino acid sequence for Sediminispirochaeta smaragdinae glucosidase, Uniparc reference UPI0001DD9790, Uniprot reference E1R331.
Seq ID No. 232: Amino acid sequence for Deinococcus proteolyticus glucosidase, Uniparc reference UPI0001FC42E9, Uniprot reference F0RPV2.
Seq ID No. 233: Amino acid sequence for Sphingobacterium sp. glucosidase, Uniparc reference UPI0002033A0A, Uniprot reference F4C226.
Seq ID No. 234: Amino acid sequence for Sphingobium chlorophenolicum glucosidase, Uniparc reference UPI0001E54133, Uniprot reference F6ET40.
Seq ID No. 235: Amino acid sequence for Caloramator australicus glucosidase, Uniparc reference UPI00021CACC4, Uniprot reference G0V3V5.
Seq ID No. 236: Amino acid sequence for Commensalibacter intestini glucosidase, Uniparc reference UPI000230E3BF, Uniprot reference G6F370.
Seq ID No. 237: Amino acid sequence for Paenibacillus sp. glucosidase, Uniparc reference UPI00024F07AC, Uniprot reference H6CIT2.
Seq ID No. 238: Amino acid sequence for Nitrolancea hollandica glucosidase, Uniparc reference UPI0002638AF3, Uniprot reference I4EIA9.
Seq ID No. 239: Amino acid sequence for Cryptococcus neoformans glucosidase, Uniparc reference UPI000392C3ED, Uniprot reference J9VVK7.
Seq ID No. 240: Amino acid sequence for Acidipropionibacterium acidipropionici glucosidase, Uniparc reference UPI0002988588, Uniprot reference K75596.
Seq ID No. 241: Amino acid sequence for Prevotella sp. glucosidase, Uniparc reference UPI0001B93465, Uniprot reference C9PT75.
Seq ID No. 242: Amino acid sequence for Rhodobacter capsulatus glucosidase, Uniparc reference UPI0001D08095, Uniprot reference D5ALU0.
Seq ID No. 243: Amino acid sequence for Stigmatella aurantiaca glucosidase, Uniparc reference UPI0001E74370, Uniprot reference E3FJ05.
Seq ID No. 244: Amino acid sequence for Sphaerochaeta globosa glucosidase, Uniparc reference UPI0002010060, Uniprot reference F0RVK3.
Seq ID No. 245: Amino acid sequence for Sphaerochaeta coccoides glucosidase, Uniparc reference UPI000207D78C, Uniprot reference F4GH96.
Seq ID No. 246: Amino acid sequence for Novosphingobium sp. glucosidase, Uniparc reference UPI00020EFBCB, Uniprot reference F6ICQ5.
Seq ID No. 247: Amino acid sequence for Arthrobotrys oligospora glucosidase, Uniparc reference UPI000225331F, Uniprot reference G1XH86.
Seq ID No. 248: Amino acid sequence for Lactococcus lactis glucosidase, Uniparc reference UPI00000C6907, Uniprot reference G6FFS4.
Seq ID No. 249: Amino acid sequence for Phaeospirillum molischianum glucosidase, Uniparc reference UPI000255313A, Uniprot reference H8FXH7.
Seq ID No. 250: Amino acid sequence for Modestobacter marinus glucosidase, Uniparc reference UPI0002609C68, Uniprot reference I4EW72.
Seq ID No. 251: Amino acid sequence for Saccharothrix espanaensis glucosidase, Uniparc reference UPI00028AC337, Uniprot reference K0K125.
Seq ID No. 252: Amino acid sequence for Cronobacter sakazakii glucosidase, Uniparc reference UPI00029BA293, Uniprot reference K8DAK0.
Seq ID No. 253: Amino acid sequence for Verticillium alfalfae glucosidase, Uniparc reference UPI0001BBDF1E, Uniprot reference C9SVX1.
Seq ID No. 254: Amino acid sequence for Bacteroides xylanisolvens glucosidase, Uniparc reference UPI0001CCFD36, Uniprot reference D6CY10.
Seq ID No. 255: Amino acid sequence for Leadbetterella byssophila glucosidase, Uniparc reference UPI0001EBD98A, Uniprot reference E4RUR0.
Seq ID No. 256: Amino acid sequence for Sphaerochaeta globosa glucosidase, Uniparc reference UPI00020102A5, Uniprot reference F0RYB6.
Seq ID No. 257: Amino acid sequence for Sphaerochaeta coccoides glucosidase, Uniparc reference UPI000207D6A0, Uniprot reference F4GLH6.
Seq ID No. 258: Amino acid sequence for Haloplasma contractile glucosidase, Uniparc reference UPI000212252C, Uniprot reference F7Q0Y2.
Seq ID No. 259: Amino acid sequence for Nitrospirillum amazonense glucosidase, Uniparc reference UPI0002265447, Uniprot reference G1Y4C7.
Seq ID No. 260: Amino acid sequence for Azospirillum brasilense glucosidase, Uniparc reference UPI00023420B8, Uniprot reference G8AWD9.
Seq ID No. 261: Amino acid sequence for Phaeospirillum molischianum glucosidase, Uniparc reference UPI000255314F, Uniprot reference H8FXJ8.
Seq ID No. 262: Amino acid sequence for Modestobacter marinus glucosidase, Uniparc reference UPI000260A2FA, Uniprot reference I4EYD5.
Seq ID No. 263: Amino acid sequence for Wickerhamomyces ciferrii glucosidase, Uniparc reference UPI000283EB8D, Uniprot reference K0KVJ2.
Seq ID No. 264: Amino acid sequence for Gloeocapsa sp. glucosidase, Uniparc reference UPI0002A5D085, Uniprot reference K9XKL8.
Seq ID No. 265: Amino acid sequence for Sphaerobacter thermophilus glucosidase, Uniparc reference UPI0001A3BCB6, Uniprot reference D1C7U8.
Seq ID No. 266: Amino acid sequence for Bacteroides xylanisolvens glucosidase, Uniparc reference UPI0001CCEF1F, Uniprot reference D6D4V2.
Seq ID No. 267: Amino acid sequence for Prevotella buccae glucosidase, Uniparc reference UPI0001F149E8, Uniprot reference E6K4W5.
Seq ID No. 268: Amino acid sequence for Grosmannia clavigera glucosidase, Uniparc reference UPI0001FF1101, Uniprot reference F0XBR0.
Seq ID No. 269: Amino acid sequence for Melampsora larici-populina glucosidase, Uniparc reference UPI00020F9774, Uniprot reference F4R4W2.
Seq ID No. 270: Amino acid sequence for Prevotella multisaccharivorax glucosidase, Uniparc reference UPI0002138E9C, Uniprot reference F8N7G1.
Seq ID No. 271: Amino acid sequence for Streptomyces zinciresistens glucosidase, Uniparc reference UPI0002255A63, Uniprot reference G2G8K4.
Seq ID No. 272: Amino acid sequence for Granulicella mallensis glucosidase, Uniparc reference UPI0001D9FC40, Uniprot reference G8NY42.
Seq ID No. 273: Amino acid sequence for Gibberella zeae glucosidase, Uniparc reference UPI00021F1FEE, Uniprot reference I1RH94.
Seq ID No. 274: Amino acid sequence for Modestobacter marinus glucosidase, Uniparc reference UPI00026090A2, Uniprot reference I4EYK6.
Seq ID No. 275: Amino acid sequence for Lactobacillus equicursoris glucosidase, Uniparc reference UPI0002869F55, Uniprot reference K0NRS8.
Seq ID No. 276: Amino acid sequence for Colletotrichum fructicola glucosidase, Uniparc reference UPI0002A93280, Uniprot reference L2F9W0.
Seq ID No. 277: Amino acid sequence for Streptosporangium roseum glucosidase, Uniparc reference UPI0001BF8AF6, Uniprot reference D2B261.
Seq ID No. 278: Amino acid sequence for Listeria grayi glucosidase, Uniparc reference UPI00019F252A, Uniprot reference D7UX19.
Seq ID No. 279: Amino acid sequence for Enterococcus italicus glucosidase, Uniparc reference UPI0001F11EFF, Uniprot reference E6LF07.
Seq ID No. 280: Amino acid sequence for Fluviicola taffensis glucosidase, Uniparc reference UPI000203D9EA, Uniprot reference F2IIT6.
Seq ID No. 281: Amino acid sequence for Shigella flexneri glucosidase, Uniparc reference UPI00020CA641, Uniprot reference F5N4W9.
Seq ID No. 282: Amino acid sequence for Actinomyces sp. glucosidase, Uniparc reference UPI0002189080, Uniprot reference F9EFW5.
Seq ID No. 283: Amino acid sequence for Verticillium dahliae glucosidase, Uniparc reference UPI00022EBE5A, Uniprot reference G2X5V6.
Seq ID No. 284: Amino acid sequence for Actinoplanes sp. glucosidase, Uniparc reference UPI00023ED5A8, Uniprot reference G8S0M0.
Seq ID No. 285: Amino acid sequence for Gibberella zeae glucosidase, Uniparc reference UPI000023EA29, Uniprot reference I1S320.
Seq ID No. 286: Amino acid sequence for Auricularia subglabra glucosidase, Uniparc reference UPI00027CE685, Uniprot reference J0WV06.
Seq ID No. 287: Amino acid sequence for Nitratireductor indicus glucosidase, Uniparc reference UPI00028E86D6, Uniprot reference K2N268.
Seq ID No. 288: Amino acid sequence for Thermoclostridium stercorarium glucosidase, Uniparc reference UPI0002AD999E, Uniprot reference L7VH66.
Seq ID No. 289: Amino acid sequence for Geobacillus sp. glucosidase, Uniparc reference UPI0002AF2DE3, Uniprot reference L8A1S2.
Seq ID No. 290: Amino acid sequence for uncultured bacterium glucosidase, Uniparc reference UPI000327C3CC, Uniprot reference M9ZC55.
Seq ID No. 291: Amino acid sequence for Burkholderia ambifaria glucosidase, Uniparc reference UPI000059ACE1, Uniprot reference Q0BAK2.
Seq ID No. 292: Amino acid sequence for Aspergillus oryzae glucosidase, Uniparc reference UPI0000676B8D, Uniprot reference Q2UIR4.
Seq ID No. 293: Amino acid sequence for Yersinia pseudotuberculosis glucosidase, Uniparc reference UPI00004269B5, Uniprot reference Q66DJ0.
Seq ID No. 294: Amino acid sequence for Wallemia ichthyophaga glucosidase, Uniparc reference UPI000331CC98, Uniprot reference R9AF64.
Seq ID No. 295: Amino acid sequence for Glarea lozoyensis glucosidase, Uniparc reference UPI0003522404, Uniprot reference S3DIM6.
Seq ID No. 296: Amino acid sequence for Moniliophthora roreri glucosidase, Uniparc reference UPI0003BF753A, Uniprot reference V2YU31.
Seq ID No. 297: Amino acid sequence for Zhouia amylolytica glucosidase, Uniparc reference UPI0003DBCB4F, Uniprot reference W2ULL3.
Seq ID No. 298: Amino acid sequence for Fusarium oxysporum glucosidase, Uniparc reference UPI0003F362FF, Uniprot reference W9NQQ3.
Seq ID No. 299: Amino acid sequence for Flavobacterium johnsoniae glucosidase, Uniparc reference UPI00006EA168, Uniprot reference A5FAA5.
Seq ID No. 300: Amino acid sequence for Oryza sativa glucosidase, Uniparc reference UPI00002394F5, Uniprot reference Q75I93.
Seq ID No. 301: Amino acid sequence for Clostridium saccharoperbutylacetonicum glucosidase, Uniparc reference UPI0002B65681, Uniprot reference M1MCD1.
Seq ID No. 302: Amino acid sequence for Rhodococcus sp. glucosidase, Uniparc reference UPI0002D21DB1, Uniprot reference N1MBN6.
Seq ID No. 303: Amino acid sequence for Burkholderia ambifaria glucosidase, Uniparc reference UPI000059C0CD, Uniprot reference Q0BCV8.
Seq ID No. 304: Amino acid sequence for Xanthomonas campestris glucosidase, Uniparc reference UPI00005CE9E7, Uniprot reference Q3BVH7.
Seq ID No. 305: Amino acid sequence for Caulobacter vibrioides glucosidase, Uniparc reference UPI00000C7604, Uniprot reference Q9A6F8.
Seq ID No. 306: Amino acid sequence for Arcticibacter svalbardensis glucosidase, Uniparc reference UPI000338772A, Uniprot reference R9GRA7.
Seq ID No. 307: Amino acid sequence for Winogradskyella psychrotolerans glucosidase, Uniparc reference UPI00035A4604, Uniprot reference S7VQ28.
Seq ID No. 308: Amino acid sequence for Methyloglobulus morosus glucosidase, Uniparc reference UPI0003C4ED6A, Uniprot reference V5DXT8.
Seq ID No. 309: Amino acid sequence for Pestalotiopsis fici glucosidase, Uniparc reference UPI0003E05C04, Uniprot reference W3WV37.
Seq ID No. 310: Amino acid sequence for Capronia coronata glucosidase, Uniparc reference UPI000434E698, Uniprot reference W9YNR7.
Seq ID No. 311: Amino acid sequence for Aspergillus aculeatus glucosidase, Uniparc reference UPI00001268FD, Uniprot reference P48825.
Seq ID No. 312: Amino acid sequence for Thermotoga neapolitana glucosidase, Uniparc reference UPI0000DD5996, Uniprot reference Q0GC07.
Seq ID No. 313: Amino acid sequence for Clostridium saccharoperbutylacetonicum glucosidase, Uniparc reference UPI0002B666E8, Uniprot reference M1MJF4.
Seq ID No. 314: Amino acid sequence for Schizosaccharomyces pombe glucosidase, Uniparc reference UPI000006B001, Uniprot reference O74799.
Seq ID No. 315: Amino acid sequence for Phaeosphaeria nodorum glucosidase, Uniparc reference UPI000161BD2B, Uniprot reference Q0TXF6.
Seq ID No. 316: Amino acid sequence for Xylella fastidiosa Dixon glucosidase, Uniparc reference UPI00003806C8, Uniprot reference Q3RGJ3.
Seq ID No. 317: Amino acid sequence for Schizosaccharomyces pombe glucosidase, Uniparc reference UPI000006A330, Uniprot reference Q9P6J6.
Seq ID No. 318: Amino acid sequence for Arcticibacter svalbardensis glucosidase, Uniparc reference UPI000337E887, Uniprot reference R9GWD6.
Seq ID No. 319: Amino acid sequence for Colletotrichum gloeosporioides glucosidase, Uniparc reference UPI0003885717, Uniprot reference T0KJI7.
Seq ID No. 320: Amino acid sequence for uncultured bacterium glucosidase, Uniparc reference UPI0003C9E340, Uniprot reference V5R1E8.
Seq ID No. 321: Amino acid sequence for Xanthomonas arboricola glucosidase, Uniparc reference UPI0003E06A61, Uniprot reference W4S7I5.
Seq ID No. 322: Amino acid sequence for Fusarium oxysporum glucosidase, Uniparc reference UPI00021EC697, Uniprot reference X0A8X8.
Seq ID No. 323: Amino acid sequence for Paenibacillus polymyxa glucosidase, Uniparc reference UPI00001108D0, Uniprot reference P22073.
Seq ID No. 324: Amino acid sequence for Kluyveromyces marxianus glucosidase, Uniparc reference UPI0001BE5ADA, Uniprot reference D1GCC6.
Seq ID No. 325: Amino acid sequence for Ilumatobacter coccineus glucosidase, Uniparc reference UPI0002C04A25, Uniprot reference M5A594.
Seq ID No. 326: Amino acid sequence for Agrobacterium sp. glucosidase, Uniparc reference UPI0000126912, Uniprot reference P12614.
Seq ID No. 327: Amino acid sequence for Cytophaga hutchinsonii glucosidase, Uniparc reference UPI000038ECF7, Uniprot reference Q11P53.
Seq ID No. 328: Amino acid sequence for Thermobifida fusca glucosidase, Uniparc reference UPI00003C5CA4, Uniprot reference Q47PF5.
Seq ID No. 329: Amino acid sequence for Botryotinia fuckeliana glucosidase, Uniparc reference UPI0000069E5E, Uniprot reference Q9UVJ6.
Seq ID No. 330: Amino acid sequence for Agarivorans albus glucosidase, Uniparc reference UPI00033994D2, Uniprot reference R9PTK2.
Seq ID No. 331: Amino acid sequence for Enterococcus sp. glucosidase, Uniparc reference UPI00038B7639, Uniprot reference T0UDU2.
Seq ID No. 332: Amino acid sequence for Salinispira pacifica glucosidase, Uniparc reference UPI0003D8A7DC, Uniprot reference V5WKT4.
Seq ID No. 333: Amino acid sequence for Bacteroides xylanisolvens glucosidase, Uniparc reference UPI0003ECF15E, Uniprot reference W6P696.
Seq ID No. 334: Amino acid sequence for Fusarium oxysporum glucosidase, Uniparc reference UPI000430029A, Uniprot reference X0AB46.
Seq ID No. 335: Amino acid sequence for Thermotoga maritima glucosidase, Uniparc reference UPI0000126906, Uniprot reference Q08638B.
Seq ID No. 336: Amino acid sequence for Neotermes koshunensis glucosidase, Uniparc reference UPI0000083EA8, Uniprot reference Q8T0W7.
Seq ID No. 337: Amino acid sequence for Thanatephorus cucumeris glucosidase, Uniparc reference UPI0002BF34B3, Uniprot reference M5CHG9.
Seq ID No. 338: Amino acid sequence for Hungateiclostridium thermocellum glucosidase, Uniparc reference UPI000053581D, Uniprot reference P14002.
Seq ID No. 339: Amino acid sequence for Koribacter versatilis glucosidase, Uniparc reference UPI0000D76A70, Uniprot reference Q1IJ89.
Seq ID No. 340: Amino acid sequence for Neosartorya fumigata glucosidase, Uniparc reference UPI00005203D8, Uniprot reference Q4WLX5.
Seq ID No. 341: Amino acid sequence for Amycolatopsis vancoresmycina glucosidase, Uniparc reference UPI00032DB8C1, Uniprot reference R1FKF0.
Seq ID No. 342: Amino acid sequence for Gibberella fujikuroi glucosidase, Uniparc reference UPI0003519C76, Uniprot reference S0EKU1.
Seq ID No. 343: Amino acid sequence for Bifidobacterium longum glucosidase, Uniparc reference UPI000390F839, Uniprot reference T2I2H5.
Seq ID No. 344: Amino acid sequence for uncultured bacterium glucosidase, Uniparc reference UPI0003DFF0DA, Uniprot reference W0FLD1.
Seq ID No. 345: Amino acid sequence for Bacteroides xylanisolvens glucosidase, Uniparc reference UPI0002D3D994, Uniprot reference W6P9J9.
Seq ID No. 346: Amino acid sequence for Fusarium oxysporum glucosidase, Uniparc reference UPI00042F8016, Uniprot reference X0HA35.
Seq ID No. 347: Amino acid sequence for Sorghum bicolor glucosidase, Uniparc reference UPI00000A7F40, Uniprot reference Q41290.
Seq ID No. 348: Amino acid sequence for Clostridium cellulovorans glucosidase, Uniparc reference UPI000050B701, Uniprot reference Q53EH2.
Seq ID No. 349: Amino acid sequence for Dacryopinax primogenitus glucosidase, Uniparc reference UPI0002C29EAA, Uniprot reference M5G1U5.
Seq ID No. 350: Amino acid sequence for Rhizobium radiobacter glucosidase, Uniparc reference UPI0000126913, Uniprot reference P27034.
Seq ID No. 351: Amino acid sequence for Phanerochaete chrysosporium glucosidase, Uniparc reference UPI00006E0CC7, Uniprot reference Q25BW5A.
Seq ID No. 352: Amino acid sequence for Enterobacter agglomerans glucosidase, Uniparc reference UPI0000126905, Uniprot reference Q59437.
Seq ID No. 353: Amino acid sequence for Candidatus microthrix glucosidase, Uniparc reference UPI00033056C4, Uniprot reference R4YWU4.
Seq ID No. 354: Amino acid sequence for Chthonomonas calidirosea glucosidase, Uniparc reference UPI0003427F29, Uniprot reference S0ET37.
Seq ID No. 355: Amino acid sequence for Acholeplasma brassicae glucosidase, Uniparc reference UPI0003B04208, Uniprot reference U4KMR7.
Seq ID No. 356: Amino acid sequence for Chania multitudinisentens glucosidase, Uniparc reference UPI0003E13BD3, Uniprot reference W0LJ28.
Seq ID No. 357: Amino acid sequence for Gibberella moniliformis glucosidase, Uniparc reference UPI0003ECCF80, Uniprot reference W7MW98.
Seq ID No. 358: Amino acid sequence for Fusarium oxysporum glucosidase, Uniparc reference UPI0004305856, Uniprot reference X0I2U8.
Seq ID No. 359: Amino acid sequence for Phanerochaete chrysosporium glucosidase, Uniparc reference UPI00006E0CC7, Uniprot reference Q25BW5B.
Seq ID No. 360: Amino acid sequence for Secale cereale glucosidase, Uniparc reference UPI00000A7EFC, Uniprot reference Q9FYS3.
Seq ID No. 361: Amino acid sequence for Anoxybacillus gonensis glucosidase, Uniparc reference UPI0002C0273E, Uniprot reference M5QUM2.
Seq ID No. 362: Amino acid sequence for Thermotoga maritima glucosidase, Uniparc reference UPI0000126906, Uniprot reference Q08638A.
Seq ID No. 363: Amino acid sequence for Rhodospirillum rubrum glucosidase, Uniparc reference UPI00003C2ACC, Uniprot reference Q2RP51.
Seq ID No. 364: Amino acid sequence for Thermotoga neapolitana glucosidase, Uniparc reference UPI00000B9013, Uniprot reference Q60038.
Seq ID No. 365: Amino acid sequence for Candidatus microthrix glucosidase, Uniparc reference UPI00032F466E, Uniprot reference R4Z6M6.
Seq ID No. 366: Amino acid sequence for Ruminiclostridium cellobioparum glucosidase, Uniparc reference UPI00032874EC, Uniprot reference S0FPI8.
Seq ID No. 367: Amino acid sequence for Acholeplasma brassicae glucosidase, Uniparc reference UPI0003B0490C, Uniprot reference U4KMV4.
Seq ID No. 368: Amino acid sequence for Klebsiella pneumoniae glucosidase, Uniparc reference UPI0003DB6EFD, Uniprot reference W1BBP5.
Seq ID No. 369: Amino acid sequence for Enterobacter sp. glucosidase, Uniparc reference UPI0003ED0A8E, Uniprot reference W7P6Y1.
Seq ID No. 370: Amino acid sequence for Rhizoctonia solani glucosidase, Uniparc reference UPI00045BB507, Uniprot reference X8JI88.
Seq ID No. 371: Amino acid sequence for Homo sapiens glucosidase, Uniparc reference UPI0000072C73, Uniprot reference Q9H227.
Seq ID No. 372: Amino acid sequence for uncultured bacterium glucosidase, Uniparc reference UPI0000DD5304, Uniprot reference Q0GMU3.
Seq ID No. 373: Amino acid sequence for Thermoanaerobacter thermohydrosulfuricus glucosidase, Uniparc reference UPI0002CA9E4F, Uniprot reference M8CQD9.
Seq ID No. 374: Amino acid sequence for Stigmatella aurantiaca glucosidase, Uniparc reference UPI0000E297D8, Uniprot reference Q091M8.
Seq ID No. 375: Amino acid sequence for Burkholderia thailandensis glucosidase, Uniparc reference UPI00006676B1, Uniprot reference Q2T7C9.
Seq ID No. 376: Amino acid sequence for Yersinia pseudotuberculosis glucosidase, Uniparc reference UPI0000426E8F, Uniprot reference Q665S2.
Seq ID No. 377: Amino acid sequence for Togninia minima glucosidase, Uniparc reference UPI00032BF71F, Uniprot reference R8BQM8.
Seq ID No. 378: Amino acid sequence for Ophiostoma piceae glucosidase, Uniparc reference UPI00035213C2, Uniprot reference S3BVJ8.
Seq ID No. 379: Amino acid sequence for Pyronema omphalodes glucosidase, Uniparc reference UPI0003B117B7, Uniprot reference U4LIL3.
Seq ID No. 380: Amino acid sequence for Ogataea parapolymorpha glucosidase, Uniparc reference UPI0001F76D1F, Uniprot reference W1QIW0.
Seq ID No. 381: Amino acid sequence for Hymenobacter swuensis glucosidase, Uniparc reference UPI0003F2059F, Uniprot reference W8F6U0.
Seq ID No. 382: Amino acid sequence for Flavobacterium johnsoniae glucosidase, Uniparc reference UPI00006E8E1B, Uniprot reference A5FEF5.
Seq ID No. 383: Amino acid sequence for Paenibacillus polymyxa glucosidase, Uniparc reference UPI000012690B, Uniprot reference P22505.
Seq ID No. 384: Amino acid sequence for Oryza sativa glucosidase, Uniparc reference UPI000009D014, Uniprot reference Q8L7J2.
Seq ID No. 385: Amino acid sequence for Oryza sativa glucosidase, Uniparc reference UPI0000E580F2, Uniprot reference B8AVF0.
Seq ID No. 386: Amino acid sequence for Nannochloris glucosidase, Uniparc reference UPI000EA7F16F, Uniprot reference A0A452CSM4.
Seq ID No. 387: Amino acid sequence for Halothermothrix orenii glucosidase, Uniparc reference UPI00006AE508, Uniprot reference B8CYA8.
Seq ID No. 388: Amino acid sequence for Neurospora crassa glucosidase, Uniparc reference UPI000018B2B4, Uniprot reference Q7RWP2.
Seq ID No. 389: Amino acid sequence for Micrococcus antarcticus glucosidase, Uniparc reference UPI000192BB5F, Uniprot reference B9V8P5.
Seq ID No. 390: Amino acid sequence for Exiguobacterium antarcticum glucosidase, Uniparc reference UPI000285E79E, Uniprot reference K0A8J9.
Seq ID No. 391: Amino acid sequence for Thermus thermophilus glucosidase, Uniparc reference UPI00000BEB61, Uniprot reference Q9RA61.
Seq ID No. 392: Amino acid sequence for Trichoderma harzianum glucosidase, Uniparc reference UPI00078BF747, Uniprot reference A0A2T4AR08.
Seq ID No. 393: Amino acid sequence for Hypocrea jecorina glucosidase, Uniparc reference UPI000006AA61, Uniprot reference Q12715.
Seq ID No. 394: Amino acid sequence for Streptomyces sp. glucosidase, Uniparc reference UPI00000B411B, Uniprot reference Q59976.
Seq ID No. 395: Amino acid sequence for Streptococcus pyogenes glucosidase, Uniparc reference UPI00000C7E56, Uniprot reference Q99YP9.
Seq ID No. 396: Amino acid sequence for Trifolium repens glucosidase, Uniparc reference UPI000012691B, Uniprot reference P26205.
Seq ID No. 397: Amino acid sequence for Talaromyces emersonii glucosidase, Uniparc reference UPI000006C8FF, Uniprot reference Q8TGI8.
Seq ID No. 398: Amino acid sequence for Hungateiclostridium thermocellum glucosidase, Uniparc reference UPI0000126903, Uniprot reference P26208.
Seq ID No. 399: Amino acid sequence for Lactobacillus plantarum glucosidase, Uniparc reference UPI000219FE3E, Uniprot reference F9ULH8.
Seq ID No. 400: Amino acid sequence for Agrobacterium tumefaciens glucosidase, Uniparc reference UPI0003F2033A, Uniprot reference A0A2I4PGZ0.
Seq ID No. 401: Codon optimised DNA encoding 6×His Cyberlindnera fabianii glucosidase, Uniparc reference UPI00049B1A8C, Uniprot reference A0A061B3J2.
Seq ID No. 402: Codon optimised DNA encoding 6×His Flavobacterium gilvum glucosidase, Uniparc reference UPI0004E3EF7B, Uniprot reference A0A085EII0.
Seq ID No. 403: Codon optimised DNA encoding 6×His Algibacter lectus glucosidase, Uniparc reference UPI00050EE490, Uniprot reference A0A090X649.
Seq ID No. 404: Codon optimised DNA encoding 6×His Microbacterium azadirachtae glucosidase, Uniparc reference UPI0005ECB51E, Uniprot reference A0A0F0LB94.
Seq ID No. 405: Codon optimised DNA encoding 6×His Actinobacteria bacterium glucosidase, Uniparc reference UPI0006588DAD, Uniprot reference A0A0J0UT37.
Seq ID No. 406: Codon optimised DNA encoding 6×His Chloroflexi bacterium glucosidase, Uniparc reference UPI0007968552, Uniprot reference A0A136KWB3.
Seq ID No. 407: Codon optimised DNA encoding 6×His Komagataeibacter rhaeticus glucosidase, Uniparc reference UPI0002080410, Uniprot reference A0A181C809.
Seq ID No. 408: Codon optimised DNA encoding 6×His Bacteroides sp. glucosidase, Uniparc reference UPI0008211BFC, Uniprot reference A0A1C5WEL8.
Seq ID No. 409: Codon optimised DNA encoding 6×His Streptomyces rubrolavendulae glucosidase, Uniparc reference UPI00085A2BD0, Uniprot reference A0A1D8FZW3.
Seq ID No. 410: Codon optimised DNA encoding 6×His Clostridium roseum glucosidase, Uniparc reference UPI00098C60F6, Uniprot reference A0A1S8KYM5.
Seq ID No. 411: Codon optimised DNA encoding 6×His uncultured bacterium glucosidase, Uniparc reference UPI0009CE0D4C, Uniprot reference A0A1V5M6V6.
Seq ID No. 412: Codon optimised DNA encoding 6×His Firmicutes bacterium glucosidase, Uniparc reference UPI0009D4127D, Uniprot reference A0A1V6AN95.
Seq ID No. 413: Codon optimised DNA encoding 6×His Anthracocystis flocculosa glucosidase, Uniparc reference UPI00045601AB, Uniprot reference A0A061H1Z3.
Seq ID No. 414: Codon optimised DNA encoding 6×His Bifidobacterium boum glucosidase, Uniparc reference UPI0004FF77C7, Uniprot reference A0A086ZKU2.
Seq ID No. 415: Codon optimised DNA encoding 6×His Jejuia pallidilutea glucosidase, Uniparc reference UPI00051EDBDE, Uniprot reference A0A098LTR2.
Seq ID No. 416: Codon optimised DNA encoding 6×His Ceratocystis fimbriata glucosidase, Uniparc reference UPI00062105AB, Uniprot reference A0A0F8B2B0.
Seq ID No. 417: Codon optimised DNA encoding 6×His Actinobacteria bacterium glucosidase, Uniparc reference UPI0006583AB1, Uniprot reference A0A0J0UVW7.
Seq ID No. 418: Codon optimised DNA encoding 6×His Rhodococcus sp. glucosidase, Uniparc reference UPI0007AABFAD, Uniprot reference A0A143QAX3.
Seq ID No. 419: Codon optimised DNA encoding 6×His Valsa mali glucosidase, Uniparc reference UPI0007F2D02D, Uniprot reference A0A194VF47.
Seq ID No. 420: Codon optimised DNA encoding 6×His uncultured Bacteroides sp. glucosidase, Uniparc reference UPI000821004D, Uniprot reference A0A1C5WSI4.
Seq ID No. 421: Codon optimised DNA encoding 6×His Eisenbergiella tayi glucosidase, Uniparc reference UPI00084089B2, Uniprot reference A0A1E3ALT2.
Seq ID No. 422: Codon optimised DNA encoding 6×His Streptomyces sp. glucosidase, Uniparc reference UPI000978E914, Uniprot reference A0A1V2MY14.
Seq ID No. 423: Codon optimised DNA encoding 6×His Firmicutes bacterium glucosidase, Uniparc reference UPI0009CBF21C, Uniprot reference A0A1V5MH90.
Seq ID No. 424: Codon optimised DNA encoding 6×His Tenericutes bacterium glucosidase, Uniparc reference UPI0009D5B1F0, Uniprot reference A0A1V6BAK3.
Seq ID No. 425: Codon optimised DNA encoding 6×His Gluconobacter oxydans glucosidase, Uniparc reference UPI0004A87350, Uniprot reference A0A067Z479.
Seq ID No. 426: Codon optimised DNA encoding 6×His Bifidobacterium catenulatum glucosidase, Uniparc reference UPI00050787A2, Uniprot reference A0A087B8Q8.
Seq ID No. 427: Codon optimised DNA encoding 6×His Bionectria ochroleuca glucosidase, Uniparc reference UPI0005965863, Uniprot reference A0A0B7K538.
Seq ID No. 428: Codon optimised DNA encoding 6×His Parcubacteria sp. glucosidase, Uniparc reference UPI0006377CA4, Uniprot reference A0A0G0GD78.
Seq ID No. 429: Codon optimised DNA encoding 6×His Microbacterium ketosireducens glucosidase, Uniparc reference UPI0006228575, Uniprot reference A0A0M2H276.
Seq ID No. 430: Codon optimised DNA encoding 6×His Roseburia faecis glucosidase, Uniparc reference UPI0006C454B4, Uniprot reference A0A173R3W4.
Seq ID No. 431: Codon optimised DNA encoding 6×His Kwoniella dejecticola glucosidase, Uniparc reference UPI0007F1D695, Uniprot reference A0A1A6A050.
Seq ID No. 432: Codon optimised DNA encoding 6×His uncultured Clostridium sp. glucosidase, Uniparc reference UPI0008232A70, Uniprot reference A0A1C6C862.
Seq ID No. 433: Codon optimised DNA encoding 6×His Cyberlindnera jadinii glucosidase, Uniparc reference UPI000866CB30, Uniprot reference A0A1E4S2F8.
Seq ID No. 434: Codon optimised DNA encoding 6×His Bacteroidetes bacterium glucosidase, Uniparc reference UPI0009D3483D, Uniprot reference A0A1V5G4W6.
Seq ID No. 435: Codon optimised DNA encoding 6×His Verrucomicrobia bacterium glucosidase, Uniparc reference UPI0009CC9AEA, Uniprot reference A0A1V5Q4R4.
Seq ID No. 436: Codon optimised DNA encoding 6×His Bacteroidetes bacterium glucosidase, Uniparc reference UPI0009C6DE73, Uniprot reference A0A1V6BV25.
Seq ID No. 437: Codon optimised DNA encoding 6×His Lichtheimia ramosa glucosidase, Uniparc reference UPI0004E051A9, Uniprot reference A0A077WUK7.
Seq ID No. 438: Codon optimised DNA encoding 6×His Bifidobacterium mongoliense glucosidase, Uniparc reference UPI0005060F52, Uniprot reference A0A087BWT7.
Seq ID No. 439: Codon optimised DNA encoding 6×His Vibrio ishigakensis glucosidase, Uniparc reference UPI0005910ED9, Uniprot reference A0A0B8NZY1.
Seq ID No. 440: Codon optimised DNA encoding 6×His Phaeomoniella chlamydospora glucosidase, Uniparc reference UPI00063B706F, Uniprot reference A0A0G2HEV5.
Seq ID No. 441: Codon optimised DNA encoding 6×His Ardenticatena maritima glucosidase, Uniparc reference UPI0006C04F59, Uniprot reference A0A0M8K5H7.
Seq ID No. 442: Codon optimised DNA encoding 6×His Coprococcus comes glucosidase, Uniparc reference UPI000197E031, Uniprot reference A0A173WPC4.
Seq ID No. 443: Codon optimised DNA encoding 6×His Nocardioides dokdonensis glucosidase, Uniparc reference UPI0007DDBAB3, Uniprot reference A0A1A9GNJ0.
Seq ID No. 444: Codon optimised DNA encoding 6×His uncultured Clostridium sp. glucosidase, Uniparc reference UPI000822F7EB, Uniprot reference A0A1C6EBC1.
Seq ID No. 445: Codon optimised DNA encoding 6×His Acetobacterium wieringae glucosidase, Uniparc reference UPI000878EB40, Uniprot reference A0A1F2PFB4.
Seq ID No. 446: Codon optimised DNA encoding 6×His Tenericutes bacterium glucosidase, Uniparc reference UPI0009D0A2C1, Uniprot reference A0A1V5HNJ3.
Seq ID No. 447: Codon optimised DNA encoding 6×His Tenericutes bacterium glucosidase, Uniparc reference UPI0009CB8A73, Uniprot reference A0A1V5UFI1.
Seq ID No. 448: Codon optimised DNA encoding 6×His uncultured bacterium glucosidase, Uniparc reference UPI0009CC90AF, Uniprot reference A0A1V6CDT2.
Seq ID No. 449: Codon optimised DNA encoding 6×His Parabacteroides distasonis glucosidase, Uniparc reference UPI0004D8E473, Uniprot reference A0A078SYD0.
Seq ID No. 450: Codon optimised DNA encoding 6×His Bifidobacterium psychraerophilum glucosidase, Uniparc reference UPI0005006C6A, Uniprot reference A0A087CJB0.
Seq ID No. 451: Codon optimised DNA encoding 6×His Hebeloma cylindrosporum glucosidase, Uniparc reference UPI00059A3BA8, Uniprot reference A0A0C2YJF2.
Seq ID No. 452: Codon optimised DNA encoding 6×His Brenneria goodwinii glucosidase, Uniparc reference UPI0006579FEA, Uniprot reference A0A0G4JRR8.
Seq ID No. 453: Codon optimised DNA encoding 6×His Aspergillus calidoustus glucosidase, Uniparc reference UPI00073C9E60, Uniprot reference A0A0U5GN20.
Seq ID No. 454: Codon optimised DNA encoding 6×His Bacteroides finegoldii glucosidase, Uniparc reference UPI0006C6E0C3, Uniprot reference A0A174BEZ2.
Seq ID No. 455: Codon optimised DNA encoding 6×His Altererythrobacter dongtanensis glucosidase, Uniparc reference UPI0008153D6D, Uniprot reference A0A1B2A943.
Seq ID No. 456: Codon optimised DNA encoding 6×His uncultured Anaerotruncus sp. glucosidase, Uniparc reference UPI0008206E7B, Uniprot reference A0A1C6FWD9.
Seq ID No. 457: Codon optimised DNA encoding 6×His Candidatus firestone glucosidase, Uniparc reference UPI0008AC0E17, Uniprot reference A0A1F5UJR6.
Seq ID No. 458: Codon optimised DNA encoding 6×His Tenericutes bacterium glucosidase, Uniparc reference UPI0009C6C8E4, Uniprot reference A0A1V5HS22.
Seq ID No. 459: Codon optimised DNA encoding 6×His Lentisphaerae bacterium glucosidase, Uniparc reference UPI0009CEE1AB, Uniprot reference A0A1V5VBL2.
Seq ID No. 460: Codon optimised DNA encoding 6×His Planctomycetes bacterium glucosidase, Uniparc reference UPI0009CAAB0A, Uniprot reference A0A1V6FZ47.
Seq ID No. 461: Codon optimised DNA encoding 6×His Pseudallescheria apiosperma glucosidase, Uniparc reference UPI0004DD62AC, Uniprot reference A0A084G332.
Seq ID No. 462: Codon optimised DNA encoding 6×His Nonlabens sediminis glucosidase, Uniparc reference UPI000507F00A, Uniprot reference A0A090Q4N8.
Seq ID No. 463: Codon optimised DNA encoding 6×His Gynuella sunshinyii glucosidase, Uniparc reference UPI0005CC42CA, Uniprot reference A0A0C5VDU3.
Seq ID No. 464: Codon optimised DNA encoding 6×His Verticillium longisporum glucosidase, Uniparc reference UPI00063E4005, Uniprot reference A0A0G4N9Q7.
Seq ID No. 465: Codon optimised DNA encoding 6×His Cellulomonas sp. glucosidase, Uniparc reference UPI00073C6CD3, Uniprot reference A0A0V8TAB5.
Seq ID No. 466: Codon optimised DNA encoding 6×His Hungatella hathewayi glucosidase, Uniparc reference UPI0006C024BB, Uniprot reference A0A174FBX7.
Seq ID No. 467: Codon optimised DNA encoding 6×His Mesorhizobium sp. glucosidase, Uniparc reference UPI000688E74D, Uniprot reference A0A1C2DG64.
Seq ID No. 468: Codon optimised DNA encoding 6×His Clostridium sp. glucosidase, Uniparc reference UPI000822FAB7, Uniprot reference A0A1C6GRT5.
Seq ID No. 469: Codon optimised DNA encoding 6×His Chlamydiales bacterium glucosidase, Uniparc reference UPI0009284E74, Uniprot reference A0A1M3CSY6.
Seq ID No. 470: Codon optimised DNA encoding 6×His Spirochaetes bacterium glucosidase, Uniparc reference UPI0009CB461D, Uniprot reference A0A1V5HUX3.
Seq ID No. 471: Codon optimised DNA encoding 6×His bacterium glucosidase, Uniparc reference UPI0009C92D6A, Uniprot reference A0A1V5VHB9.
Seq ID No. 472: Codon optimised DNA encoding 6×His Thermotogae bacterium glucosidase, Uniparc reference UPI0009D12B86, Uniprot reference A0A1V6H2W5.
Seq ID No. 473: Codon optimised DNA encoding 6×His Pseudallescheria apiosperma glucosidase, Uniparc reference UPI0004DCBED3, Uniprot reference A0A084GGE2.
Seq ID No. 474: Codon optimised DNA encoding 6×His Algibacter lectus glucosidase, Uniparc reference UPI00050E0BE3, Uniprot reference A0A090VF17.
Seq ID No. 475: Codon optimised DNA encoding 6×His Paxillus involutus glucosidase, Uniparc reference UPI0005B075C8, Uniprot reference A0A0C9TWP5.
Seq ID No. 476: Codon optimised DNA encoding 6×His Verticillium longisporum glucosidase, Uniparc reference UPI00063DF006, Uniprot reference A0A0G4NA55.
Seq ID No. 477: Codon optimised DNA encoding 6×His Mucilaginibacter gotjawali glucosidase, Uniparc reference UPI00076F8EA4, Uniprot reference A0A110B1H1.
Seq ID No. 478: Codon optimised DNA encoding 6×His Bacteroides uniformis glucosidase, Uniparc reference UPI0006C0FFEF, Uniprot reference A0A174IWW4.
Seq ID No. 479: Codon optimised DNA encoding 6×His Coprococcus sp. glucosidase, Uniparc reference UPI0008222B77, Uniprot reference A0A1C5W0N6.
Seq ID No. 480: Codon optimised DNA encoding 6×His Blautia sp. glucosidase, Uniparc reference UPI0006C36823, Uniprot reference A0A1C6K2X5.
Seq ID No. 481: Codon optimised DNA encoding 6×His Cellulomonas sp. glucosidase, Uniparc reference UPI00092B3CD4, Uniprot reference A0A1M3ELH4.
Seq ID No. 482: Codon optimised DNA encoding 6×His bacterium glucosidase, Uniparc reference UPI0009C85428, Uniprot reference A0A1V5J984.
Seq ID No. 483: Codon optimised DNA encoding 6×His Spirochaetes bacterium glucosidase, Uniparc reference UPI0009CA1D5C, Uniprot reference A0A1V5WHQ7.
Seq ID No. 484: Codon optimised DNA encoding 6×His Tenericutes bacterium glucosidase, Uniparc reference UPI0009C7041F, Uniprot reference A0A1V6IJ57.
Seq ID No. 485: Codon optimised DNA encoding 6×His Flavobacterium gilvum glucosidase, Uniparc reference UPI0004E2A41F, Uniprot reference A0A085EG29.
Seq ID No. 486: Codon optimised DNA encoding 6×His Algibacter lectus glucosidase, Uniparc reference UPI0005102470, Uniprot reference A0A090WWZ1.
Seq ID No. 487: Codon optimised DNA encoding 6×His Hydnomerulius pinastri glucosidase, Uniparc reference UPI0005B0F0AE, Uniprot reference A0A0C9WDY0.
Seq ID No. 488: Codon optimised DNA encoding 6×His Nocardia farcinica glucosidase, Uniparc reference UPI00065C2666, Uniprot reference A0A0H5NWN2.
Seq ID No. 489: Codon optimised DNA encoding 6×His Bacteroides cellulosilyticus glucosidase, Uniparc reference UPI000760375B, Uniprot reference A0A125MG18.
Seq ID No. 490: Codon optimised DNA encoding 6×His Fonsecaea erecta glucosidase, Uniparc reference UPI0007DF4250, Uniprot reference A0A178ZBN2.
Seq ID No. 491: Codon optimised DNA encoding 6×His Bacteroides sp. glucosidase, Uniparc reference UPI0008209852, Uniprot reference A0A1C5W9N9.
Seq ID No. 492: Codon optimised DNA encoding 6×His Tannerella forsythia glucosidase, Uniparc reference UPI00086C34C1, Uniprot reference A0A1D3UGH8.
Seq ID No. 493: Codon optimised DNA encoding 6×His Microbacterium esteraromaticum glucosidase, Uniparc reference UPI00097E83BB, Uniprot reference A0A1R4KI92.
Seq ID No. 494: Codon optimised DNA encoding 6×His bacterium glucosidase, Uniparc reference UPI0009D255E5, Uniprot reference A0A1V5LJK9.
Seq ID No. 495: Codon optimised DNA encoding 6×His Candidatus hydrogenedentes glucosidase, Uniparc reference UPI0009C5A3CF, Uniprot reference A0A1V5Z2L2.
Seq ID No. 496: Codon optimised DNA encoding 6×His Bacteroidetes bacterium glucosidase, Uniparc reference UPI0009C55799, Uniprot reference A0A1V6J4J8.
Seq ID No. 497: Codon optimised DNA encoding 6×His Penicillium solitum glucosidase, Uniparc reference UPI0009D4067F, Uniprot reference A0A1V6RQ41.
Seq ID No. 498: Codon optimised DNA encoding 6×His Weissella soli glucosidase, Uniparc reference UPI0008737AA2, Uniprot reference A0A288Q8I2.
Seq ID No. 499: Codon optimised DNA encoding 6×His Acetatifactor muris glucosidase, Uniparc reference UPI000CAC57D4, Uniprot reference A0A2K4ZN91.
Seq ID No. 500: Codon optimised DNA encoding 6×His Corynespora cassiicola glucosidase, Uniparc reference UPI000D237A4A, Uniprot reference A0A2T2NYD4.
Seq ID No. 501: Codon optimised DNA encoding 6×His Meira miltonrushii glucosidase, Uniparc reference UPI000D77C91D, Uniprot reference A0A316V6M3.
Seq ID No. 502: Codon optimised DNA encoding 6×His Bacteroides fragilis glucosidase, Uniparc reference UPI00004E1F76, Uniprot reference A0A380YVC7.
Seq ID No. 503: Codon optimised DNA encoding 6×His Malassezia restricta glucosidase, Uniparc reference UPI000F0C30E8, Uniprot reference A0A3G2SB79.
Seq ID No. 504: Codon optimised DNA encoding 6×His Fusarium euwallaceae glucosidase, Uniparc reference UPI000FFFEFB2, Uniprot reference A0A430LYA2.
Seq ID No. 505: Codon optimised DNA encoding 6×His Psathyrella aberdarensis glucosidase, Uniparc reference UPI0010251887, Uniprot reference A0A4Q2E070.
Seq ID No. 506: Codon optimised DNA encoding 6×His Aeromonas hydrophila glucosidase, Uniparc reference UPI0000E69509, Uniprot reference A0KLP6.
Seq ID No. 507: Codon optimised DNA encoding 6×His Saccharopolyspora erythraea glucosidase, Uniparc reference UPI00000B86CB, Uniprot reference A4F7P9.
Seq ID No. 508: Codon optimised DNA encoding 6×His Streptomyces sviceus glucosidase, Uniparc reference UPI000180240E, Uniprot reference B5I181.
Seq ID No. 509: Codon optimised DNA encoding 6×His Naematelia encephala glucosidase, Uniparc reference UPI000A250F78, Uniprot reference A0A1Y2AWB7.
Seq ID No. 510: Codon optimised DNA encoding 6×His Hartmannibacter diazotrophicus glucosidase, Uniparc reference UPI000C0221F1, Uniprot reference A0A2C9D6I2.
Seq ID No. 511: Codon optimised DNA encoding 6×His Pontimonas salivibrio glucosidase, Uniparc reference UPI000CEB5AB1, Uniprot reference A0A2L2BPE2.
Seq ID No. 512: Codon optimised DNA encoding 6×His Cadophora sp. glucosidase, Uniparc reference UPI000D5B9C38, Uniprot reference A0A2V1CH24.
Seq ID No. 513: Codon optimised DNA encoding 6×His Meira miltonrushii glucosidase, Uniparc reference UPI000D779558, Uniprot reference A0A316V8S0.
Seq ID No. 514: Codon optimised DNA encoding 6×His Monilinia fructigena glucosidase, Uniparc reference UPI000DC42E3D, Uniprot reference A0A395IJW4.
Seq ID No. 515: Codon optimised DNA encoding 6×His Hortaea werneckii glucosidase, Uniparc reference UPI000F3E476C, Uniprot reference A0A3M6XGS0.
Seq ID No. 516: Codon optimised DNA encoding 6×His Streptomyces netropsis glucosidase, Uniparc reference UPI00101460D7, Uniprot reference A0A445N7U0.
Seq ID No. 517: Codon optimised DNA encoding 6×His Aureobasidium pullulans glucosidase, Uniparc reference UPI001139C6A8, Uniprot reference A0A4S9IFI0.
Seq ID No. 518: Codon optimised DNA encoding 6×His Aspergillus clavatus glucosidase, Uniparc reference UPI0000EA5CFF, Uniprot reference A1CTN9.
Seq ID No. 519: Codon optimised DNA encoding 6×His Clavibacter michiganensis glucosidase, Uniparc reference UPI0001523037, Uniprot reference A5CT94.
Seq ID No. 520: Codon optimised DNA encoding 6×His Penicillium rubens glucosidase, Uniparc reference UPI0001831CF5, Uniprot reference B6H7R5.
Seq ID No. 521: Codon optimised DNA encoding 6×His Lachnoclostridium sp. glucosidase, Uniparc reference UPI000B365547, Uniprot reference A0A1Y4NTL9.
Seq ID No. 522: Codon optimised DNA encoding 6×His Rhodobacteraceae bacterium glucosidase, Uniparc reference UPI000C09BF88, Uniprot reference A0A2D5IXB9.
Seq ID No. 523: Codon optimised DNA encoding 6×His Bacteroides fragilis glucosidase, Uniparc reference UPI0004B5EEF2, Uniprot reference A0A2M9UUC4.
Seq ID No. 524: Codon optimised DNA encoding 6×His Aspergillus indologenus glucosidase, Uniparc reference UPI000D7FE1ED, Uniprot reference A0A2V5IY78.
Seq ID No. 525: Codon optimised DNA encoding 6×His Acaromyces ingoldii glucosidase, Uniparc reference UPI000D802B25, Uniprot reference A0A316YR39.
Seq ID No. 526: Codon optimised DNA encoding 6×His Monilinia fructigena glucosidase, Uniparc reference UPI000DC60823, Uniprot reference A0A395J1U5.
Seq ID No. 527: Codon optimised DNA encoding 6×His Paenibacillus xylanexedens glucosidase, Uniparc reference UPI000F52D773, Uniprot reference A0A3N6CA02.
Seq ID No. 528: Codon optimised DNA encoding 6×His Actinomyces howellii glucosidase, Uniparc reference UPI000F6DAAAE, Uniprot reference A0A448HIG0.
Seq ID No. 529: Codon optimised DNA encoding 6×His Friedmanniomyces endolithicus glucosidase, Uniparc reference UPI00113D19DF, Uniprot reference A0A4V5N9I4.
Seq ID No. 530: Codon optimised DNA encoding 6×His Neosartorya fischeri glucosidase, Uniparc reference UPI0000EA8672, Uniprot reference A1DNS0.
Seq ID No. 531: Codon optimised DNA encoding 6×His Pseudomonas aeruginosa glucosidase, Uniparc reference UPI0000D7314B, Uniprot reference A6V4K6.
Seq ID No. 532: Codon optimised DNA encoding 6×His Talaromyces stipitatus glucosidase, Uniparc reference UPI00018E7266, Uniprot reference B8MF24.
Seq ID No. 533: Codon optimised DNA encoding 6×His Aquimixticola soesokkakensis glucosidase, Uniparc reference UPI000A1A5FD7, Uniprot reference A0A1Y5RVF8.
Seq ID No. 534: Codon optimised DNA encoding 6×His Rhodobacterales bacterium glucosidase, Uniparc reference UPI000C98D37C, Uniprot reference A0A2D9YGV1.
Seq ID No. 535: Codon optimised DNA encoding 6×His Methylorubrum extorquens glucosidase, Uniparc reference UPI0006F9793E, Uniprot reference A0A2N9AS40.
Seq ID No. 536: Codon optimised DNA encoding 6×His Clostridium perfringens glucosidase, Uniparc reference UPI00000CF7C8, Uniprot reference A0A2X2YBP0.
Seq ID No. 537: Codon optimised DNA encoding 6×His Acholeplasmatales bacterium glucosidase, Uniparc reference UPI0008AEBEA3, Uniprot reference A0A348NID6.
Seq ID No. 538: Codon optimised DNA encoding 6×His Bacteroidetes bacterium glucosidase, Uniparc reference UPI000EC3C979, Uniprot reference A0A3B8VHE8.
Seq ID No. 539: Codon optimised DNA encoding 6×His Clostridium carnis glucosidase, Uniparc reference UPI000F637E38, Uniprot reference A0A3P6K8E8.
Seq ID No. 540: Codon optimised DNA encoding 6×His Mycolicibacterium flavescens glucosidase, Uniparc reference UPI000B93B5C9, Uniprot reference A0A448HNB5.
Seq ID No. 541: Codon optimised DNA encoding 6×His Streptococcus gallolyticus glucosidase, Uniparc reference UPI000F6EFA71, Uniprot reference A0A4V6LJ94.
Seq ID No. 542: Codon optimised DNA encoding 6×His Yersinia enterocolitica glucosidase, Uniparc reference UPI0000EB54CC, Uniprot reference A1JNB7.
Seq ID No. 543: Codon optimised DNA encoding 6×His Anaeromyxobacter sp. glucosidase, Uniparc reference UPI0000ED8A80, Uniprot reference A7HFG4.
Seq ID No. 544: Codon optimised DNA encoding 6×His Talaromyces stipitatus glucosidase, Uniparc reference UPI00018E7D70, Uniprot reference B8MK55.
Seq ID No. 545: Codon optimised DNA encoding 6×His Hortaea werneckii glucosidase, Uniparc reference UPI000A2E3FAA, Uniprot reference A0A1Z5SL14.
Seq ID No. 546: Codon optimised DNA encoding 6×His Micavibrio sp. glucosidase, Uniparc reference UPI000C529025, Uniprot reference A0A2E2Q8X2.
Seq ID No. 547: Codon optimised DNA encoding 6×His Acidobacteriia bacterium glucosidase, Uniparc reference UPI000CE6B996, Uniprot reference A0A2N9MBS0.
Seq ID No. 548: Codon optimised DNA encoding 6×His Corynebacterium jeikeium glucosidase, Uniparc reference UPI000DA3A972, Uniprot reference A0A2X4T570.
Seq ID No. 549: Codon optimised DNA encoding 6×His Clostridiaceae bacterium glucosidase, Uniparc reference UPI000E8D37A1, Uniprot reference A0A353PZH8.
Seq ID No. 550: Codon optimised DNA encoding 6×His Anaerolineaceae bacterium glucosidase, Uniparc reference UPI000748C096, Uniprot reference A0A3B9PA35.
Seq ID No. 551: Codon optimised DNA encoding 6×His Gymnopilus dilepis glucosidase, Uniparc reference UPI000FF41956, Uniprot reference A0A409WSY0.
Seq ID No. 552: Codon optimised DNA encoding 6×His Kocuria rosea glucosidase, Uniparc reference UPI000F7105D4, Uniprot reference A0A448R8N0.
Seq ID No. 553: Codon optimised DNA encoding 6×His Teredinibacter sp. glucosidase, Uniparc reference UPI0011696FAB, Uniprot reference A0A509DWZ3.
Seq ID No. 554: Codon optimised DNA encoding 6×His Aspergillus niger glucosidase, Uniparc reference UPI0000EFB564, Uniprot reference A2QS42.
Seq ID No. 555: Codon optimised DNA encoding 6×His Laccaria bicolor glucosidase, Uniparc reference UPI000164423D, Uniprot reference B0D734.
Seq ID No. 556: Codon optimised DNA encoding 6×His Podosphaera parvula glucosidase, Uniparc reference UPI00017357F6, Uniprot reference B9XH33.
Seq ID No. 557: Codon optimised DNA encoding 6×His Megamonas hypermegale glucosidase, Uniparc reference UPI00042469F6, Uniprot reference A0A239TGH2.
Seq ID No. 558: Codon optimised DNA encoding 6×His Armillaria gallica glucosidase, Uniparc reference UPI000BC209C3, Uniprot reference A0A2H3E300.
Seq ID No. 559: Codon optimised DNA encoding 6×His Micromonospora sp. glucosidase, Uniparc reference UPI000D2EAE87, Uniprot reference A0A2P8AV03.
Seq ID No. 560: Codon optimised DNA encoding 6×His Klebsiella oxytoca glucosidase, Uniparc reference UPI000DA286EE, Uniprot reference A0A2X5CJC5.
Seq ID No. 561: Codon optimised DNA encoding 6×His Candidatus ozemobacter glucosidase, Uniparc reference UPI000DFAEF6C, Uniprot reference A0A367ZIH8.
Seq ID No. 562: Codon optimised DNA encoding 6×His Coleophoma crateriformis glucosidase, Uniparc reference UPI000E399EE8, Uniprot reference A0A3D8R2C2.
Seq ID No. 563: Codon optimised DNA encoding 6×His Apiotrichum porosum glucosidase, Uniparc reference UPI000FBC01E3, Uniprot reference A0A427XHS2.
Seq ID No. 564: Codon optimised DNA encoding 6×His Acholeplasma hippikon glucosidase, Uniparc reference UPI00068E4E50, Uniprot reference A0A449BJ27.
Seq ID No. 565: Codon optimised DNA encoding 6×His Streptomyces spectabilis glucosidase, Uniparc reference UPI001185F074, Uniprot reference A0A516RGT1.
Seq ID No. 566: Codon optimised DNA encoding 6×His Aspergillus niger glucosidase, Uniparc reference UPI0000EFCED2, Uniprot reference A2R8G2.
Seq ID No. 567: Codon optimised DNA encoding 6×His Xanthomonas campestris glucosidase, Uniparc reference UPI00000D8BFA, Uniprot reference B0RYA0.
Seq ID No. 568: Codon optimised DNA encoding 6×His Lactobacillus paracasei glucosidase, Uniparc reference UPI00019C9CD7, Uniprot reference C2FDL2.
Seq ID No. 569: Codon optimised DNA encoding 6×His Bifiguratus adelaidae glucosidase, Uniparc reference UPI000BC490A3, Uniprot reference A0A261XUH4.
Seq ID No. 570: Codon optimised DNA encoding 6×His bacterium glucosidase, Uniparc reference UPI000CC31AE2, Uniprot reference A0A2H5YYA1.
Seq ID No. 571: Codon optimised DNA encoding 6×His Actinomadura parvosata glucosidase, Uniparc reference UPI000D26C9C6, Uniprot reference A0A2P9IY35.
Seq ID No. 572: Codon optimised DNA encoding 6×His Melissococcus plutonius glucosidase, Uniparc reference UPI00024F22B8, Uniprot reference A0A2Z5Y4P3.
Seq ID No. 573: Codon optimised DNA encoding 6×His Enterococcus durans glucosidase, Uniparc reference UPI000E020871, Uniprot reference A0A377KJS3.
Seq ID No. 574: Codon optimised DNA encoding 6×His Malassezia restricta glucosidase, Uniparc reference UPI000DD17A7D, Uniprot reference A0A3G2S2J6.
Seq ID No. 575: Codon optimised DNA encoding 6×His Apiotrichum porosum glucosidase, Uniparc reference UPI000FA2DE87, Uniprot reference A0A427XZQ0.
Seq ID No. 576: Codon optimised DNA encoding 6×His Streptomonospora sp. glucosidase, Uniparc reference UPI0010355193, Uniprot reference A0A4P6PWP5.
Seq ID No. 577: Codon optimised DNA encoding 6×His Lactobacillus gasseri glucosidase, Uniparc reference UPI00119640A1, Uniprot reference A0A558LH47.
Seq ID No. 578: Codon optimised DNA encoding 6×His Aspergillus niger glucosidase, Uniparc reference UPI0000EFD0AA, Uniprot reference A2RAJ1.
Seq ID No. 579: Codon optimised DNA encoding 6×His Neosartorya fumigata glucosidase, Uniparc reference UPI000170BF91, Uniprot reference B0XXG1.
Seq ID No. 580: Codon optimised DNA encoding 6×His Eubacterium eligens glucosidase, Uniparc reference UPI0001A5B234, Uniprot reference C4Z6T5.
Seq ID No. 581: Codon optimised DNA encoding 6×His Bifiguratus adelaidae glucosidase, Uniparc reference UPI000BC64A75, Uniprot reference A0A261XVM8.
Seq ID No. 582: Codon optimised DNA encoding 6×His bacterium glucosidase, Uniparc reference UPI000CAA854E, Uniprot reference A0A2H6EX57.
Seq ID No. 583: Codon optimised DNA encoding 6×His Corynespora cassiicola glucosidase, Uniparc reference UPI000D24EC2C, Uniprot reference A0A2T2N5H9.
Seq ID No. 584: Codon optimised DNA encoding 6×His Pseudomicrostroma glucosiphilum glucosidase, Uniparc reference UPI000D77A0B7, Uniprot reference A0A316UK83.
Seq ID No. 585: Codon optimised DNA encoding 6×His Staphylococcus saprophyticus glucosidase, Uniparc reference UPI0002DAAFCC, Uniprot reference A0A380HD31.
Seq ID No. 586: Codon optimised DNA encoding 6×His Malassezia restricta glucosidase, Uniparc reference UPI000F0C6B8E, Uniprot reference A0A3G2S932.
Seq ID No. 587: Codon optimised DNA encoding 6×His Saitozyma podzolica glucosidase, Uniparc reference UPI000FBA70E2, Uniprot reference A0A427YCL1.
Seq ID No. 588: Codon optimised DNA encoding 6×His Tremella mesenterica glucosidase, Uniparc reference UPI00102822B4, Uniprot reference A0A4Q1BRC6.
Seq ID No. 589: Codon optimised DNA encoding 6×His Arthrobacter sp. glucosidase, Uniparc reference UPI0000527506, Uniprot reference A0JZ86.
Seq ID No. 590: Codon optimised DNA encoding 6×His Scheffersomyces stipitis glucosidase, Uniparc reference UPI000157388C, Uniprot reference A3LRB0.
Seq ID No. 591: Codon optimised DNA encoding 6×His Leptothrix cholodnii glucosidase, Uniparc reference UPI0001712E02, Uniprot reference B1XZK8.
Seq ID No. 592: Codon optimised DNA encoding 6×His Thauera sp. glucosidase, Uniparc reference UPI000166883C, Uniprot reference C4ZLL7.
Seq ID No. 593: Codon optimised DNA encoding 6×His Kosmotoga olearia glucosidase, Uniparc reference UPI00018494AB, Uniprot reference C5CDW5.
Seq ID No. 594: Codon optimised DNA encoding 6×His Roseburia intestinalis glucosidase, Uniparc reference UPI0001CD671F, Uniprot reference D4L3Y2.
Seq ID No. 595: Codon optimised DNA encoding 6×His Streptococcus equinus glucosidase, Uniparc reference UPI0001E0DC00, Uniprot reference E0PDF8.
Seq ID No. 596: Codon optimised DNA encoding 6×His Streptococcus cristatus glucosidase, Uniparc reference UPI0001F80C4B, Uniprot reference E8JUK5.
Seq ID No. 597: Codon optimised DNA encoding 6×His Cellulosilyticum lentocellum glucosidase, Uniparc reference UPI0001D2DBBC, Uniprot reference F2JLH3.
Seq ID No. 598: Codon optimised DNA encoding 6×His Streptococcus gallolyticus glucosidase, Uniparc reference UPI0001C48657, Uniprot reference F5WYI5.
Seq ID No. 599: Codon optimised DNA encoding 6×His Ketogulonicigenium vulgare glucosidase, Uniparc reference UPI00021D443A, Uniprot reference F9Y8Z7.
Seq ID No. 600: Codon optimised DNA encoding 6×His Spathaspora passalidarum glucosidase, Uniparc reference UPI000228255D, Uniprot reference G3AGX1.
Seq ID No. 601: Codon optimised DNA encoding 6×His Niastella koreensis glucosidase, Uniparc reference UPI00023F6F5F, Uniprot reference G8T9J3.
Seq ID No. 602: Codon optimised DNA encoding 6×His Cellvibrio sp. glucosidase, Uniparc reference UPI000260108C, Uniprot reference I3IDC0.
Seq ID No. 603: Codon optimised DNA encoding 6×His Flavobacterium sp. glucosidase, Uniparc reference UPI000272D1E0, Uniprot reference J1ACA0.
Seq ID No. 604: Codon optimised DNA encoding 6×His Macrophomina phaseolina glucosidase, Uniparc reference UPI00028E7FE1, Uniprot reference K2S5D3.
Seq ID No. 605: Codon optimised DNA encoding 6×His Kosmotoga olearia glucosidase, Uniparc reference UPI00018483A2, Uniprot reference C5CHI5.
Seq ID No. 606: Codon optimised DNA encoding 6×His Blautia obeum glucosidase, Uniparc reference UPI0001CD5918, Uniprot reference D4LRF6.
Seq ID No. 607: Codon optimised DNA encoding 6×His Bifidobacterium dentium glucosidase, Uniparc reference UPI0001E18CDA, Uniprot reference E0Q541.
Seq ID No. 608: Codon optimised DNA encoding 6×His Anaerolinea thermophila glucosidase, Uniparc reference UPI0001F55F8F, Uniprot reference E8N5R8.
Seq ID No. 609: Codon optimised DNA encoding 6×His Coriobacterium glomerans glucosidase, Uniparc reference UPI0002050DA2, Uniprot reference F2N7E4.
Seq ID No. 610: Codon optimised DNA encoding 6×His Microlunatus phosphovorus glucosidase, Uniparc reference UPI000210C886, Uniprot reference F5XJQ3.
Seq ID No. 611: Codon optimised DNA encoding 6×His Streptomyces sp. glucosidase, Uniparc reference UPI0001C18877, Uniprot reference G0Q1U8.
Seq ID No. 612: Codon optimised DNA encoding 6×His Spathaspora passalidarum glucosidase, Uniparc reference UPI0002282B61, Uniprot reference G3AIV6.
Seq ID No. 613: Codon optimised DNA encoding 6×His Glarea lozoyensis glucosidase, Uniparc reference UPI0002402F4A, Uniprot reference H0ET34.
Seq ID No. 614: Codon optimised DNA encoding 6×His Glaciozyma antarctica glucosidase, Uniparc reference UPI0002633B36, Uniprot reference I3UJK0.
Seq ID No. 615: Codon optimised DNA encoding 6×His Arthrobacter sp. glucosidase, Uniparc reference UPI00027DFD35, Uniprot reference J7LN00.
Seq ID No. 616: Codon optimised DNA encoding 6×His Fusarium pseudograminearum glucosidase, Uniparc reference UPI00028D698C, Uniprot reference K3VMA9.
Seq ID No. 617: Codon optimised DNA encoding 6×His Hypocrea rufa glucosidase, Uniparc reference UPI000006AA61, Uniprot reference C6GGC9.
Seq ID No. 618: Codon optimised DNA encoding 6×His Ruminococcus torques glucosidase, Uniparc reference UPI0001CDA964, Uniprot reference D4M6W6.
Seq ID No. 619: Codon optimised DNA encoding 6×His Bifidobacterium dentium glucosidase, Uniparc reference UPI0001E17370, Uniprot reference E0Q9Z7.
Seq ID No. 620: Codon optimised DNA encoding 6×His Bacteroides salanitronis glucosidase, Uniparc reference UPI0001FC71F6, Uniprot reference F0R2D7.
Seq ID No. 621: Codon optimised DNA encoding 6×His Bacteroides coprosuis glucosidase, Uniparc reference UPI00020E6D19, Uniprot reference F3ZQ40.
Seq ID No. 622: Codon optimised DNA encoding 6×His Marinomonas posidonica glucosidase, Uniparc reference UPI00020D4CC6, Uniprot reference F6CWF6.
Seq ID No. 623: Codon optimised DNA encoding 6×His Chaetomium thermophilum glucosidase, Uniparc reference UPI000227E8ED, Uniprot reference G0SE64.
Seq ID No. 624: Codon optimised DNA encoding 6×His Tetragenococcus halophilus glucosidase, Uniparc reference UPI00022B9A15, Uniprot reference G4L5K1.
Seq ID No. 625: Codon optimised DNA encoding 6×His Paenibacillus sp. glucosidase, Uniparc reference UPI00024F0867, Uniprot reference H6CCZ8.
Seq ID No. 626: Codon optimised DNA encoding 6×His Turneriella parva glucosidase, Uniparc reference UPI000265AA4B, Uniprot reference 14B8U7.
Seq ID No. 627: Codon optimised DNA encoding 6×His Arthrobacter sp. glucosidase, Uniparc reference UPI00027DFDB2, Uniprot reference J7LQK9.
Seq ID No. 628: Codon optimised DNA encoding 6×His Agaricus bisporus glucosidase, Uniparc reference UPI00029074B1, Uniprot reference K5W7V1.
Seq ID No. 629: Codon optimised DNA encoding 6×His Nectria haematococca glucosidase, Uniparc reference UPI0001B67634, Uniprot reference C7YIP3.
Seq ID No. 630: Codon optimised DNA encoding 6×His Bacteroides xylanisolvens glucosidase, Uniparc reference UPI0001A25287, Uniprot reference D4VSZ0.
Seq ID No. 631: Codon optimised DNA encoding 6×His Sediminispirochaeta smaragdinae glucosidase, Uniparc reference UPI0001DD9790, Uniprot reference E1R331.
Seq ID No. 632: Codon optimised DNA encoding 6×His Deinococcus proteolyticus glucosidase, Uniparc reference UPI0001FC42E9, Uniprot reference F0RPV2.
Seq ID No. 633: Codon optimised DNA encoding 6×His Sphingobacterium sp. glucosidase, Uniparc reference UPI0002033A0A, Uniprot reference F4C226.
Seq ID No. 634: Codon optimised DNA encoding 6×His Sphingobium chlorophenolicum glucosidase, Uniparc reference UPI0001E54133, Uniprot reference F6ET40.
Seq ID No. 635: Codon optimised DNA encoding 6×His Caloramator australicus glucosidase, Uniparc reference UPI00021CACC4, Uniprot reference G0V3V5.
Seq ID No. 636: Codon optimised DNA encoding 6×His Commensalibacter intestini glucosidase, Uniparc reference UPI000230E3BF, Uniprot reference G6F370.
Seq ID No. 637: Codon optimised DNA encoding 6×His Paenibacillus sp. glucosidase, Uniparc reference UPI00024F07AC, Uniprot reference H6CIT2.
Seq ID No. 638: Codon optimised DNA encoding 6×His Nitrolancea hollandica glucosidase, Uniparc reference UPI0002638AF3, Uniprot reference I4EIA9.
Seq ID No. 639: Codon optimised DNA encoding 6×His Cryptococcus neoformans glucosidase, Uniparc reference UPI000392C3ED, Uniprot reference J9VVK7.
Seq ID No. 640: Codon optimised DNA encoding 6×His Acidipropionibacterium acidipropionici glucosidase, Uniparc reference UPI0002988588, Uniprot reference K75596.
Seq ID No. 641: Codon optimised DNA encoding 6×His Prevotella sp. glucosidase, Uniparc reference UPI0001B93465, Uniprot reference C9PT75.
Seq ID No. 642: Codon optimised DNA encoding 6×His Rhodobacter capsulatus glucosidase, Uniparc reference UPI0001D08095, Uniprot reference D5ALU0.
Seq ID No. 643: Codon optimised DNA encoding 6×His Stigmatella aurantiaca glucosidase, Uniparc reference UPI0001E74370, Uniprot reference E3FJ05.
Seq ID No. 644: Codon optimised DNA encoding 6×His Sphaerochaeta globosa glucosidase, Uniparc reference UPI0002010060, Uniprot reference F0RVK3.
Seq ID No. 645: Codon optimised DNA encoding 6×His Sphaerochaeta coccoides glucosidase, Uniparc reference UPI000207D78C, Uniprot reference F4GH96.
Seq ID No. 646: Codon optimised DNA encoding 6×His Novosphingobium sp. glucosidase, Uniparc reference UPI00020EFBCB, Uniprot reference F6ICQ5.
Seq ID No. 647: Codon optimised DNA encoding 6×His Arthrobotrys oligospora glucosidase, Uniparc reference UPI000225331F, Uniprot reference G1XH86.
Seq ID No. 648: Codon optimised DNA encoding 6×His Lactococcus lactis glucosidase, Uniparc reference UPI00000C6907, Uniprot reference G6FFS4.
Seq ID No. 649: Codon optimised DNA encoding 6×His Phaeospirillum molischianum glucosidase, Uniparc reference UPI000255313A, Uniprot reference H8FXH7.
Seq ID No. 650: Codon optimised DNA encoding 6×His Modestobacter marinus glucosidase, Uniparc reference UPI0002609C68, Uniprot reference I4EW72.
Seq ID No. 651: Codon optimised DNA encoding 6×His Saccharothrix espanaensis glucosidase, Uniparc reference UPI00028AC337, Uniprot reference K0K125.
Seq ID No. 652: Codon optimised DNA encoding 6×His Cronobacter sakazakii glucosidase, Uniparc reference UPI00029BA293, Uniprot reference K8DAK0.
Seq ID No. 653: Codon optimised DNA encoding 6×His Verticillium alfalfae glucosidase, Uniparc reference UPI0001BBDF1E, Uniprot reference C9SVX1.
Seq ID No. 654: Codon optimised DNA encoding 6×His Bacteroides xylanisolvens glucosidase, Uniparc reference UPI0001CCFD36, Uniprot reference D6CY10.
Seq ID No. 655: Codon optimised DNA encoding 6×His Leadbetterella byssophila glucosidase, Uniparc reference UPI0001EBD98A, Uniprot reference E4RUR0.
Seq ID No. 656: Codon optimised DNA encoding 6×His Sphaerochaeta globosa glucosidase, Uniparc reference UPI00020102A5, Uniprot reference F0RYB6.
Seq ID No. 657: Codon optimised DNA encoding 6×His Sphaerochaeta coccoides glucosidase, Uniparc reference UPI000207D6A0, Uniprot reference F4GLH6.
Seq ID No. 658: Codon optimised DNA encoding 6×His Haloplasma contractile glucosidase, Uniparc reference UPI000212252C, Uniprot reference F7Q0Y2.
Seq ID No. 659: Codon optimised DNA encoding 6×His Nitrospirillum amazonense glucosidase, Uniparc reference UPI0002265447, Uniprot reference G1Y407.
Seq ID No. 660: Codon optimised DNA encoding 6×His Azospirillum brasilense glucosidase, Uniparc reference UPI00023420B8, Uniprot reference G8AWD9.
Seq ID No. 661: Codon optimised DNA encoding 6×His Phaeospirillum molischianum glucosidase, Uniparc reference UPI000255314F, Uniprot reference H8FXJ8.
Seq ID No. 662: Codon optimised DNA encoding 6×His Modestobacter marinus glucosidase, Uniparc reference UPI000260A2FA, Uniprot reference I4EYD5.
Seq ID No. 663: Codon optimised DNA encoding 6×His Wickerhamomyces ciferrii glucosidase, Uniparc reference UPI000283EB8D, Uniprot reference K0KVJ2.
Seq ID No. 664: Codon optimised DNA encoding 6×His Gloeocapsa sp. glucosidase, Uniparc reference UPI0002A5D085, Uniprot reference K9XKL8.
Seq ID No. 665: Codon optimised DNA encoding 6×His Sphaerobacter thermophilus glucosidase, Uniparc reference UPI0001A3BCB6, Uniprot reference D1C7U8.
Seq ID No. 666: Codon optimised DNA encoding 6×His Bacteroides xylanisolvens glucosidase, Uniparc reference UPI0001CCEF1F, Uniprot reference D6D4V2.
Seq ID No. 667: Codon optimised DNA encoding 6×His Prevotella buccae glucosidase, Uniparc reference UPI0001F149E8, Uniprot reference E6K4W5.
Seq ID No. 668: Codon optimised DNA encoding 6×His Grosmannia clavigera glucosidase, Uniparc reference UPI0001FF1101, Uniprot reference F0XBR0.
Seq ID No. 669: Codon optimised DNA encoding 6×His Melampsora larici-populina glucosidase, Uniparc reference UPI00020F9774, Uniprot reference F4R4W2.
Seq ID No. 670: Codon optimised DNA encoding 6×His Prevotella multisaccharivorax glucosidase, Uniparc reference UPI0002138E9C, Uniprot reference F8N7G1.
Seq ID No. 671: Codon optimised DNA encoding 6×His Streptomyces zinciresistens glucosidase, Uniparc reference UPI0002255A63, Uniprot reference G2G8K4.
Seq ID No. 672: Codon optimised DNA encoding 6×His Granulicella mallensis glucosidase, Uniparc reference UPI0001D9FC40, Uniprot reference G8NY42.
Seq ID No. 673: Codon optimised DNA encoding 6×His Gibberella zeae glucosidase, Uniparc reference UPI00021F1FEE, Uniprot reference I1RH94.
Seq ID No. 674: Codon optimised DNA encoding 6×His Modestobacter marinus glucosidase, Uniparc reference UPI00026090A2, Uniprot reference I4EYK6.
Seq ID No. 675: Codon optimised DNA encoding 6×His Lactobacillus equicursoris glucosidase, Uniparc reference UPI0002869F55, Uniprot reference K0NRS8.
Seq ID No. 676: Codon optimised DNA encoding 6×His Colletotrichum fructicola glucosidase, Uniparc reference UPI0002A93280, Uniprot reference L2F9W0.
Seq ID No. 677: Codon optimised DNA encoding 6×His Streptosporangium roseum glucosidase, Uniparc reference UPI0001BF8AF6, Uniprot reference D2B261.
Seq ID No. 678: Codon optimised DNA encoding 6×His Listeria grayi glucosidase, Uniparc reference UPI00019F252A, Uniprot reference D7UX19.
Seq ID No. 679: Codon optimised DNA encoding 6×His Enterococcus italicus glucosidase, Uniparc reference UPI0001F11EFF, Uniprot reference E6LF07.
Seq ID No. 680: Codon optimised DNA encoding 6×His Fluviicola taffensis glucosidase, Uniparc reference UPI000203D9EA, Uniprot reference F2IIT6.
Seq ID No. 681: Codon optimised DNA encoding 6×His Shigella flexneri glucosidase, Uniparc reference UPI00020CA641, Uniprot reference F5N4W9.
Seq ID No. 682: Codon optimised DNA encoding 6×His Actinomyces sp. glucosidase, Uniparc reference UPI0002189080, Uniprot reference F9EFW5.
Seq ID No. 683: Codon optimised DNA encoding 6×His Verticillium dahliae glucosidase, Uniparc reference UPI00022EBE5A, Uniprot reference G2X5V6.
Seq ID No. 684: Codon optimised DNA encoding 6×His Actinoplanes sp. glucosidase, Uniparc reference UPI00023ED5A8, Uniprot reference G8S0M0.
Seq ID No. 685: Codon optimised DNA encoding 6×His Gibberella zeae glucosidase, Uniparc reference UPI000023EA29, Uniprot reference I1S320.
Seq ID No. 686: Codon optimised DNA encoding 6×His Auricularia subglabra glucosidase, Uniparc reference UPI00027CE685, Uniprot reference J0WV06.
Seq ID No. 687: Codon optimised DNA encoding 6×His Nitratireductor indicus glucosidase, Uniparc reference UPI00028E86D6, Uniprot reference K2N268.
Seq ID No. 688: Codon optimised DNA encoding 6×His Thermoclostridium stercorarium glucosidase, Uniparc reference UPI0002AD999E, Uniprot reference L7VH66.
Seq ID No. 689: Codon optimised DNA encoding 6×His Geobacillus sp. glucosidase, Uniparc reference UPI0002AF2DE3, Uniprot reference L8A1S2.
Seq ID No. 690: Codon optimised DNA encoding 6×His uncultured bacterium glucosidase, Uniparc reference UPI000327C3CC, Uniprot reference M9ZC55.
Seq ID No. 691: Codon optimised DNA encoding 6×His Burkholderia ambifaria glucosidase, Uniparc reference UPI000059ACE1, Uniprot reference Q0BAK2.
Seq ID No. 692: Codon optimised DNA encoding 6×His Aspergillus oryzae glucosidase, Uniparc reference UPI0000676B8D, Uniprot reference Q2UIR4.
Seq ID No. 693: Codon optimised DNA encoding 6×His Yersinia pseudotuberculosis glucosidase, Uniparc reference UPI00004269B5, Uniprot reference Q66DJ0.
Seq ID No. 694: Codon optimised DNA encoding 6×His Wallemia ichthyophaga glucosidase, Uniparc reference UPI000331CC98, Uniprot reference R9AF64.
Seq ID No. 695: Codon optimised DNA encoding 6×His Glarea lozoyensis glucosidase, Uniparc reference UPI0003522404, Uniprot reference S3DIM6.
Seq ID No. 696: Codon optimised DNA encoding 6×His Moniliophthora roreri glucosidase, Uniparc reference UPI0003BF753A, Uniprot reference V2YU31.
Seq ID No. 697: Codon optimised DNA encoding 6×His Zhouia amylolytica glucosidase, Uniparc reference UPI0003DBCB4F, Uniprot reference W2ULL3.
Seq ID No. 698: Codon optimised DNA encoding 6×His Fusarium oxysporum glucosidase, Uniparc reference UPI0003F362FF, Uniprot reference W9NQQ3.
Seq ID No. 699: Codon optimised DNA encoding 6×His Flavobacterium johnsoniae glucosidase, Uniparc reference UPI00006EA168, Uniprot reference A5FAA5.
Seq ID No. 700: Codon optimised DNA encoding 6×His Oryza sativa glucosidase, Uniparc reference UPI00002394F5, Uniprot reference Q75I93.
Seq ID No. 701: Codon optimised DNA encoding 6×His Clostridium saccharoperbutylacetonicum glucosidase, Uniparc reference UPI0002B65681, Uniprot reference M1MCD1.
Seq ID No. 702: Codon optimised DNA encoding 6×His Rhodococcus sp. glucosidase, Uniparc reference UPI0002D21DB1, Uniprot reference N1MBN6.
Seq ID No. 703: Codon optimised DNA encoding 6×His Burkholderia ambifaria glucosidase, Uniparc reference UPI000059C0CD, Uniprot reference Q0BCV8.
Seq ID No. 704: Codon optimised DNA encoding 6×His Xanthomonas campestris glucosidase, Uniparc reference UPI00005CE9E7, Uniprot reference Q3BVH7.
Seq ID No. 705: Codon optimised DNA encoding 6×His Caulobacter vibrioides glucosidase, Uniparc reference UPI00000C7604, Uniprot reference Q9A6F8.
Seq ID No. 706: Codon optimised DNA encoding 6×His Arcticibacter svalbardensis glucosidase, Uniparc reference UPI000338772A, Uniprot reference R9GRA7.
Seq ID No. 707: Codon optimised DNA encoding 6×His Winogradskyella psychrotolerans glucosidase, Uniparc reference UPI00035A4604, Uniprot reference S7VQ28.
Seq ID No. 708: Codon optimised DNA encoding 6×His Methyloglobulus morosus glucosidase, Uniparc reference UPI0003C4ED6A, Uniprot reference V5DXT8.
Seq ID No. 709: Codon optimised DNA encoding 6×His Pestalotiopsis fici glucosidase, Uniparc reference UPI0003E05C04, Uniprot reference W3WV37.
Seq ID No. 710: Codon optimised DNA encoding 6×His Capronia coronata glucosidase, Uniparc reference UPI000434E698, Uniprot reference W9YNR7.
Seq ID No. 711: Codon optimised DNA encoding 6×His Aspergillus aculeatus glucosidase, Uniparc reference UPI00001268FD, Uniprot reference P48825.
Seq ID No. 712: Codon optimised DNA encoding 6×His Thermotoga neapolitana glucosidase, Uniparc reference UPI0000DD5996, Uniprot reference Q0GC07.
Seq ID No. 713: Codon optimised DNA encoding 6×His Clostridium saccharoperbutylacetonicum glucosidase, Uniparc reference UPI0002B666E8, Uniprot reference M1MJF4.
Seq ID No. 714: Codon optimised DNA encoding 6×His Schizosaccharomyces pombe glucosidase, Uniparc reference UPI000006B001, Uniprot reference O74799.
Seq ID No. 715: Codon optimised DNA encoding 6×His Phaeosphaeria nodorum glucosidase, Uniparc reference UPI000161BD2B, Uniprot reference Q0TXF6.
Seq ID No. 716: Codon optimised DNA encoding 6×His Xylella fastidiosa Dixon glucosidase, Uniparc reference UPI00003806C8, Uniprot reference Q3RGJ3.
Seq ID No. 717: Codon optimised DNA encoding 6×His Schizosaccharomyces pombe glucosidase, Uniparc reference UPI000006A330, Uniprot reference Q9P6J6.
Seq ID No. 718: Codon optimised DNA encoding 6×His Arcticibacter svalbardensis glucosidase, Uniparc reference UPI000337E887, Uniprot reference R9GWD6.
Seq ID No. 719: Codon optimised DNA encoding 6×His Colletotrichum gloeosporioides glucosidase, Uniparc reference UPI0003885717, Uniprot reference T0KJI7.
Seq ID No. 720: Codon optimised DNA encoding 6×His uncultured bacterium glucosidase, Uniparc reference UPI0003C9E340, Uniprot reference V5R1E8.
Seq ID No. 721: Codon optimised DNA encoding 6×His Xanthomonas arboricola glucosidase, Uniparc reference UPI0003E06A61, Uniprot reference W4S7I5.
Seq ID No. 722: Codon optimised DNA encoding 6×His Fusarium oxysporum glucosidase, Uniparc reference UPI00021EC697, Uniprot reference X0A8X8.
Seq ID No. 723: Codon optimised DNA encoding 6×His Paenibacillus polymyxa glucosidase, Uniparc reference UPI00001108D0, Uniprot reference P22073.
Seq ID No. 724: Codon optimised DNA encoding 6×His Kluyveromyces marxianus glucosidase, Uniparc reference UPI0001BE5ADA, Uniprot reference D1GCC6.
Seq ID No. 725: Codon optimised DNA encoding 6×His Ilumatobacter coccineus glucosidase, Uniparc reference UPI0002C04A25, Uniprot reference M5A594.
Seq ID No. 726: Codon optimised DNA encoding 6×His Agrobacterium sp. glucosidase, Uniparc reference UPI0000126912, Uniprot reference P12614.
Seq ID No. 727: Codon optimised DNA encoding 6×His Cytophaga hutchinsonii glucosidase, Uniparc reference UPI000038ECF7, Uniprot reference Q11P53.
Seq ID No. 728: Codon optimised DNA encoding 6×His Thermobifida fusca glucosidase, Uniparc reference UPI00003C5CA4, Uniprot reference Q47PF5.
Seq ID No. 729: Codon optimised DNA encoding 6×His Botryotinia fuckeliana glucosidase, Uniparc reference UPI0000069E5E, Uniprot reference Q9UVJ6.
Seq ID No. 730: Codon optimised DNA encoding 6×His Agarivorans albus glucosidase, Uniparc reference UPI00033994D2, Uniprot reference R9PTK2.
Seq ID No. 731: Codon optimised DNA encoding 6×His Enterococcus sp. glucosidase, Uniparc reference UPI00038B7639, Uniprot reference T0UDU2.
Seq ID No. 732: Codon optimised DNA encoding 6×His Salinispira pacifica glucosidase, Uniparc reference UPI0003D8A7DC, Uniprot reference V5WKT4.
Seq ID No. 733: Codon optimised DNA encoding 6×His Bacteroides xylanisolvens glucosidase, Uniparc reference UPI0003ECF15E, Uniprot reference W6P696.
Seq ID No. 734: Codon optimised DNA encoding 6×His Fusarium oxysporum glucosidase, Uniparc reference UPI000430029A, Uniprot reference X0AB46.
Seq ID No. 735: Codon optimised DNA encoding 6×His Thermotoga maritima glucosidase, Uniparc reference UPI0000126906, Uniprot reference Q08638B.
Seq ID No. 736: Codon optimised DNA encoding 6×His Neotermes koshunensis glucosidase, Uniparc reference UPI0000083EA8, Uniprot reference Q8T0W7.
Seq ID No. 737: Codon optimised DNA encoding 6×His Thanatephorus cucumeris glucosidase, Uniparc reference UPI0002BF34B3, Uniprot reference M5CHG9.
Seq ID No. 738: Codon optimised DNA encoding 6×His Hungateiclostridium thermocellum glucosidase, Uniparc reference UPI000053581D, Uniprot reference P14002.
Seq ID No. 739: Codon optimised DNA encoding 6×His Koribacter versatilis glucosidase, Uniparc reference UPI0000D76A70, Uniprot reference Q1IJ89.
Seq ID No. 740: Codon optimised DNA encoding 6×His Neosartorya fumigata glucosidase, Uniparc reference UPI00005203D8, Uniprot reference Q4WLX5.
Seq ID No. 741: Codon optimised DNA encoding 6×His Amycolatopsis vancoresmycina glucosidase, Uniparc reference UPI00032DB8C1, Uniprot reference R1FKF0.
Seq ID No. 742: Codon optimised DNA encoding 6×His Gibberella fujikuroi glucosidase, Uniparc reference UPI0003519C76, Uniprot reference S0EKU1.
Seq ID No. 743: Codon optimised DNA encoding 6×His Bifidobacterium longum glucosidase, Uniparc reference UPI000390F839, Uniprot reference T2I2H5.
Seq ID No. 744: Codon optimised DNA encoding 6×His uncultured bacterium glucosidase, Uniparc reference UPI0003DFF0DA, Uniprot reference W0FLD1.
Seq ID No. 745: Codon optimised DNA encoding 6×His Bacteroides xylanisolvens glucosidase, Uniparc reference UPI0002D3D994, Uniprot reference W6P9J9.
Seq ID No. 746: Codon optimised DNA encoding 6×His Fusarium oxysporum glucosidase, Uniparc reference UPI00042F8016, Uniprot reference X0HA35.
Seq ID No. 747: Codon optimised DNA encoding 6×His Sorghum bicolor glucosidase, Uniparc reference UPI00000A7F40, Uniprot reference Q41290.
Seq ID No. 748: Codon optimised DNA encoding 6×His Clostridium cellulovorans glucosidase, Uniparc reference UPI000050B701, Uniprot reference Q53EH2.
Seq ID No. 749: Codon optimised DNA encoding 6×His Dacryopinax primogenitus glucosidase, Uniparc reference UPI0002C29EAA, Uniprot reference M5G1U5.
Seq ID No. 750: Codon optimised DNA encoding 6×His Rhizobium radiobacter glucosidase, Uniparc reference UPI0000126913, Uniprot reference P27034.
Seq ID No. 751: Codon optimised DNA encoding 6×His Phanerochaete chrysosporium glucosidase, Uniparc reference UPI00006E0CC7, Uniprot reference Q25BW5A.
Seq ID No. 752: Codon optimised DNA encoding 6×His Enterobacter agglomerans glucosidase, Uniparc reference UPI0000126905, Uniprot reference Q59437.
Seq ID No. 753: Codon optimised DNA encoding 6×His Candidatus microthrix glucosidase, Uniparc reference UPI00033056C4, Uniprot reference R4YWU4.
Seq ID No. 754: Codon optimised DNA encoding 6×His Chthonomonas calidirosea glucosidase, Uniparc reference UPI0003427F29, Uniprot reference S0ET37.
Seq ID No. 755: Codon optimised DNA encoding 6×His Acholeplasma brassicae glucosidase, Uniparc reference UPI0003B04208, Uniprot reference U4KMR7.
Seq ID No. 756: Codon optimised DNA encoding 6×His Chania multitudinisentens glucosidase, Uniparc reference UPI0003E13BD3, Uniprot reference W0LJ28.
Seq ID No. 757: Codon optimised DNA encoding 6×His Gibberella moniliformis glucosidase, Uniparc reference UPI0003ECCF80, Uniprot reference W7MW98.
Seq ID No. 758: Codon optimised DNA encoding 6×His Fusarium oxysporum glucosidase, Uniparc reference UPI0004305856, Uniprot reference X0I2U8.
Seq ID No. 759: Codon optimised DNA encoding 6×His Phanerochaete chrysosporium glucosidase, Uniparc reference UPI00006E0CC7, Uniprot reference Q25BW5B.
Seq ID No. 760: Codon optimised DNA encoding 6×His Secale cereale glucosidase, Uniparc reference UPI00000A7EFC, Uniprot reference Q9FYS3.
Seq ID No. 761: Codon optimised DNA encoding 6×His Anoxybacillus gonensis glucosidase, Uniparc reference UPI0002C0273E, Uniprot reference M5QUM2.
Seq ID No. 762: Codon optimised DNA encoding 6×His Thermotoga maritima glucosidase, Uniparc reference UPI0000126906, Uniprot reference Q08638A.
Seq ID No. 763: Codon optimised DNA encoding 6×His Rhodospirillum rubrum glucosidase, Uniparc reference UPI00003C2ACC, Uniprot reference Q2RP51.
Seq ID No. 764: Codon optimised DNA encoding 6×His Thermotoga neapolitana glucosidase, Uniparc reference UPI00000B9013, Uniprot reference Q60038.
Seq ID No. 765: Codon optimised DNA encoding 6×His Candidatus microthrix glucosidase, Uniparc reference UPI00032F466E, Uniprot reference R4Z6M6.
Seq ID No. 766: Codon optimised DNA encoding 6×His Ruminiclostridium cellobioparum glucosidase, Uniparc reference UPI00032874EC, Uniprot reference S0FPI8.
Seq ID No. 767: Codon optimised DNA encoding 6×His Acholeplasma brassicae glucosidase, Uniparc reference UPI0003B0490C, Uniprot reference U4KMV4.
Seq ID No. 768: Codon optimised DNA encoding 6×His Klebsiella pneumoniae glucosidase, Uniparc reference UPI0003DB6EFD, Uniprot reference W1BBP5.
Seq ID No. 769: Codon optimised DNA encoding 6×His Enterobacter sp. glucosidase, Uniparc reference UPI0003ED0A8E, Uniprot reference W7P6Y1.
Seq ID No. 770: Codon optimised DNA encoding 6×His Rhizoctonia solani glucosidase, Uniparc reference UPI00045BB507, Uniprot reference X8JI88.
Seq ID No. 771: Codon optimised DNA encoding 6×His Homo sapiens glucosidase, Uniparc reference UPI0000072C73, Uniprot reference Q9H227.
Seq ID No. 772: Codon optimised DNA encoding 6×His uncultured bacterium glucosidase, Uniparc reference UPI0000DD5304, Uniprot reference Q0GMU3.
Seq ID No. 773: Codon optimised DNA encoding 6×His Thermoanaerobacter thermohydrosulfuricus glucosidase, Uniparc reference UPI0002CA9E4F, Uniprot reference M8CQD9.
Seq ID No. 774: Codon optimised DNA encoding 6×His Stigmatella aurantiaca glucosidase, Uniparc reference UPI0000E297D8, Uniprot reference Q091M8.
Seq ID No. 775: Codon optimised DNA encoding 6×His Burkholderia thailandensis glucosidase, Uniparc reference UPI00006676B1, Uniprot reference Q2T7C9.
Seq ID No. 776: Codon optimised DNA encoding 6×His Yersinia pseudotuberculosis glucosidase, Uniparc reference UPI0000426E8F, Uniprot reference Q665S2.
Seq ID No. 777: Codon optimised DNA encoding 6×His Togninia minima glucosidase, Uniparc reference UPI00032BF71F, Uniprot reference R8BQM8.
Seq ID No. 778: Codon optimised DNA encoding 6×His Ophiostoma piceae glucosidase, Uniparc reference UPI00035213C2, Uniprot reference S3BVJ8.
Seq ID No. 779: Codon optimised DNA encoding 6×His Pyronema omphalodes glucosidase, Uniparc reference UPI0003B117B7, Uniprot reference U4LIL3.
Seq ID No. 780: Codon optimised DNA encoding 6×His Ogataea parapolymorpha glucosidase, Uniparc reference UPI0001F76D1F, Uniprot reference W1QIW0.
Seq ID No. 781: Codon optimised DNA encoding 6×His Hymenobacter swuensis glucosidase, Uniparc reference UPI0003F2059F, Uniprot reference W8F6U0.
Seq ID No. 782: Codon optimised DNA encoding 6×His Flavobacterium johnsoniae glucosidase, Uniparc reference UPI00006E8E1B, Uniprot reference A5FEF5.
Seq ID No. 783: Codon optimised DNA encoding 6×His Paenibacillus polymyxa glucosidase, Uniparc reference UPI000012690B, Uniprot reference P22505.
Seq ID No. 784: Codon optimised DNA encoding 6×His Oryza sativa glucosidase, Uniparc reference UPI000009D014, Uniprot reference Q8L7J2.
Seq ID No. 785: Codon optimised DNA encoding 6×His Oryza sativa glucosidase, Uniparc reference UPI0000E580F2, Uniprot reference B8AVF0.
Seq ID No. 786: Codon optimised DNA encoding 6×His Nannochloris glucosidase, Uniparc reference UPI000EA7F16F, Uniprot reference A0A452CSM4.
Seq ID No. 787: Codon optimised DNA encoding 6×His Halothermothrix orenii glucosidase, Uniparc reference UPI00006AE508, Uniprot reference B8CYA8.
Seq ID No. 788: Codon optimised DNA encoding 6×His Neurospora crassa glucosidase, Uniparc reference UPI000018B2B4, Uniprot reference Q7RWP2.
Seq ID No. 789: Codon optimised DNA encoding 6×His Micrococcus antarcticus glucosidase, Uniparc reference UPI000192BB5F, Uniprot reference B9V8P5.
Seq ID No. 790: Codon optimised DNA encoding 6×His Exiguobacterium antarcticum glucosidase, Uniparc reference UPI000285E79E, Uniprot reference K0A8J9.
Seq ID No. 791: Codon optimised DNA encoding 6×His Thermus thermophilus glucosidase, Uniparc reference UPI00000BEB61, Uniprot reference Q9RA61.
Seq ID No. 792: Codon optimised DNA encoding 6×His Trichoderma harzianum glucosidase, Uniparc reference UPI00078BF747, Uniprot reference A0A2T4AR08.
Seq ID No. 793: Codon optimised DNA encoding 6×His Hypocrea jecorina glucosidase, Uniparc reference UPI000006AA61, Uniprot reference Q12715.
Seq ID No. 794: Codon optimised DNA encoding 6×His Streptomyces sp. glucosidase, Uniparc reference UPI00000B411B, Uniprot reference Q59976.
Seq ID No. 795: Codon optimised DNA encoding 6×His Streptococcus pyogenes glucosidase, Uniparc reference UPI00000C7E56, Uniprot reference Q99YP9.
Seq ID No. 796: Codon optimised DNA encoding 6×His Trifolium repens glucosidase, Uniparc reference UPI000012691B, Uniprot reference P26205.
Seq ID No. 797: Codon optimised DNA encoding 6×His Talaromyces emersonii glucosidase, Uniparc reference UPI000006C8FF, Uniprot reference Q8TGI8.
Seq ID No. 798: Codon optimised DNA encoding 6×His Hungateiclostridium thermocellum glucosidase, Uniparc reference UPI0000126903, Uniprot reference P26208.
Seq ID No. 799: Codon optimised DNA encoding 6×His Lactobacillus plantarum glucosidase, Uniparc reference UPI000219FE3E, Uniprot reference F9ULH8.
Seq ID No. 800: Codon optimised DNA encoding 6×His Agrobacterium tumefaciens glucosidase, Uniparc reference UPI0003F2033A, Uniprot reference A0A2I4PGZ0.
Seq ID No. 801: Amino acid sequence for Bifidobacterium actinocoloniiforme glucosidase, Uniparc reference UPI000503B26C, Uniprot reference A0A086YYS8.
Seq ID No. 802: Amino acid sequence for Bifidobacterium psychraerophilum glucosidase, Uniparc reference UPI000502B461, Uniprot reference A0A087CD28.
Seq ID No. 803: Amino acid sequence for Penicillium italicum glucosidase, Uniparc reference UPI00052B8681, Uniprot reference A0A0A2K704.
Seq ID No. 804: Amino acid sequence for Microbacterium trichothecenolyticum glucosidase, Uniparc reference UPI0005ED0AE6, Uniprot reference A0A0M2HDB3.
Seq ID No. 805: Amino acid sequence for uncultured Clostridium sp. glucosidase, Uniparc reference UPI0008206F38, Uniprot reference A0A1C6I6A3.
Seq ID No. 806: Amino acid sequence for Bifiguratus adelaidae glucosidase, Uniparc reference UPI000BC57319, Uniprot reference A0A261Y7Q8.
Seq ID No. 807: Amino acid sequence for Paenibacillus thiaminolyticus glucosidase, Uniparc reference UPI000B3B73CD, Uniprot reference A0A378ZIK3.
Seq ID No. 808: Amino acid sequence for Microbacterium lemovicicum glucosidase, Uniparc reference UPI000F8FB9BA, Uniprot reference A0A3S9WE68.
Seq ID No. 809: Amino acid sequence for Rhodococcus erythropolis glucosidase, Uniparc reference UPI00019923DC, Uniprot reference C1A1N4.
Seq ID No. 810: Amino acid sequence for Cutibacterium avidum glucosidase, Uniparc reference UPI00022C19B5, Uniprot reference G4CZU8.
Seq ID No. 811: Amino acid sequence for Clavibacter michiganensis glucosidase, Uniparc reference UPI0002C5A938, Uniprot reference M5BD10.
Seq ID No. 8I2: Amino acid sequence for Microbacterium sp. glucosidase, Uniparc reference UPI0003DE3509, Uniprot reference W0ZC23.
Seq ID No. 813: Amino acid sequence for Bifidobacterium actinocoloniiforme glucosidase, Uniparc reference UPI000507F38A, Uniprot reference A0A086YZL4.
Seq ID No. 814: Amino acid sequence for Bifidobacterium reuteri glucosidase, Uniparc reference UPI000506119A, Uniprot reference A0A087CR26.
Seq ID No. 815: Amino acid sequence for Propionibacterium freudenreichii glucosidase, Uniparc reference UPI0005A5CAC2, Uniprot reference A0A0A8RX48.
Seq ID No. 816: Amino acid sequence for Microbacterium hydrocarbonoxydans glucosidase, Uniparc reference UPI0005EC18F9, Uniprot reference A0A0M2HRU0.
Seq ID No. 817: Amino acid sequence for Pseudonocardia sp. glucosidase, Uniparc reference UPI00094B6D3B, Uniprot reference A0A1Q8KSV8.
Seq ID No. 818: Amino acid sequence for Tuber aestivum glucosidase, Uniparc reference UPI000BC13DF0, Uniprot reference A0A292PKV5.
Seq ID No. 819: Amino acid sequence for Propionibacterium australiense glucosidase, Uniparc reference UPI000E5B4EA5, Uniprot reference A0A383S7A9.
Seq ID No. 820: Amino acid sequence for Fusarium sp. glucosidase, Uniparc reference UPI001004B2C9, Uniprot reference A0A428T6E2.
Seq ID No. 821: Amino acid sequence for Nectria haematococca glucosidase, Uniparc reference UPI0001B69B5C, Uniprot reference C7ZBV0.
Seq ID No. 822: Amino acid sequence for Actinoplanes sp. glucosidase, Uniparc reference UPI00023EBB15, Uniprot reference G8S3E7.
Seq ID No. 823: Amino acid sequence for Streptomyces fulvissimus glucosidase, Uniparc reference UPI0003289BC6, Uniprot reference N0CMW2.
Seq ID No. 824: Amino acid sequence for Pestalotiopsis fici glucosidase, Uniparc reference UPI0003E02BF1, Uniprot reference W3WXF1.
Seq ID No. 825: Amino acid sequence for Bifidobacterium bohemicum glucosidase, Uniparc reference UPI0005025F2E, Uniprot reference A0A086ZGP0.
Seq ID No. 826: Amino acid sequence for Bifidobacterium saeculare glucosidase, Uniparc reference UPI0005084E52, Uniprot reference A0A087D0Q2.
Seq ID No. 827: Amino acid sequence for Bionectria ochroleuca glucosidase, Uniparc reference UPI00059674D6, Uniprot reference A0A0B7JW24.
Seq ID No. 828: Amino acid sequence for Bifidobacterium pseudocatenulatum glucosidase, Uniparc reference UPI0006C6D6F9, Uniprot reference A0A174AU04.
Seq ID No. 829: Amino acid sequence for Pseudonocardia sp. glucosidase, Uniparc reference UPI00094ABAB3, Uniprot reference A0A1Q8LPB4.
Seq ID No. 830: Amino acid sequence for bacterium glucosidase, Uniparc reference UPI000CB49A0B, Uniprot reference A0A2H5Z8Q4.
Seq ID No. 831: Amino acid sequence for Coleophoma crateriformis glucosidase, Uniparc reference UPI000E38A995, Uniprot reference A0A3D8Q771.
Seq ID No. 832: Amino acid sequence for Arthrobotrys oligospora glucosidase, Uniparc reference UPI001102DFA3, Uniprot reference A0A4Z0Y5Y8.
Seq ID No. 833: Amino acid sequence for Pyrenophora teres glucosidase, Uniparc reference UPI0001ECDCBD, Uniprot reference E3RFS2.
Seq ID No. 834: Amino acid sequence for Gordonia polyisoprenivorans glucosidase, Uniparc reference UPI00024F2A26, Uniprot reference H6MTQ7.
Seq ID No. 835: Amino acid sequence for Stigmatella aurantiaca glucosidase, Uniparc reference UPI0000E28E5D, Uniprot reference Q08521.
Seq ID No. 836: Amino acid sequence for Pestalotiopsis fici glucosidase, Uniparc reference UPI0003E03A52, Uniprot reference W3WZ03.
Seq ID No. 837: Amino acid sequence for Bifidobacterium magnum glucosidase, Uniparc reference UPI0003B7B6EE, Uniprot reference A0A087BEN9.
Seq ID No. 838: Amino acid sequence for Bifidobacterium stellenboschense glucosidase, Uniparc reference UPI000503F283, Uniprot reference A0A087DFL8.
Seq ID No. 839: Amino acid sequence for Bionectria ochroleuca glucosidase, Uniparc reference UPI00059673EE, Uniprot reference A0A0B7K316.
Seq ID No. 840: Amino acid sequence for Hungatella hathewayi glucosidase, Uniparc reference UPI0006C069F3, Uniprot reference A0A174LVE3.
Seq ID No. 841: Amino acid sequence for Mycetocola reblochoni glucosidase, Uniparc reference UPI00097EB800, Uniprot reference A0A1R4J2F9.
Seq ID No. 842: Amino acid sequence for Nonomuraea sp. glucosidase, Uniparc reference UPI0009ABD7B3, Uniprot reference A0A2P9IX34.
Seq ID No. 843: Amino acid sequence for Coleophoma crateriformis glucosidase, Uniparc reference UPI000E391DF6, Uniprot reference A0A3D8T9C2.
Seq ID No. 844: Amino acid sequence for Paenarthrobacter aurescens glucosidase, Uniparc reference UPI0000EC83AE, Uniprot reference A1R2K1.
Seq ID No. 845: Amino acid sequence for Kitasatospora setae glucosidase, Uniparc reference UPI0001F21F08, Uniprot reference E4N4F6.
Seq ID No. 846: Amino acid sequence for Nocardiopsis alba glucosidase, Uniparc reference UPI00027E251E, Uniprot reference J7L3Z8.
Seq ID No. 847: Amino acid sequence for Rhodococcus jostii glucosidase, Uniparc reference UPI0000DBA338, Uniprot reference Q0SCI4.
Seq ID No. 848: Amino acid sequence for uncultured microorganism glucosidase, Uniparc reference UPI0003EC942D, Uniprot reference W5X324.
Seq ID No. 849: Amino acid sequence for Bifidobacterium merycicum glucosidase, Uniparc reference UPI0005084B63, Uniprot reference A0A087BJ88.
Seq ID No. 850: Amino acid sequence for Bifidobacterium scardovii glucosidase, Uniparc reference UPI0005018B5B, Uniprot reference A0A087DGT3.
Seq ID No. 851: Amino acid sequence for Fusarium oxysporum glucosidase, Uniparc reference UPI00021EC81E, Uniprot reference A0A0C4DJL4.
Seq ID No. 852: Amino acid sequence for Paraphaeosphaeria sporulosa glucosidase, Uniparc reference UPI0007CE199C, Uniprot reference A0A177BXH1.
Seq ID No. 853: Amino acid sequence for Clostridium oryzae glucosidase, Uniparc reference UPI0009A50F8D, Uniprot reference A0A1V4IY77.
Seq ID No. 854: Amino acid sequence for Corynespora cassiicola glucosidase, Uniparc reference UPI000D22F5EB, Uniprot reference A0A2T2N4T6.
Seq ID No. 855: Amino acid sequence for Choiromyces venosus glucosidase, Uniparc reference UPI000F7332F8, Uniprot reference A0A3N4J9R6.
Seq ID No. 856: Amino acid sequence for Saccharopolyspora erythraea glucosidase, Uniparc reference UPI0000F550EC, Uniprot reference A4F982.
Seq ID No. 857: Amino acid sequence for Streptomyces venezuelae glucosidase, Uniparc reference UPI000204906E, Uniprot reference F2R0Y2.
Seq ID No. 858: Amino acid sequence for Arthrobacter sp. glucosidase, Uniparc reference UPI00027DFDB0, Uniprot reference J7LVB1.
Seq ID No. 859: Amino acid sequence for Thermobrachium celere glucosidase, Uniparc reference UPI00033420CB, Uniprot reference R7RMQ6.
Seq ID No. 860: Amino acid sequence for Drechslerella stenobrocha glucosidase, Uniparc reference UPI0003EA65B9, Uniprot reference W7HT93.
Seq ID No. 861: Amino acid sequence for Bifidobacterium minimum glucosidase, Uniparc reference UPI0003B38542, Uniprot reference A0A087BMR5.
Seq ID No. 862: Amino acid sequence for Bifidobacterium thermacidophilum glucosidase, Uniparc reference UPI000409825E, Uniprot reference A0A087E3K8.
Seq ID No. 863: Amino acid sequence for Verruconis gallopava glucosidase, Uniparc reference UPI0005C0229F, Uniprot reference A0A0D1X9G0.
Seq ID No. 864: Amino acid sequence for Stagonospora sp. glucosidase, Uniparc reference UPI0007CECAD5, Uniprot reference A0A178AER7.
Seq ID No. 865: Amino acid sequence for Firmicutes bacterium glucosidase, Uniparc reference UPI0009D58C49, Uniprot reference A0A1V6ALF9.
Seq ID No. 866: Amino acid sequence for Tuber borchii glucosidase, Uniparc reference UPI000D50EED1, Uniprot reference A0A2T6ZNU7.
Seq ID No. 867: Amino acid sequence for Morchella conica glucosidase, Uniparc reference UPI000F728167, Uniprot reference A0A3N4KUR3.
Seq ID No. 868: Amino acid sequence for Dictyoglomus thermophilum glucosidase, Uniparc reference UPI00018152EB, Uniprot reference B5YC96.
Seq ID No. 869: Amino acid sequence for Microlunatus phosphovorus glucosidase, Uniparc reference UPI000210C01C, Uniprot reference F5XL24.
Seq ID No. 870: Amino acid sequence for Bifidobacterium asteroides glucosidase, Uniparc reference UPI00028BB1B6, Uniprot reference K4IPD2.
Seq ID No. 871: Amino acid sequence for Dactylellina haptotyla glucosidase, Uniparc reference UPI00035AE576, Uniprot reference S8BQ60.
Seq ID No. 872: Amino acid sequence for Fusarium oxysporum glucosidase, Uniparc reference UPI0003F2D7DD, Uniprot reference W9JF54.
Seq ID No. 873: Amino acid sequence for Bifidobacterium longum glucosidase, Uniparc reference UPI0005067FF9, Uniprot reference A0A087BRY2.
Seq ID No. 874: Amino acid sequence for Bifidobacterium tsurumiense glucosidase, Uniparc reference UPI0004105550, Uniprot reference A0A087EK01.
Seq ID No. 875: Amino acid sequence for Exophiala spinifera glucosidase, Uniparc reference UPI0005BF9DF9, Uniprot reference A0A0D1YG50.
Seq ID No. 876: Amino acid sequence for Pyrenochaeta sp. glucosidase, Uniparc reference UPI0007CE7DBA, Uniprot reference A0A178E5I1.
Seq ID No. 877: Amino acid sequence for Firmicutes bacterium glucosidase, Uniparc reference UPI0009CDA3F1, Uniprot reference A0A1V6FLK2.
Seq ID No. 878: Amino acid sequence for Cadophora sp. glucosidase, Uniparc reference UPI000D5A9E68, Uniprot reference A0A2V1B599.
Seq ID No. 879: Amino acid sequence for Morchella conica glucosidase, Uniparc reference UPI000F7330CD, Uniprot reference A0A3N4L4M3.
Seq ID No. 880: Amino acid sequence for Bifidobacterium animalis glucosidase, Uniparc reference UPI000189C68F, Uniprot reference B8DV42.
Seq ID No. 881: Amino acid sequence for Treponema azotonutricium glucosidase, Uniparc reference UPI00020ED2C9, Uniprot reference F5YGD5.
Seq ID No. 882: Amino acid sequence for Acidipropionibacterium acidipropionici glucosidase, Uniparc reference UPI0002988429, Uniprot reference K7S0E5.
Seq ID No. 883: Amino acid sequence for Salinispira pacifica glucosidase, Uniparc reference UPI0003D93613, Uniprot reference V5WNB6.
Seq ID No. 884: Amino acid sequence for Bifidobacterium mongoliense glucosidase, Uniparc reference UPI000503588E, Uniprot reference A0A087C760.
Seq ID No. 885: Amino acid sequence for Bifidobacterium indicum glucosidase, Uniparc reference UPI000499F5D1, Uniprot reference A0A087VUW4.
Seq ID No. 886: Amino acid sequence for Brachyspira suanatina glucosidase, Uniparc reference UPI000659257F, Uniprot reference A0A0G4K5C2.
Seq ID No. 887: Amino acid sequence for Phialocephala scopiformis glucosidase, Uniparc reference UPI0007F2FD44, Uniprot reference A0A194X8Q9.
Seq ID No. 888: Amino acid sequence for uncultured Microbacterium sp. glucosidase, Uniparc reference UPI000A2BB4A5, Uniprot reference A0A1Y5P895.
Seq ID No. 889: Amino acid sequence for Periconia macrospinosa glucosidase, Uniparc reference UPI000D5BC9BD, Uniprot reference A0A2V1E673.
Seq ID No. 890: Amino acid sequence for Arthrobacter ulcerisalmonis glucosidase, Uniparc reference UPI000F3DC30B, Uniprot reference A0A3P5WTE2.
Seq ID No. 891: Amino acid sequence for Pseudarthrobacter chlorophenolicus glucosidase, Uniparc reference UPI0001664880, Uniprot reference B8H9A2.
Seq ID No. 892: Amino acid sequence for Haloplasma contractile glucosidase, Uniparc reference UPI0002120C63, Uniprot reference F7PUF3.
Seq ID No. 893: Amino acid sequence for Cochliobolus heterostrophus glucosidase, Uniparc reference UPI0002B73341, Uniprot reference M2ULB2.
Seq ID No. 894: Amino acid sequence for Microbacterium sp. glucosidase, Uniparc reference UPI0003DE58EA, Uniprot reference W0Z818.
Seq ID No. 895: Codon optimised DNA encoding 6×His Bifidobacterium actinocoloniiforme glucosidase, Uniparc reference UPI000503B26C, Uniprot reference A0A086YYS8.
Seq ID No. 896: Codon optimised DNA encoding 6×His Bifidobacterium psychraerophilum glucosidase, Uniparc reference UPI000502B461, Uniprot reference A0A087CD28.
Seq ID No. 897: Codon optimised DNA encoding 6×His Penicillium italicum glucosidase, Uniparc reference UPI00052B8681, Uniprot reference A0A0A2K704.
Seq ID No. 898: Codon optimised DNA encoding 6×His Microbacterium trichothecenolyticum glucosidase, Uniparc reference UPI0005ED0AE6, Uniprot reference A0A0M2HDB3.
Seq ID No. 899: Codon optimised DNA encoding 6×His uncultured Clostridium sp. glucosidase, Uniparc reference UPI0008206F38, Uniprot reference A0A1C6I6A3.
Seq ID No. 900: Codon optimised DNA encoding 6×His Bifiguratus adelaidae glucosidase, Uniparc reference UPI000BC57319, Uniprot reference A0A261Y7Q8.
Seq ID No. 901: Codon optimised DNA encoding 6×His Paenibacillus thiaminolyticus glucosidase, Uniparc reference UPI000B3B73CD, Uniprot reference A0A378ZIK3.
Seq ID No. 902: Codon optimised DNA encoding 6×His Microbacterium lemovicicum glucosidase, Uniparc reference UPI000F8FB9BA, Uniprot reference A0A3S9WE68.
Seq ID No. 903: Codon optimised DNA encoding 6×His Rhodococcus erythropolis glucosidase, Uniparc reference UPI00019923DC, Uniprot reference C1A1N4.
Seq ID No. 904: Codon optimised DNA encoding 6×His Cutibacterium avidum glucosidase, Uniparc reference UPI00022C19B5, Uniprot reference G4CZU8.
Seq ID No. 905: Codon optimised DNA encoding 6×His Clavibacter michiganensis glucosidase, Uniparc reference UPI0002C5A938, Uniprot reference M5BD10.
Seq ID No. 906: Codon optimised DNA encoding 6×His Microbacterium sp. glucosidase, Uniparc reference UPI0003DE3509, Uniprot reference W0ZC23.
Seq ID No. 907: Codon optimised DNA encoding 6×His Bifidobacterium actinocoloniiforme glucosidase, Uniparc reference UPI000507F38A, Uniprot reference A0A086YZL4.
Seq ID No. 908: Codon optimised DNA encoding 6×His Bifidobacterium reuteri glucosidase, Uniparc reference UPI000506119A, Uniprot reference A0A087CR26.
Seq ID No. 909: Codon optimised DNA encoding 6×His Propionibacterium freudenreichii glucosidase, Uniparc reference UPI0005A5CAC2, Uniprot reference A0A0A8RX48.
Seq ID No. 910: Codon optimised DNA encoding 6×His Microbacterium hydrocarbonoxydans glucosidase, Uniparc reference UPI0005EC18F9, Uniprot reference A0A0M2HRU0.
Seq ID No. 911: Codon optimised DNA encoding 6×His Pseudonocardia sp. glucosidase, Uniparc reference UPI00094B6D3B, Uniprot reference A0A1Q8KSV8.
Seq ID No. 912: Codon optimised DNA encoding 6×His Tuber aestivum glucosidase, Uniparc reference UPI000BC13DF0, Uniprot reference A0A292PKV5.
Seq ID No. 913: Codon optimised DNA encoding 6×His Propionibacterium australiense glucosidase, Uniparc reference UPI000E5B4EA5, Uniprot reference A0A383S7A9.
Seq ID No. 914: Codon optimised DNA encoding 6×His Fusarium sp. glucosidase, Uniparc reference UPI001004B2C9, Uniprot reference A0A428T6E2.
Seq ID No. 915: Codon optimised DNA encoding 6×His Nectria haematococca glucosidase, Uniparc reference UPI0001B69B5C, Uniprot reference C7ZBV0.
Seq ID No. 916: Codon optimised DNA encoding 6×His Actinoplanes sp. glucosidase, Uniparc reference UPI00023EBB15, Uniprot reference G8S3E7.
Seq ID No. 917: Codon optimised DNA encoding 6×His Streptomyces fulvissimus glucosidase, Uniparc reference UPI0003289BC6, Uniprot reference N0CMW2.
Seq ID No. 918: Codon optimised DNA encoding 6×His Pestalotiopsis fici glucosidase, Uniparc reference UPI0003E02BF1, Uniprot reference W3WXF1.
Seq ID No. 919: Codon optimised DNA encoding 6×His Bifidobacterium bohemicum glucosidase, Uniparc reference UPI0005025F2E, Uniprot reference A0A086ZGP0.
Seq ID No. 920: Codon optimised DNA encoding 6×His Bifidobacterium saeculare glucosidase, Uniparc reference UPI0005084E52, Uniprot reference A0A087D0Q2.
Seq ID No. 921: Codon optimised DNA encoding 6×His Bionectria ochroleuca glucosidase, Uniparc reference UPI00059674D6, Uniprot reference A0A0B7JW24.
Seq ID No. 922: Codon optimised DNA encoding 6×His Bifidobacterium pseudocatenulatum glucosidase, Uniparc reference UPI0006C6D6F9, Uniprot reference A0A174AU04.
Seq ID No. 923: Codon optimised DNA encoding 6×His Pseudonocardia sp. glucosidase, Uniparc reference UPI00094ABAB3, Uniprot reference A0A1Q8LPB4.
Seq ID No. 924: Codon optimised DNA encoding 6×His bacterium glucosidase, Uniparc reference UPI000CB49A0B, Uniprot reference A0A2H5Z8Q4.
Seq ID No. 925: Codon optimised DNA encoding 6×His Coleophoma crateriformis glucosidase, Uniparc reference UPI000E38A995, Uniprot reference A0A3D8Q771.
Seq ID No. 926: Codon optimised DNA encoding 6×His Arthrobotrys oligospora glucosidase, Uniparc reference UPI001102DFA3, Uniprot reference A0A4Z0Y5Y8.
Seq ID No. 927: Codon optimised DNA encoding 6×His Pyrenophora teres glucosidase, Uniparc reference UPI0001ECDCBD, Uniprot reference E3RFS2.
Seq ID No. 928: Codon optimised DNA encoding 6×His Gordonia polyisoprenivorans glucosidase, Uniparc reference UPI00024F2A26, Uniprot reference H6MTQ7.
Seq ID No. 929: Codon optimised DNA encoding 6×His Stigmatella aurantiaca glucosidase, Uniparc reference UPI0000E28E5D, Uniprot reference Q08521.
Seq ID No. 930: Codon optimised DNA encoding 6×His Pestalotiopsis fici glucosidase, Uniparc reference UPI0003E03A52, Uniprot reference W3WZ03.
Seq ID No. 931: Codon optimised DNA encoding 6×His Bifidobacterium magnum glucosidase, Uniparc reference UPI0003B7B6EE, Uniprot reference A0A087BEN9.
Seq ID No. 932: Codon optimised DNA encoding 6×His Bifidobacterium stellenboschense glucosidase, Uniparc reference UPI000503F283, Uniprot reference A0A087DFL8.
Seq ID No. 933: Codon optimised DNA encoding 6×His Bionectria ochroleuca glucosidase, Uniparc reference UPI00059673EE, Uniprot reference A0A0B7K316.
Seq ID No. 934: Codon optimised DNA encoding 6×His Hungatella hathewayi glucosidase, Uniparc reference UPI0006C069F3, Uniprot reference A0A174LVE3.
Seq ID No. 935: Codon optimised DNA encoding 6×His Mycetocola reblochoni glucosidase, Uniparc reference UPI00097EB800, Uniprot reference A0A1R4J2F9.
Seq ID No. 936: Codon optimised DNA encoding 6×His Nonomuraea sp. glucosidase, Uniparc reference UPI0009ABD7B3, Uniprot reference A0A2P9IX34.
Seq ID No. 937: Codon optimised DNA encoding 6×His Coleophoma crateriformis glucosidase, Uniparc reference UPI000E391DF6, Uniprot reference A0A3D8T9C2.
Seq ID No. 938: Codon optimised DNA encoding 6×His Paenarthrobacter aurescens glucosidase, Uniparc reference UPI0000EC83AE, Uniprot reference A1R2K1.
Seq ID No. 939: Codon optimised DNA encoding 6×His Kitasatospora setae glucosidase, Uniparc reference UPI0001F21F08, Uniprot reference E4N4F6.
Seq ID No. 940: Codon optimised DNA encoding 6×His Nocardiopsis alba glucosidase, Uniparc reference UPI00027E251E, Uniprot reference J7L3Z8.
Seq ID No. 941: Codon optimised DNA encoding 6×His Rhodococcus jostii glucosidase, Uniparc reference UPI0000DBA338, Uniprot reference Q05014.
Seq ID No. 942: Codon optimised DNA encoding 6×His uncultured microorganism glucosidase, Uniparc reference UPI0003EC942D, Uniprot reference W5X324.
Seq ID No. 943: Codon optimised DNA encoding 6×His Bifidobacterium merycicum glucosidase, Uniparc reference UPI0005084B63, Uniprot reference A0A087BJ88.
Seq ID No. 944: Codon optimised DNA encoding 6×His Bifidobacterium scardovii glucosidase, Uniparc reference UPI0005018B5B, Uniprot reference A0A087DGT3.
Seq ID No. 945: Codon optimised DNA encoding 6×His Fusarium oxysporum glucosidase, Uniparc reference UPI00021EC81E, Uniprot reference A0A0C4DJL4.
Seq ID No. 946: Codon optimised DNA encoding 6×His Paraphaeosphaeria sporulosa glucosidase, Uniparc reference UPI0007CE199C, Uniprot reference A0A177BXH1.
Seq ID No. 947: Codon optimised DNA encoding 6×His Clostridium oryzae glucosidase, Uniparc reference UPI0009A50F8D, Uniprot reference A0A1V4IY77.
Seq ID No. 948: Codon optimised DNA encoding 6×His Corynespora cassiicola glucosidase, Uniparc reference UPI000D22F5EB, Uniprot reference A0A2T2N4T6.
Seq ID No. 949: Codon optimised DNA encoding 6×His Choiromyces venosus glucosidase, Uniparc reference UPI000F7332F8, Uniprot reference A0A3N4J9R6.
Seq ID No. 950: Codon optimised DNA encoding 6×His Saccharopolyspora erythraea glucosidase, Uniparc reference UPI0000F550EC, Uniprot reference A4F982.
Seq ID No. 951: Codon optimised DNA encoding 6×His Streptomyces venezuelae glucosidase, Uniparc reference UPI000204906E, Uniprot reference F2R0Y2.
Seq ID No. 952: Codon optimised DNA encoding 6×His Arthrobacter sp. glucosidase, Uniparc reference UPI00027DFDB0, Uniprot reference J7LVB1.
Seq ID No. 953: Codon optimised DNA encoding 6×His Thermobrachium celere glucosidase, Uniparc reference UPI00033420CB, Uniprot reference R7RMQ6.
Seq ID No. 954: Codon optimised DNA encoding 6×His Drechslerella stenobrocha glucosidase, Uniparc reference UPI0003EA65B9, Uniprot reference W7HT93.
Seq ID No. 955: Codon optimised DNA encoding 6×His Bifidobacterium minimum glucosidase, Uniparc reference UPI0003B38542, Uniprot reference A0A087BMR5.
Seq ID No. 956: Codon optimised DNA encoding 6×His Bifidobacterium thermacidophilum glucosidase, Uniparc reference UPI000409825E, Uniprot reference A0A087E3K8.
Seq ID No. 957: Codon optimised DNA encoding 6×His Verruconis gallopava glucosidase, Uniparc reference UPI0005C0229F, Uniprot reference A0A0D1X9G0.
Seq ID No. 958: Codon optimised DNA encoding 6×His Stagonospora sp. glucosidase, Uniparc reference UPI0007CECAD5, Uniprot reference A0A178AER7.
Seq ID No. 959: Codon optimised DNA encoding 6×His Firmicutes bacterium glucosidase, Uniparc reference UPI0009D58C49, Uniprot reference A0A1V6ALF9.
Seq ID No. 960: Codon optimised DNA encoding 6×His Tuber borchii glucosidase, Uniparc reference UPI000D50EED1, Uniprot reference A0A2T6ZNU7.
Seq ID No. 961: Codon optimised DNA encoding 6×His Morchella conica glucosidase, Uniparc reference UPI000F728167, Uniprot reference A0A3N4KUR3.
Seq ID No. 962: Codon optimised DNA encoding 6×His Dictyoglomus thermophilum glucosidase, Uniparc reference UPI00018152EB, Uniprot reference B5YC96.
Seq ID No. 963: Codon optimised DNA encoding 6×His Microlunatus phosphovorus glucosidase, Uniparc reference UPI000210C01C, Uniprot reference F5XL24.
Seq ID No. 964: Codon optimised DNA encoding 6×His Bifidobacterium asteroides glucosidase, Uniparc reference UPI00028BB1B6, Uniprot reference K4IPD2.
Seq ID No. 965: Codon optimised DNA encoding 6×His Dactylellina haptotyla glucosidase, Uniparc reference UPI00035AE576, Uniprot reference S8BQ60.
Seq ID No. 966: Codon optimised DNA encoding 6×His Fusarium oxysporum glucosidase, Uniparc reference UPI0003F2D7DD, Uniprot reference W9JF54.
Seq ID No. 967: Codon optimised DNA encoding 6×His Bifidobacterium longum glucosidase, Uniparc reference UPI0005067FF9, Uniprot reference A0A087BRY2.
Seq ID No. 968: Codon optimised DNA encoding 6×His Bifidobacterium tsurumiense glucosidase, Uniparc reference UPI0004105550, Uniprot reference A0A087EK01.
Seq ID No. 969: Codon optimised DNA encoding 6×His Exophiala spinifera glucosidase, Uniparc reference UPI0005BF9DF9, Uniprot reference A0A0D1YG50.
Seq ID No. 970: Codon optimised DNA encoding 6×His Pyrenochaeta sp. glucosidase, Uniparc reference UPI0007CE7DBA, Uniprot reference A0A178E5I1.
Seq ID No. 971: Codon optimised DNA encoding 6×His Firmicutes bacterium glucosidase, Uniparc reference UPI0009CDA3F1, Uniprot reference A0A1V6FLK2.
Seq ID No. 972: Codon optimised DNA encoding 6×His Cadophora sp. glucosidase, Uniparc reference UPI000D5A9E68, Uniprot reference A0A2V1B599.
Seq ID No. 973: Codon optimised DNA encoding 6×His Morchella conica glucosidase, Uniparc reference UPI000F7330CD, Uniprot reference A0A3N4L4M3.
Seq ID No. 974: Codon optimised DNA encoding 6×His Bifidobacterium animalis glucosidase, Uniparc reference UPI000189C68F, Uniprot reference B8DV42.
Seq ID No. 975: Codon optimised DNA encoding 6×His Treponema azotonutricium glucosidase, Uniparc reference UPI00020ED2C9, Uniprot reference F5YGD5.
Seq ID No. 976: Codon optimised DNA encoding 6×His Acidipropionibacterium acidipropionici glucosidase, Uniparc reference UPI0002988429, Uniprot reference K7S0E5.
Seq ID No. 977: Codon optimised DNA encoding 6×His Salinispira pacifica glucosidase, Uniparc reference UPI0003D93613, Uniprot reference V5WNB6.
Seq ID No. 978: Codon optimised DNA encoding 6×His Bifidobacterium mongoliense glucosidase, Uniparc reference UPI000503588E, Uniprot reference A0A087C760.
Seq ID No. 979: Codon optimised DNA encoding 6×His Bifidobacterium indicum glucosidase, Uniparc reference UPI000499F5D1, Uniprot reference A0A087VUW4.
Seq ID No. 980: Codon optimised DNA encoding 6×His Brachyspira suanatina glucosidase, Uniparc reference UPI000659257F, Uniprot reference A0A0G4K5C2.
Seq ID No. 981: Codon optimised DNA encoding 6×His Phialocephala scopiformis glucosidase, Uniparc reference UPI0007F2FD44, Uniprot reference A0A194X8Q9.
Seq ID No. 982: Codon optimised DNA encoding 6×His uncultured Microbacterium sp. glucosidase, Uniparc reference UPI000A2BB4A5, Uniprot reference A0A1Y5P895.
Seq ID No. 983: Codon optimised DNA encoding 6×His Periconia macrospinosa glucosidase, Uniparc reference UPI000D5BC9BD, Uniprot reference A0A2V1E673.
Seq ID No. 984: Codon optimised DNA encoding 6×His Arthrobacter ulcerisalmonis glucosidase, Uniparc reference UPI000F3DC30B, Uniprot reference A0A3P5WTE2.
Seq ID No. 985: Codon optimised DNA encoding 6×His Pseudarthrobacter chlorophenolicus glucosidase, Uniparc reference UPI0001664880, Uniprot reference B8H9A2.
Seq ID No. 986: Codon optimised DNA encoding 6×His Haloplasma contractile glucosidase, Uniparc reference UPI0002120C63, Uniprot reference F7PUF3.
Seq ID No. 987: Codon optimised DNA encoding 6×His Cochliobolus heterostrophus glucosidase, Uniparc reference UPI0002B73341, Uniprot reference M2ULB2.
Seq ID No. 988: Codon optimised DNA encoding 6×His Microbacterium sp. glucosidase, Uniparc reference UPI0003DE58EA, Uniprot reference W0Z818.
Seq ID No. 989: Amino acid sequence for Rhodothermus marinus rhamnosidase, Uniparc reference UPI0001A31108, Uniprot reference D0MFR0.
Seq ID No. 990: Amino acid sequence for Streptomyces bingchenggensis rhamnosidase, Uniparc reference UPI0001D90BFE, Uniprot reference D7C463.
Seq ID No. 991: Amino acid sequence for Spirosoma linguale rhamnosidase, Uniparc reference UPI0001A3AEAC, Uniprot reference D2QL60.
Seq ID No. 992: Amino acid sequence for Roseburia intestinalis rhamnosidase, Uniparc reference UPI0001CD6D48, Uniprot reference D4L2K8.
Seq ID No. 993: Amino acid sequence for Draconibacterium orientale rhamnosidase, Uniparc reference UPI000442EF22, Uniprot reference X5DG83.
Seq ID No. 994: Amino acid sequence for Catenulispora acidiphila rhamnosidase, Uniparc reference UPI00019E052B, Uniprot reference C7QC24.
Seq ID No. 995: Amino acid sequence for Bacteroides thetaiotaomicron rhamnosidase, Uniparc reference UPI000005ADE1, Uniprot reference Q8A916.
Seq ID No. 996: Amino acid sequence for Opitutus terrae rhamnosidase, Uniparc reference UPI000172B2E3, Uniprot reference B1ZRE4.
Seq ID No. 997: Amino acid sequence for Lachnoclostridium phytofermentans rhamnosidase, Uniparc reference UPI00015FE0BE, Uniprot reference A9KJP8.
Seq ID No. 998: Amino acid sequence for Rhodanobacter denitrificans rhamnosidase, Uniparc reference UPI00022DA3ED, Uniprot reference M4NH01.
Seq ID No. 999: Amino acid sequence for Prevotella ruminicola rhamnosidase, Uniparc reference UPI0001D07633, Uniprot reference D5ETD9.
Seq ID No. 1000: Amino acid sequence for Aspergillus terreus rhamnosidase, Uniparc reference UPI000259E388, Uniprot reference I0AZ41.
Seq ID No. 1001: Amino acid sequence for Brachybacterium faecium rhamnosidase, Uniparc reference UPI0001A461B6, Uniprot reference C7MA58.
Seq ID No. 1002: Amino acid sequence for Flavobacterium johnsoniae rhamnosidase, Uniparc reference UPI00006E4A0D, Uniprot reference A5FCH3.
Seq ID No. 1003: Amino acid sequence for Rahnella aquatilis rhamnosidase, Uniparc reference UPI000245C507, Uniprot reference H2IYR2.
Seq ID No. 1004: Amino acid sequence for Bifidobacterium moukalabense rhamnosidase, Uniparc reference UPI0003E4E711, Uniprot reference W4N6H0.
Seq ID No. 1005: Amino acid sequence for Enterococcus casseliflavus rhamnosidase, Uniparc reference UPI000353BD9F, Uniprot reference S4BBS9.
Seq ID No. 1006: Amino acid sequence for Geobacillus sp. rhamnosidase, Uniparc reference UPI0001789C0D, Uniprot reference D3EED1.
Seq ID No. 1007: Amino acid sequence for Modestobacter marinus rhamnosidase, Uniparc reference UPI000260A2FE, Uniprot reference I4EYD9.
Seq ID No. 1008: Amino acid sequence for Pedobacter heparinus rhamnosidase, Uniparc reference UPI00019EF0EE, Uniprot reference C6XYM6.
Seq ID No. 1009: Amino acid sequence for Dyadobacter fermentans rhamnosidase, Uniparc reference UPI00019B5915, Uniprot reference C6VZL3.
Seq ID No. 1010: Amino acid sequence for Paenibacillus mucilaginosus rhamnosidase, Uniparc reference UPI000212C156, Uniprot reference F8FQQ3.
Seq ID No. 1011: Amino acid sequence for Paenibacillus sp. rhamnosidase, Uniparc reference UPI0003E2544D, Uniprot reference W4D866.
Seq ID No. 1012: Amino acid sequence for Bacteroides thetaiotaomicron rhamnosidase, Uniparc reference UPI000005ADD2, Uniprot reference A0A0P0FM19.
Seq ID No. 1013: Amino acid sequence for Chloroflexus aurantiacus rhamnosidase, Uniparc reference UPI00005BA60B, Uniprot reference A9WDK5.
Seq ID No. 1014: Amino acid sequence for Thermoclostridium stercorarium rhamnosidase, Uniparc reference UPI00000B098C, Uniprot reference Q9S3L0.
Seq ID No. 1015: Amino acid sequence for Bifidobacterium moukalabense rhamnosidase, Uniparc reference UPI0003E5C314, Uniprot reference W4N612.
Seq ID No. 1016: Amino acid sequence for Olsenella profusa rhamnosidase, Uniparc reference UPI0003AE032C, Uniprot reference U2USP4.
Seq ID No. 1017: Amino acid sequence for Kribbella flavida rhamnosidase, Uniparc reference UPI00019BDB13, Uniprot reference D2PMT5.
Seq ID No. 1018: Amino acid sequence for Caulobacter vibrioides rhamnosidase, Uniparc reference UPI00000C7226, Uniprot reference Q9A9K2.
Seq ID No. 1019: Amino acid sequence for Bacteroides thetaiotaomicron rhamnosidase, Uniparc reference UPI000005BA09, Uniprot reference Q8A076.
Seq ID No. 1020: Amino acid sequence for Rhodonellum psychrophilum rhamnosidase, Uniparc reference UPI0003745394, Uniprot reference U5BUY4.
Seq ID No. 1021: Amino acid sequence for Paenibacillus sp. rhamnosidase, Uniparc reference UPI0004F6D660, Uniprot reference A0A089M3T2.
Seq ID No. 1022: Amino acid sequence for Catenovulum agarivorans rhamnosidase, Uniparc reference UPI0003ED860D, Uniprot reference W7QMH5.
Seq ID No. 1023: Amino acid sequence for Zobellia galactanivorans rhamnosidase, Uniparc reference UPI000217D8B1, Uniprot reference G0L382.
Seq ID No. 1024: Amino acid sequence for Bacteroides thetaiotaomicron rhamnosidase, Uniparc reference UPI000005B845, Uniprot reference Q8A1H5.
Seq ID No. 1025: Amino acid sequence for Bacteroides xylanisolvens rhamnosidase, Uniparc reference UPI0001CD02E9, Uniprot reference D6CYE5.
Seq ID No. 1026: Amino acid sequence for Pseudarthrobacter chlorophenolicus rhamnosidase, Uniparc reference UPI00018E07C6, Uniprot reference B8HAH3.
Seq ID No. 1027: Amino acid sequence for Dictyoglomus thermophilum rhamnosidase, Uniparc reference UPI0001815896, Uniprot reference B5YC64.
Seq ID No. 1028: Amino acid sequence for Formosa agariphila rhamnosidase, Uniparc reference UPI00039231C1, Uniprot reference T2KPL4.
Seq ID No. 1029: Amino acid sequence for Rhodococcus jostii rhamnosidase, Uniparc reference UPI0000DBA6EB, Uniprot reference Q0S9T4.
Seq ID No. 1030: Amino acid sequence for Lactobacillus crispatus rhamnosidase, Uniparc reference UPI0001D10896, Uniprot reference D5GZ45.
Seq ID No. 1031: Amino acid sequence for Pedobacter heparinus rhamnosidase, Uniparc reference UPI0001B17DE6, Uniprot reference C6XVU2.
Seq ID No. 1032: Amino acid sequence for Spirosoma linguale rhamnosidase, Uniparc reference UPI0001A3C989, Uniprot reference D2QUA5.
Seq ID No. 1033: Amino acid sequence for Pedobacter heparinus rhamnosidase, Uniparc reference UPI00019EE3EB, Uniprot reference C6XU05.
Seq ID No. 1034: Amino acid sequence for Paenibacillus mucilaginosus rhamnosidase, Uniparc reference UPI0003432C11, Uniprot reference R9ULQ4.
Seq ID No. 1035: Amino acid sequence for Caulobacter segnis rhamnosidase, Uniparc reference UPI0001BC0C05, Uniprot reference D5VGD9.
Seq ID No. 1036: Amino acid sequence for Bacteroides cellulosilyticus rhamnosidase, Uniparc reference UPI0001969377, Uniprot reference E2N9B1.
Seq ID No. 1037: Amino acid sequence for Pedobacter heparinus rhamnosidase, Uniparc reference UPI0001B17C60, Uniprot reference C6Y153.
Seq ID No. 1038: Amino acid sequence for Formosa agariphila rhamnosidase, Uniparc reference UPI000571C0C2, Uniprot reference T2KNB2.
Seq ID No. 1039: Amino acid sequence for Lactobacillus acidophilus rhamnosidase, Uniparc reference UPI00004C6D41, Uniprot reference Q5FJ31.
Seq ID No. 1040: Amino acid sequence for Rhodopirellula baltica rhamnosidase, Uniparc reference UPI00001AC07D, Uniprot reference Q7UYD5.
Seq ID No. 1041: Amino acid sequence for Frankia inefficax rhamnosidase, Uniparc reference UPI0001BF9A6C, Uniprot reference E3IY10.
Seq ID No. 1042: Amino acid sequence for Streptomyces scabiei rhamnosidase, Uniparc reference UPI0001B7FF91, Uniprot reference C9Z376.
Seq ID No. 1043: Amino acid sequence for Flavobacterium johnsoniae rhamnosidase, Uniparc reference UPI00006E5F74, Uniprot reference A5FC22.
Seq ID No. 1044: Amino acid sequence for Streptomyces sp. rhamnosidase, Uniparc reference UPI00034E666D, Uniprot reference S2YWB5.
Seq ID No. 1045: Amino acid sequence for Acidobacterium capsulatum rhamnosidase, Uniparc reference UPI000198DF25, Uniprot reference C1F149.
Seq ID No. 1046: Amino acid sequence for Catenovulum agarivorans rhamnosidase, Uniparc reference UPI0003ED82D3, Uniprot reference W7QYP5.
Seq ID No. 1047: Amino acid sequence for Brachybacterium faecium rhamnosidase, Uniparc reference UPI0001A461B7, Uniprot reference C7MA59.
Seq ID No. 1048: Amino acid sequence for Klebsiella oxytoca rhamnosidase, Uniparc reference UPI000243A177, Uniprot reference A0A0J9X262.
Seq ID No. 1049: Amino acid sequence for Chitinophaga pinensis rhamnosidase, Uniparc reference UPI0001B24769, Uniprot reference C7PA70.
Seq ID No. 1050: Amino acid sequence for Streptomyces bottropensis rhamnosidase, Uniparc reference UPI0002BCAF6B, Uniprot reference M3FYL9.
Seq ID No. 1051: Amino acid sequence for Subdoligranulum variabile rhamnosidase, Uniparc reference UPI0001966B28, Uniprot reference D1PKC7.
Seq ID No. 1052: Amino acid sequence for Microbacterium testaceum rhamnosidase, Uniparc reference UPI0001F8A51D, Uniprot reference E8NDD8.
Seq ID No. 1053: Amino acid sequence for Solibacter usitatus rhamnosidase, Uniparc reference UPI000053767B, Uniprot reference Q01V09.
Seq ID No. 1054: Amino acid sequence for Streptosporangium roseum rhamnosidase, Uniparc reference UPI0001A3EFE6, Uniprot reference D2B240.
Seq ID No. 1055: Amino acid sequence for alpha proteobacterium rhamnosidase, Uniparc reference UPI0006CE1E82, Uniprot reference A0A0N1BME3.
Seq ID No. 1056: Amino acid sequence for Solitalea canadensis rhamnosidase, Uniparc reference UPI000247229E, Uniprot reference H8KPI7.
Seq ID No. 1057: Amino acid sequence for Parabacteroides goldsteinii rhamnosidase, Uniparc reference UPI0002CB9583, Uniprot reference S0GSF0.
Seq ID No. 1058: Amino acid sequence for Cyclobacterium marinum rhamnosidase, Uniparc reference UPI00021B9B33, Uniprot reference G0J630.
Seq ID No. 1059: Amino acid sequence for Solibacter usitatus rhamnosidase, Uniparc reference UPI0000533669, Uniprot reference Q01TX2.
Seq ID No. 1060: Amino acid sequence for Lunatimonas lonarensis rhamnosidase, Uniparc reference UPI00032D8F6D, Uniprot reference R7ZW70.
Seq ID No. 1061: Amino acid sequence for Rhizobium leguminosarum rhamnosidase, Uniparc reference UPI0000D713F2, Uniprot reference Q1M7P3.
Seq ID No. 1062: Amino acid sequence for Streptosporangium roseum rhamnosidase, Uniparc reference UPI0001A3EBEB, Uniprot reference D2AYU9.
Seq ID No. 1063: Amino acid sequence for Parabacteroides distasonis rhamnosidase, Uniparc reference UPI000156F115, Uniprot reference A6LBL4.
Seq ID No. 1064: Amino acid sequence for Lachnospiraceae bacterium rhamnosidase, Uniparc reference UPI0003375A10, Uniprot reference R9K6L6.
Seq ID No. 1065: Amino acid sequence for Chitinophaga pinensis rhamnosidase, Uniparc reference UPI0001A2F0FA, Uniprot reference C7P9Y8.
Seq ID No. 1066: Amino acid sequence for Caulobacter segnis rhamnosidase, Uniparc reference UPI0001B00015, Uniprot reference D5VGC3.
Seq ID No. 1067: Amino acid sequence for Pedobacter heparinus rhamnosidase, Uniparc reference UPI00019EF6E1, Uniprot reference C6Y145.
Seq ID No. 1068: Amino acid sequence for Pedobacter heparinus rhamnosidase, Uniparc reference UPI00019EE1A9, Uniprot reference C6Y2X3.
Seq ID No. 1069: Amino acid sequence for Deltaproteobacteria bacterium rhamnosidase, Uniparc reference UPI000C8D4928, Uniprot reference A0A2D5SK32.
Seq ID No. 1070: Amino acid sequence for Thermobaculum terrenum rhamnosidase, Uniparc reference UPI00019BFDCE, Uniprot reference D1CHL4.
Seq ID No. 1071: Amino acid sequence for Opitutus terrae rhamnosidase, Uniparc reference UPI000172B62A, Uniprot reference B1ZY35.
Seq ID No. 1072: Amino acid sequence for Kribbella flavida rhamnosidase, Uniparc reference UPI00019BFABB, Uniprot reference D2PXQ4.
Seq ID No. 1073: Amino acid sequence for Streptomyces scabiei rhamnosidase, Uniparc reference UPI0001B80091, Uniprot reference C9Z391.
Seq ID No. 1074: Amino acid sequence for Actinoplanes sp. rhamnosidase, Uniparc reference UPI00023EC5D1, Uniprot reference G8S540.
Seq ID No. 1075: Amino acid sequence for Asticcacaulis sp. rhamnosidase, Uniparc reference UPI0003C3CD2B, Uniprot reference V4NSJ1.
Seq ID No. 1076: Amino acid sequence for Kribbella flavida rhamnosidase, Uniparc reference UPI00019BF65D, Uniprot reference D2PT74.
Seq ID No. 1077: Amino acid sequence for Bacillus sp. rhamnosidase, Uniparc reference UPI00000BC760, Uniprot reference Q93RE7.
Seq ID No. 1078: Amino acid sequence for Flavobacterium johnsoniae rhamnosidase, Uniparc reference UPI00006E5FAB, Uniprot reference A5FCG3.
Seq ID No. 1079: Amino acid sequence for Lunatimonas lonarensis rhamnosidase, Uniparc reference UPI00032EEB9C, Uniprot reference R7ZS84.
Seq ID No. 1080: Amino acid sequence for Eisenbergiella massiliensis rhamnosidase, Uniparc reference UPI0004B2D794, Uniprot reference A0A3E3IGR6.
Seq ID No. 1081: Amino acid sequence for Catenovulum agarivorans rhamnosidase, Uniparc reference UPI0003ED7515, Uniprot reference W7QF25.
Seq ID No. 1083: Codon optimised DNA encoding 6×His Rhodothermus marinus rhamnosidase, Uniparc reference UPI0001A31108, Uniprot reference D0MFR0.
Seq ID No. 1084: Codon optimised DNA encoding 6×His Streptomyces bingchenggensis rhamnosidase, Uniparc reference UPI0001D90BFE, Uniprot reference D7C463.
Seq ID No. 1085: Codon optimised DNA encoding 6×His Spirosoma linguale rhamnosidase, Uniparc reference UPI0001A3AEAC, Uniprot reference D2QL60.
Seq ID No. 1086: Codon optimised DNA encoding 6×His Roseburia intestinalis rhamnosidase, Uniparc reference UPI0001CD6D48, Uniprot reference D4L2K8.
Seq ID No. 1087: Codon optimised DNA encoding 6×His Draconibacterium orientale rhamnosidase, Uniparc reference UPI000442EF22, Uniprot reference X5DG83.
Seq ID No. 1088: Codon optimised DNA encoding 6×His Catenulispora acidiphila rhamnosidase, Uniparc reference UPI00019E052B, Uniprot reference C7QC24.
Seq ID No. 1089: Codon optimised DNA encoding 6×His Bacteroides thetaiotaomicron rhamnosidase, Uniparc reference UPI000005ADE1, Uniprot reference Q8A916.
Seq ID No. 1090: Codon optimised DNA encoding 6×His Opitutus terrae rhamnosidase, Uniparc reference UPI000172B2E3, Uniprot reference B1ZRE4.
Seq ID No. 1091: Codon optimised DNA encoding 6×His Lachnoclostridium phytofermentans rhamnosidase, Uniparc reference UPI00015FE0BE, Uniprot reference A9KJ P8.
Seq ID No. 1092: Codon optimised DNA encoding 6×His Rhodanobacter denitrificans rhamnosidase, Uniparc reference UPI00022DA3ED, Uniprot reference M4NH01.
Seq ID No. 1093: Codon optimised DNA encoding 6×His Prevotella ruminicola rhamnosidase, Uniparc reference UPI0001D07633, Uniprot reference D5ETD9.
Seq ID No. 1094: Codon optimised DNA encoding 6×His Aspergillus terreus rhamnosidase, Uniparc reference UPI000259E388, Uniprot reference I0AZ41.
Seq ID No. 1095: Codon optimised DNA encoding 6×His Brachybacterium faecium rhamnosidase, Uniparc reference UPI0001A461B6, Uniprot reference C7MA58.
Seq ID No. 1096: Codon optimised DNA encoding 6×His Flavobacterium johnsoniae rhamnosidase, Uniparc reference UPI00006E4A0D, Uniprot reference A5FCH3.
Seq ID No. 1097: Codon optimised DNA encoding 6×His Rahnella aquatilis rhamnosidase, Uniparc reference UPI000245C507, Uniprot reference H2IYR2.
Seq ID No. 1098: Codon optimised DNA encoding 6×His Bifidobacterium moukalabense rhamnosidase, Uniparc reference UPI0003E4E711, Uniprot reference W4N6H0.
Seq ID No. 1099: Codon optimised DNA encoding 6×His Enterococcus casseliflavus rhamnosidase, Uniparc reference UPI000353BD9F, Uniprot reference S4BBS9.
Seq ID No. 1100: Codon optimised DNA encoding 6×His Geobacillus sp. rhamnosidase, Uniparc reference UPI0001789C0D, Uniprot reference D3EED1.
Seq ID No. 1101: Codon optimised DNA encoding 6×His Modestobacter marinus rhamnosidase, Uniparc reference UPI000260A2FE, Uniprot reference I4EYD9.
Seq ID No. 1102: Codon optimised DNA encoding 6×His Pedobacter heparinus rhamnosidase, Uniparc reference UPI00019EF0EE, Uniprot reference C6XYM6.
Seq ID No. 1103: Codon optimised DNA encoding 6×His Dyadobacter fermentans rhamnosidase, Uniparc reference UPI00019B5915, Uniprot reference C6VZL3.
Seq ID No. 1104: Codon optimised DNA encoding 6×His Paenibacillus mucilaginosus rhamnosidase, Uniparc reference UPI000212C156, Uniprot reference F8FQQ3.
Seq ID No. 1105: Codon optimised DNA encoding 6×His Paenibacillus sp. rhamnosidase, Uniparc reference UPI0003E2544D, Uniprot reference W4D866.
Seq ID No. 1106: Codon optimised DNA encoding 6×His Bacteroides thetaiotaomicron rhamnosidase, Uniparc reference UPI000005ADD2, Uniprot reference A0A0P0FM19.
Seq ID No. 1107: Codon optimised DNA encoding 6×His Chloroflexus aurantiacus rhamnosidase, Uniparc reference UPI00005BA60B, Uniprot reference A9WDK5.
Seq ID No. 1108: Codon optimised DNA encoding 6×His Thermoclostridium stercorarium rhamnosidase, Uniparc reference UPI00000B098C, Uniprot reference Q9S3L0.
Seq ID No. 1109: Codon optimised DNA encoding 6×His Bifidobacterium moukalabense rhamnosidase, Uniparc reference UPI0003E5C314, Uniprot reference W4N6I2.
Seq ID No. 1110: Codon optimised DNA encoding 6×His Olsenella profusa rhamnosidase, Uniparc reference UPI0003AE032C, Uniprot reference U2USP4.
Seq ID No. 1111: Codon optimised DNA encoding 6×His Kribbella flavida rhamnosidase, Uniparc reference UPI00019BDB13, Uniprot reference D2PMT5.
Seq ID No. 1112: Codon optimised DNA encoding 6×His Caulobacter vibrioides rhamnosidase, Uniparc reference UPI00000C7226, Uniprot reference Q9A9K2.
Seq ID No. 1113: Codon optimised DNA encoding 6×His Bacteroides thetaiotaomicron rhamnosidase, Uniparc reference UPI000005BA09, Uniprot reference Q8A076.
Seq ID No. 1114: Codon optimised DNA encoding 6×His Rhodonellum psychrophilum rhamnosidase, Uniparc reference UPI0003745394, Uniprot reference U5BUY4.
Seq ID No. 1115: Codon optimised DNA encoding 6×His Paenibacillus sp. rhamnosidase, Uniparc reference UPI0004F6D660, Uniprot reference A0A089M3T2.
Seq ID No. 1116: Codon optimised DNA encoding 6×His Catenovulum agarivorans rhamnosidase, Uniparc reference UPI0003ED860D, Uniprot reference W7QMH5.
Seq ID No. 1117: Codon optimised DNA encoding 6×His Zobellia galactanivorans rhamnosidase, Uniparc reference UPI000217D8B1, Uniprot reference G0L382.
Seq ID No. 1118: Codon optimised DNA encoding 6×His Bacteroides thetaiotaomicron rhamnosidase, Uniparc reference UPI000005B845, Uniprot reference Q8A1H5.
Seq ID No. 1119: Codon optimised DNA encoding 6×His Bacteroides xylanisolvens rhamnosidase, Uniparc reference UPI0001CD02E9, Uniprot reference D6CYE5.
Seq ID No. 1120: Codon optimised DNA encoding 6×His Pseudarthrobacter chlorophenolicus rhamnosidase, Uniparc reference UPI00018E07C6, Uniprot reference B8HAH3.
Seq ID No. 1121: Codon optimised DNA encoding 6×His Dictyoglomus thermophilum rhamnosidase, Uniparc reference UPI0001815896, Uniprot reference B5YC64.
Seq ID No. 1122: Codon optimised DNA encoding 6×His Formosa agariphila rhamnosidase, Uniparc reference UPI00039231C1, Uniprot reference T2KPL4.
Seq ID No. 1123: Codon optimised DNA encoding 6×His Rhodococcus jostii rhamnosidase, Uniparc reference UPI0000DBA6EB, Uniprot reference Q0S9T4.
Seq ID No. 1124: Codon optimised DNA encoding 6×His Lactobacillus crispatus rhamnosidase, Uniparc reference UPI0001D10896, Uniprot reference D5GZ45.
Seq ID No. 1125: Codon optimised DNA encoding 6×His Pedobacter heparinus rhamnosidase, Uniparc reference UPI0001B17DE6, Uniprot reference C6XVU2.
Seq ID No. 1126: Codon optimised DNA encoding 6×His Spirosoma linguale rhamnosidase, Uniparc reference UPI0001A3C989, Uniprot reference D2QUA5.
Seq ID No. 1127: Codon optimised DNA encoding 6×His Pedobacter heparinus rhamnosidase, Uniparc reference UPI00019EE3EB, Uniprot reference C6XU05.
Seq ID No. 1128: Codon optimised DNA encoding 6×His Paenibacillus mucilaginosus rhamnosidase, Uniparc reference UPI0003432C11, Uniprot reference R9ULQ4.
Seq ID No. 1129: Codon optimised DNA encoding 6×His Caulobacter segnis rhamnosidase, Uniparc reference UPI0001BC0C05, Uniprot reference D5VGD9.
Seq ID No. 1130: Codon optimised DNA encoding 6×His Bacteroides cellulosilyticus rhamnosidase, Uniparc reference UPI0001969377, Uniprot reference E2N9B1.
Seq ID No. 1131: Codon optimised DNA encoding 6×His Pedobacter heparinus rhamnosidase, Uniparc reference UPI0001B17C60, Uniprot reference C6Y153.
Seq ID No. 1132: Codon optimised DNA encoding 6×His Formosa agariphila rhamnosidase, Uniparc reference UPI000571C0C2, Uniprot reference T2KNB2.
Seq ID No. 1133: Codon optimised DNA encoding 6×His Lactobacillus acidophilus rhamnosidase, Uniparc reference UPI00004C6D41, Uniprot reference Q5FJ31.
Seq ID No. 1134: Codon optimised DNA encoding 6×His Rhodopirellula baltica rhamnosidase, Uniparc reference UPI00001AC07D, Uniprot reference Q7UYD5.
Seq ID No. 1135: Codon optimised DNA encoding 6×His Frankia inefficax rhamnosidase, Uniparc reference UPI0001BF9A6C, Uniprot reference E3IY10.
Seq ID No. 1136: Codon optimised DNA encoding 6×His Streptomyces scabiei rhamnosidase, Uniparc reference UPI0001B7FF91, Uniprot reference C9Z376.
Seq ID No. 1137: Codon optimised DNA encoding 6×His Flavobacterium johnsoniae rhamnosidase, Uniparc reference UPI00006E5F74, Uniprot reference A5FC22.
Seq ID No. 1138: Codon optimised DNA encoding 6×His Streptomyces sp. rhamnosidase, Uniparc reference UPI00034E666D, Uniprot reference S2YWB5.
Seq ID No. 1139: Codon optimised DNA encoding 6×His Acidobacterium capsulatum rhamnosidase, Uniparc reference UPI000198DF25, Uniprot reference C1F149.
Seq ID No. 1140: Codon optimised DNA encoding 6×His Catenovulum agarivorans rhamnosidase, Uniparc reference UPI0003ED82D3, Uniprot reference W7QYP5.
Seq ID No. 1141: Codon optimised DNA encoding 6×His Brachybacterium faecium rhamnosidase, Uniparc reference UPI0001A461B7, Uniprot reference C7MA59.
Seq ID No. 1142: Codon optimised DNA encoding 6×His Klebsiella oxytoca rhamnosidase, Uniparc reference UPI000243A177, Uniprot reference A0A0J9X262.
Seq ID No. 1143: Codon optimised DNA encoding 6×His Chitinophaga pinensis rhamnosidase, Uniparc reference UPI0001B24769, Uniprot reference C7PA70.
Seq ID No. 1144: Codon optimised DNA encoding 6×His Streptomyces bottropensis rhamnosidase, Uniparc reference UPI0002BCAF6B, Uniprot reference M3FYL9.
Seq ID No. 1145: Codon optimised DNA encoding 6×His Subdoligranulum variabile rhamnosidase, Uniparc reference UPI0001966B28, Uniprot reference D1PKC7.
Seq ID No. 1146: Codon optimised DNA encoding 6×His Microbacterium testaceum rhamnosidase, Uniparc reference UPI0001F8A51D, Uniprot reference E8NDD8.
Seq ID No. 1147: Codon optimised DNA encoding 6×His Solibacter usitatus rhamnosidase, Uniparc reference UPI000053767B, Uniprot reference Q01V09.
Seq ID No. 1148: Codon optimised DNA encoding 6×His Streptosporangium roseum rhamnosidase, Uniparc reference UPI0001A3EFE6, Uniprot reference D2B240.
Seq ID No. 1149: Codon optimised DNA encoding 6×His alpha proteobacterium rhamnosidase, Uniparc reference UPI0006CE1E82, Uniprot reference A0A0N1BME3.
Seq ID No. 1150: Codon optimised DNA encoding 6×His Solitalea canadensis rhamnosidase, Uniparc reference UPI000247229E, Uniprot reference H8KPI7.
Seq ID No. 1151: Codon optimised DNA encoding 6×His Parabacteroides goldsteinii rhamnosidase, Uniparc reference UPI0002CB9583, Uniprot reference S0GSF0.
Seq ID No. 1152: Codon optimised DNA encoding 6×His Cyclobacterium marinum rhamnosidase, Uniparc reference UPI00021B9B33, Uniprot reference G0J630.
Seq ID No. 1153: Codon optimised DNA encoding 6×His Solibacter usitatus rhamnosidase, Uniparc reference UPI0000533669, Uniprot reference Q01TX2.
Seq ID No. 1154: Codon optimised DNA encoding 6×His Lunatimonas lonarensis rhamnosidase, Uniparc reference UPI00032D8F6D, Uniprot reference R7ZW70.
Seq ID No. 1155: Codon optimised DNA encoding 6×His Rhizobium leguminosarum rhamnosidase, Uniparc reference UPI0000D713F2, Uniprot reference Q1M7P3.
Seq ID No. 1156: Codon optimised DNA encoding 6×His Streptosporangium roseum rhamnosidase, Uniparc reference UPI0001A3EBEB, Uniprot reference D2AYU9.
Seq ID No. 1157: Codon optimised DNA encoding 6×His Parabacteroides distasonis rhamnosidase, Uniparc reference UPI000156F115, Uniprot reference A6LBL4.
Seq ID No. 1158: Codon optimised DNA encoding 6×His Lachnospiraceae bacterium rhamnosidase, Uniparc reference UPI0003375A10, Uniprot reference R9K6L6.
Seq ID No. 1159: Codon optimised DNA encoding 6×His Chitinophaga pinensis rhamnosidase, Uniparc reference UPI0001A2F0FA, Uniprot reference C7P9Y8.
Seq ID No. 1160: Codon optimised DNA encoding 6×His Caulobacter segnis rhamnosidase, Uniparc reference UPI0001BC0C15, Uniprot reference D5VGC3.
Seq ID No. 1161: Codon optimised DNA encoding 6×His Pedobacter heparinus rhamnosidase, Uniparc reference UPI00019EF6E1, Uniprot reference C6Y145.
Seq ID No. 1162: Codon optimised DNA encoding 6×His Pedobacter heparinus rhamnosidase, Uniparc reference UPI00019EE1A9, Uniprot reference C6Y2X3.
Seq ID No. 1163: Codon optimised DNA encoding 6×His Deltaproteobacteria bacterium rhamnosidase, Uniparc reference UPI000C8D4928, Uniprot reference A0A2D5SK32.
Seq ID No. 1164: Codon optimised DNA encoding 6×His Thermobaculum terrenum rhamnosidase, Uniparc reference UPI00019BFDCE, Uniprot reference D1CHL4.
Seq ID No. 1165: Codon optimised DNA encoding 6×His Opitutus terrae rhamnosidase, Uniparc reference UPI000172B62A, Uniprot reference B1ZY35.
Seq ID No. 1166: Codon optimised DNA encoding 6×His Kribbella flavida rhamnosidase, Uniparc reference UPI00019BFABB, Uniprot reference D2PXQ4.
Seq ID No. 1167: Codon optimised DNA encoding 6×His Streptomyces scabiei rhamnosidase, Uniparc reference UPI0001B80091, Uniprot reference C9Z391.
Seq ID No. 1168: Codon optimised DNA encoding 6×His Actinoplanes sp. rhamnosidase, Uniparc reference UPI00023EC5D1, Uniprot reference G8S540.
Seq ID No. 1169: Codon optimised DNA encoding 6×His Asticcacaulis sp. rhamnosidase, Uniparc reference UPI0003C3CD2B, Uniprot reference V4NSJ1.
Seq ID No. 1170: Codon optimised DNA encoding 6×His Kribbella flavida rhamnosidase, Uniparc reference UPI00019BF65D, Uniprot reference D2PT74.
Seq ID No. 1171: Codon optimised DNA encoding 6×His Bacillus sp. rhamnosidase, Uniparc reference UPI00000BC760, Uniprot reference Q93RE7.
Seq ID No. 1172: Codon optimised DNA encoding 6×His Flavobacterium johnsoniae rhamnosidase, Uniparc reference UPI00006E5FAB, Uniprot reference A5FCG3.
Seq ID No. 1173: Codon optimised DNA encoding 6×His Lunatimonas lonarensis rhamnosidase, Uniparc reference UPI00032EEB9C, Uniprot reference R7ZS84.
Seq ID No. 1174: Codon optimised DNA encoding 6×His Eisenbergiella massiliensis rhamnosidase, Uniparc reference UPI0004B2D794, Uniprot reference A0A3E3IGR6.
Seq ID No. 1175: Codon optimised DNA encoding 6×His Catenovulum agarivorans rhamnosidase, Uniparc reference UPI0003ED7515, Uniprot reference W7QF25.
Seq ID No. 1176: Codon optimised DNA encoding 6×His Streptomyces avermitilis rhamnosidase, Uniparc reference UPI0000184198, Uniprot reference Q82PP4.
Seq ID No. 1177: N-terminal His-tag/linker
Seq ID No. 1178: C-terminal linker/His tag
Seq ID No. 1179: Amino acid sequence for Variant G1.
Seq ID No. 1180: Amino acid sequence for Variant G2.
Seq ID No. 1181: Amino acid sequence for Variant G3.
Seq ID No. 1182: Amino acid sequence for Variant G4.
Seq ID No. 1183: Amino acid sequence for Variant G5.
Seq ID No. 1184: Codon optimised DNA encoding N-terminal 6×His Variant G1.
Seq ID No. 1185: Codon optimised DNA encoding N-terminal 6×His Variant G2.
Seq ID No. 1186: Codon optimised DNA encoding N-terminal 6×His Variant G3.
Seq ID No. 1187: Codon optimised DNA encoding N-terminal 6×His Variant G4.
Seq ID No. 1188: Codon optimised DNA encoding N-terminal 6×His Variant G5.
Seq ID No. 1189: Amino acid sequence for Variant R1.
Seq ID No. 1190: Amino acid sequence for Variant R2.
Seq ID No. 1191: Amino acid sequence for Variant R3.
Seq ID No. 1192: Amino acid sequence for Variant R4.
Seq ID No. 1193: Amino acid sequence for Variant R5.
Seq ID No. 1194: Codon optimised DNA encoding C-terminal 6×His Variant R1.
Seq ID No. 1195: Codon optimised DNA encoding C-terminal 6×His Variant R2.
Seq ID No. 1196: Codon optimised DNA encoding C-terminal 6×His Variant R3.
Seq ID No. 1197: Codon optimised DNA encoding C-terminal 6×His Variant R4.
Seq ID No. 1198: Codon optimised DNA encoding C-terminal 6×His Variant R5.

### DETAILED DESCRIPTION OF THE INVENTION

As mentioned previously, saponins are steroid or terpenoid glycosides which have a broad range of uses. Current approaches to obtaining certain saponins in suitable quantities and of suitable purities are limiting. The present inventors have surprisingly found that enzymatic modification of saponins can facilitate improved availability of saponins of interest and/or facilitate removal of undesired saponin components. The present invention therefore provides methods for the enzymatic modification of saponins, products made by such methods, uses of said products and associated aspects. In methods of the invention a starting saponin (i.e. a saponin to be modified by an enzyme) is converted into a product saponin (i.e. the saponin resulting from enzymatic modification of the starting saponin).

Engineered glucosidase polypeptides of the present invention may be used in methods for the enzymatic modification of saponins.

Engineered rhamnosidase polypeptides of the present invention may be used in methods for the enzymatic modification of saponins.

### Objectives

The present invention can be applied to achieve a plurality of objectives, such as: (i) improving the yield of saponins of interest obtainable from a given starting material; (ii) broadening the range of starting materials suitable for obtaining saponins of interest; and/or (iii) convenient removal of undesired saponins from saponins of interest.

Where supply is constrained for a starting material from which a saponin of interest is isolated, achieving the maximum yield of the saponin of interest is clearly important. Independent of the efficiency of extraction and separation processes which would generally be adapted for optimal isolation of existing saponin of interest, the present invention may be applied to increase the amount of a saponin of interest which may be obtained from a given starting material. Enzymatic modification of other saponins present in the starting material to form a saponin of interest can increase the amount of the saponin of interest which may be obtained.

Saponins may be obtained from a broad range of starting materials. The presence of specific saponins and their levels in plant material may depend on a range of factors such as a plant species, tissue, age, season, environmental conditions and the like. Variation may be observed between plants (such as trees) of the same species (see, for example, WO2018057031). The burden associated with extraction and/or isolation of a saponin of interest may mean that certain potential sources of the saponin of interest are not commercially viable, due to the saponin of interest being present at relatively low levels. Enzymatic modification of other saponins present in the starting material to form a saponin of interest can expand the range of viable starting materials for obtaining the saponin of interest.

It is well understood that different saponins may have different activity profiles—both positive/desired activities and negative/undesired activities. Some uses of saponins require a high degree of purification and separating a saponin of interest from other saponins, particularly those of similar structure or physical properties, can be burdensome. Enzymatic modification of such other saponins may alter their physical properties and may thereby facilitate separation from a saponin of interest. Other uses of saponins may not require a high degree of purity per se, nevertheless it may still be desirable to remove or reduce the amount of a particular saponin component (or components) within a saponin mixture without burdensome chromatographic methods. Enzymatic modification can facilitate removal or reduction in the level of a particular saponin component within a saponin mixture without the need for chromatographic means.

### Saponins

The methods of the present invention require a starting saponin (i.e. a saponin which is intended to be enzymatically modified). The starting saponin may be a naturally occurring saponin (i.e. a steroid or terpenoid glycoside found in nature) or an artificially created saponin (i.e. a steroid or terpenoid glycoside not found in nature).

In some embodiments the starting saponin is a steroid glycoside, in other embodiments the starting saponin is a terpenoid glycoside, especially a triterpenoid glycoside.

Naturally occurring starting saponins may be obtained by extraction or may be prepared synthetically (fully or semi-synthetically).

Naturally occurring starting saponins include those obtainable from, such as obtained from, plants of the genera Gypsophilia, Saponaria or Quillaja (Bomford, 1992). Especially of interest are starting saponins obtainable from plants of Quillaja species. Particular starting saponins of interest include those obtainable from Quillaja brasiliensis or Quillaja saponaria. In one embodiment the starting saponin is obtainable from Quillaja saponaria, such as obtained from Quillaja saponaria. In one embodiment the starting saponin is obtainable from Quillaja brasiliensis, such as obtained from Quillaja brasiliensis.

In certain embodiments the starting saponin is a quillaic acid glycoside. In certain embodiments the starting saponin is a phytolaccinic acid glycoside. In certain embodiments the starting saponin is an echinocystic acid glycoside. In certain embodiments the starting saponin is a 22-beta-hydroxylated quillaic acid glycoside. In certain embodiments the starting saponin is an gypsogenin glycoside.

Analysis of water/methanol extracts of Quillaja saponaria bark by liquid chromatography/mass spectrometry has revealed over 100 saponins (Nyberg, 2000; Nyberg, 2003; Kite, 2004). Quillaja brasiliensis extracts have also been described, with many saponin components in Quillaja brasiliensis extracts corresponding to saponins found in Quillaja saponaria extracts. (Wallace, 2017; Wallace, 2019).

The following text describes particular quillaic acid derived starting and product saponins which are grouped by ‘family’. Each family has one or more common structural features which characterise the family relative to other families. Individual components within each family also display certain structural features which characterise the component relative to other components of the family, including: xylose or rhamnose chemotype—the presence of a xylose or rhamnose residue in the C3 saccharide; A or B isomers—A having the acyl chain linked through the 4-position of the D-fucose, B at having the acyl chain linked through the 3-position of the D-fucose; V1 and V2—the presence of a terminal apiose or xylose residue in the C28 saccharide (in other components of a family this terminal residue may also be absent). The text focuses on components which typically have a significant presence in Quillaja saponaria aqueous extracts, but it will be appreciated that (i) other components of a family also exist and (ii) the proportions of different components of a family may vary both between families and between different saponin sources (Kite, 2004). The specific extraction method used may also influence the proportions of different components obtained.

A and B isomers may be separable using chromatographic techniques. However, under suitable solvent conditions these isomers will revert to equilibrium proportions (see e.g. Cleland, 1996). Xylose and rhamnose chemotypes typically elute closely, depending on chromatographic technique the rhamnose chemotype may form a minor peak closely preceding or overlapping with the main peak for the family.

Those skilled in the art will also recognise that the structures described contain ionisable groups and under appropriate circumstances may exist in dissociated forms or as salts. Structures are generally shown with the glucuronate moiety in ionised form and the indicated molecular weight is calculated directly from the ion shown (corresponding to the monoisotopic m/z observed with negative mode electrospray mass spectrometry), however, all non-dissociated, dissociated and salt forms are intended to be encompassed by the recited definitions. Salts are desirably pharmaceutically acceptable, although non-pharmaceutically acceptable salts can nevertheless be useful during manufacture of pharmaceuticals or for non-pharmaceutical uses.

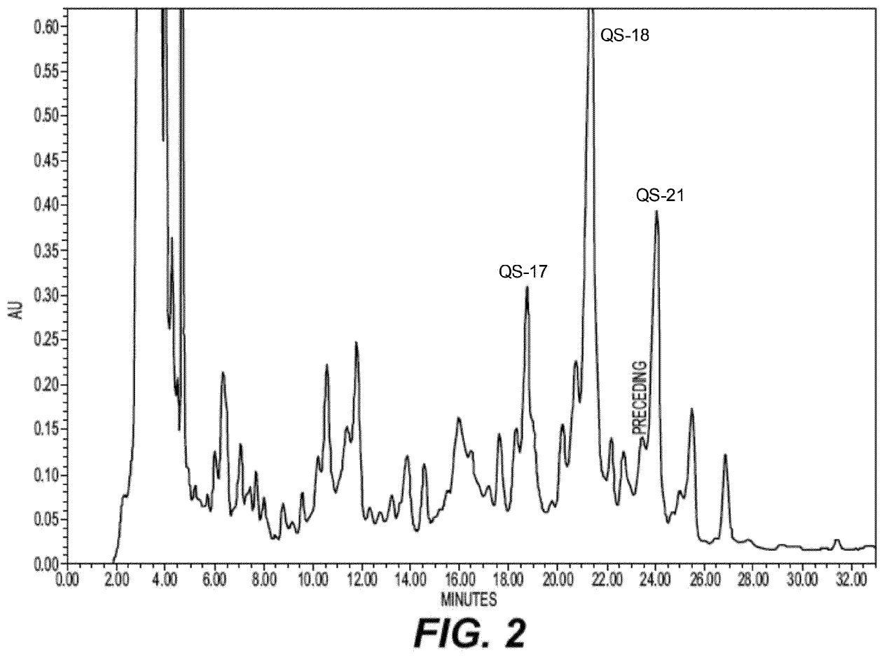

Starting saponins obtainable from Quillaja saponaria include:
QS-18 family components (i.e. triterpenoid glycosides having beta-O-glucopyranosylation at the C3 position of the L-rhamnose moiety of QS-21 family components), such as:
   ‘QS-18 2150 A component’, being the triterpenoid glycosides identified as part of the QS-18 main peak in FIG. 2 and having a m/z of 2150 with negative mode electrospray mass spectrometry. The QS-18 2150 A component is believed to be identified in Kite 2004 as Peak 76 and corresponds to the A-isomer xylose chemotype structures B4 (apiose isomer) and B6 (xylose isomer) characterised in Nyberg 2000 and Nyberg 2003. The QS-18 2150 A component may consist of QS-18 2150 A V1 (i.e. apiose isomer):

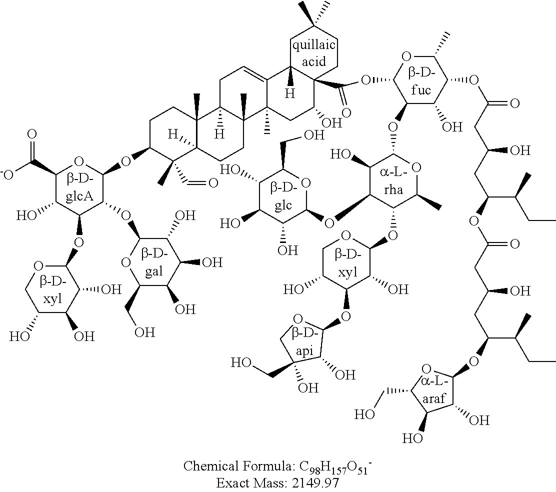

and/or QS-18 2150 A V2 (i.e. xylose isomer):

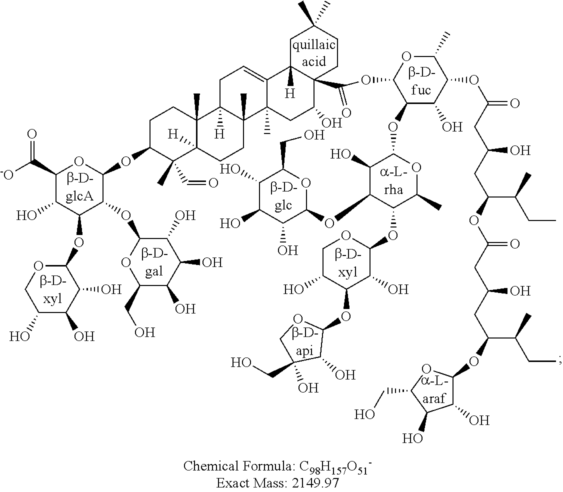

‘QS-18 2018 A component’, being the triterpenoid glycosides identified as part of the QS-18 main peak in FIG. 2 and having m/z of 2018 with negative mode electrospray mass spectrometry. The QS-18 A 2018 component is believed to be identified in Kite 2004 as Peak 73 and corresponds to the A-isomer xylose chemotype structure B2 characterised in Nyberg 2000 and Nyberg 2003. The QS-18 2018 A component may consist of QS-18 2018 A:

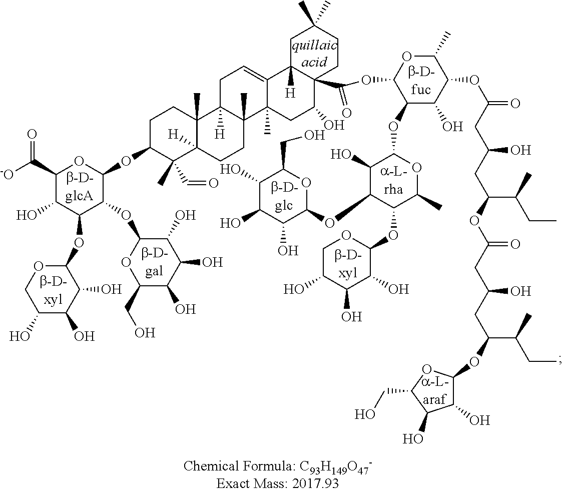

‘QS-18 2164 A component’, being the triterpenoid glycosides identified as part of the QS-18 main peak in FIG. 2 and having a m/z of 2164 with negative mode electrospray mass spectrometry. The QS-18 2164 A component is believed to be identified in Kite 2004 as Peak 74 and corresponds to the A-isomer rhamnose chemotype structures B3 (apiose isomer) and B5 (xylose isomer) characterised in Nyberg 2000 and Nyberg 2003. The QS-18 2164 A component may consist of QS-18 2164 A V1 (i.e. apiose isomer):

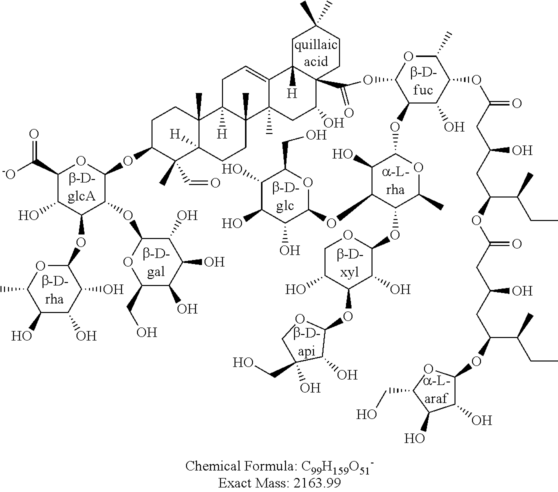

and/or QS-18 2164 A V2 (i.e. xylose isomer):

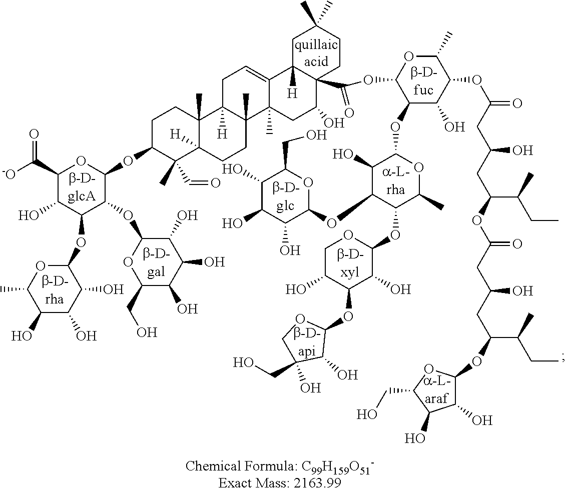

‘QS-18 2150 B component’, being triterpenoid glycosides having a m/z of 2150 with negative mode electrospray mass spectrometry. The QS-18 2150 B component corresponds to the B-isomer xylose chemotype structures B4a (apiose isomer) and B6a (xylose isomer) characterised in Nyberg 2000 and Nyberg 2003. The QS-18 2150 B component may consist of QS-18 2150 B V1 (i.e. apiose isomer):

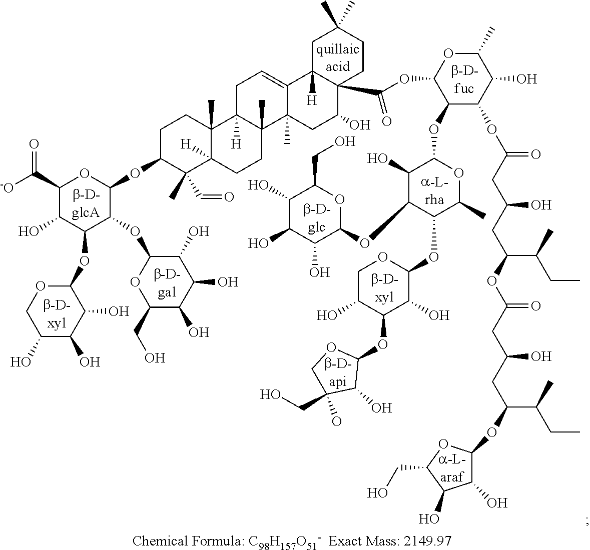

and/or QS-18 2150 B V2 (i.e. xylose isomer):

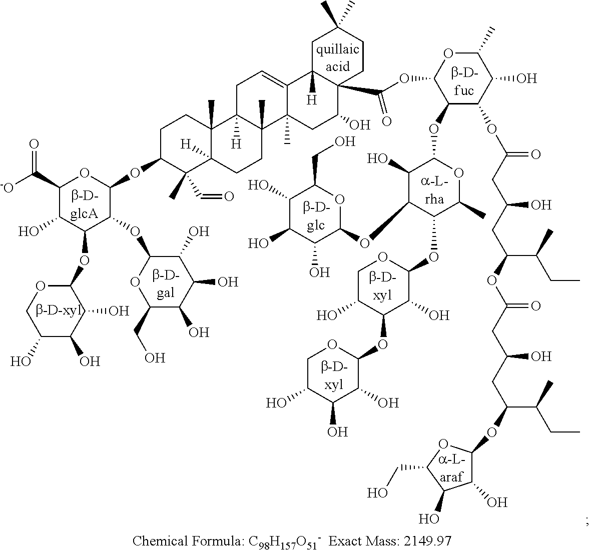

‘QS-18 2018 B component’, being triterpenoid glycosides having a m/z of 2018 with negative mode electrospray mass spectrometry. The QS-18 2018 B component corresponds to the B-isomer xylose chemotype structure B2a characterised in Nyberg 2000 and Nyberg 2003. The QS-18 2018 B component may consist of QS-18 2018 B:

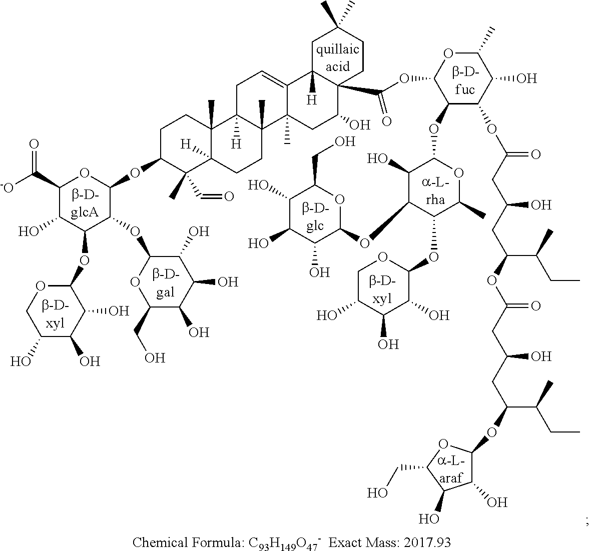

‘QS-18 2164 B component’, being triterpenoid glycosides having a m/z of 2164 with negative mode electrospray mass spectrometry. The QS-18 2164 B corresponds to the B-isomer rhamnose chemotype structures B3a (apiose isomer) and B5a (xylose isomer). The QS-18 2164 B component may consist of QS-18 2164 B V1 (i.e. apiose isomer):

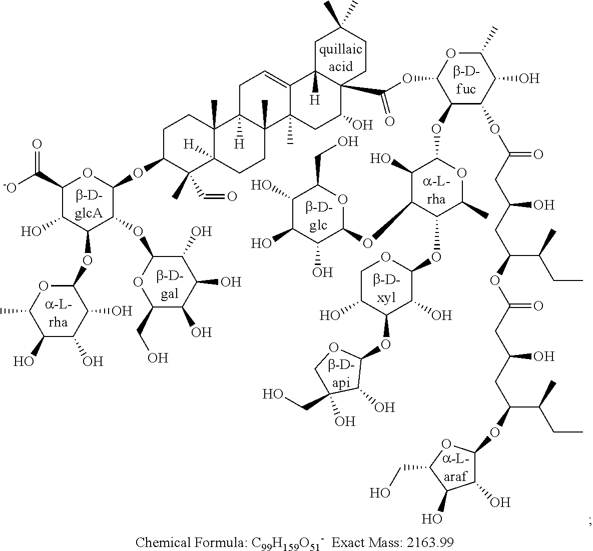

and/or QS-18 2164 B V2 (i.e. xylose isomer):

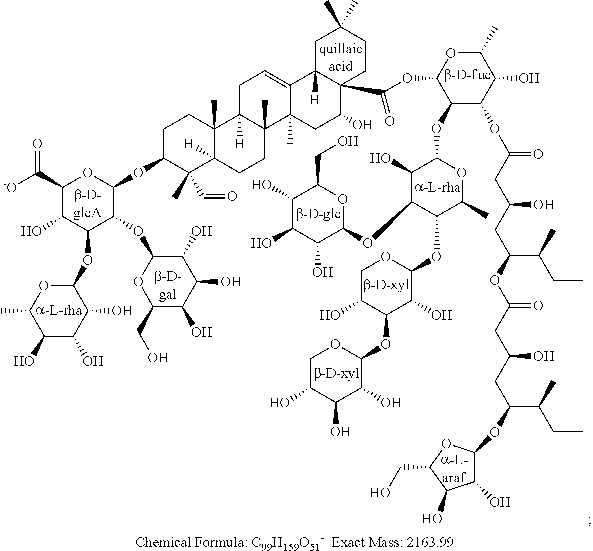

desglucosyl-QS-17 family components (i.e. triterpenoid glycosides having alpha-O-rhamnosylation at the C2 position of the arabinofuranose moiety of QS-21 family components but lacking the glycosylation of QS-17 family components), such as:
   ‘desglucosyl-QS-17 2134 A component’, being triterpenoid glycosides having a m/z of 2134 with negative mode electrospray mass spectrometry. The desglucosyl-QS-17 2134 A component is believed to be identified in Kite 2004 as Peak 75 and corresponds to A-isomers of the xylose chemotype. The desglucosyl-QS-17 2134 A component may consist of desglucosyl-QS-17 2134 A V1 (i.e. apiose isomer):

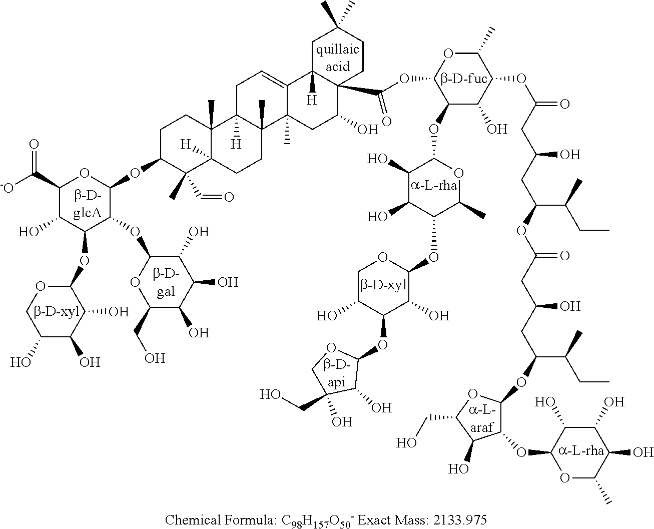

and/or desglucosyl-QS-17 2134 A V2 (i.e. xylose isomer):

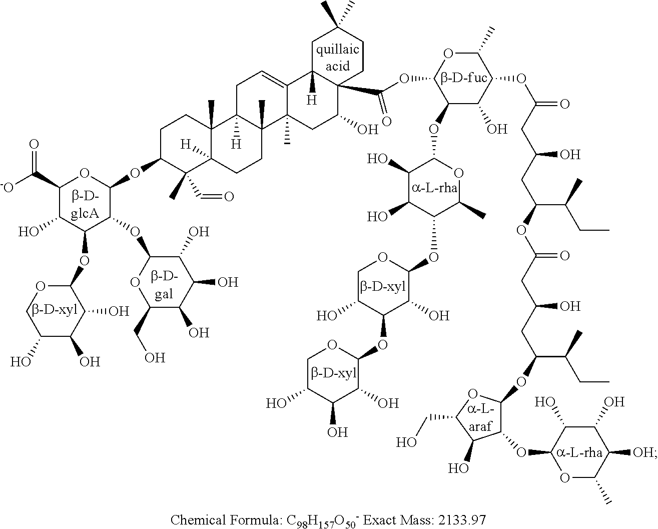

‘desglucosyl-QS-17 2002 A component’, being triterpenoid glycosides having a m/z of 2002 with negative mode electrospray mass spectrometry. The desglucosyl-QS-17 2002 A component corresponds to the A-isomer xylose chemotype and may consist of desglucosyl-QS-17 2002 A:

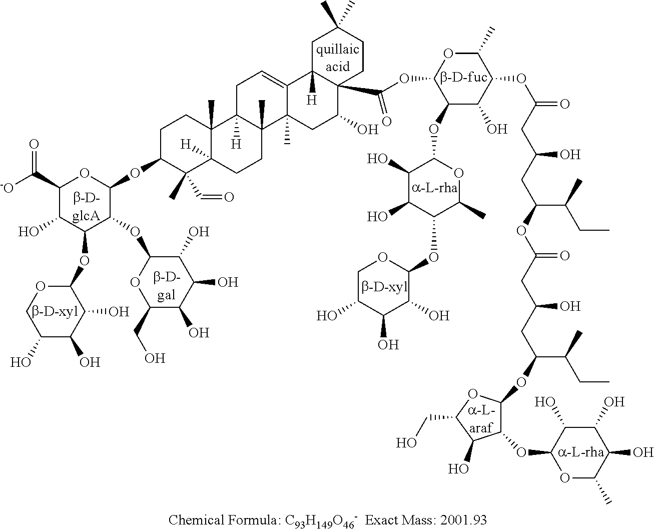

‘desglucosyl-QS-17 2148 A component’, being triterpenoid glycosides having a m/z of 2148 with negative mode electrospray mass spectrometry. The desglucosyl-QS-17 2148 A component is believed to be identified in Kite 2004 as Peaks 70 and 72 and corresponds to the A-isomer rhamnose chemotype. The desglucosyl-QS-17 2148 A component may consist of desglucosyl-QS-17 2148 A V1 (i.e. apiose isomer):

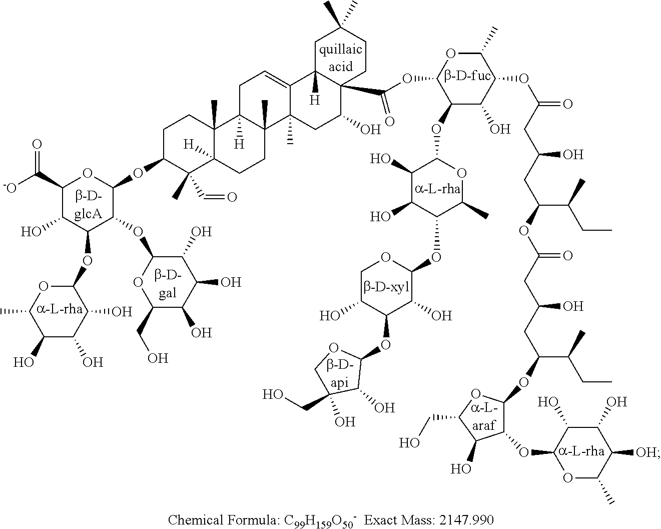

and/or desglucosyl-QS-17 2148 A V2 (i.e. xylose isomer):

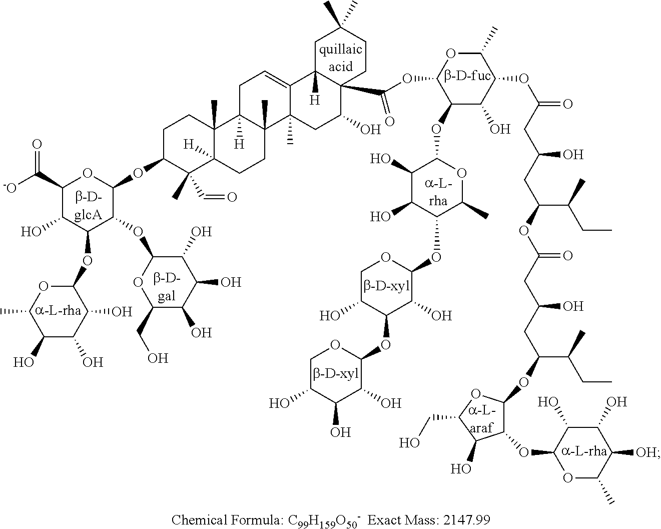

‘desglucosyl-QS-17 2134 B component’, being triterpenoid glycosides having a m/z of 2134 with negative mode electrospray mass spectrometry. The desglucosyl-QS-17 2134 B component is believed to be identified in Kite 2004 as Peak 67 and corresponds to B-isomers of the xylose chemotype. The desglucosyl-QS-17 2134 B component may consist of desglucosyl-QS-17 2134 B V1 (i.e. apiose isomer):

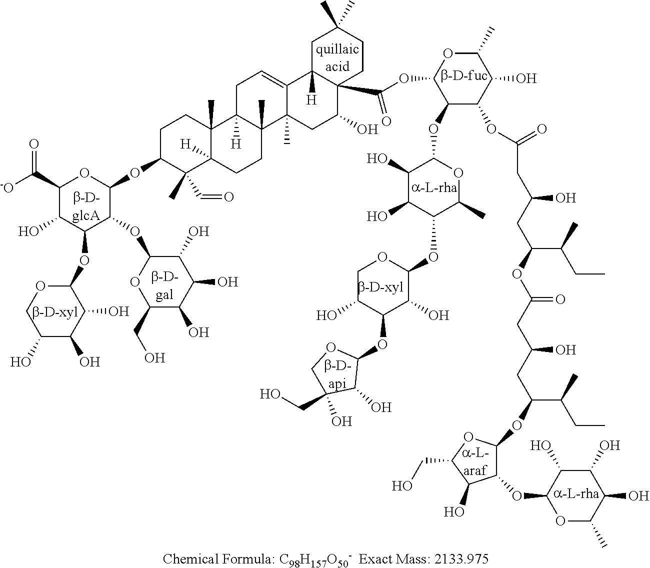

and/or desglucosyl-QS-17 2134 B V2 (i.e. xylose isomer):

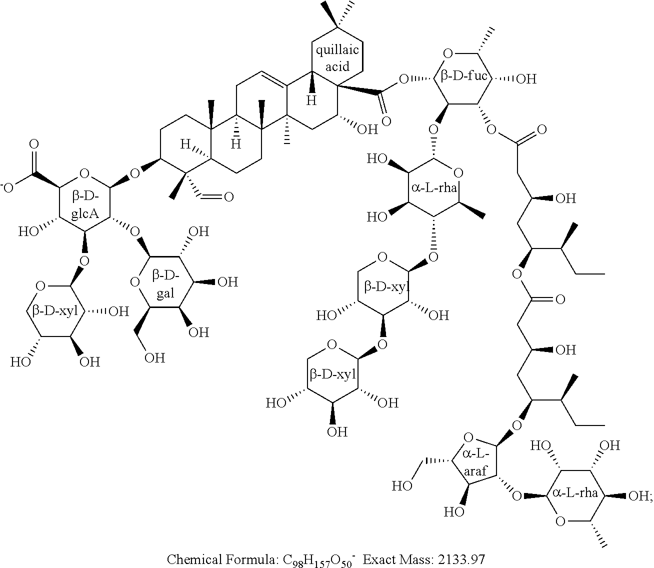

‘desglucosyl-QS-17 2002 B component’, being triterpenoid glycosides having a m/z of 2002 with negative mode electrospray mass spectrometry. The desglucosyl-QS-17 2002 B component corresponds to the B-isomer of the xylose chemotype and may consist of desglucosyl-QS-17 2002 B:

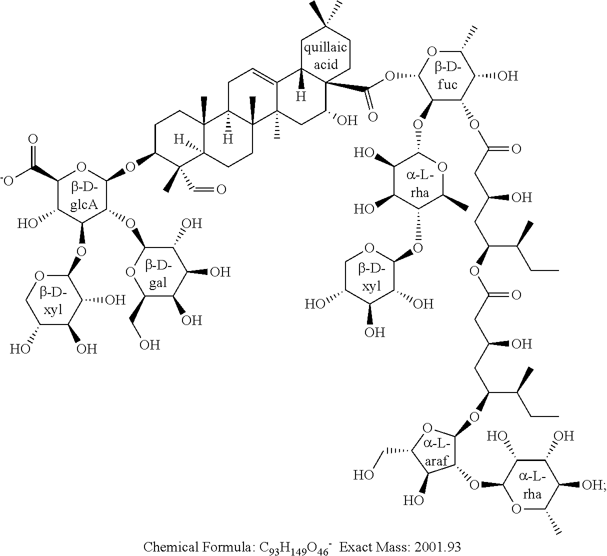

‘desglucosyl-QS-17 2148 B component’, being triterpenoid glycosides having a m/z of 2148 with negative mode electrospray mass spectrometry. The desglucosyl-QS-17 2148 B component is believed to be identified in Kite 2004 as Peak 65 and corresponds to B-isomers of the rhamnose chemotype. The desglucosyl-QS-17 2148 B component may consist of desglucosyl-QS-17 2148 B V1 (i.e. apiose isomer):

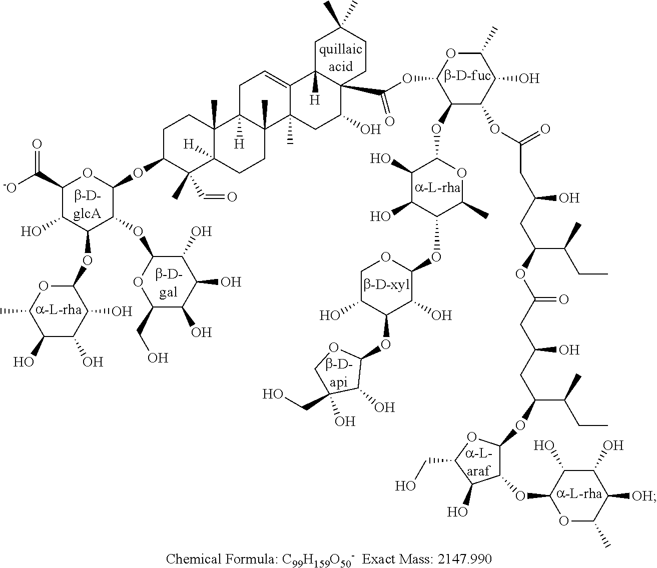

and/or desglucosyl-QS-17 2148 B V2 (i.e. xylose isomer):

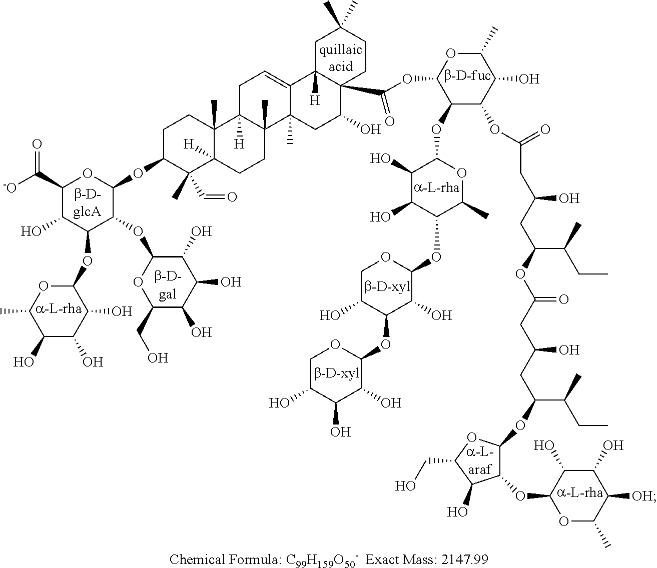

QS-17 family components (i.e. triterpenoid glycosides having beta-O-glucopyranosylation at the C3 position of the L-rhamnose moiety and alpha-O-rhamnosylation at the C2 position of the arabinofuranose moiety of QS-21 family components), such as:
   ‘QS-17 2296 A component’, being the triterpenoid glycosides identified as part of the QS-17 main peak in FIG. 2 and having a m/z of 2296 with negative mode electrospray mass spectrometry. The QS-17 2296 A component is believed to be identified in Kite 2004 as Peak 59 and corresponds to the A-isomers of xylose chemotype structure QS-Ill. The QS-17 2296 A component may consist of QS-17 2296 A V1 (i.e. apiose isomer):

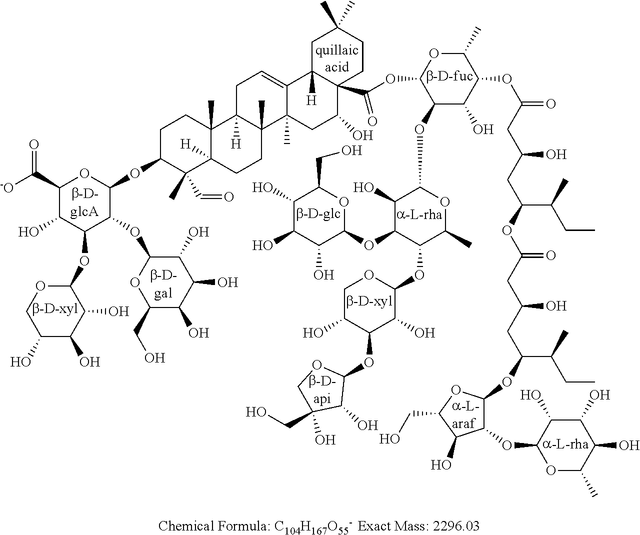

and/or QS-17 2296 A V2 (i.e. xylose isomer):

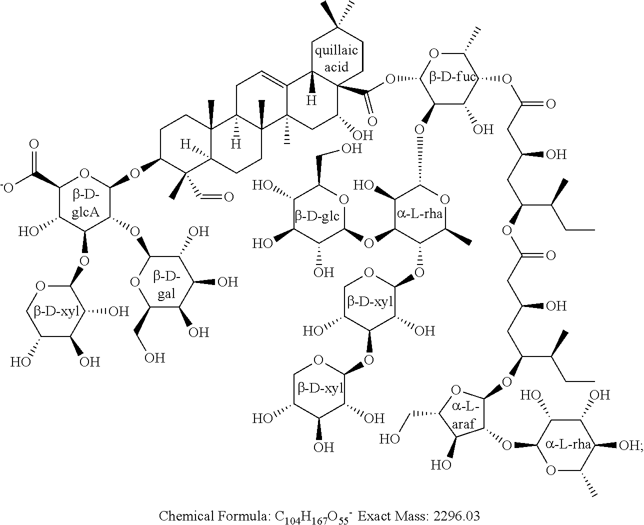

‘QS-17 2164 A component’, being the triterpenoid glycosides identified as part of the QS-17 main peak in FIG. 2 and having a m/z of 2164 with negative mode electrospray mass spectrometry. The QS-17 2164 A component is believed to be identified in Kite 2004 as Peak 58 and corresponds to the A-isomer xylose chemotype. The QS-17 2164 A component may consist of QS-17 2164 A:

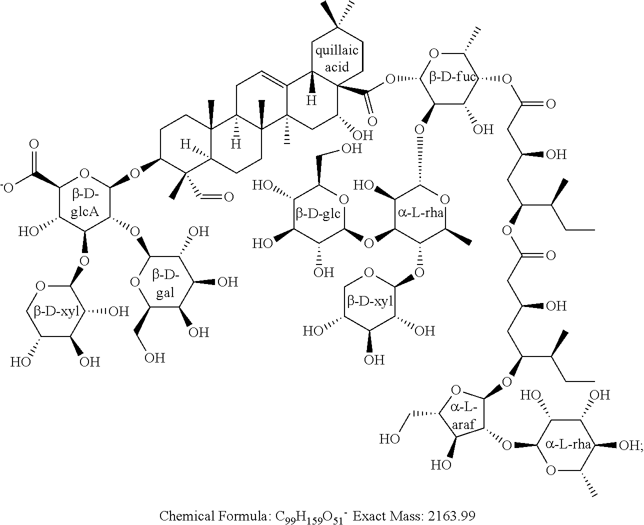

‘QS-17 2310 A component’, being the triterpenoid glycosides identified as part of the QS-17 main peak in FIG. 2 and having a m/z of 2310 with negative mode electrospray mass spectrometry. The QS-17 2310 A component is believed to be identified in Kite 2004 as Peak 57 and corresponds to A-isomers of the rhamnose chemotype. The QS-17 2310 A component may consist of QS-17 2310 A V1 (i.e. apiose isomer):

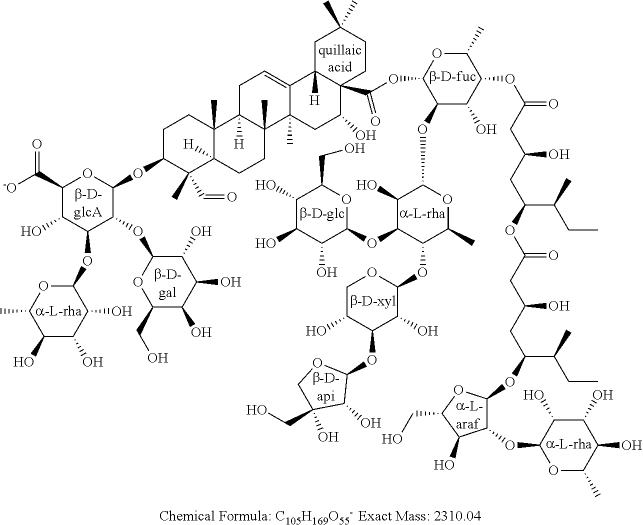

and/or QS-17 2310 A V2 (i.e. xylose isomer):

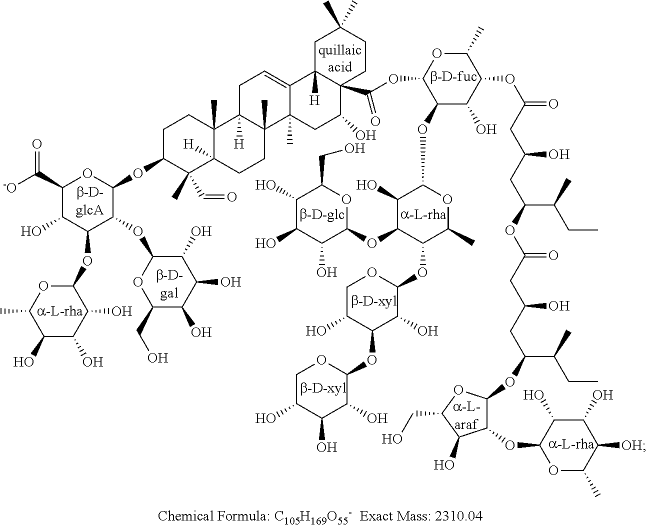

‘QS-17 2296 B component’, being triterpenoid glycosides having a m/z of 2296 with negative mode electrospray mass spectrometry. The QS-17 2296 B component corresponds to the B-isomers of xylose chemotype structure QS-III in Kite 2004. The QS-17 2296 B component may consist of QS-17 2296 B V1 (i.e. apiose isomer):

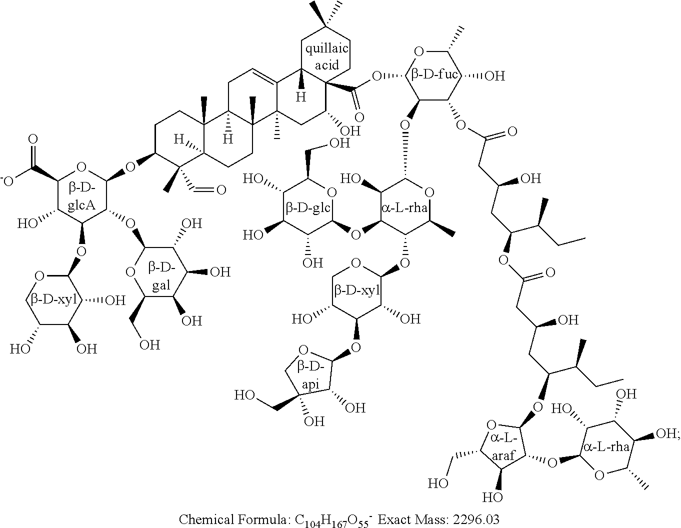

and/or QS-17 2296 B V2 (i.e. xylose isomer):

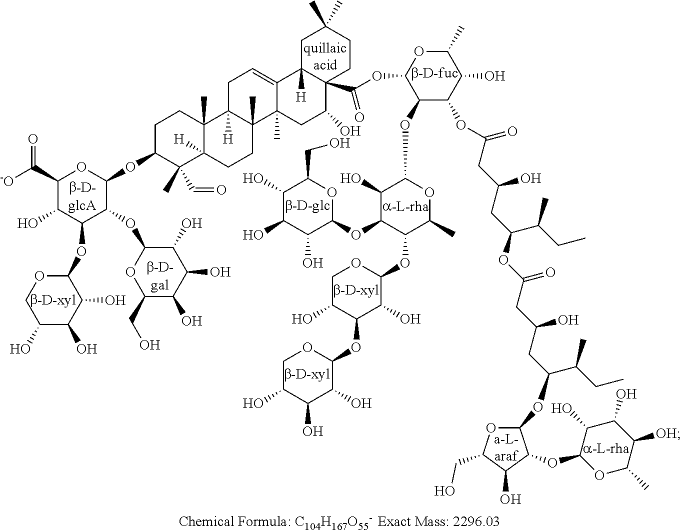

‘QS-17 2164 B component’, being triterpenoid glycosides having a m/z of 2164 with negative mode electrospray mass spectrometry. The QS-17 2164 B component corresponds to the B-isomer xylose chemotype. The QS-17 2164 B component and may consist of QS-17 2164 B:

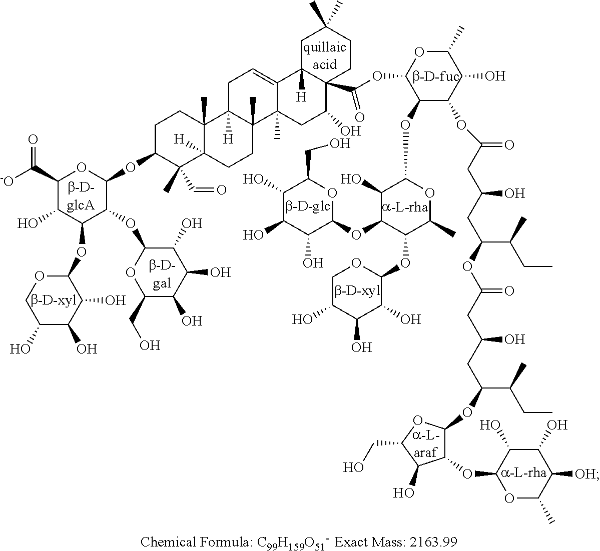

‘QS-17 2310 B component’, being triterpenoid having a m/z of 2310 with negative mode electrospray mass spectrometry. The QS-17 2310 B component corresponds to B-isomers of the rhamnose chemotype. The QS-17 2310 B component may consist of QS-17 2310 B V1 (i.e. apiose isomer):

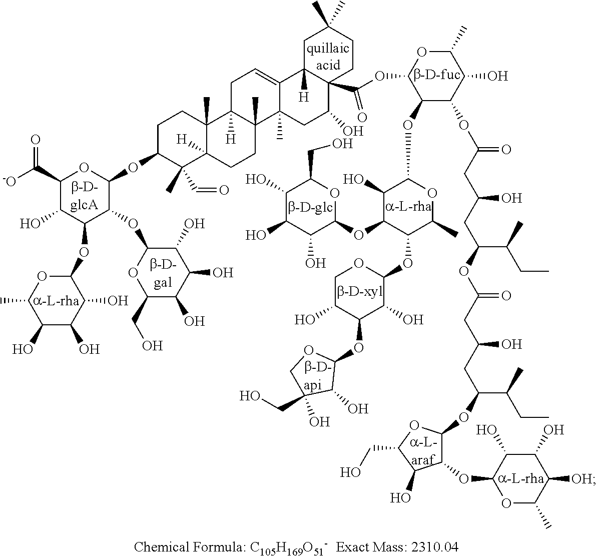

and/or QS-17 2310 B V2 (i.e. xylose isomer);

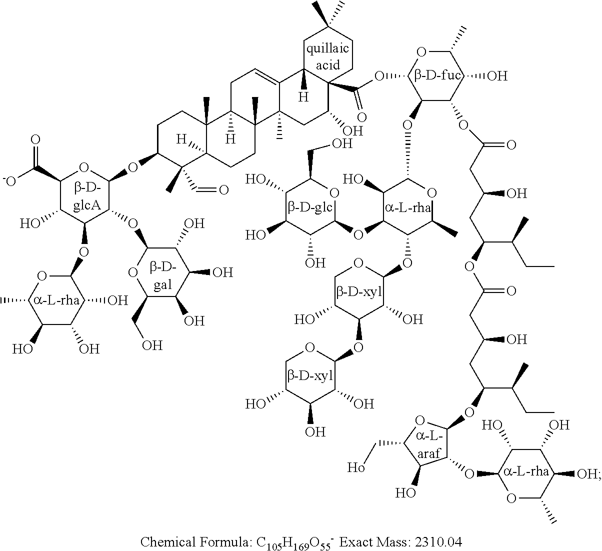

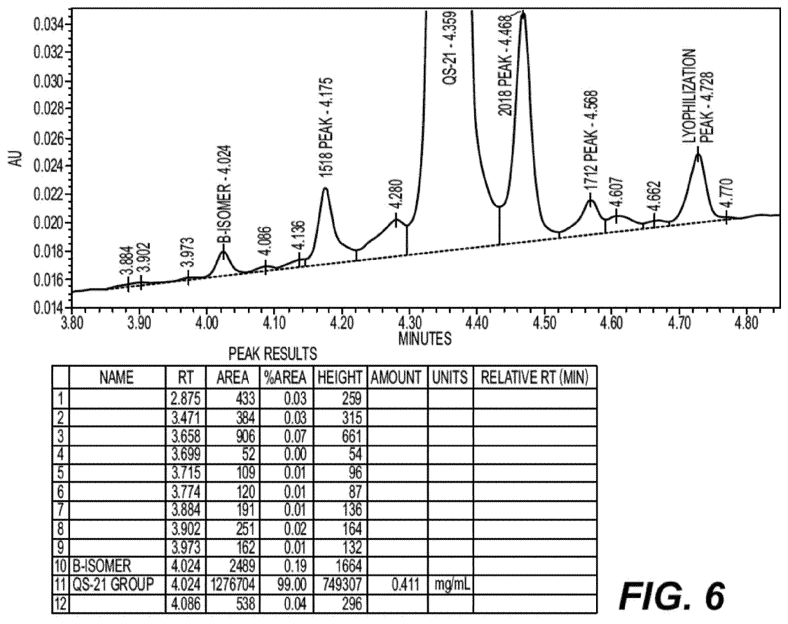

desarabinofuranosyl-QS-18 family components (i.e. triterpenoid glycosides having beta-O-glucopyranosylation at the C3 position of the L-rhamnose moiety and but lacking the arabinofuranose moiety of QS-21 family components). The desarabinofuranosyl-QS-18 family components are present in relatively low amounts in extracts, meaning that they have not been subjected to detailed characterisation. Desarabinofuranosyl-QS-18 family components can be challenging to isolate from QS-21 family components. Desarabinofuranosyl-QS-18 family components include:
   desarabinofuranosyl-QS-18 2018 A component (i.e. triterpenoid glycosides identified as part of the ‘2018 Peak’ in FIG. 6. Suitably the desarabinofuranosyl-QS-18 2018 A component in the UPLC-UV/MS methods described herein has a retention time of approximately 4.5 min, the primary component of the peak having a m/z of 2018 with negative mode electrospray mass spectrometry. The desarabinofuranosyl-QS-18 2018 A component may also be identified in the UPLC-UV methods described herein with a retention time of approximately 5.8 min. The desarabinofuranosyl-QS-18 2018 A component is believed to be identified in Kite 2004 as Peak 90 and corresponds to A-isomers of the xylose chemotype. Putative structures have been identified for the primary desarabinofuranosyl-QS-18 2018 A components using MS/MS. The desarabinofuranosyl-QS-18 2018 A component may consist of desarabinofuranosyl-QS-18 2018 A V1 (i.e. apiose isomer):

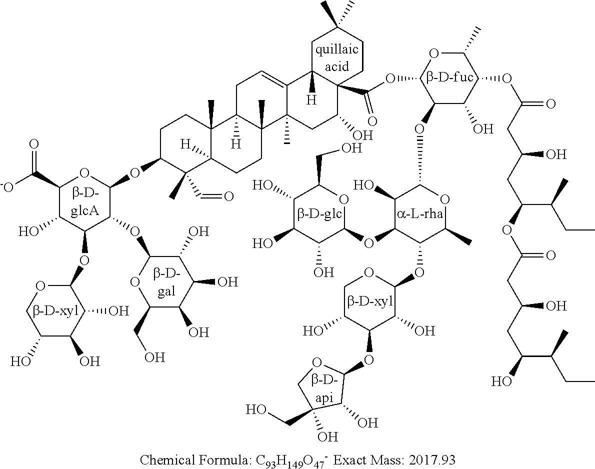

and/or desarabinofuranosyl-QS-18 2018 A V2 (i.e. xylose isomer):

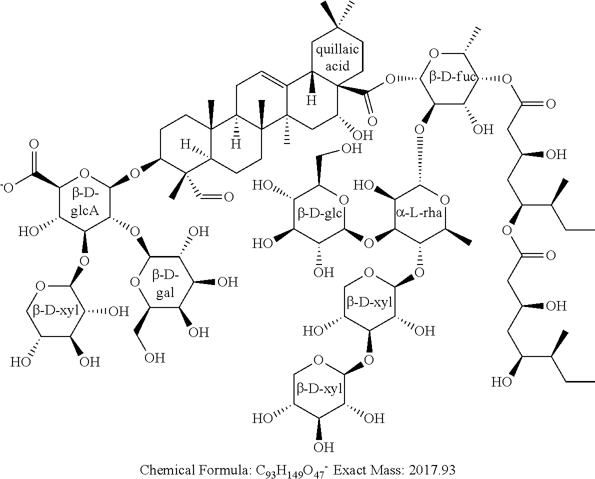

desarabinofuranosyl-QS-18 1886 A component (i.e. triterpenoid glycosides identified as part of the ‘2018 Peak’ in FIG. 6. Suitably the desarabinofuranosyl-QS-18 1886 A component in the UPLC-UV/MS methods described herein has a retention time of approximately 4.5 min and a m/z of 1886 with negative mode electrospray mass spectrometry. The desarabinofuranosyl-QS-18 1886 A component and corresponds to the A-isomer xylose chemotype may also be identified in the UPLC-UV methods described herein with a retention time of approximately 5.8 min. The desarabinofuranosyl-QS-18 1886 A component may consist of:

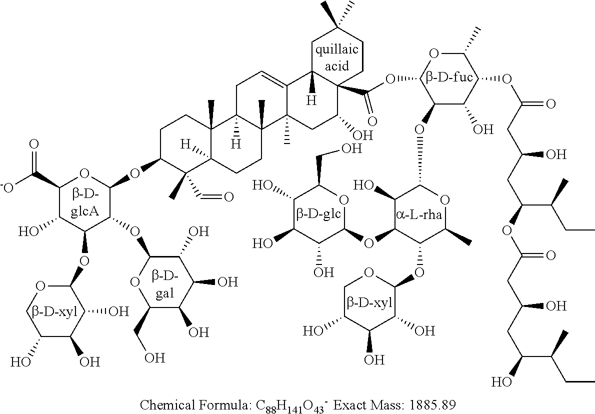

desarabinofuranosyl-QS-18 2032 A component (i.e. triterpenoid glycosides identified as part of the ‘2018 Peak’ in FIG. 6. Suitably the desarabinofuranosyl-QS-18 2032 A component in the UPLC-UV/MS methods described herein has a retention time of approximately 4.5 min and a m/z of 2032 with negative mode electrospray mass spectrometry. The desarabinofuranosyl-QS-18 2032 A component corresponds to A-isomers of the rhamnose chemotype and may also be identified in the UPLC-UV methods described herein with a retention time of approximately 5.8 min. The desarabinofuranosyl-QS-18 2032 A component may consist of desarabinofuranosyl-QS-18 2032 A V1 (i.e. apiose isomer):

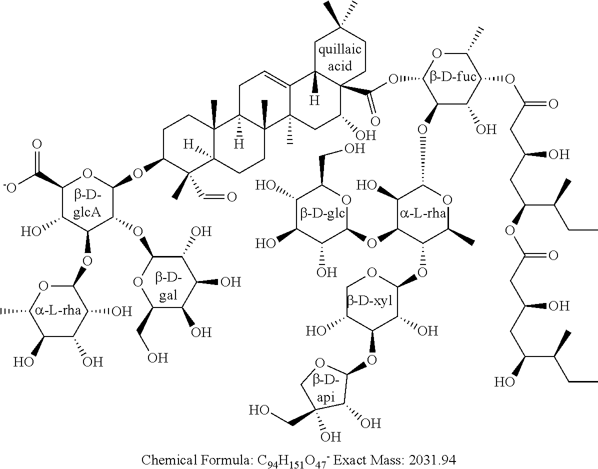

and/or desarabinofuranosyl-QS-18 2032 A V2 (i.e. xylose isomer):

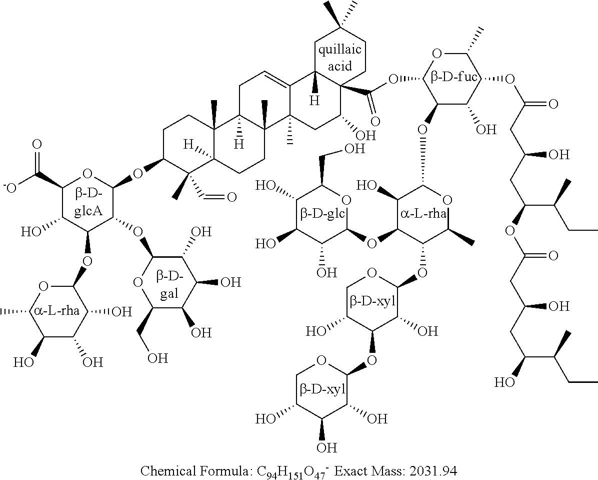

desarabinofuranosyl-QS-18 2018 B component, i.e. triterpenoid glycosides having a m/z of 2018 with negative mode electrospray mass spectrometry. The desarabinofuranosyl-QS-18 2018 B component corresponds to B-isomers of the xylose chemotype. Desarabinofuranosyl-QS-18 2018 B component may consist of desarabinofuranosyl-QS-18 2018 B V1 (i.e. apiose isomer):

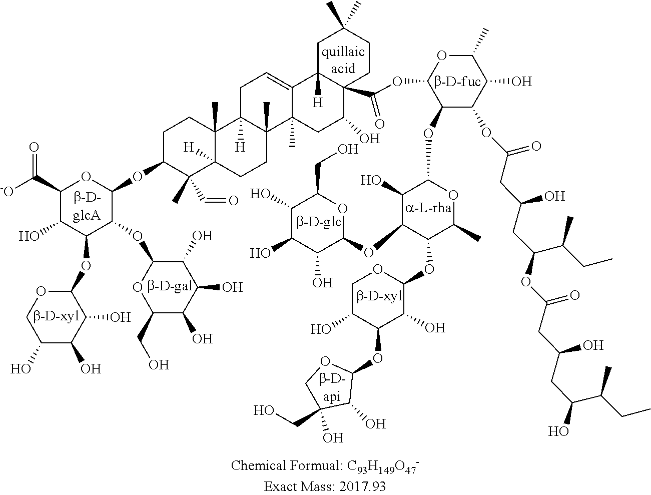

and/or desarabinofuranosyl-QS-18 2018 B V2 (i.e. xylose isomer):

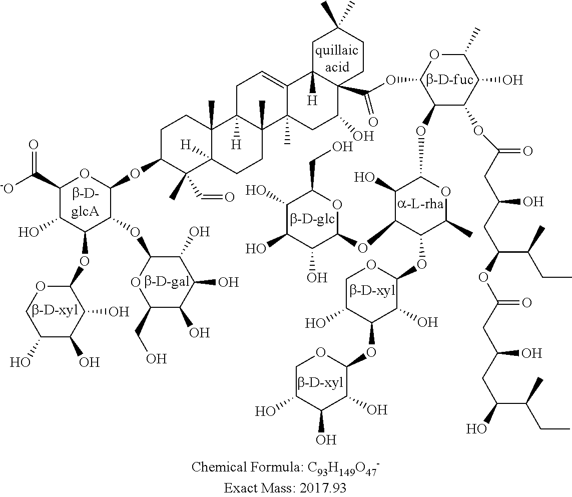

desarabinofuranosyl-QS-18 1886 B component, i.e. triterpenoid glycosides identified having a m/z of 1886 with negative mode electrospray mass spectrometry. The desarabinofuranosyl-QS-18 1886 B component corresponds to the B-isomer xylose chemotype. The desarabinofuranosyl-QS-18 1886 B component may consist of:

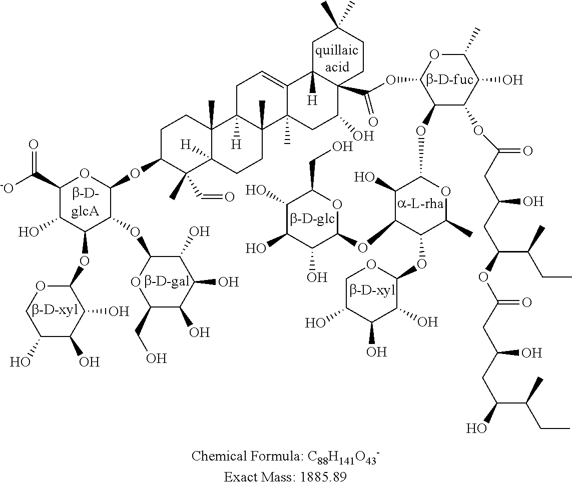

desarabinofuranosyl-QS-18 2032 B component, i.e. triterpenoid glycosides having a m/z of 2032 with negative mode electrospray mass spectrometry. The desarabinofuranosyl-QS-18 2032 B component corresponds to B-isomers of the rhamnose chemotype. The desarabinofuranosyl-QS-18 2032 B component may consist of desarabinofuranosyl-QS-18 2032 B V1 (i.e. apiose isomer):

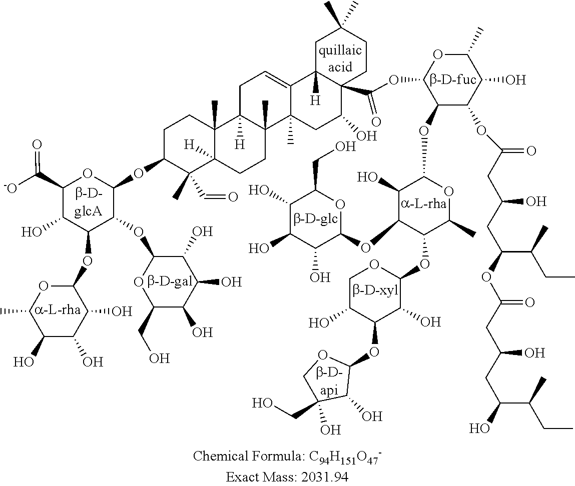

and/or desarabinofuranosyl-QS-18 2032 B V2 (i.e. xylose isomer):

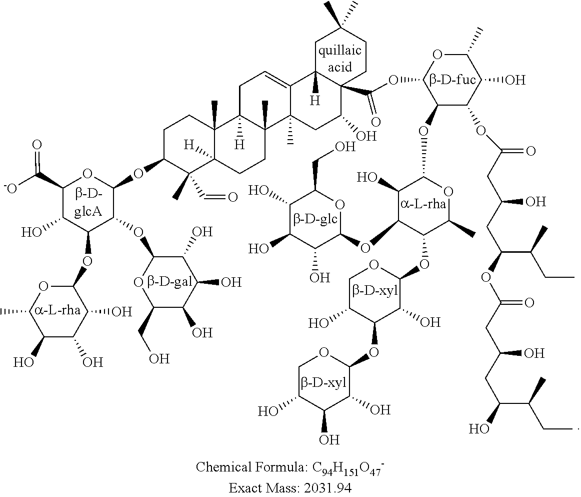

acetylated desglucosyl-QS-17 family components (i.e. triterpenoid glycosides having alpha-O-rhamnosylation at the C2 position of the arabinofuranose moiety and acetylation of the C3 position of the fucose of QS-21 family components), such as:
   ‘acetylated desglucosyl-QS-17 2176 A component’, being triterpenoid glycosides having a m/z of 2176 with negative mode electrospray mass spectrometry. The acetylated desglucosyl-QS-17 2176 A component corresponds to A-isomers of the xylose chemotype. The acetylated desglucosyl-QS-17 2176 A component may consist of acetylated desglucosyl-QS-17 2176 A V1 (i.e. apiose isomer):

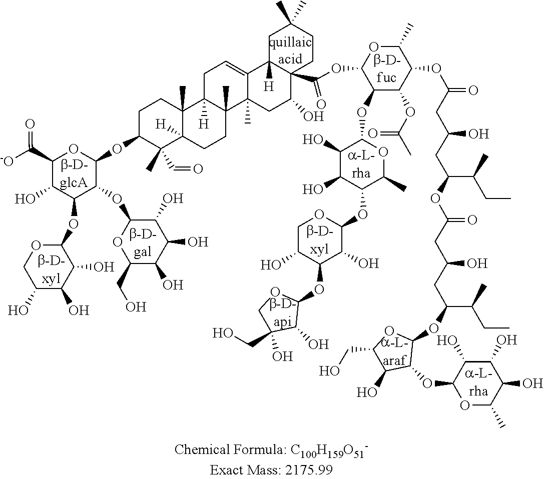

and/or acetylated desglucosyl-QS-17 2176 A V2 (i.e. xylose isomer):

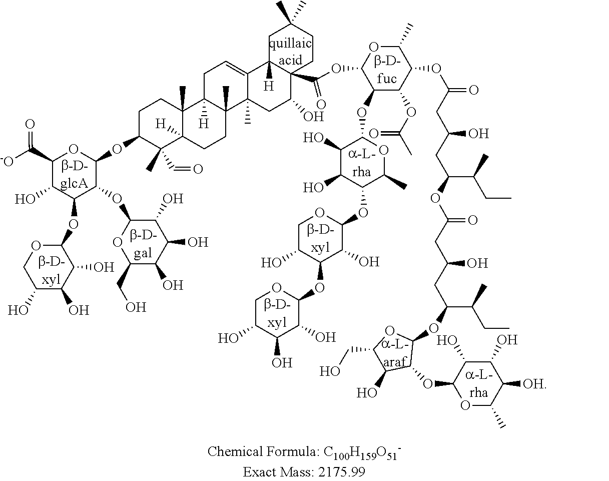

‘acetylated desglucosyl-QS-17 2044 A component’, being triterpenoid glycosides having m/z of 2044 with negative mode electrospray mass spectrometry. The acetylated desglucosyl-QS-17 2044 A component corresponds to the A-isomer xylose chemotype. The acetylated desglucosyl-QS-17 2044 A component may consist of acetylated desglucosyl-QS-17 2044 A:

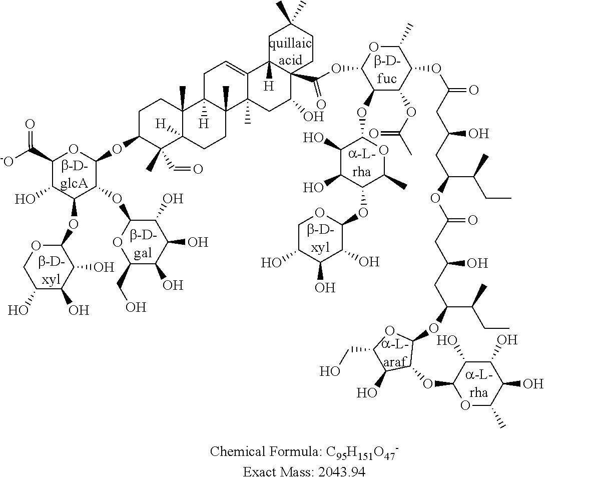

‘acetylated desglucosyl-QS-17 2190 A component’, being triterpenoid glycosides having a m/z of 2190 with negative mode electrospray mass spectrometry. The acetylated desglucosyl-QS-17 2190 A corresponds to the A-isomer rhamnose chemotype. The acetylated desglucosyl-QS-17 2190 A component may consist of acetylated desglucosyl-QS-17 2190 A V1 (i.e. apiose isomer):

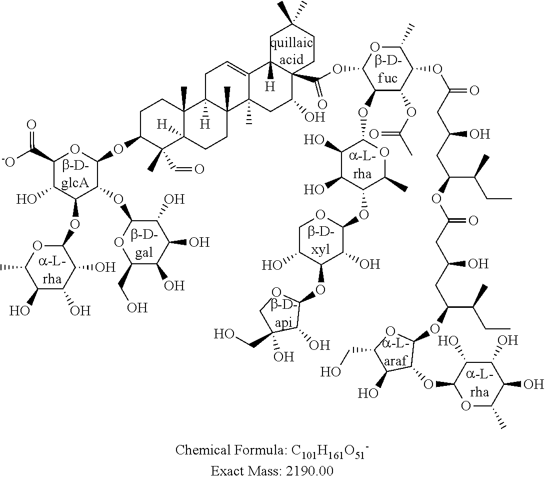

and/or acetylated desglucosyl-QS-17 2190 A V2 (i.e. xylose isomer):

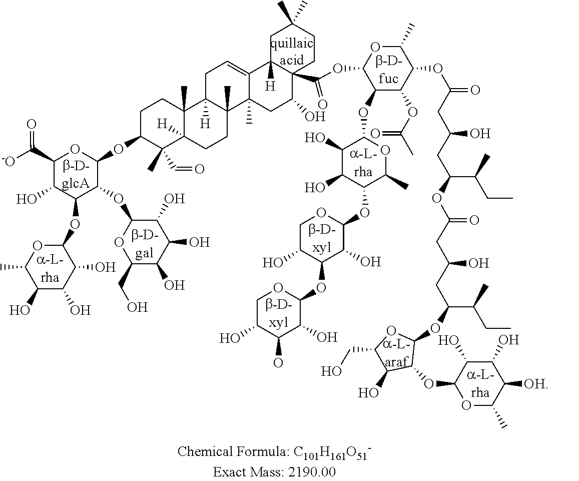

Starting saponins of direct relevance to the engineered glucosidase polypeptides are those having cleavable glucose residues, nevertheless, the engineered glucosidase polypeptides may be utilised in conjunction with additional enzymes capable of cleaving other sugar residues. Particular starting saponins of relevance to the engineered glucosidase polypeptides include:
QS-18 family components;
QS-17 family components; and
desarabinofuranosyl-QS-18 family components.

Starting saponins of direct relevance to the engineered rhamnosidase polypeptides are those having cleavable rhamnose residues, nevertheless, the engineered rhamnosidase polypeptides may be utilised in conjunction with additional enzymes capable to cleaving other sugar residues. Particular starting saponins of relevance to the engineered rhamnosidase polypeptides include:
desglucosyl-QS-17 family components;
QS-17 family components; and
acetylated desglucosyl-QS-17 family components.

The methods of the present invention enzymatically modify a starting saponin to provide a product saponin. The product saponin may be a naturally occurring saponin (i.e. a steroid or terpenoid glycoside found in nature, though the product saponin is itself obtained by the methods of the invention) or an artificially created saponin (i.e. a steroid or terpenoid glycoside not found in nature).

In some embodiments the product saponin is a steroid glycoside, in other embodiments the product saponin is a terpenoid glycoside, especially a triterpenoid glycoside.

Naturally occurring product saponins include those obtainable from plants of the genera Gypsophilia, Saponaria or Quillaja (Bomford, 1992). Especially of interest are product saponins obtainable from plants of Quillaja species. Particular product saponins of interest include those obtainable from Quillaja brasiliensis or Quillaja saponaria. In one embodiment the product saponin is obtainable from Quillaja saponaria. In one embodiment the product saponin is obtainable from Quillaja brasiliensis.

In certain embodiments the product saponin is a quillaic acid glycoside.

Product saponins obtainable from Quillaja saponaria include:
QS-18 family components (i.e. triterpenoid glycosides having beta-O-glucopyranosylation at the C3 position of the L-rhamnose moiety of QS-21 family components), such as:
   ‘QS-18 2150 A component’. The QS-18 2150 A component may consist of QS-18 2150 A V1 (i.e. apiose isomer):

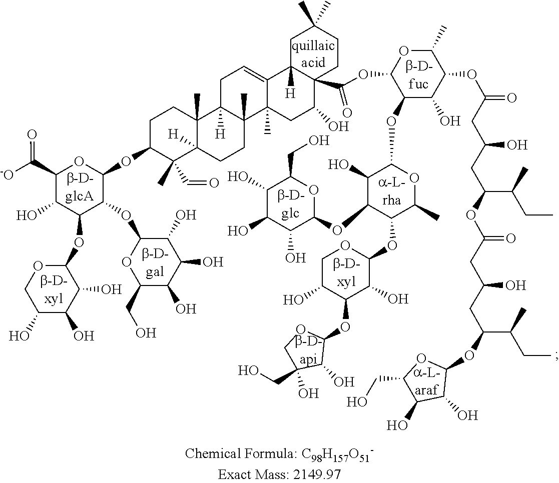

and/or QS-18 2150 A V2 (i.e. xylose isomer):

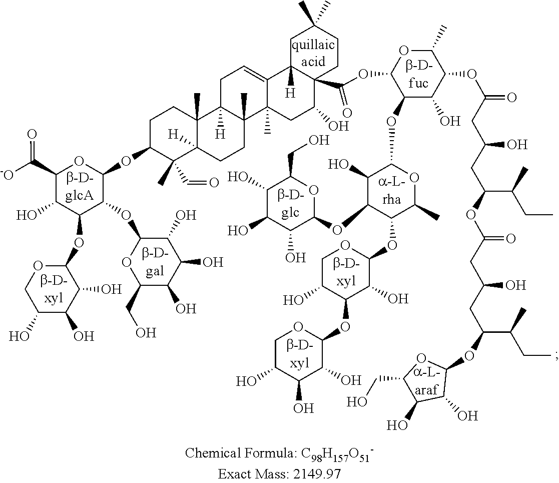

‘QS-18 2018 A component’. The QS-18 2018 A component may consist of QS-18 2018 A:

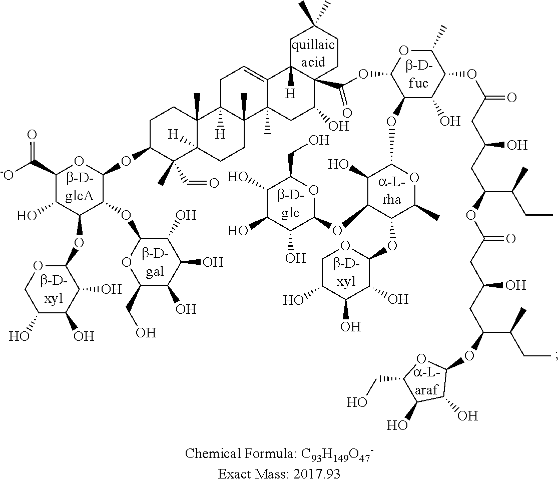

‘QS-18 2164 A component’. The QS-18 2164 A component may consist of QS-18 2164 A V1 (i.e. apiose isomer):

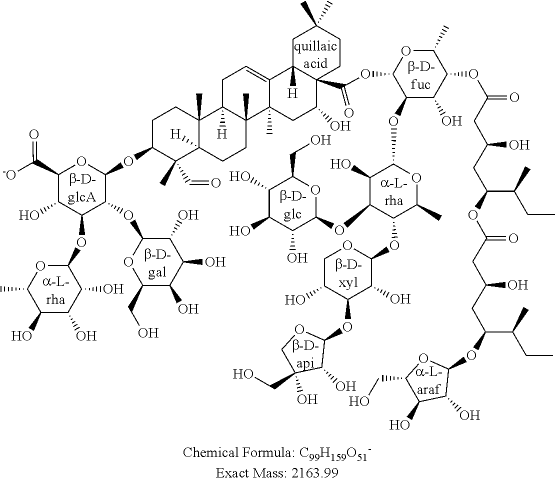

and/or QS-18 2164 A V2 (i.e. xylose isomer):

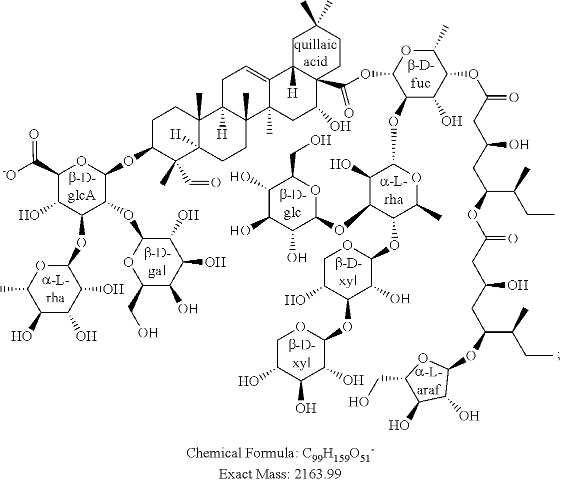

‘QS-18 2150 B component’. The QS-18 2150 B component may consist of QS-18 2150 B V1 (i.e. apiose isomer):

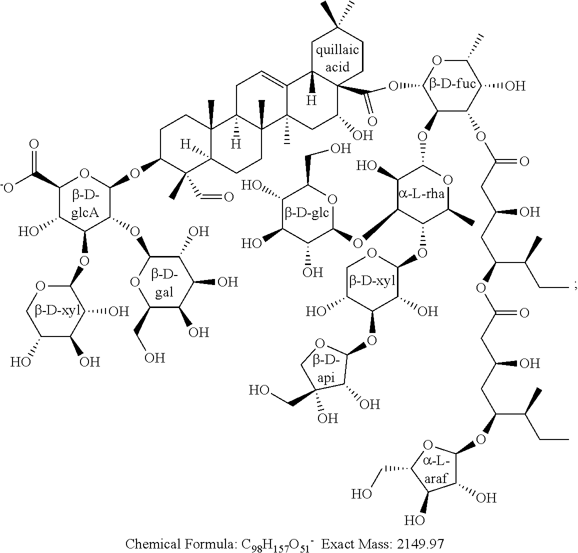

and/or QS-18 2150 B V2 (i.e. xylose isomer):

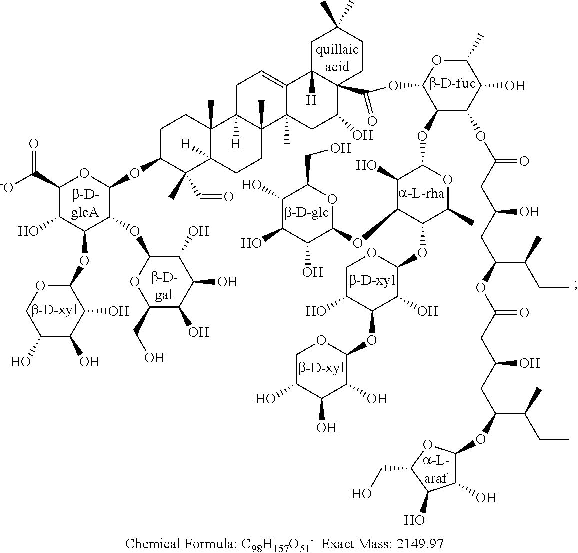

‘QS-18 2018 B component’. The QS-18 2018 B component may consist of QS-18 2018 B:

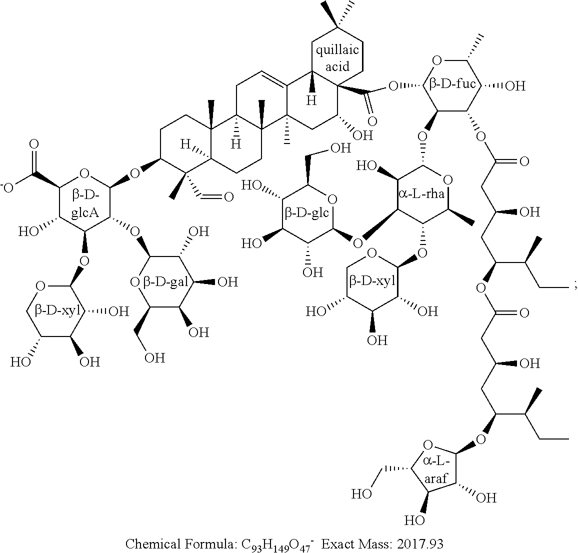

‘QS-18 2164 B component’. The QS-18 2164 B component may consist of QS-18 2164 B V1 (i.e. apiose isomer):

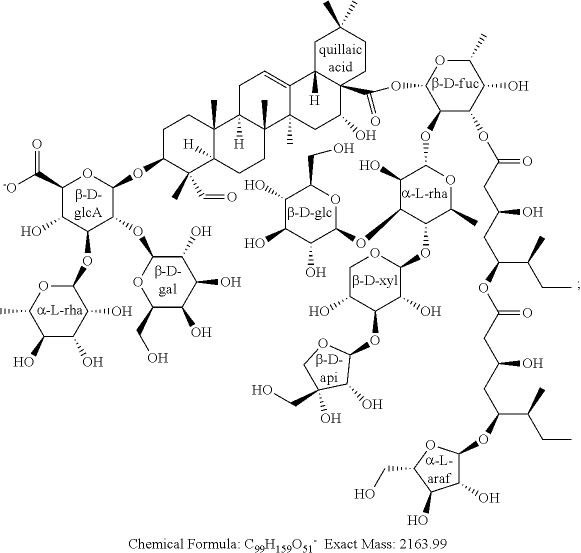

and/or QS-18 2164 B V2 (i.e. xylose isomer):

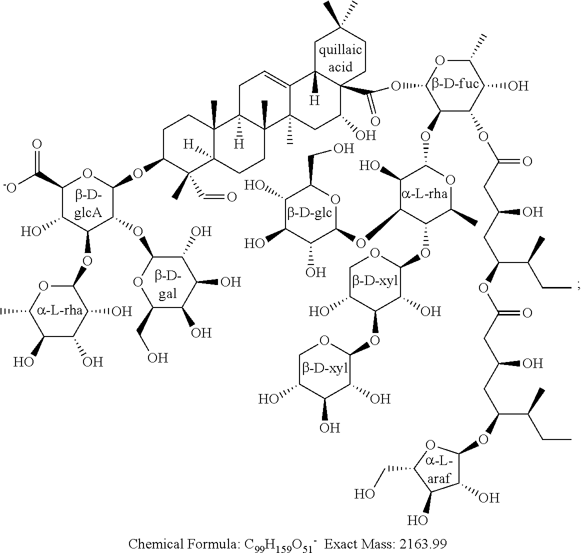

desglucosyl-QS-17 family components (i.e. triterpenoid glycosides having alpha-O-rhamnosylation at the C2 position of the arabinofuranose moiety of QS-21 family components), such as:
   ‘desglucosyl-QS-17 2134 A component’. The desglucosyl-QS-17 2134 A component may consist of desglucosyl-QS-17 2134 A V1 (i.e. apiose isomer):

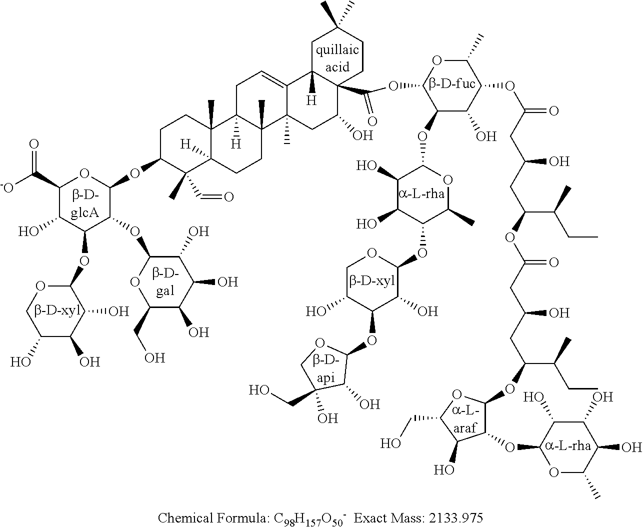

and/or desglucosyl-QS-17 2134 A V2 (i.e. xylose isomer):

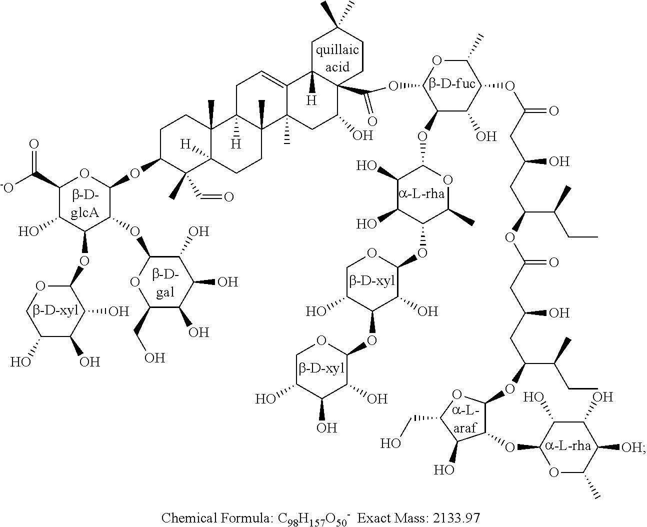

‘desglucosyl-QS-17 2002 A component’. The desglucosyl-QS-17 2002 A component may consist of desglucosyl-QS-17 2002 A:

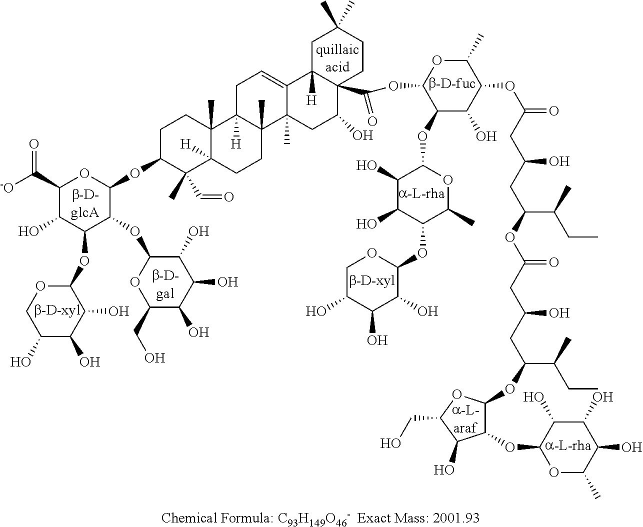

‘desglucosyl-QS-17 2148 A component’. The desglucosyl-QS-17 2148 A component may consist of desglucosyl-QS-17 2148 A V1 (i.e. apiose isomer):

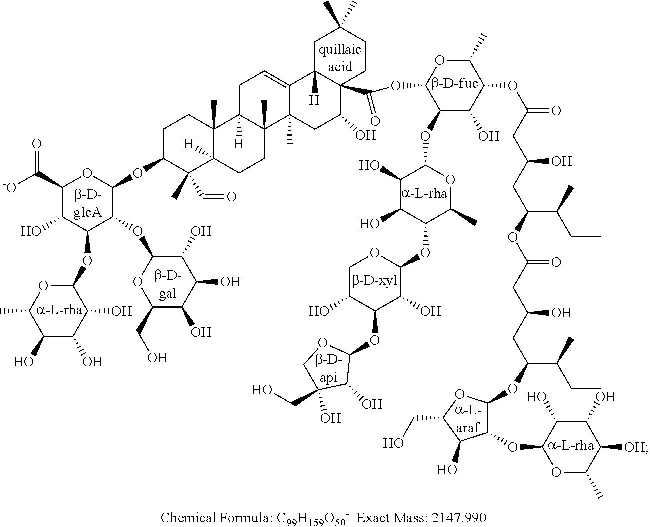

and/or desglucosyl-QS-17 2148 A V2 (i.e. xylose isomer):

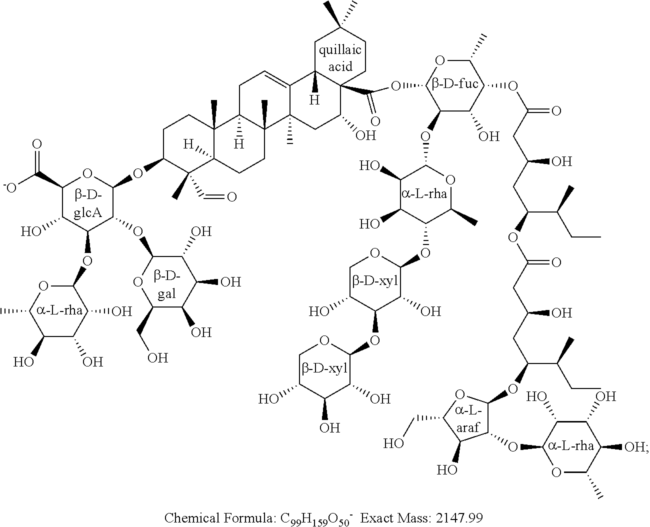

‘desglucosyl-QS-17 2134 B component’. The desglucosyl-QS-17 2134 B component may consist of desglucosyl-QS-17 2134 B V1 (i.e. apiose isomer):

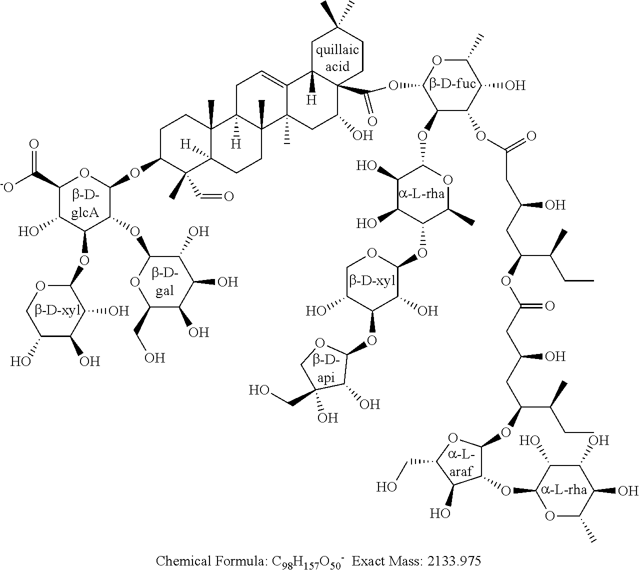

and/or desglucosyl-QS-17 2134 B V2 (i.e. xylose isomer):

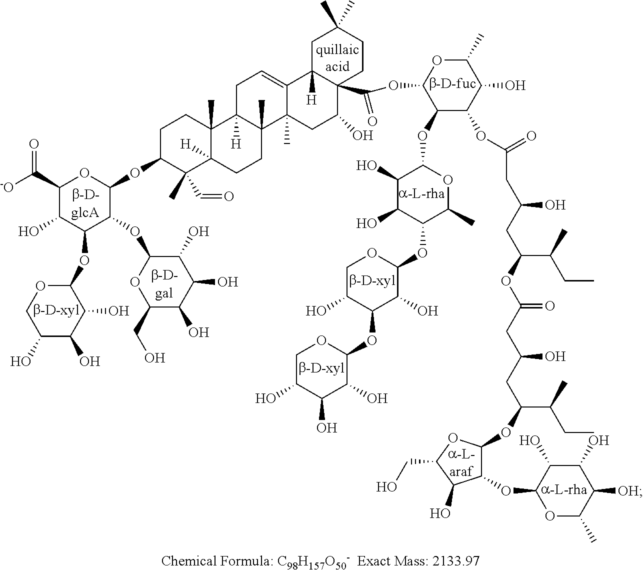

‘desglucosyl-QS-17 2002 B component’. The desglucosyl-QS-17 2002 B component may consist of desglucosyl-QS-17 2002 B:

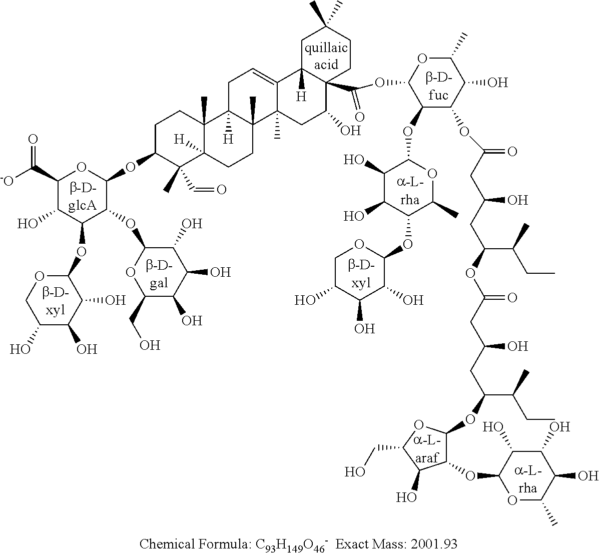

‘desglucosyl-QS-17 2148 B component’. The desglucosyl-QS-17 2148 B component may consist of desglucosyl-QS-17 2148 B V1 (i.e. apiose isomer):

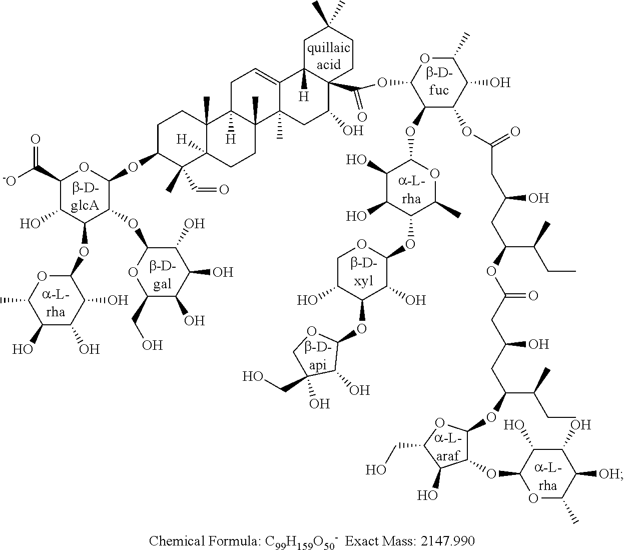

and/or desglucosyl-QS-17 2148 B V2 (i.e. xylose isomer):

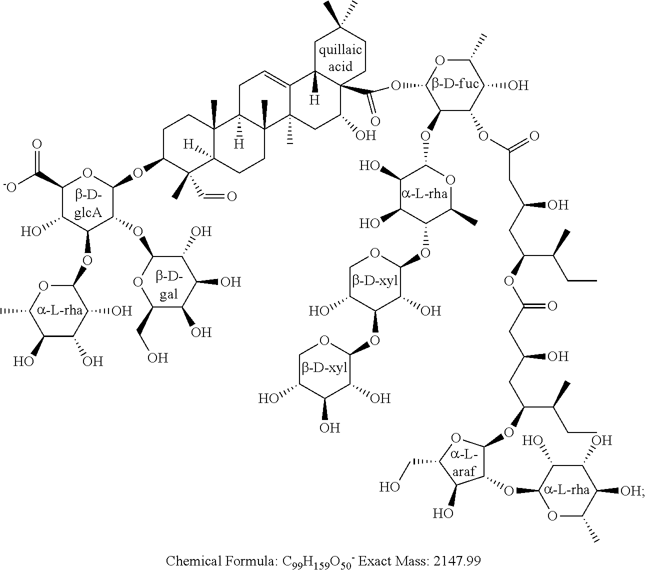

QS-21 family components, such as:
   ‘QS-21 1988 A component’, being the triterpenoid glycosides identified as part of the QS-21 main peak in FIG. 6 and having a m/z of 1988 with negative mode electrospray mass spectrometry. Suitably the QS-21 1988 A component in the UPLC-UV/MS methods described herein has a retention time of approximately 4.4 min and a m/z of 1988 with negative mode electrospray mass spectrometry. The QS-21 1988 A component is believed to be identified in Kite 2004 as Peak 88 and corresponds to the A-isomer xylose chemotype structures S6 (apiose isomer) and S4 (xylose isomer) characterised in Nyberg 2000 and Nyberg 2003. The QS-21 1988 A component may consist of QS-21 1988 A V1 (i.e. apiose isomer):

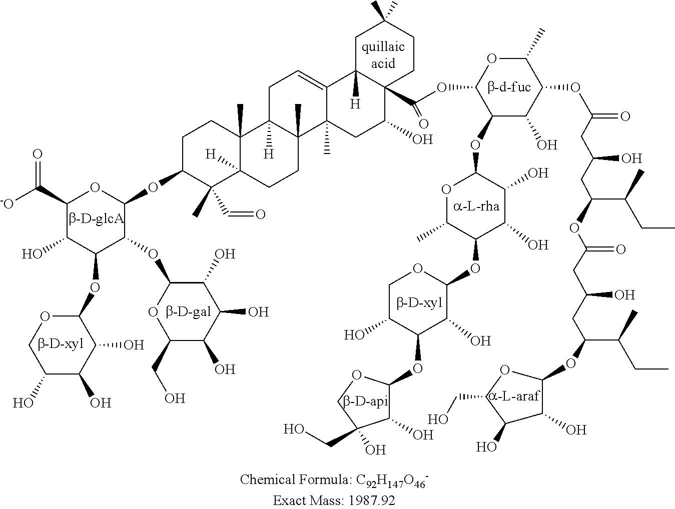

and QS-21 1988 A V2 (i.e. xylose isomer):

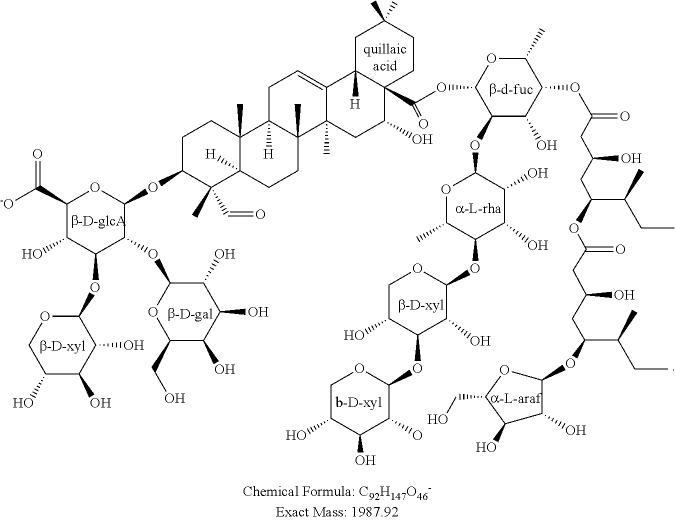

‘QS-21 1856 A component’, being the triterpenoid glycosides identified as part of the QS-21 main peak in FIG. 6 and having a m/z of 1856 with negative mode electrospray mass spectrometry. Suitably the QS-21 1856 A component in the UPLC-UV/MS methods described herein has a retention time of approximately 4.4 min and a m/z of 1856 with negative mode electrospray mass spectrometry. The QS-21 1856 A component is believed to be identified in Kite 2004 as Peak 86 and corresponds to the A-isomer xylose chemotype structure S2 characterised in Nyberg 2000 and Nyberg 2003. The QS-21 1856 A component may consist of:

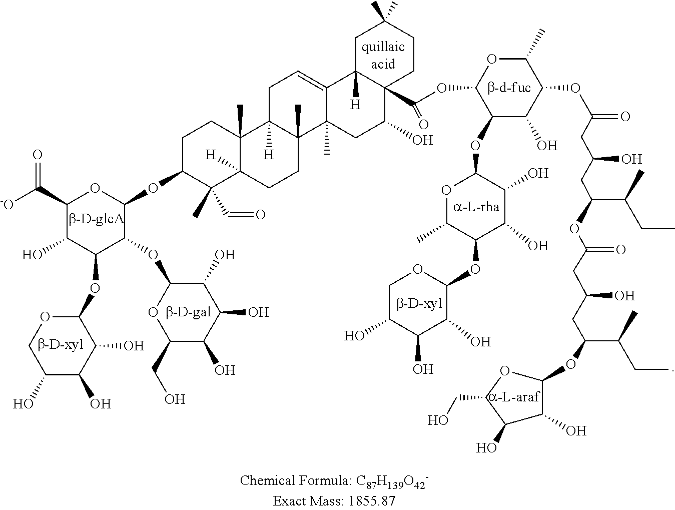

‘QS-21 2002 A component’, being the triterpenoid glycosides identified as part of the QS-21 main peak in FIG. 6 and having a m/z of 2002 with negative mode electrospray mass spectrometry. Suitably the QS-21 2002 A component in the UPLC-UV/MS methods described herein has a retention time of approximately 4.4 min and a m/z of 2002 with negative mode electrospray mass spectrometry. The QS-21 2002 A component is believed to be identified in Kite 2004 as Peak 85 and corresponds to the A-isomer rhamnose chemotype of structures S3 and S5 characterised in Nyberg 2000 and Nyberg 2003. The QS-21 2002 A component may consist of QS-21 2002 A V1 (i.e. apiose isomer):

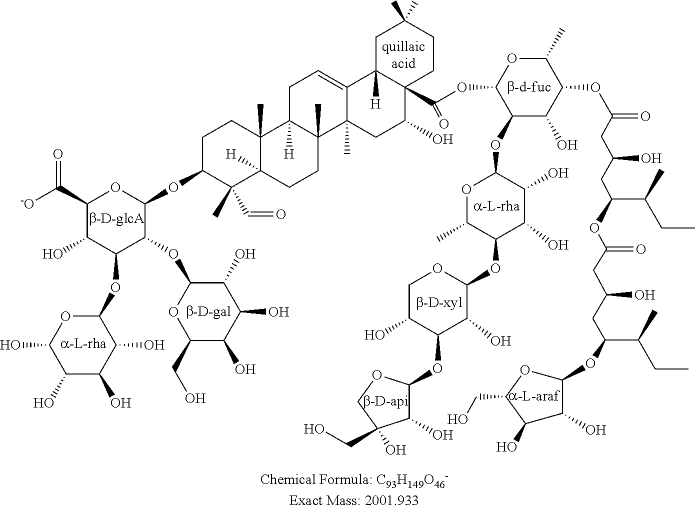

and QS-21 2002 A V2 (i.e. xylose isomer):

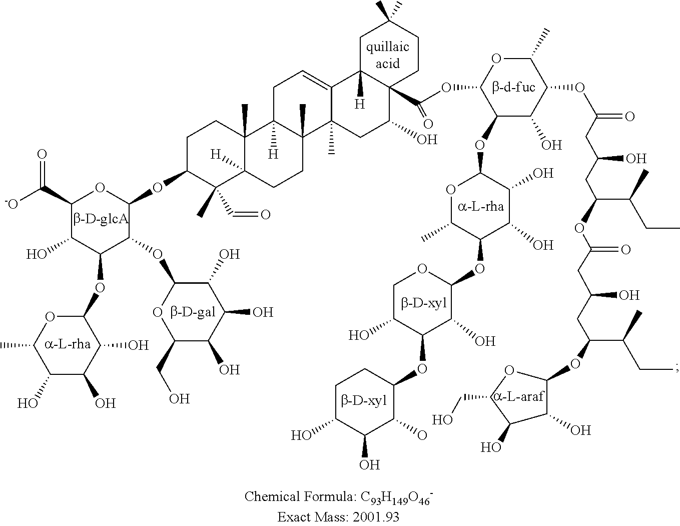

‘QS-21 1988 B component’, being the triterpenoid glycosides identified as part of the B-isomer peak in FIG. 6 and having a m/z of 1988 with negative mode electrospray mass spectrometry. Suitably the QS-21 1988 B component in the UPLC-UV/MS methods described herein has a retention time of approximately 4.0 min and a m/z of 1988 with negative mode electrospray mass spectrometry. The QS-21 1988 B component corresponds to the B-isomer xylose chemotype structures S6a (apiose isomer) and S4a (xylose isomer) characterised in Nyberg 2000 and Nyberg 2003. The QS-21 1988 B component may consist of QS-21 1988 B V1 (i.e. apiose isomer):

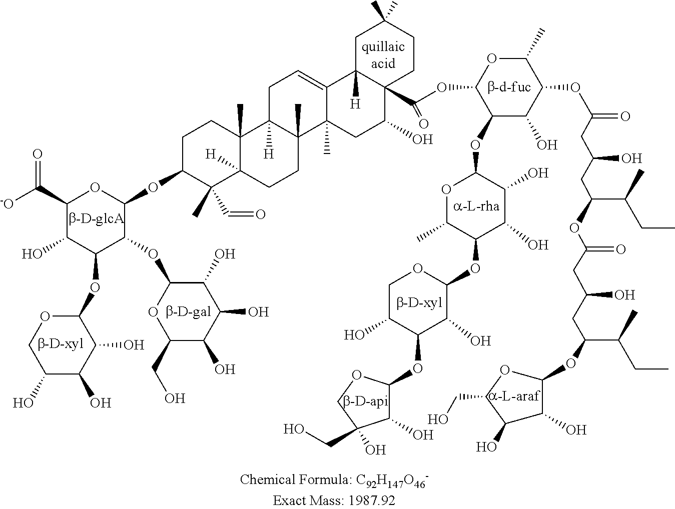

and QS-21 1988 B V2 (i.e. xylose isomer):

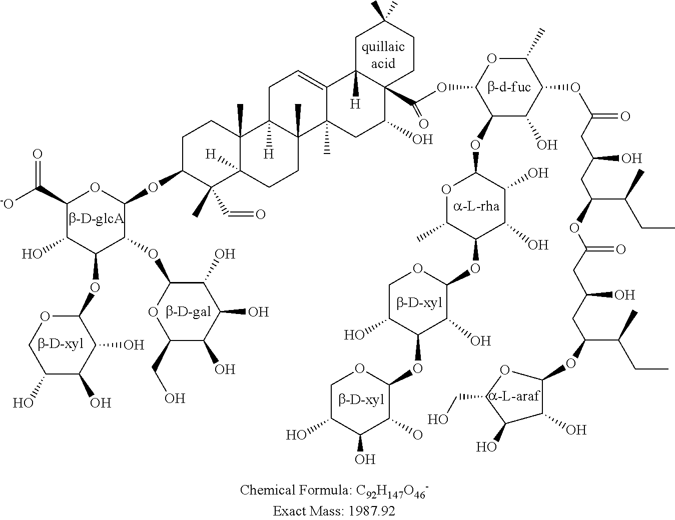

‘QS-21 1856 B component’, being the triterpenoid glycosides identified as part of the B-isomer peak in FIG. 6 and having a m/z of 1856 with negative mode electrospray mass spectrometry. The QS-21 1856 B component corresponds to the B-isomer xylose chemotype structure S2a characterised in Nyberg 2000 and Nyberg 2003. The QS-21 1856 B component may consist of:

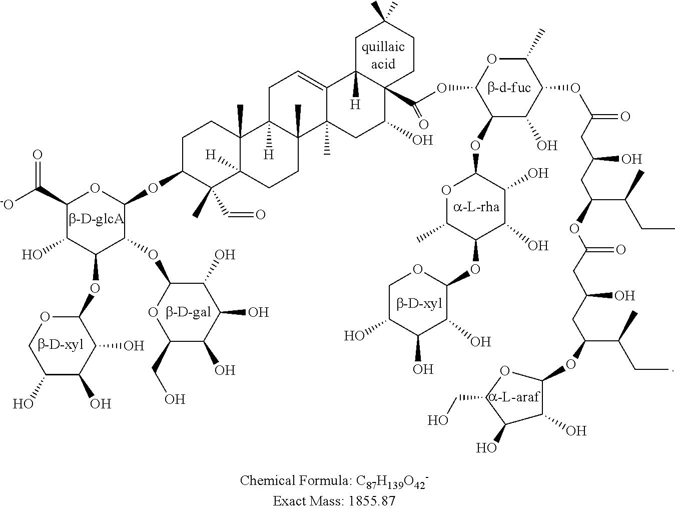

‘QS-21 2002 B component’, being the triterpenoid glycosides having a m/z of 2002 with negative mode electrospray mass spectrometry. The QS-21 2002 B component corresponds to the B-isomer rhamnose chemotype of structures S3a and S5a characterised in Nyberg 2000 and Nyberg 2003. The QS-21 2002 B component may consist of QS-21 2002 B component V1 (i.e. apiose isomer):

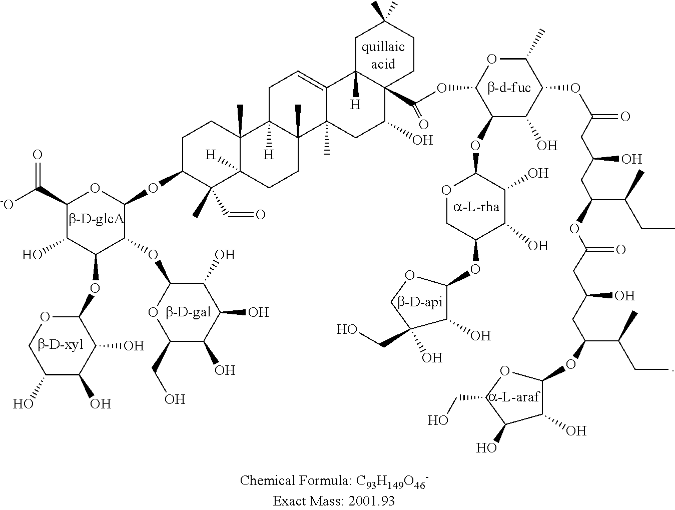

and QS-21 2002 B V2 (i.e. xylose isomer):

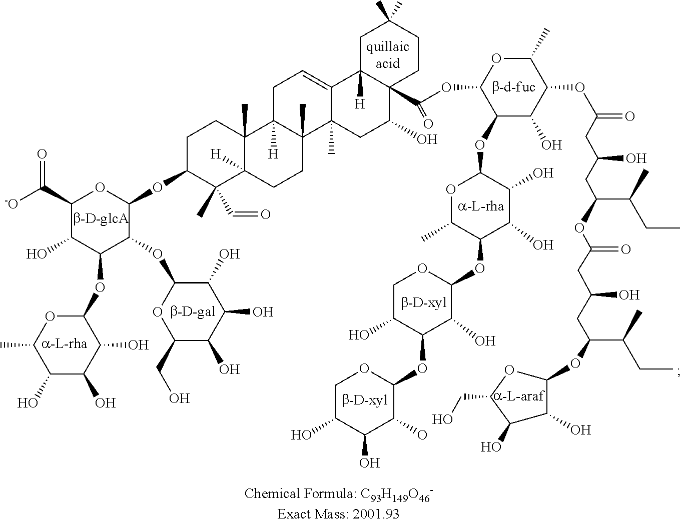

desarabinofuranosyl-QS-21 family components (i.e. triterpenoid glycosides lacking the arabinofuranose moiety of QS-21 family components). The desarabinofuranosyl-QS-21 family components are present in relatively low amounts in extracts, meaning that they have not been subjected to detailed characterisation. Desarabinofuranosyl-QS-21 family components include:
   desarabinofuranosyl-QS-21 1856 A component (i.e. triterpenoid glycosides identified as part of the ‘Lyophilization Peak’ in FIG. 6. Suitably the desarabinofuranosyl-QS-21 1856 A component in the UPLC-UV/MS methods described herein has a retention time of approximately 4.7 min, the primary component of the peak having a m/z of 1856 with negative mode electrospray mass spectrometry. The desarabinofuranosyl-QS-21 1856 A component is believed to be identified in Kite 2004 as Peak 96. Putative structures have been identified for the primary desarabinofuranosyl-QS-21 1856 A components using MS/MS. The desarabinofuranosyl-QS-21 1856 A component may consist of desarabinofuranosyl-QS-21 1856 A V1 (i.e. apiose isomer):

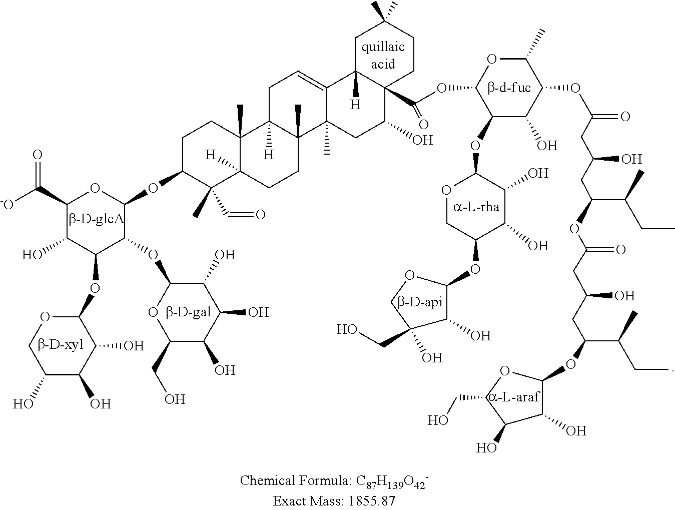

and/or desarabinofuranosyl-QS-21 1856 A V2 (i.e. xylose isomer):

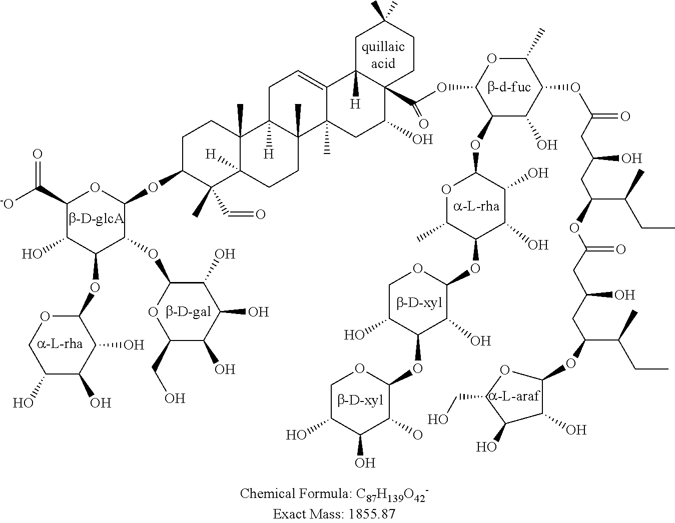

desarabinofuranosyl-QS-21 1712 A component (i.e. triterpenoid glycosides identified as part of the ‘Lyophilization Peak’ in FIG. 6. Suitably the desarabinofuranosyl-QS-21 1712 A component in the UPLC-UV/MS methods described herein has a retention time of approximately 4.7 min and a m/z of 1712 with negative mode electrospray mass spectrometry. The desarabinofuranosyl-QS-21 1712 A component may consist of desarabinofuranosyl-QS-21 1712 A:

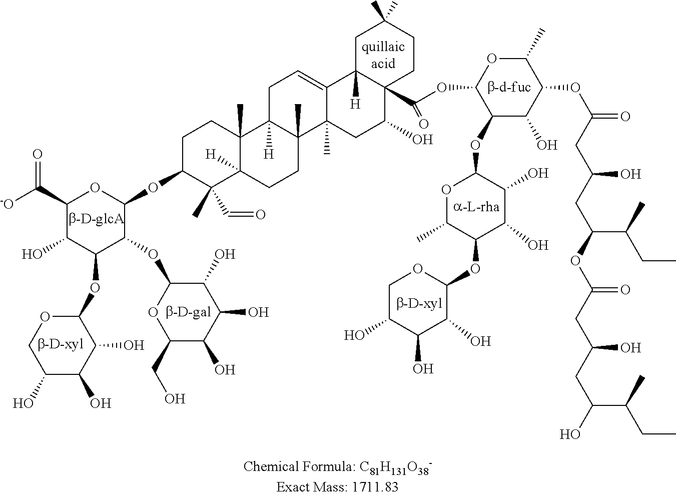

desarabinofuranosyl-QS-21 1870 A component, i.e. triterpenoid glycosides having a m/z of 1870 with negative mode electrospray mass spectrometry. The desarabinofuranosyl-QS-21 1870 A component may consist of desarabinofuranosyl-QS-21 1870 A V1 (i.e. apiose isomer):

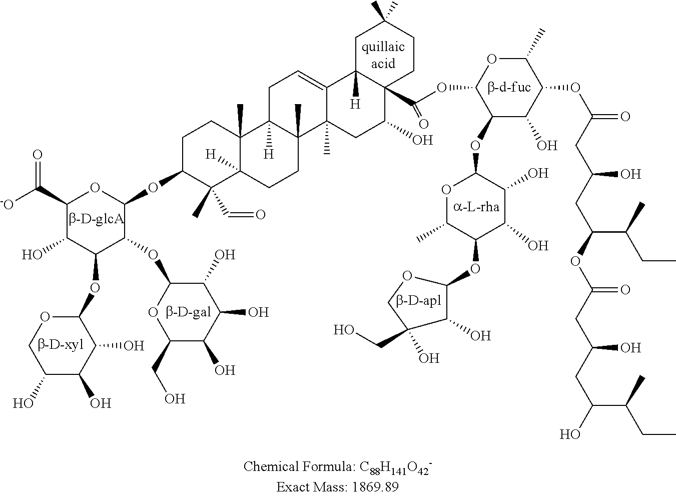

and/or desarabinofuranosyl-QS-21 1870 A V2 (i.e. xylose isomer):

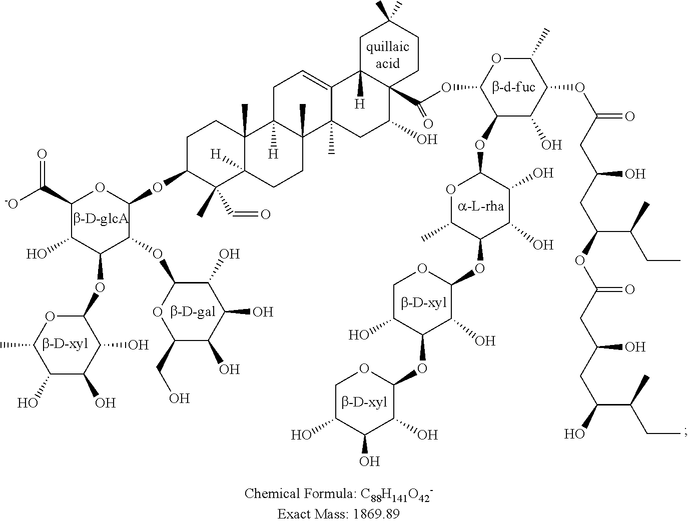

desarabinofuranosyl-QS-21 1856 B component, i.e. triterpenoid glycosides having a m/z of 1856 with negative mode electrospray mass spectrometry. The desarabinofuranosyl-QS-21 1856 B component may consist of desarabinofuranosyl-QS-21 1856 B V1 (i.e. apiose isomer):

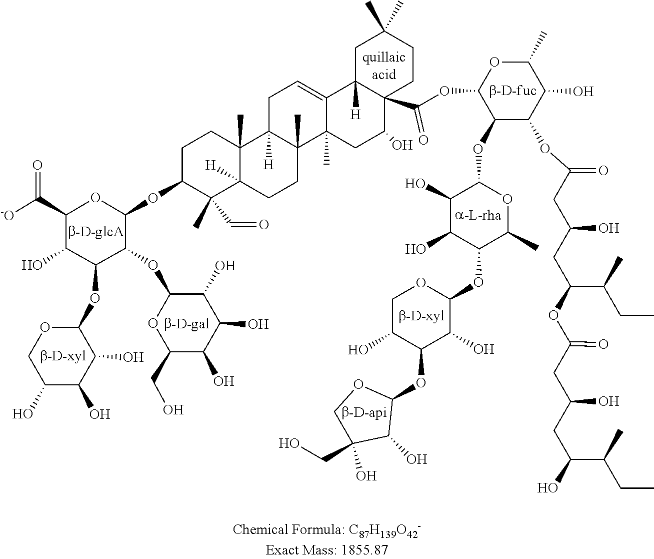

and/or desarabinofuranosyl-QS-21 1856 B V2 (i.e. xylose isomer):

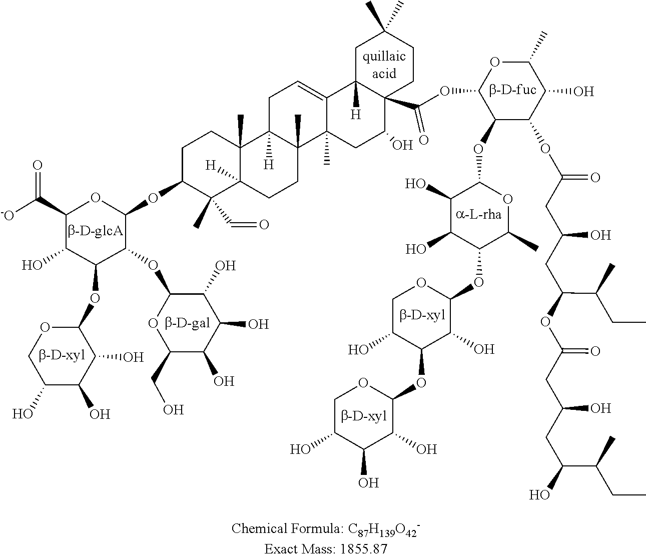

desarabinofuranosyl-QS-21 1712 B component, i.e. triterpenoid glycosides having a m/z of 1712 with negative mode electrospray mass spectrometry. The desarabinofuranosyl-QS-21 1712 B component may consist of desarabinofuranosyl-QS-21 1712 B:

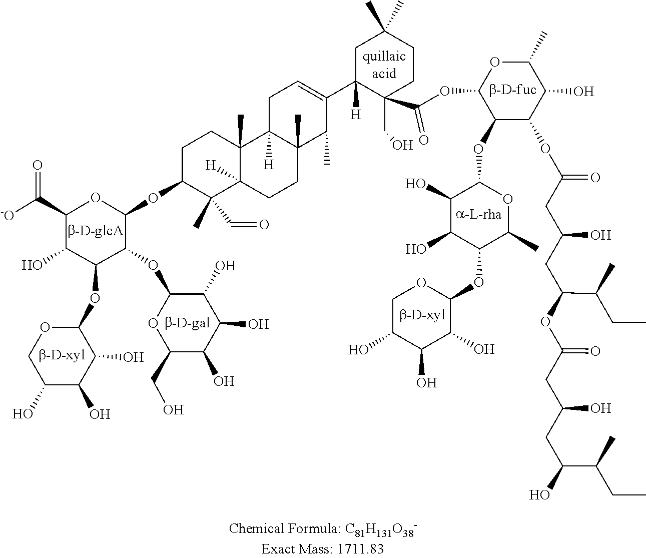

desarabinofuranosyl-QS-21 1870 B component, i.e. triterpenoid glycosides having a m/z of 1870 with negative mode electrospray mass spectrometry. The desarabinofuranosyl-QS-21 1870 B component may consist of desarabinofuranosyl-QS-21 1870 B V1 (i.e. apiose isomer):

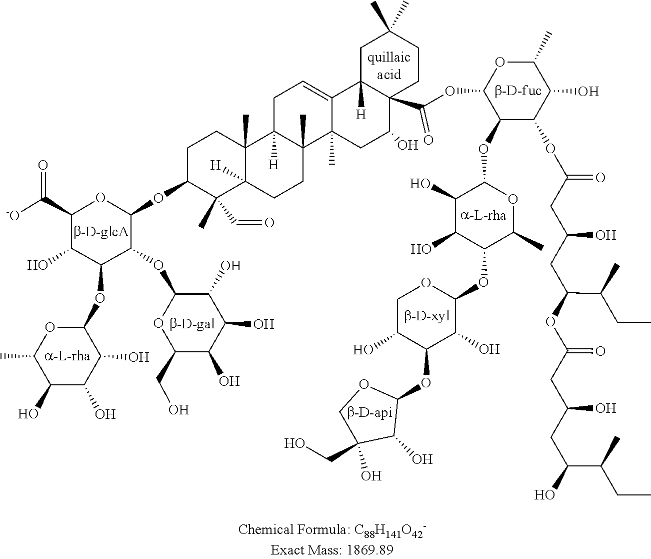

and/or desarabinofuranosyl-QS-21 1870 B V2 (i.e. xylose isomer):

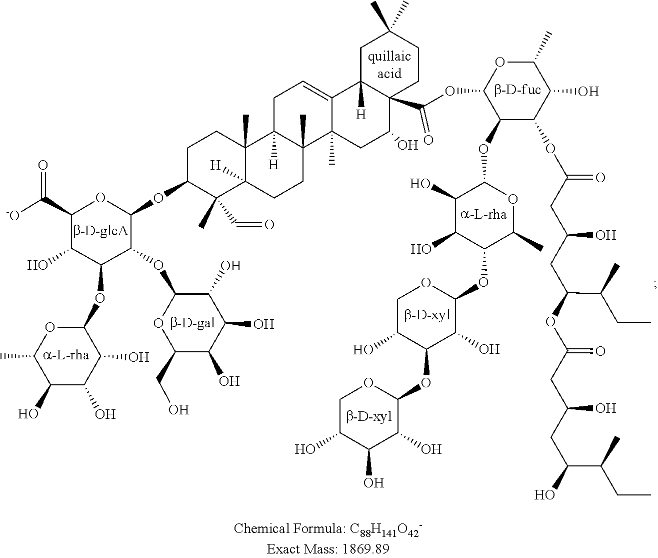

acetylated QS-21 family components (i.e. triterpenoid glycosides having acetylation of the C3 position of the fucose of QS-21 family components), such as:
   ‘acetylated QS-21 2030 A component’, being triterpenoid glycosides having a m/z of 2030 with negative mode electrospray mass spectrometry. The acetylated QS-21 2030 A corresponds to the A-isomer xylose chemotype. The acetylated QS-21 2030 A component may consist of acetylated QS-21 2030 A V1 (i.e. apiose isomer):

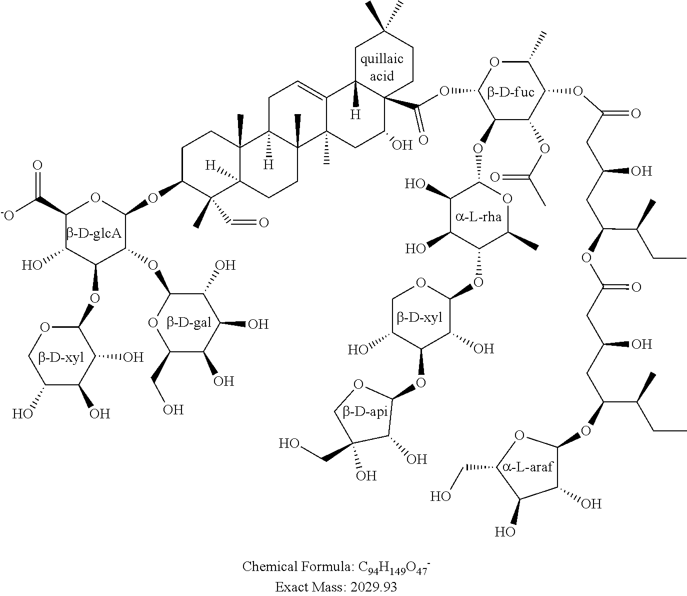

and/or acetylated QS-21 2030 A V2 (i.e. xylose isomer):

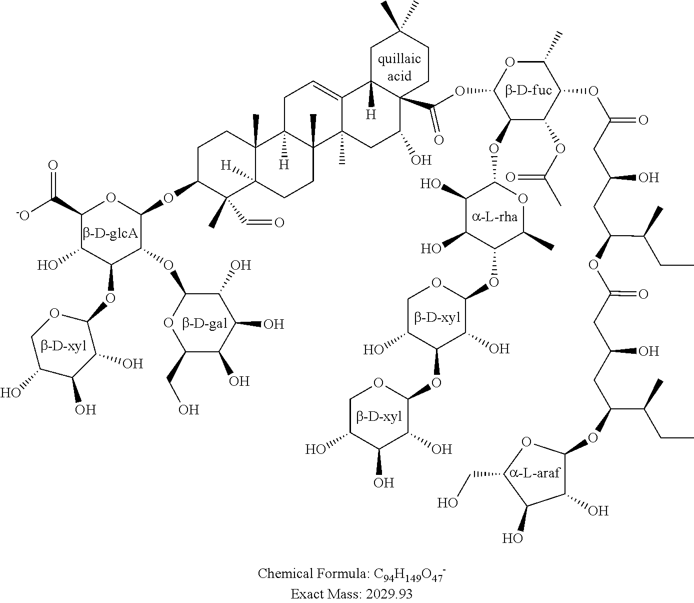

‘acetylated QS-21 1898 A component’, being triterpenoid glycosides having a m/z of 1898 with negative mode electrospray mass spectrometry. The acetylated QS-21 1898 A corresponds to the A-isomer xylose chemotype. The acetylated QS-21 1898 A component may consist of acetylated QS-21 1898 A:

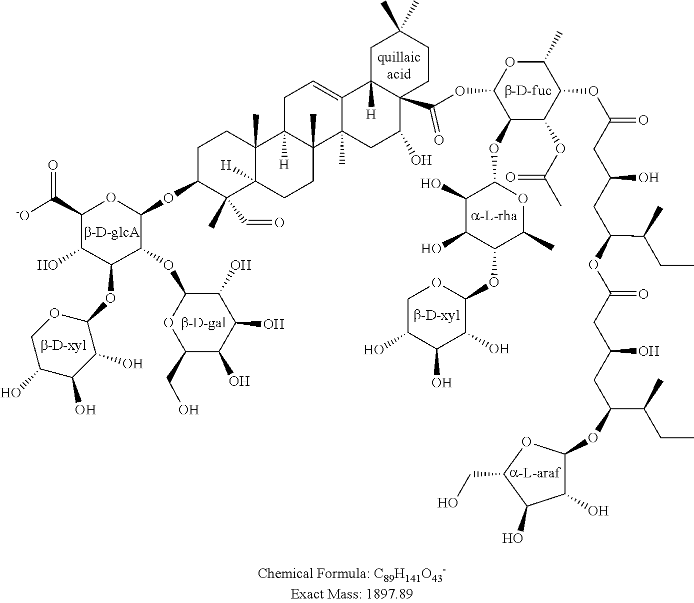

‘acetylated QS-21 2044 A component’, being triterpenoid glycosides having a m/z of 2044 with negative mode electrospray mass spectrometry. The acetylated QS-21 2044 A corresponds to A-isomers of the rhamnose chemotype. The acetylated QS-21 2044 A component may consist of acetylated QS-21 2044 A V1 (i.e. apiose isomer):

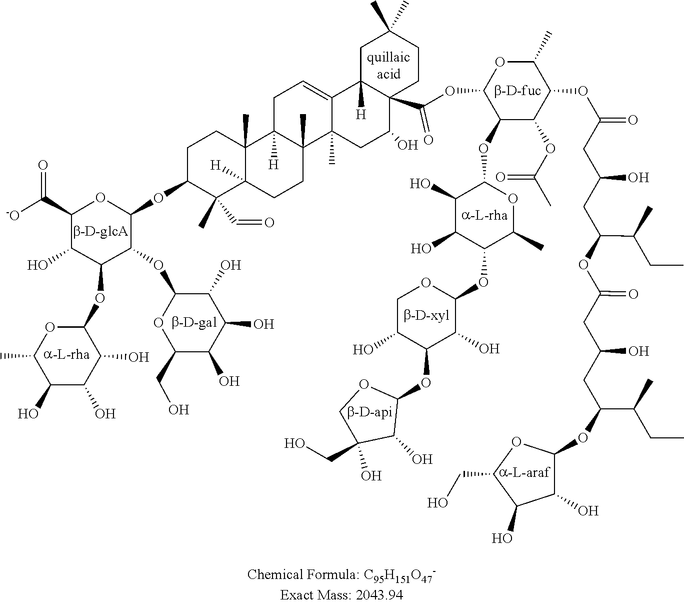

and/or acetylated QS-21 2044 A V2 (i.e. xylose isomer):

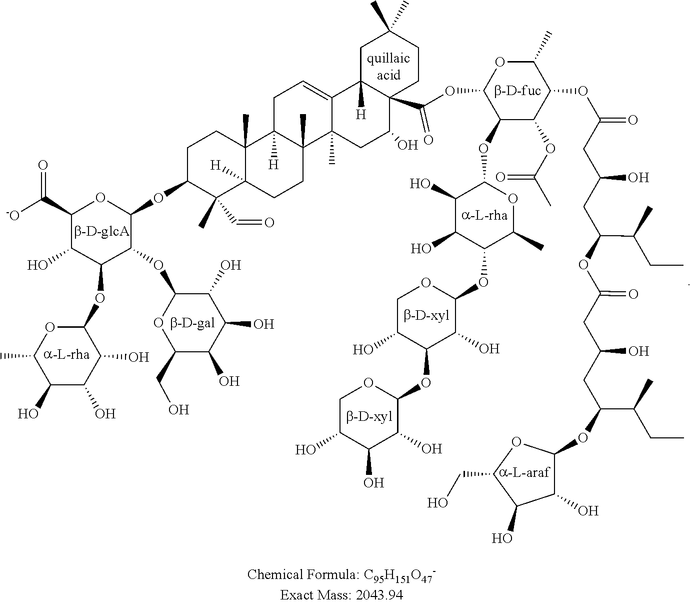

Product saponins of direct relevance to the engineered glucosidase polypeptides are those where a glucose residue has been cleaved relative to a starting saponin. Nevertheless, the engineered glucosidase polypeptides may be utilised in conjunction with additional enzymes capable to cleaving other sugar residues. Particular product saponins of relevance to the engineered glucosidase polypeptides include:
desglucosyl-QS-17 family components;
QS-21 family components; and
desarabinofuranosyl-QS-21 family components.

Product saponins of direct relevance to the engineered rhamnosidase polypeptides are those where a rhamnose residue has been cleaved relative to a starting saponin. Nevertheless, the engineered rhamnosidase polypeptides may be utilised in conjunction with additional enzymes capable to cleaving other sugar residues. Particular product saponins of relevance to the engineered rhamnosidase polypeptides include:
QS-18 family components;
QS-21 family components; and
acetylated QS-21 family components.

The term QS-18 family components as used herein means the xylose chemotype QS-18 2150 component (A and B isomers, and apiose and xylose isomers: QS-18 2150 A V1, QS-18 2150 A V2, QS-18 2150 B V1 and QS-18 2150 B V2), the xylose chemotype QS-18 2018 component (A and B isomers: QS-18 2018 A and QS-18 2018 B), the rhamnose chemotype QS-18 2164 component (A and B isomers, and apiose and xylose isomers: QS-18 2164 A V1, QS-18 2164 A V2, QS-18 2164 B V1 and QS-18 2164 B V2).

The term desglucosyl-QS-17 family components as used herein means the xylose chemotype desglucosyl-QS-17 2134 component (A and B isomers, and apiose and xylose isomers: desglucosyl-QS-17 2134 A V1, desglucosyl-QS-17 2134 A V2, desglucosyl-QS-17 2134 B V1 and desglucosyl-QS-17 2134 B V2), the xylose chemotype desglucosyl-QS-17 2002 component (A and B isomers: desglucosyl-QS-17 2002 A and desglucosyl-QS-17 2002 B), the rhamnose chemotype desglucosyl-QS-17 2148 component (A and B isomers, and apiose and xylose isomers: desglucosyl-QS-17 2148 A V1, desglucosyl-QS-17 2148 A V2, desglucosyl-QS-17 2148 B V1 and desglucosyl-QS-17 2148 B V2).

The term QS-17 family components as used herein means the xylose chemotype QS-17 2296 component (A and B isomers, and apiose and xylose isomers: QS-17 2296 A V1, QS-17 2296 A V2, QS-17 2296 B V1 and QS-17 2296 B V2), the xylose chemotype QS-17 2164 component (A and B isomers: QS-17 2164 A and QS-17 2164 B), the rhamnose chemotype QS-17 2310 component (A and B isomers, and apiose and xylose isomers: QS-17 2310 A V1, QS-17 2310 A V2, QS-17 2310 B V1 and QS-17 2310 B V2).

The term QS-21 family components as used herein means the xylose chemotype QS-21 1988 component (A and B isomers, and apiose and xylose isomers: QS-21 1988 A V1, QS-21 1988 A V2, QS-21 1988 B V1 and QS-21 1988 B V2), the xylose chemotype QS-21 1856 component (A and B isomers: QS-21 1856 A and QS-21 1856 B), the rhamnose chemotype QS-21 2002 component (A and B isomers, and apiose and xylose isomers: QS-21 2002 A V1, QS-21 2002 A V2, QS-21 2002 B V1 and QS-21 2002 B V2).

The term desarabinofuranosyl-QS-18 family components as used herein means the xylose chemotype desarabinofuranosyl-QS-18 2018 component (A and B isomers, and apiose and xylose isomers: desarabinofuranosyl-QS-18 2018 A V1, desarabinofuranosyl-QS-18 2018 A V2, desarabinofuranosyl-QS-18 2018 B V1 and desarabinofuranosyl-QS-18 2018 B V2), the xylose chemotype desarabinofuranosyl-QS-18 1886 component (A and B isomers: desarabinofuranosyl-QS-18 1886 A and desarabinofuranosyl-QS-18 1886 B), the rhamnose chemotype desarabinofuranosyl-QS-18 2032 component (A and B isomers, and apiose and xylose isomers: desarabinofuranosyl-QS-18 2032 A V1, desarabinofuranosyl-QS-18 2032 A V2, desarabinofuranosyl-QS-18 2032 B V1 and desarabinofuranosyl-QS-18 2032 B V2).

The term acetylated desglucosyl-QS-17 family components as used herein means xylose chemotype acetylated desglucosyl-QS-17 2176 component (apiose and xylose isomers: acetylated desglucosyl-QS-17 2176 A V1 and acetylated desglucosyl-QS-17 2176 A V2), the xylose chemotype acetylated desglucosyl-QS-17 2044 A component, the rhamnose chemotype acetylated desglucosyl-QS-17 2190 component (apiose and xylose isomers: acetylated desglucosyl-QS-17 2190 A V1 and acetylated desglucosyl-QS-17 2190 A V2).

The term desarabinofuranosyl-QS-21 family components as used herein means xylose chemotype desarabinofuranosyl-QS-21 1856 component (A and B isomers, and apiose and xylose isomers: desarabinofuranosyl-QS-21 1856 A V1, desarabinofuranosyl-QS-21 1856 A V2, desarabinofuranosyl-QS-21 1856 B V1 and desarabinofuranosyl-QS-21 1856 B V2), the xylose chemotype desarabinofuranosyl-QS-21 1712 component (A and B isomers: desarabinofuranosyl-QS-21 1712 A and desarabinofuranosyl-QS-21 1712 B), the rhamnose chemotype desarabinofuranosyl-QS-21 1870 component (A and B isomers, and apiose and xylose isomers: desarabinofuranosyl-QS-21 1870 A V1, desarabinofuranosyl-QS-21 1870 A V2, desarabinofuranosyl-QS-21 1870 B V1 and desarabinofuranosyl-QS-21 1870 B V2).

The term acetylated QS-21 family components as used herein means xylose chemotype acetylated QS-21 2030 component (apiose and xylose isomers: acetylated QS-21 2030 A V1 and acetylated QS-21 2030 A V2), the xylose chemotype acetylated QS-21 1898 A component, the rhamnose chemotype acetylated QS-21 2044 component (apiose and xylose isomers: acetylated QS-21 2044 A V1 and acetylated QS-21 2044 A V2).

### Starting Materials

Suitably a starting saponin is obtained by extraction from a starting material. The starting material may be plant material obtained from plants of the genera Gypsophilia, Saponaria or Quillaja (Bomford, 1992), such as plant material obtained from plants of Quillaja species. Particular plant material includes that obtained from Quillaja brasiliensis or Quillaja saponaria. In one embodiment the plant material is obtained from Quillaja saponaria. In one embodiment the plant material is obtained from Quillaja brasiliensis.

Extraction may be from complete plants. Alternatively, extraction may be from selected plant tissues. Extraction from selected plant tissues may be from plant material including wood or bark, such as from plant material which is wood or bark. In some embodiments, extraction is from plant material including bark, such as from plant material which is bark.

Extraction may be from plant material obtained from an adult plant. Alternatively, extraction may be from plant material obtained from a young plant, such as plants of less than 5 years old, such as less than 3 years old. (Schlotterbeck, 2015; WO2018057031)

Extraction may be performed using water or lower alcohols (e.g. methanol or ethanol) as solvents, including mixtures thereof. In one embodiment the starting saponin is obtained by aqueous extraction (e.g. using solvent comprising at least 80% v/v water, especially at least 90% v/v water, such as at least 95% v/v water). In one embodiment the starting saponin is obtained by methanol extraction (e.g. using solvent comprising at least 80% v/v methanol, especially at least 90% v/v methanol, such as at least 95% v/v methanol). In one embodiment the starting saponin is obtained by ethanol extraction (e.g. using solvent comprising at least 80% v/v ethanol, especially at least 90% v/v ethanol, such as at least 95% v/v ethanol). In one embodiment the starting saponin is obtained by methanol/ethanol extraction (e.g. using solvent comprising at least 20% v/v methanol, especially at least 30% v/v methanol, such as at least 40% v/v methanol and at least 20% ethanol, especially at least 30% v/v ethanol, such as at least 40% v/v ethanol). In one embodiment the starting saponin is obtained by water/ethanol extraction (e.g. using solvent comprising at least 20% v/v water, especially at least 30% v/v water, such as at least 40% v/v water and at least 20% ethanol, especially at least 30% v/v ethanol, such as at least 40% v/v ethanol. In one embodiment the starting saponin is obtained by water/methanol extraction (e.g. using solvent comprising at least 20% v/v water, especially at least 30% v/v water, such as at least 40% v/v water and at least 20% methanol, especially at least 30% v/v methanol, such as at least 40% v/v methanol).

Methods of the invention may be applied to starting saponin in a range of contexts. A starting saponin may be in the form of a minor component in a saponin containing composition (ignoring solvents, if any), such as a minor component of a plant material extract. A starting saponin may be in the form of a major component in a saponin containing composition, such as a major component in a plant material extract. A starting saponin may be in the form of a minor component in a processed, such as partially purified, plant material extract. A starting saponin may be in the form of a major component in a processed, such as partially purified, plant material extract. In some embodiments the starting saponin is substantially purified at the time of enzymatic modification.

Purification refers to the isolation of a component from other components. Partial purification therefore means the isolation of a components, to some degree, from other components. Substantial purification means the substantial isolation of a component from other components, such as wherein the component comprises at least 50% w/w, especially as at least 70%, particularly at least 80%, for example at least 90% of the component content (50%, 70%, 80% and 90% purity, respectively). Partial purification, in relation to an extract, means the isolation of the starting saponin, to some degree, from other extracted components. Substantially purified, in relation to an extract, means the substantial isolation of the starting saponin from other extracted components, such as wherein the starting saponin comprises at least 50% w/w, especially as at least 70%, particularly at least 80%, for example at least 90% of the extracted component content. Partial or substantial purification can be undertaken through various means including chromatography, filtration over semi-permeable membranes, treatment with selective adsorbants such as polyvinylpolypyrrolidone (PVPP) and the like.

Although a starting saponin may be a specific chemical entity, in many circumstances involving saponins obtained by extraction a plurality of starting saponins may be present, these being enzymatically modified to provide their corresponding product saponins. As mentioned above for individual saponins, the invention may be applied to a plurality of starting saponins in a range of contexts mutatis mutandis. A plurality of starting saponins comprising related starting saponins may undergo equivalent enzymatic modification concurrently. A plurality of starting saponins comprising distinguishable starting saponins may undergo different enzymatic modifications concurrently (in the presence of more than one enzyme) or in series (sequential treatment with separate enzymes). A plurality of starting saponins may contain both related and distinguishable starting saponins.

Methods of the invention may be applied to a starting saponin in the form of a component of:
crude extract, such as water and/or lower alcohol extract, especially aqueous extract;
crude bark extract (CBE), such as water and/or lower alcohol bark extract, especially aqueous bark extract;
partially purified extract, such as water and/or lower alcohol extract, especially aqueous extract;
partially purified bark extract, such as water and/or lower alcohol bark extract, especially aqueous bark extract;
PVPP treated extract, such as PVPP treated water and/or lower alcohol extract, especially PVPP treated aqueous extract;
PVPP treated bark extract (TBE), such as PVPP treated water and/or lower alcohol bark extract, especially PVPP treated aqueous bark extract;
Quil A;
Fraction A;
Fraction B (see Nyberg 2003);
Fraction C;
QS-7;
QS-17;
QS-18; or
QS-21.

Methods of the invention may be applied to a starting saponin in a composition comprising:
QS-7 family components and QS-18 family components;
QS-7 family components and QS-17 family components;
QS-17 family components and QS-18 family components;
QS-7 family components, QS-17 family components and QS-18 family components.

As with other QS families, the QS-7 family components contains a plurality of related structures including xylose and rhamnose chemotypes, xylose and apiose isomers, A and B isomers:

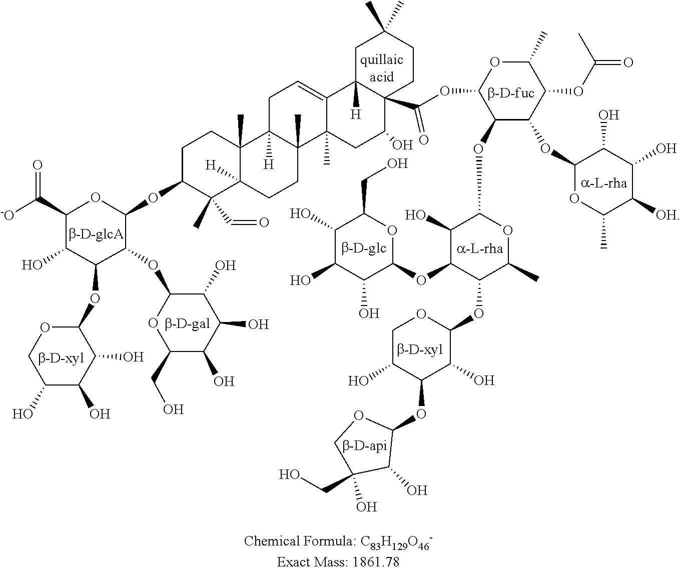

Certain QS-7 family compounds may lack glucose, or the rhamnose attached to the beta-D-fuc.

### Enzymatic Modifications

The present invention provides the enzymatic modification of saponins. Enzymatic modifications envisaged in the present invention include the conversion of a starting saponin into a product saponin by the removal of one or more sugar residues from the starting saponin. Suitably the enzymatic modifications envisaged in the present invention are the conversion of a starting saponin into a product saponin by the removal of one or more sugar residues from the starting saponin.

In certain embodiments the enzymatic modification involves the removal of a single sugar residue i.e. removal of a terminal sugar residue (‘exo’ action) from a starting saponin. In other embodiments enzymatic conversion involves the removal of a plurality of sugar residues from a starting saponin i.e. cleavage at a saccharide linkage other than in a terminal location (‘endo’ action), resulting in removal of a plurality of sugar residues (such as 2, 3 or 4 sugar residues) attached through said saccharide linkage.

Particular sugar residues which may be removed comprise (such as consist of):
glucose, in particular a terminal glucose, especially a beta-glucose, such as a beta-glucose from a quillaic acid glycoside, for example the beta-D-glucose residue highlighted below:
rhamnose, in particular a terminal rhamnose, especially an alpha-rhamnose, such as an alpha-rhamnose from a quillaic acid glycoside, for example the alpha-L-rhamnose residues highlighted below:
in particular

Particular single sugar enzymatic conversions of interest include:
QS-18 family components to QS-21 family components, such as:
   QS-18 2150 component (i.e. QS-18 2150 A and/or QS-18 2150 B) to QS-21 1988 component, such as:
      QS-18 2150 A component to QS-21 1988 A component, such as:
         QS-18 2150 A V1 to QS-21 1988 A V1
         QS-18 2150 A V2 to QS-21 1988 A V2
      QS-18 2150 B component to QS-21 1988 B component, such as:
         QS-18 2150 B V1 to QS-21 1988 B V1
         QS-18 2150 B V2 to QS-21 1998 B V2
      QS-18 2150 V1 component (i.e. QS-18 2150 A V1 and/or QS-18 2150 B V1) to QS-21 1988 V1 component, such as:
         QS-18 2150 A V1 to QS-21 1988 A V1
         QS-18 2150 B V1 to QS-21 1988 B V1
      QS-18 2150 V2 component (i.e. QS-18 2150 A V2 and/or QS-18 2150 B V2) to QS-21 1988 V2 component, such as:
         QS-18 2150 A V2 to QS-21 1988 A V2
         QS-18 2150 B V2 to QS-21 1988 B V2
   QS-18 2018 component (i.e. QS-18 2018 A and/or QS-18 2018 B) to QS-21 1856 component, such as:
      QS-18 2018 A component to QS-21 1856 A component
      QS-18 2018 B component to QS-21 1856 B component
   QS-18 2164 component (i.e. QS-18 2164 A and/or QS-18 2164 B) to QS-21 2002 component, such as:
      QS-18 2164 A component to QS-21 2002 A component, such as:
         QS-18 2164 A V1 to QS-21 2002 A V1
         QS-18 2164 A V2 to QS-21 2002 A V2
      QS-18 2164 B component to QS-21 2002 B component, such as:
         QS-18 2164 B V1 to QS-21 2002 B V1
         QS-18 2164 B V2 to QS-21 2002 B V2
      QS-18 2164 V1 component (i.e. QS-18 2164 A V1 and/or QS-18 2164 B V1) to QS-21 2002 V1 component, such as:
         QS-18 2164 A V1 to QS-21 2002 A V1
         QS-18 2164 B V1 to QS-21 2002 B V1
      QS-18 2164 V2 component (i.e. QS-18 2164 A V2 and/or QS-18 2164 B V2) to QS-21 2002 V2 component, such as:
         QS-18 2164 A V2 to QS-21 2002 A V2
         QS-18 2164 B V2 to QS-21 2002 B V2
desglucosyl-QS-17 family components to QS-21 family components, such as:
   desglucosyl-QS-17 2134 component (i.e. desglucosyl-QS-17 2134 A and/or desglucosyl-QS-17 2134 B) to QS-21 1988 component, such as:
      desglucosyl-QS-17 2134 A component to QS-21 1988 A component, such as:
         desglucosyl-QS-17 2134 A V1 to QS-21 1988 A V1
         desglucosyl-QS-17 2134 A V2 to QS-21 1988 A V2
      desglucosyl-QS-17 2134 B component to QS-21 1988 B component, such as:
         desglucosyl-QS-17 2134 B V1 to QS-21 1988 B V1
         desglucosyl-QS-17 2134 B V2 to QS-21 1988 B V2
      desglucosyl-QS-17 2134 V1 component (i.e. desglucosyl-QS-17 2134 A V1 and/or desglucosyl-QS-17 2134 B V1) to QS-21 1988 V1 component, such as:
         desglucosyl-QS-17 2134 A V1 to QS-21 1988 A V1
         desglucosyl-QS-17 2134 B V1 to QS-21 1988 B V1
      desglucosyl-QS-17 2134 V2 component (i.e. desglucosyl-QS-17 2134 A V2 and/or desglucosyl-QS-17 2134 B V2) to QS-21 1988 V2 component, such as:
         desglucosyl-QS-17 2134 A V2 to QS-21 1988 A V2
         desglucosyl-QS-17 2134 B V2 to QS-21 1988 B V2
   desglucosyl-QS-17 2002 component (i.e. desglucosyl-QS-17 2002 A and/or desglucosyl-QS-17 2002 B) to QS-21 1856 component, such as:
      desglucosyl-QS-17 2002 A component to QS-21 1856 A component
      desglucosyl-QS-17 2002 B component to QS-21 1856 B component
   desglucosyl-QS-17 2148 component (i.e. desglucosyl-QS-17 2148 A and/or desglucosyl-QS-17 2148 B) to QS-21 2002 component, such as:
      desglucosyl-QS-17 2148 A component to QS-21 2002 A component, such as:
         desglucosyl-QS-17 2148 A V1 to QS-21 2002 A V1
         desglucosyl-QS-17 2148 A V2 to QS-21 2002 A V2
      desglucosyl-QS-17 2148 B component to QS-21 2002 B component, such as:
         desglucosyl-QS-17 2148 B V1 to QS-21 2002 B V1
         desglucosyl-QS-17 2148 B V2 to QS-21 2002 B V2
      desglucosyl-QS-17 2134 V1 component (i.e. desglucosyl-QS-17 2134 A V1 and/or desglucosyl-QS-17 2134 B V1) to QS-21 1988 V1 component, such as:
         desglucosyl-QS-17 2148 A V1 to QS-21 2002 A V1
         desglucosyl-QS-17 2148 B V1 to QS-21 2002 B V1
      desglucosyl-QS-17 2134 V2 component (i.e. desglucosyl-QS-17 2134 A V2 and/or desglucosyl-QS-17 2134 B V2) to QS-21 1988 V1 component, such as:
         desglucosyl-QS-17 2148 A V2 to QS-21 2002 A V2
         desglucosyl-QS-17 2148 B V2 to QS-21 2002 B V2
QS-17 family components to QS-18 family components, such as:
   QS-17 2296 component (i.e. QS-17 2296 A and/or QS-17 2296 B) to QS-18 2150 component, such as:
      QS-17 2296 A component to QS-18 2150 A component, such as:
         QS-17 2296 A V1 to QS-18 2150 A V1
         QS-17 2296 A V2 to QS-18 2150 A V2
      QS-17 2296 B component to QS-18 2150 B component, such as:
         QS-17 2296 B V1 to QS-18 2150 B V1
         QS-17 2296 B V2 to QS-18 2150 B V2
      QS-17 2296 V1 component (i.e. QS-17 2296 A V1 and/or QS-17 2296 B V1) to QS-18 2150 V1 component, such as:
         QS-17 2296 A V1 to QS-18 2150 A V1
         QS-17 2296 B V1 to QS-18 2150 B V1
      QS-17 2296 V2 component (i.e. QS-17 2296 A V2 and/or QS-17 2296 B V1) to QS-18 2150 V2 component, such as:
         QS-17 2296 A V2 to QS-18 2150 A V2
         QS-17 2296 B V2 to QS-18 2150 B V2
   QS-17 2164 component (i.e. QS-17 2164 A and/or QS-17 2164 B) to QS-18 2018 component, such as:
      QS-17 2164 A component to QS-18 2018 A component
      QS-17 2164 B component to QS-18 2018 B component
   QS-17 2310 component (i.e. QS-17 2310 A and/or QS-17 2310 B) to QS-18 2164 component, such as:
      QS-17 2310 A component to QS-18 2164 A component, such as:
         QS-17 2310 A V1 to QS-18 2164 A V1
         QS-17 2310 A V2 to QS-18 2164 A V2
      QS-17 2310 B component to QS-18 2164 B component, such as:
         QS-17 2310 B V1 to QS-18 2164 B V1
         QS-17 2310 B V2 to QS-18 2164 B V2
      QS-17 2310 V1 component (i.e. QS-17 2310 A V1 and/or QS-17 2310 B V1) to QS-18 2164 V1 component, such as:
         QS-17 2310 A V1 to QS-18 2164 A V1
         QS-17 2310 B V1 to QS-18 2164 B V1
      QS-17 2310 V2 component (i.e. QS-17 2310 A V2 and/or QS-17 2310 B V2) to QS-18 2164 V2 component, such as:
         QS-17 2310 A V2 to QS-18 2164 A V2
         QS-17 2310 B V2 to QS-18 2164 B V2
QS-17 family components to desglucosyl-QS-17 family components, such as:
   QS-17 2296 component (i.e. QS-17 2296 A and/or QS-17 2296 B) to desglucosyl-QS-17 2134 component, such as:
      QS-17 2296 A component to desglucosyl-QS-17 2134 A component, such as:
         QS-17 2296 A V1 to desglucosyl-QS-17 2134 A V1
         QS-17 2296 A V2 to desglucosyl-QS-17 2134 A V2
      QS-17 2296 B component to desglucosyl-QS-17 2134 B component, such as:
         QS-17 2296 B V1 to desglucosyl-QS-17 2134 B V1
         QS-17 2296 B V2 to desglucosyl-QS-17 2134 B V2
      QS-17 2296 V1 component (i.e. QS-17 2296 A V1 and/or QS-17 2296 B V1) to desglucosyl-QS-17 2134 V1 component, such as:
         QS-17 2296 A V1 to desglucosyl-QS-17 2134 A V1
         QS-17 2296 B V1 to desglucosyl-QS-17 2134 B V1
      QS-17 2296 V2 component (i.e. QS-17 2296 A V2 and/or QS-17 2296 B V1) to desglucosyl-QS-17 2134 V2 component, such as:
         QS-17 2296 A V2 to desglucosyl-QS-17 2134 A V2
         QS-17 2296 B V2 to desglucosyl-QS-17 2134 B V2
   QS-17 2164 component (i.e. QS-17 2164 A and/or QS-17 2164 B) to desglucosyl-QS-17 2002 component, such as:
      QS-17 2164 A component to desglucosyl-QS-17 2002 A
      QS-17 2164 B component to desglucosyl-QS-17 2002 B
   QS-17 2310 component (i.e. QS-17 2310 A and/or QS-17 2310 B) to desglucosyl-QS-17 2148 component, such as:
      QS-17 2310 A component to desglucosyl-QS-17 2148 A component, such as:
         QS-17 2310 A V1 to desglucosyl-QS-17 2148 A V1
         QS-17 2310 A V2 to desglucosyl-QS-17 2148 A V2
      QS-17 2310 B component to QS-21, such as:
         QS-17 2310 B V1 to desglucosyl-QS-17 2148 B V1
         QS-17 2310 B V2 to desglucosyl-QS-17 2148 B V2
      QS-17 2310 V1 component (i.e. QS-17 2310 A V1 and/or QS-17 2310 B V1) to desglucosyl-QS-17 2148 V1 component, such as:
         QS-17 2310 A V1 to desglucosyl-QS-17 2148 A V1
         QS-17 2310 B V1 to desglucosyl-QS-17 2148 B V1
      QS-17 2310 V2 component (i.e. QS-17 2310 A V2 and/or QS-17 2310 B V2) to desglucosyl-QS-17 2148 V2 component, such as:
         QS-17 2310 A V2 to desglucosyl-QS-17 2148 A V2
         QS-17 2310 B V2 to desglucosyl-QS-17 2148 B V2
            Other single sugar enzymatic conversions of interest include:
desarabinofuranosyl-QS-18 family components to desarabinofuranosyl-QS-21 family components, such as:
   desarabinofuranosyl-QS-18 2018 component (i.e. desarabinofuranosyl-QS-18 2018 A and/or desarabinofuranosyl-QS-18 2018 B) to desarabinofuranosyl-QS-21 1856 component, such as:
      desarabinofuranosyl-QS-18 2018 A component to desarabinofuranosyl-QS-21 1856 A component, such as:
         desarabinofuranosyl-QS-18 2018 A V1 to desarabinofuranosyl-QS-21 1856 A V1
         desarabinofuranosyl-QS-18 2018 A V2 to desarabinofuranosyl-QS-21 1856 A V2
      desarabinofuranosyl-QS-18 2018 B component to desarabinofuranosyl-QS-21 1856 B component, such as:
         desarabinofuranosyl-QS-18 2018 B V1 to desarabinofuranosyl-QS-21 1856 B V1
         desarabinofuranosyl-QS-18 2018 B V2 to desarabinofuranosyl-QS-21 1856 B V2
      desarabinofuranosyl-QS-18 2018 V1 component (i.e. desarabinofuranosyl-QS-18 2018 A V1 and/or desarabinofuranosyl-QS-18 2018 B V1) to desarabinofuranosyl-QS-21 1856 V1 component, such as:
         desarabinofuranosyl-QS-18 2018 A V1 to desarabinofuranosyl-QS-21 1856 A V1
         desarabinofuranosyl-QS-18 2018 B V1 to desarabinofuranosyl-QS-21 1856 B V1
      desarabinofuranosyl-QS-18 2018 V2 component (i.e. desarabinofuranosyl-QS-18 2018 A V2 and/or desarabinofuranosyl-QS-18 2018 B V2) to desarabinofuranosyl-QS-21 1856 V2 component, such as:
         desarabinofuranosyl-QS-18 2018 A V2 to desarabinofuranosyl-QS-21 1856 A V2
         desarabinofuranosyl-QS-18 2018 B V2 to desarabinofuranosyl-QS-21 1856 B V2
   desarabinofuranosyl-QS-18 1886 component (i.e. desarabinofuranosyl-QS-18 1886 A and/or desarabinofuranosyl-QS-18 1886 B) to desarabinofuranosyl-QS-21 1712 component, such as:
      desarabinofuranosyl-QS-18 1886 A component to desarabinofuranosyl-QS-21 1712 A
      desarabinofuranosyl-QS-18 1886 B component to desarabinofuranosyl-QS-21 1712 B
   desarabinofuranosyl-QS-18 2032 component (i.e. desarabinofuranosyl-QS-18 2032 A and/or desarabinofuranosyl-QS-18 2032 B) to desarabinofuranosyl-QS-21 1870 component, such as:
      desarabinofuranosyl-QS-18 2032 A component to desarabinofuranosyl-QS-21 1870 A component, such as:
         desarabinofuranosyl-QS-18 2032 A V1 to desarabinofuranosyl-QS-21 1870 A V1
         desarabinofuranosyl-QS-18 2032 A V2 to desarabinofuranosyl-QS-21 1870 A V2
      desarabinofuranosyl-QS-18 2032 B component to QS-21, such as:
         desarabinofuranosyl-QS-18 2032 B V1 to desarabinofuranosyl-QS-21 1870 B V1
         QS-17 desarabinofuranosyl-QS-18 2032 B V2 to desarabinofuranosyl-QS-21 1870 B V2
      desarabinofuranosyl-QS-18 2032 V1 component (i.e. desarabinofuranosyl-QS-18 2032 A V1 and/or desarabinofuranosyl-QS-18 2032 B V1) to desarabinofuranosyl-QS-21 1870 V1 component, such as:
         desarabinofuranosyl-QS-18 2032 A V1 to desarabinofuranosyl-QS-21 1870 A V1
         desarabinofuranosyl-QS-18 2032 B V1 to desarabinofuranosyl-QS-21 1870 B V1
      desarabinofuranosyl-QS-18 2032 V2 component (i.e. desarabinofuranosyl-QS-18 2032 A V2 and/or desarabinofuranosyl-QS-18 2032 B V2) to desarabinofuranosyl-QS-21 1870 V2 component, such as:
         desarabinofuranosyl-QS-18 2032 A V2 to desarabinofuranosyl-QS-21 1870 A V2
         desarabinofuranosyl-QS-18 2032 B V2 to desarabinofuranosyl-QS-21 1870 B V2
acetylated desglucosyl-QS-17 components to acetylated QS-21 family components, such as:
   acetylated desglucosyl-QS-17 2176 A to acetylated QS-21 2030 A, such as:
      acetylated desglucosyl-QS-17 2176 A V1 to acetylated QS-21 2030 A V1
      acetylated desglucosyl-QS-17 2176 A V2 to acetylated QS-21 2030 A V2
   acetylated desglucosyl-QS-17 2044 A to acetylated QS-21 1898 A component
   acetylated desglucosyl-QS-17 2190 A to acetylated QS-21 2044 A, such as:
      acetylated desglucosyl-QS-17 2190 A V1 to acetylated QS-21 2044 A V1
      acetylated desglucosyl-QS-17 2190 A V2 to acetylated QS-21 2044 A V2.

Enzymatic conversions may be applied to a single starting saponin or a plurality of starting saponins in parallel. It will be appreciated that a process may comprise or consist of the conversions specified above, depending on the composition of the starting material and the enzymes used. Furthermore, while a process may be limited to the use of a single enzyme intended to remove a particular sugar residue or group of sugar residues from (i) a single starting saponin, (ii) a family of starting saponins, or (iii) from a plurality of families of starting saponins; processes may also use a plurality of enzymes intended to remove a plurality of sugar residues from (i) a single starting saponin, (ii) a family of starting saponins, or (iii) from a plurality of families of starting saponins. Processes involving multiple enzymes may be undertaken in series (i.e. a single enzyme is applied to saponin material at any time) or in parallel (i.e. more than one enzyme is applied to saponin material at any time, such as two or three enzymes, in particular two enzymes), or combinations thereof.

Processes involving the removal of multiple sugar residues may involve the removal of single (but different) sugar residues from multiple starting saponins and/or the removal of multiple sugar residues from particular starting saponins (such as 2, 3 or 4 residues, in particular 2 or 3, especially 2 residues). Removal of multiple sugar residues from particular starting saponins may involve any combination of removal of single residues and/or removal of a plurality of residues in a single cleavage.

Exemplary processes may comprise (such as consist of) the removal of glucose and rhamnose, in particular an alpha-rhamnose residue and a beta-glucose residue, such as the alpha-L-rhamnose residue and the beta-D-glucose residue from quillaic acid glycosides:

Particular multi-sugar enzymatic conversions of interest include:
QS-17 family components to QS-21 family components, such as:
   QS-17 2296 component (i.e. QS-17 2296 A and/or QS-17 2296 B) to QS-21 1988 component, such as:
      QS-17 2296 A component to QS-21 1988 A component, such as:
         QS-17 2296 A V1 to QS-21 1988 A V1
         QS-17 2296 A V2 to QS-21 1988 A V2
      QS-17 2296 B component to QS-21 1988 B component, such as:
         QS-17 2296 B V1 to QS-21 1988 B V1
         QS-17 2296 B V2 to QS-21 1988 B V2
      QS-17 2296 V1 component (i.e. QS-17 2296 A V1 and/or QS-17 2296 B V1) to QS-21 1988 V1 component, such as:
         QS-17 2296 A V1 to QS-21 1988 A V1
         QS-17 2296 B V1 to QS-21 1988 B V1
      QS-17 2296 V2 component (i.e. QS-17 2296 A V2 and/or QS-17 2296 B V2) to QS-21 1988 V2 component, such as:
         QS-17 2296 A V2 to QS-21 1988 A V2
         QS-17 2296 B V2 to QS-21 1988 B V2
   QS-17 2164 component (i.e. QS-17 2164 A and/or QS-17 2164 B) to QS-21 1856 component, such as:
      QS-17 2164 A component to QS-21 1856 A component
      QS-17 2164 B component to QS-21 1856 B component
   QS-17 2310 component (i.e. QS-17 2310 A and/or QS-17 2310 B) to QS-21 2002 component, such as:
      QS-17 2310 A component to QS-21 2002 A component, such as:
         QS-17 2310 A V1 to QS-21 2002 A V1
         QS-17 2310 A V2 to QS-21 2002 A V2
      QS-17 2310 B component to QS-21 2002 B component, such as:
         QS-17 2310 B V1 to QS-21 2002 B V1
         QS-17 2310 B V2 to QS-21 2002 B V2
      QS-17 2310 V1 component (i.e. QS-17 2310 A V1 and/or QS-17 2310 B V1) to QS-21 2002 V1, such as:
         QS-17 2310 A V1 to QS-21 2002 A V1
         QS-17 2310 B V1 to QS-21 2002 B V1
      QS-17 2310 V2 component (i.e. QS-17 2310 A V2 and/or QS-17 2310 B V2) to QS-21 2002 V2, such as:
         QS-17 2310 A V2 to QS-21 2002 A V2
         QS-17 2310 B V2 to QS-21 2002 B V2.

Extracts may contain complex mixtures of saponin components and consequently may experience a plurality of conversions when multiple enzymes are present. For example, a starting mixture containing QS-17, QS-18 and desglucosyl-QS-17 components which is treated with an appropriate beta-glucosidase and alpha-rhamnosidase in parallel may undergo conversions including:
QS-18 family components to QS-21 family components, especially QS-18 2150 component to QS-21 1988 component;
desglucosyl-QS-17 family components to QS-21 family components, especially desglucosyl-QS-17 2134 component to QS-21 1988 component;
QS-17 family components to desglucosyl-QS-17 family components to QS-21 components, especially QS-17 2296 component to desglucosyl-QS-17 2134 component to QS-21 1988 component; and
QS-17 family components to QS-18 family components to QS-21 family components, especially QS-17 2296 component to QS-18 2150 component to QS-21 1988 component.

### Enzyme Selection

Extensive protein or DNA databases of natural and artificial glycosidases are available. Candidate enzymes may be selected and screened to assess suitability for achieving a particular conversion under particular reaction conditions. Suitability of an enzyme will depend on a number of factors including:
target sugar (e.g. glucose, rhamnose)
target sugar anomer (alpha or beta);
target sugar enantiomer (D or L);
target sugar location (endo or exo); and
target sugar environment (e.g. chemical/physical, impacting accessibility and reactivity).

Additional factors which facilitate effective conversions include:
rate of conversion;
environmental sensitivity—including pH, temperature, substrate, product and contaminant concentration tolerance; and
specificity for target sugar, including in respect of other sugar residues, other anomers, other sugar residue locations, and between different residues of the same sugar anomer and location within a substrate (if multiple such residues are present).

Those skilled in the art will appreciate that the level and type of specificity required of an enzyme will depend on the objective to be achieved and the general circumstances.

Conversion of QS-18 family components to QS-21 family components requires an enzyme demonstrating beta exo glucosidase activity.

Conversion of QS-17 family components to desglucosyl-QS-17 family components requires an enzyme demonstrating beta exo glucosidase activity.

Conversion of desglucosyl-QS-17 family components to QS-21 family components requires an enzyme demonstrating alpha exo rhamnosidase activity.

Conversion of QS-17 family components to QS-18 family components requires an enzyme demonstrating alpha exo rhamnosidase activity.

It may be noted that many Quillaja saponaria starting saponins of interest contain only one glucose residue. Many Quillaja saponaria starting saponins of interest contain a plurality of rhamnose residues, therefore selectivity for specific rhamnose residues is of greater importance practically. For example, conversion of desglucosyl-QS-17 family components to QS-21 components or QS-17 family components to QS-18 family components requires specificity for exo-rhamnosidase action over endo-rhamnosidase action. Furthermore, rhamnosidase specificity for the alpha-O-rhamnosylation at the C2 position of the arabinofuranose moiety over other terminal rhamnose residues (e.g. in the rhamnose chemotype components) may also be desirable. In certain embodiments it may be desirable to remove the terminal rhamnose from rhamnose chemotype components (alone or in conjunction with any alpha-O-rhamnosylation at the C2 position of the arabinofuranose moiety), to better facilitate their chromatographic separation from xylose chemotype components.

In one embodiment saponin starting material is subjected to enzymatic modification by a single enzyme. The single enzyme may be a glucosidase, in particular a beta exo glucosidase. A single enzyme glucosidase may be an engineered glucosidase polypeptide of the present invention. Alternatively, the single enzyme is a rhamnosidase, in particular an alpha exo rhamnosidase. A single enzyme rhamnosidase may an engineered rhamnosidase polypeptide of the present invention.

Preferred enzymes are those which efficiently enzymatically convert a starting saponin(s) to the desired product saponin(s) while demonstrating limited or no undesired conversion(s) of other saponin components present.

In one embodiment saponin starting material is subjected to enzymatic modification by more than one enzyme, such as by two or three enzymes, especially by two enzymes. Enzymatic modification by more than one enzyme may involve sequential/series enzymatic modification. Alternatively, enzymatic modification by more than one enzyme may involve concurrent/parallel enzymatic modification. Enzymatic modification by at least three enzymes may involve a combination of sequential/series (modification by one enzyme) and concurrent/parallel (modification by at least two other enzymes) enzymatic modification, in any order. Where a plurality of enzymes are provided, these may be as distinct proteins or may be in the form of one or more fusion proteins.

An enzyme of interest is a glucosidase, such as a beta exo glucosidase. A glucosidase may be an engineered glucosidase polypeptide of the present invention. Another enzyme of interest is a rhamnosidase, such as an alpha exo rhamnosidase. A rhamnosidase may an engineered rhamnosidase polypeptide of the present invention. Enzyme combinations of interest include those comprising, such as consisting of, a glucosidase and a rhamnosidase, in particular a beta exo glucosidase and an alpha exo rhamnosidase. Enzymatic modification involving a glucosidase and a rhamnosidase, in particular a beta exo glucosidase and an alpha exo rhamnosidase, may be undertaken: sequentially with glucosidase (e.g. beta exo glucosidase) followed by rhamnosidase (e.g. alpha exo rhamnosidase), sequentially with rhamnosidase (e.g. alpha exo rhamnosidase) followed by glucosidase (e.g. beta exo glucosidase) or, conveniently, concurrently with both glucosidase (e.g. beta exo glucosidase) and rhamnosidase (e.g. alpha exo rhamnosidase). Particular enzyme combinations of interest are those comprising, such as consisting of, an engineered glucosidase of the present invention and an engineered rhamnosidase polypeptide of the present invention.

Enzymes utilised will typically be of external origin to saponin material i.e. not naturally found within the source of saponins obtained by extraction.

Enzymes may be native, i.e. naturally occurring glycosidases, or alternatively may be non-naturally occurring glycosidases. In one embodiment a glucosidase enzyme is a naturally occurring glucosidase (e.g. exo glucosidase, such as beta exo glucosidase). In a second embodiment a glucosidase enzyme is a non-naturally occurring glucosidase (e.g. exo glucosidase, such as beta exo glucosidase). In one embodiment a rhamnosidase enzyme is a naturally occurring rhamnosidase (e.g. exo rhamnosidase, such as alpha exo rhamnosidase). In a second embodiment a rhamnosidase enzyme is a non-naturally occurring rhamnosidase (e.g. exo rhamnosidase, such as alpha exo rhamnosidase).

Enzymes may be modified relative to a reference enzyme (‘engineered’). Point mutations, either singly or in combination, introduced by engineering may provide benefits such as increased activity, increased specificity, increased stability, increased expression or other the like. Assays to confirm the properties of the enzymes are well known to those skilled in the field. For example, activity may be quantified by methods such as those shown in the examples (see Examples 4 to 7) or by analogous methods.

Different enzymes may show different sensitivity to environmental conditions, such as pH, temperature, substrate concentration, product concentration, solvent composition, presence of contaminants and the like. Such parameters may be taken into consideration during screening of candidate enzymes for the desired activity.

Candidate enzymes having beta glucosidase activity include those in EC3.2.1.21.

Beta exo glucosidases of interest include those described in Table 7, especially SEQ ID Nos. 262, 208, 63, 229, 250, 5, 101, 207, 169, 247, 302, 324, 319, 9, 240, 325 and 338, and functional variants thereof. Particular beta exo glucosidases of interest include SEQ ID Nos. 262, 208, 63, 229, 250, 5, 101, 207, 169, 247, 302, 324 and 319, and functional variants thereof, such as SEQ ID Nos. 262, 208, 63, 229, 250, 5, 101 and 207, and functional variants thereof.

Another group of beta exo glucosidases of interest include those described in Table 9, especially SEQ ID Nos. 850, 879, 868, 826, 804, 888, 881, 891, 816, 827, 857, 853, 842, 814, 886, 885, 838, 829, 808, 828, 870, 873, 844, 882, 874, 825, 824, 823, 810, 894, 849, 803, 890, 841, 832, 830, 845, 871, 837, 883 and 809, and functional variants thereof. Particular beta exo glucosidases of interest include SEQ ID Nos. 850, 879, 868, 826, 804, 888, 881, 891, 816, 827, 857, 853, 842, 814, 886, 885, 838, 829, 808, 828, 870, 873, 844, 882, 874, 825, 824, 823, 810, 894, 849, 803, 890 and 841, and functional variants thereof, such as SEQ ID Nos. 850, 879, 868, 826, 804, 888, 881, 891, 816, 827, 857, 853, 842, 814, 886, 885, 838, 829, 808, 828, 870, 873, 844, 882, 874, 825, 824, 823, 810 and 894, and functional variants thereof.

SEQ ID No. 262, and functional variants thereof, are particularly desirable beta exo glucosidases. In one embodiment the beta exo glucosidase comprises, such as consists of: (i) SEQ ID. 262; or (ii) a functional variant thereof having at least 80% identity to SEQ ID. 262, especially at least 90%, in particular at least 95%, such as at least 96%, at least 97%, at least 98%, for example at least 99% identity; or (iii) a functional fragment of at least 100, especially at least 200, particularly at least 300, such as at least 400, for example at least 500 contiguous amino acids of SEQ ID. 262.

Candidate enzymes having alpha rhamnosidase activity include those in EC3.2.1.40.

Alpha exo rhamnosidases of interest include SEQ ID Nos. 992, 1003, 1052, 1073, 1017, 1055, 1075, 1001, 1007, 1061, 1079, 1027, 1039, 1041, 989, 1053, 1018, 1066, 1082, 1076, 993, 1077, 1046, 1015, 1063, 1054, 1074, 1067 and 1033, and functional variants thereof. Particular alpha exo rhamnosidases of interest include SEQ ID Nos. 992, 1003, 1052, 1073, 1017, 1055, 1075, 1001, 1007, 1061, 1079, 1027, 1039, 1041, 989, 1053, 1018, 1066, 1082, 1076, 993 and 1077, and functional variants thereof, such as SEQ ID Nos. 992, 1003, 1052, 1073, 1017, 1055, 1075, 1001, 1007, 1061, 1079, 1027, 1039, 1041 and 989, and functional variants thereof.

SEQ ID No. 1017, and functional variants thereof, are particularly desirable exo rhamnosidases. In one embodiment the alpha exo rhamnosidase comprises, such as consists of: (i) SEQ ID. 1017; or (ii) a functional variant thereof having at least 80% identity to SEQ ID. 1017, especially at least 90%, in particular at least 95%, such as at least 96%, at least 97%, at least 98%, for example at least 99% identity; or (iii) a functional fragment of at least 100, especially at least 200, particularly at least 300, such as at least 400, for example at least 500 contiguous amino acids of SEQ ID. 1017.

Functional variants of interest in the present application include those comprising, such as consisting of: (i) a sequence having at least 80% identity to the reference sequence, especially at least 90%, in particular at least 95%, such as at least 96%, at least 97%, at least 98%, for example at least 99% identity; or (ii) a fragment of at least 100, especially at least 200, particularly at least 300, such as at least 400, for example at least 500 contiguous amino acids of the reference sequence.

Certain desirable functional variants of interest include those comprising, such as consisting of, a sequence having 1 to 20 additions, deletions and/or substitutions relative to the reference sequence, especially 1 to 15 additions, deletions and/or substitutions, particularly 1 to additions, deletions and/or substitutions, such as 1 to 5 additions, deletions and/or substitutions.

The degree of sequence identity may be determined using by the homology alignment algorithm of Needleman and Wunsch, the ClustalW program or the BLASTP algorithm, using default settings. An algorithm using global alignment (Needleman and Wunsch) is preferred.

“Percentage of sequence identity,” “percent identity,” and “percent identical” are used herein to refer to comparisons between polynucleotide sequences or polypeptide sequences, and are determined by comparing two optimally aligned sequences over a comparison window, wherein the portion of the polynucleotide or polypeptide sequence in the comparison window may comprise additions or deletions (i.e., gaps) as compared to the reference sequence for optimal alignment of the two sequences. The percentage is calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Determination of optimal alignment and percent sequence identity is performed using the BLAST and BLAST 2.0 algorithms (see, e.g., Altschul, 1990; Altschul, 1997). Software for performing BLAST analyses is publicly available through the National Center for Biotechnology Information website. Briefly, the BLAST analyses involve first identifying high scoring sequence pairs (HSPs) by identifying short words of length W in the query sequence, which either match or satisfy some positive-valued threshold score T when aligned with a word of the same length in a database sequence. T is referred to as, the neighborhood word score threshold (Altschul, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (N) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff, 1989).

Numerous other algorithms are available that function similarly to BLAST in providing percent identity for two sequences. Optimal alignment of sequences for comparison can be conducted, e.g., by the local homology algorithm of Smith, 1981, by the homology alignment algorithm of Needleman, 1970, by the search for similarity method of Pearson, 1988, by computerized implementations of these algorithms (GAP, BESTFIT, FASTA, and TFASTA in the GCG Wisconsin Software Package), or by visual inspection (see generally, Current Protocols in Molecular Biology, 1995)). Additionally, determination of sequence alignment and percent sequence identity can employ the BESTFIT or GAP programs in the GCG Wisconsin Software package (Accelrys, Madison WI), using default parameters provided. The ClustalW program is also suitable for determining identity.

Modestobacter marinus glucosidase (Uniparc reference UPI000260A2FA, Uniprot reference I4EYD5-SEQ ID No. 262 herein) is a naturally occurring glucosidase demonstrating beta exo glucosidase activity and, for example, is capable of the conversion of QS-18 family components to QS-21 family components. Despite its potent activity, the present inventors have found that the properties of wild type Modestobacter marinus glucosidase may be altered by the introduction of one or more mutations.

The present invention provides an engineered glucosidase polypeptide comprising, such as consisting of, an amino acid sequence that is at least 80%, 85%, 90%, 95%, 96%, 97%, 98%, or 99% identical to the amino acid sequence of SEQ ID No. 262, or a functional fragment thereof, wherein the engineered glucosidase polypeptide includes at least one residue substitution from:
F44Y;
V60L;
G117A;
F170N;
V263G or V263L;
N351H or N351Q;
A355H, A355I, A355L, A355M, A355R, A355T or A355W;
A356P;
R357A, R357C, R357K, R357M or R357Q;
G362C;
T365A, T365N or T365S;
L367C;
V394R;
V395Y;
Q396E, Q396G, Q396N, Q396P, Q396R, Q396S or Q396Y;
F430W;
R435F;
V438T;
V440F;
F442M or F442Q;
G444T;
A473F or A473R;
L474C, L474I or L474V;
I475F;
L492C, L492G, L492H, L492I, L492N, L492Q, L492V, L492W or L492Y;
Q493F or Q493H;
P494H or P494I;
S495I, S495K or S495Q;
G496P or G496W;
D498A, D498E, D498F, D498I, D498K, D498L, D498N, D498P, D498R, D498S, D498T or D498V;
A502R;
M504G or M504R;
L507A or L507R;
T508M;
L529M;
F535P;
A536D or A536E;
A537R;
F541A, F541I, F541L, F541M or F541V;
L542I;
Q543G or Q543L;
E547L; and
Y585W.

The glucosidases will contain one to forty-two of the substitutions, such as two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six to thirty or thirty-one to forty-three substitutions.

The present invention also provides an engineered glucosidase polypeptide comprising, such as consisting of, an amino acid sequence that is at least 80%, 85%, 90%, 95%, 96%, 97%, 98%, or 99% identical to the amino acid sequence of SEQ ID No. 262, or a functional fragment thereof, wherein the engineered glucosidase polypeptide includes at least one residue substitution from:
F44Y;
V263L;
N351H;
A355H, A355M or A355W;
R357M;
T365N;
L367C;
Q396R;
V438T;
F442Q;
L474C;
I475F;
L492V, L492N or L492H,
M504R;
L507R; and
F541I.

The glucosidases will contain one, two, three, four, five, six, seven, eight, nine, ten, eleven, twelve, thirteen, fourteen, fifteen or all sixteen substitutions.

The engineered glucosidase polypeptide may comprise, such as consist of, an amino acid sequence that is at least 80%, 85%, 90%, 95%, 96%, 97%, 98%, or 99% identical to the amino acid sequence of SEQ ID No. 262, or a functional fragment thereof, wherein the engineered glucosidase polypeptide includes at least one residue substitution from: F44Y, V263L, A355W, R357M, T365N, L367C, Q396R, F442Q, L474C, I475F and F541I.

Suitably the engineered glucosidase polypeptide comprises, such as consists of, an amino acid sequence that is at least 80%, 85%, 90%, 95%, 96%, 97%, 98%, or 99% identical to the amino acid sequence of SEQ ID No. 262, or a functional fragment thereof, wherein the engineered glucosidase polypeptide includes the residue substitutions: F44Y, V263L, A355W, R357M, T365N, L367C, Q396R, F442Q, L474C, I475F and F541I.

The present invention provides a polypeptide comprising an amino acid sequence of sequence of SEQ ID No. 262 with one to twenty-five mutations selected from the list consisting of:
(i) F44Y
(ii) V263L
(iii) N351H
(iv) A355H, A355I, A355L, A355M, A355R, A355T or A355W
(v) A356P
(vi) R357M
(vii) T365N
(viii) L3670
(ix) F442Q
(x) G443D
(xi) A473F
(xii) L474O
(xiii) I475F
(xiv) L492H, L492N, L492V
(xv) P494I
(xvi) G496P
(xvii) D498P
(xviii) M504R
(xix) L507R
(xx) F535P
(xxi) A537R
(xxii) F541I
(xxiii) L542I;
(xxiv) E547L and
(xxv) E588K.

Variant glucosidases will contain one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four or all twenty-five mutations.

In some embodiments an engineered glucosidase is not a polypeptide comprising an amino acid sequence of sequence of SEQ ID No. 262 with one to twenty-five mutations selected from the list consisting of:
(i) F44Y
(ii) V263L
(iii) N351H
(iv) A355H, A355I, A355L, A355M, A355R, A355T or A355W
(v) A356P
(vi) R357M
(vii) T365N
(viii) L367C
(ix) F442Q
(x) G443D
(xi) A473F
(xii) L474C
(xiii) I475F
(xiv) L492H, L492N, L492V
(xv) P494I
(xvi) G496P
(xvii) D498P
(xviii) M504R
(xix) L507R
(xx) F535P
(xxi) A537R
(xxii) F541I
(xxiii) L542I
(xxiv) E547L and
(xxv) E588K.

The above-mentioned engineered glucosidase polypeptides may also be referred to herein as examples of ‘variant glucosidases’.

A variant glucosidase may contain F44Y.

A variant glucosidase may contain V60L.

A variant glucosidase may contain G117A.

A variant glucosidase may contain F170N.

A variant glucosidase may contain V263G or V263L, in particular V263L.

A variant glucosidase may contain N351H or N351Q, in particular N351H.

A variant glucosidase may contain A355H, A355I, A355L, A355M, A355R, A355T or A355W. In some embodiments a variant glucosidase contains A355H. In some embodiments a variant glucosidase contains A355I. In some embodiments a variant glucosidase contains A355L. In some embodiments a variant glucosidase contains A355M. In some embodiments a variant glucosidase contains A355R. In some embodiments a variant glucosidase contains A355T. In some embodiments a variant glucosidase contains A355W.

A variant glucosidase may contain A356P.

A variant glucosidase may contain R357A, R357C, R357K, R357M or R357Q, in particular R357M.

A variant glucosidase may contain G362C.

A variant glucosidase may contain T365A, T365N or T365S, in particular T365N.

A variant glucosidase may contain L367C.

A variant glucosidase may contain V394R.

A variant glucosidase may contain V395Y.

A variant glucosidase may contain Q396E, Q396G, Q396N, Q396P, Q396R, Q396S or Q396Y, in particular Q396R.

A variant glucosidase may contain F430W.

A variant glucosidase may contain R435F.

A variant glucosidase may contain V438T.

A variant glucosidase may contain V440F.

A variant glucosidase may contain F442M or F442Q, in particular F442Q.

A variant glucosidase may contain G443D.

A variant glucosidase may contain G444T.

A variant glucosidase may contain A473F or A473R, in particular A473F.

A variant glucosidase may contain L474O, L474I or L474V, in particular L474C.

A variant glucosidase may contain I475F.

A variant glucosidase may contain L4920, L492G, L492H, L492I, L492N, L492Q, L492V, L492W or L492Y, in particular L492H, L492N, L492V. In some embodiments a variant glucosidase contains L492H. In some embodiments a variant glucosidase contains L492N. In some embodiments a variant glucosidase contains L492V.

A variant glucosidase may contain Q493F or Q493H.

A variant glucosidase may contain P494H or P494I, in particular P494I.

A variant glucosidase may contain S495I, S495K or S495Q.

A variant glucosidase may contain G496P or G496W, in particular G496P.

A variant glucosidase may contain D498A, D498E, D498F, D498I, D498K, D498L, D498N, D498P, D498R, D498S, D498T or D498V, in particular D498P.

A variant glucosidase may contain A502R.

A variant glucosidase may contain M504G or M504R, in particular M504R.

A variant glucosidase may contain L507A or L507R, in particular L507R.

A variant glucosidase may contain T508M.

A variant glucosidase may contain L529M.

A variant glucosidase may contain F535P.

A variant glucosidase may contain A536D or A536E.

A variant glucosidase may contain A537R.

A variant glucosidase may contain F541A, F541I, F541L, F541M or F541V, in particular F541I.

A variant glucosidase may contain L542I.

A variant glucosidase may contain Q543G or Q543L.

A variant glucosidase may contain E547L.

A variant glucosidase may contain Y585W.

A variant glucosidase may contain E588K.

Variant glucosidases may comprise R357M, T365N, A473F, L474O and I475F.

Variant glucosidases may comprise F44Y, R357M, T365N, F442Q, A473F, L474O and I475F.

Variant glucosidases may comprise F44Y, V263L, R357M, T365N, F442Q, A473F, L474C, I475F and F541I.

Variant glucosidases may comprise F44Y, V263L, A355W, R357M, T365N, L367C, Q396R, F442Q, L474O, I475F and F541I.

Variant glucosidases may comprise F44Y, V263L, R357M, T365N, F442Q, L474O, I475F, F541I and zero to seventeen mutations selected from the list consisting of:
(iii) N351H
(iv) A355H, A355I, A355L, A355M, A355R, A355T or A355W
(v) A356P
(viii) L367C
(x) G443D
(xi) A473F
(xiv) L492H, L492N, L492V
(xv) P494I
(xvi) G496P
(xvii) D498P
(xviii) M504R
(xix) L507R
(xx) F535P
(xxi) A537R
(xxiii) L542I
(xxiv) E547L and
(xxv) E588K.

A variant glucosidase may comprise a “tag,” a sequence of amino acids that allows for the isolation and/or identification of the polypeptide. For example, adding an affinity tag can be useful in purification. Exemplary affinity tags that can be used include histidine (HIS) tags (e.g., hexa histidine-tag, or 6×His-Tag), FLAG-TAG, and HA tags. Tags may be located N-terminally or C-terminally and may be directly connected or attached via a linking sequence. SEQ ID No. 1177 provides a sequence for an exemplary 6×His-Tag with linker sequence which may be N-terminally attached. SEQ ID No. 1178 provides a sequence for an exemplary 6×His-Tag with linker sequence which may be C-terminally attached.

In certain embodiments, the tags used herein are removable, e.g., removal by chemical agents or by enzymatic means, once they are no longer needed, e.g., after the polypeptide has been purified.

A variant glucosidase may comprise 1000 residues or fewer, especially 950 residues or fewer, in particular 900 residues or fewer, such as 850 residues or fewer.

A variant glucosidase may consist of an amino acid sequence of SEQ ID No. 262 with one to twenty-five mutations selected from the list consisting of:
(i) F44Y
(ii) V263L
(iii) N351H
(iv) A355H, A355I, A355L, A355M, A355R, A355T or A355W
(v) A356P
(vi) R357M
(vii) T365N
(viii) L367C
(ix) F442Q
(x) G443D
(xi) A473F
(xii) L474C
(xiii) I475F
(xiv) L492H, L492N, L492V
(xv) P494I
(xvi) G496P
(xvii) D498P
(xviii) M504R
(xix) L507R
(xx) F535P
(xxi) A537R
(xxii) F541I
(xxiii) L542I
(xxiv) E547L and
(xxv) E588K.

Variant glucosidases desirably demonstrate a FIOP (Fold Improvement Over Parent) relative to SEQ ID No. 262 of at least 1.05, especially at least 2, in particular at least 10, such as at least 50. FIOP may be determined by the methods described in Example 4.

Kribbella flavida rhamnosidase (Uniparc reference UPI00019BDB13, Uniprot reference D2PMT5—SEQ ID No. 1017 herein) is a naturally occurring rhamnosidase demonstrating alpha exo rhamnosidase activity and, for example, is capable of the conversion of desglucosyl-QS-17 family components to QS-21 family components. Despite its potent activity, the present inventors have found that the properties of wild type Kribbella flavida rhamnosidase may be altered by the introduction of one or more mutations.

The present invention provides an engineered rhamnosidase polypeptide comprising, such as consisting of, an amino acid sequence that is at least 80%, 85%, 90%, 95%, 96%, 97%, 98%, or 99% identical to the amino acid sequence of SEQ ID No. 1017, or a functional fragment thereof, wherein the engineered rhamnosidase polypeptide includes at least one residue substitution from:
(i) A56C
(ii) A143P
(iii) Q181H, Q181R or Q181S
(v) G215S
(vi) F216M
(vii) G218D or G218N
(viii) K219G
(ix) A238M
(x) T252Y
(xi) T311W
(xii) V326C
(xiii) G357C
(xiv) S369C, S369I, S369K or S369M
(xv) I487M, I487Q or I487V
(xvi) K492N
(xvii) V499T
(xviii) G508S
(xix) R543C
(xx) L557Y
(xxi) G634A
(xxii) S635N
(xxiii) A690C and
(xxiv) Q921H.

Consequently, the present invention provides a polypeptide comprising an amino acid sequence of sequence of SEQ ID No. 1017 with one to twenty-four mutations selected from the list consisting of:
(i) A56C
(ii) A143P
(iii) Q181H, Q181R or Q181S
(iv) L214M
(v) G215S
(vi) F216M
(vii) G218D or G218N
(viii) K219G
(ix) A238M
(x) T252Y
(xi) T311W
(xii) V326C
(xiii) G357C
(xiv) S369C, S369I, S369K or S369M
(xv) I487M, I487Q or I487V
(xvi) K492N
(xvii) V499T
(xviii) G508S
(xix) R543C
(xx) L557Y
(xxi) G634A
(xxii) S635N
(xxiii) A690C and
(xxiv) Q921H.

Such polypeptides may be referred to herein as ‘variant rhamnosidases’.

Variant rhamnosidases will contain one, two, three, four, five, six, seven, eight, nine, ten, eleven, twelve, thirteen, fourteen, fifteen, sixteen, seventeen, eighteen, nineteen, twenty, twenty-one, twenty-two, twenty-three or all twenty-four mutations.

A variant rhamnosidase may contain A56C.

A variant rhamnosidase may contain A143P.

A variant rhamnosidase may contain Q181H, Q181R or Q181S. In some embodiments a variant rhamnosidase contains Q181H. In some embodiments a variant rhamnosidase contains Q181R. In some embodiments a variant rhamnosidase contains Q181S.

A variant rhamnosidase may contain L214M.

A variant rhamnosidase may contain G215S.

A variant rhamnosidase may contain F216M.

A variant rhamnosidase may contain G218D or G218N. In some embodiments a variant rhamnosidase contains G218D. In some embodiments a variant rhamnosidase contains G218N.

A variant rhamnosidase may contain K219G.

A variant rhamnosidase may contain A238M.

A variant rhamnosidase may contain T252Y.

A variant rhamnosidase may contain T311W.

A variant rhamnosidase may contain V326C.

A variant rhamnosidase may contain G357C.

A variant rhamnosidase may contain S369C, S369I, S369K or S369M. In some embodiments a variant rhamnosidase contains S369C. In some embodiments a variant rhamnosidase contains S369I. In some embodiments a variant rhamnosidase contains S369K. In some embodiments a variant rhamnosidase contains S369M.

A variant rhamnosidase may contain I487M, I487Q or I487V. In some embodiments a variant rhamnosidase contains I487M. In some embodiments a variant rhamnosidase contains I487Q. In some embodiments a variant rhamnosidase contains I487V.

A variant rhamnosidase may contain K492N.

A variant rhamnosidase may contain V499T.

A variant rhamnosidase may contain G508S.

A variant rhamnosidase may contain R543C.

A variant rhamnosidase may contain L557Y.

A variant rhamnosidase may contain G634A.

A variant rhamnosidase may contain S635N.

A variant rhamnosidase may contain A690C.

A variant rhamnosidase may contain Q921H.

Variant rhamnosidases may comprise A143P, L214M, K219G and Q921H.

Variant rhamnosidases may comprise A143P, L214M, K219G, G357C and Q921H.

Variant rhamnosidases may comprise A143P, L214M, G215S, G218N, K219G, G357C, G508S, G634A and Q921H.

Variant rhamnosidases may comprise A143P, L214M, G215S, G218D, K219G, G357C, G508S, G634A, A690C and Q921H.

Variant rhamnosidases may comprise A143P, L214M, G215S, K219G, G357C, G508S, G634A and Q921H and one to sixteen mutations selected from the list consisting of:
(i) A56C
(iii) Q181H, Q181R or Q181S
(vi) F216M
(vii) G218D or G218N
(ix) A238M
(x) T252Y
(xi) T311W
(xii) V326C
(xiv) S369C, S369I, S369K or S369M
(xv) I487M, I487Q or I487V
(xvi) K492N
(xvii) V499T
(xix) R543C
(xx) L557Y
(xxii) S635N and
(xxiii) A690C.

Variant rhamnosidases may comprise A143P, L214M, G215S, K219G, G357C, G508S, G634A, Q921H, G218D or G218N, and one to fifteen mutations selected from the list consisting of:
(i) A56C
(iii) Q181H, Q181R or Q181S
(vi) F216M
(ix) A238M
(x) T252Y
(xi) T311W
(xii) V326C
(xiv) S369C, S369I, S369K or S369M
(xv) I487M, I487Q or I487V
(xvi) K492N
(xvii) V499T
(xix) R543C
(xx) L557Y
(xxii) S635N and
(xxiii) A690C.

A variant rhamnosidase may comprise a “tag,” a sequence of amino acids that allows for the isolation and/or identification of the polypeptide. For example, adding an affinity tag can be useful in purification. Exemplary affinity tags that can be used include histidine (HIS) tags (e.g., hexa histidine-tag, or 6×His-Tag), FLAG-TAG, and HA tags. Tags may be located N-terminally or C-terminally and may be directly connected or attached via a linking sequence. SEQ ID No. 1177 provides a sequence for an exemplary 6×His-Tag with linker sequence which may be N-terminally attached. SEQ ID No. 1178 provides a sequence for an exemplary 6×His-Tag with linker sequence which may be C-terminally attached.

In certain embodiments, the tags used herein are removable, e.g., removal by chemical agents or by enzymatic means, once they are no longer needed, e.g., after the polypeptide has been purified.

A variant rhamnosidase may comprise 1100 residues or fewer, especially 1050 residues or fewer, in particular 1000 residues or fewer, such as 950 residues or fewer.

A variant rhamnosidase may consist of an amino acid sequence of SEQ ID No. 1017 with one to twenty-four mutations selected from the list consisting of:
(i) A56C
(ii) A143P
(iii) Q181H, Q181R or Q181S
(iv) L214M
(v) G215S
(vi) F216M
(vii) G218D or G218N
(viii) K219G
(ix) A238M
(x) T252Y
(xi) T311W
(xii) V326C
(xiii) G357C
(xiv) S369C, S369I, S369K or S369M
(xv) I487M, I487Q or I487V
(xvi) K492N
(xvii) V499T
(xviii) G508S
(xix) R543C
(xx) L557Y
(xxi) G634A
(xxii) S635N
(xxiii) A690C and
(xxiv) Q921H.

Variant rhamnosidases desirably demonstrate a FIOP relative to SEQ ID No. 1017 of at least 1.05, especially at least 2, in particular at least 10, such as at least 50. FIOP may be determined by the methods described in Example 4.

Function, in respect of functional variants, requires that the glycosidase activity is not notably reduced as a result of sequence variation, typically at least 50% of glycosidase activity, especially at least 75% activity, particularly at least 90%, such as at least 100% activity is maintained for at least one saponin modification reaction under at least one set of conditions (activity being determined by rate of modification of starting saponin to product saponin). Variants may be created with the intention of improving the glycosidase in some manner (e.g. conversion rate; specificity, which may be increased or reduced depending on needs; tolerance to environmental conditions, such as pH, substrate concentration, product concentration, other contaminants and the like; stability, thermal or chemical; production, such as facilitating expression or purification of the glycosidase either pre- or post-saponin modification). Variants need not be improved in all respects and may simply demonstrate a different balance of characteristics relative to the reference sequence.

Glycosidases will typically be 2000 amino acids or fewer, such as 1500 amino acids or fewer.

Suitably, glycosidases are soluble.

Glycosidases may be immobilised, such as by attachment to solid (e.g. polymer) particles. Immobilisation of glycosidases may facilitate separation from a reaction mixture, improve thermal stability and/or tolerance to environmental conditions.

Glycosidases may comprise a “tag,” a sequence of amino acids that allows for the isolation and/or identification of the polypeptide. For example, adding an affinity tag can be useful in purification. Exemplary affinity tags that can be used include histidine (HIS) tags (e.g., hexa histidine-tag, or 6×His-Tag), FLAG-TAG, and HA tags. Tags may be located N-terminally or C-terminally and may be directly connected or attached via a linking sequence. SEQ ID No. 1177 provides a sequence for an exemplary 6×His-Tag with linker sequence which may be N-terminally attached. SEQ ID No. 1178 provides a sequence for an exemplary 6×His-Tag with linker sequence which may be C-terminally attached.

### Reaction Conditions

Any suitable reaction conditions may be used. Optimal conditions will depend on a range of factors including the identity of the starting saponin, product saponin, enzyme utilised and the like.

The reaction requires treatment of a starting saponin(s) with a glycosidase(s). Appropriate glycosidases may be added to a saponin containing composition in a range of forms such as solution (typically aqueous), suspension (typically aqueous) or solid. Glycosidases may be in a purified, partially purified (such as clarified cell lysate) or unpurified form (crude cell lysate or unlysed cells). The use of partially purified or unpurified forms may be of interest when source cells (e.g. recombinant host cells, such as E. coli) express the enzyme to an extent that desired activity sufficiently exceeds any deleterious impact arising from other host cell contaminants. Desirably the glycosidase(s) are added in the form of clarified lysates. Glycosidases may be freshly prepared (e.g. clarified lysate) or taken from storage, such as thawed frozen liquid (e.g. clarified lysate) or reconstituted dried material (e.g. freeze-dried clarified lysate). Where a plurality of glycosidases is used in parallel, these will typically be expressed in different host cells to ensure adequate process control. A plurality of glycosidases used in parallel may be added together or separately (in the same or different forms).

Glycosidases may be produced using a protein secretion system, such as Bacillus licheniformis.

The weight of a glycosidase present may be in the range of 0.0001 mg to 25 mg per ml, especially 0.0001 mg to 5 mg per ml, in particular 0.0001 mg to 1 mg per ml, such as 0.001 mg to 0.5 mg per ml. When provided in the form of dried clarified lysate, the weight of a glycosidase present may be in the range of 0.01 mg to 100 mg of lysate per ml, especially 0.01 mg to 30 mg per ml, in particular 0.01 mg to 5 mg per ml, such as 0.01 mg to 1 mg per ml.

Any appropriate pH may be used, though typically between pH 4 to 9, especially pH 5 to 8, and in particular pH 5.5 to 7.5 such as pH 5.5 to 6.5. Where a plurality of glycosidases is used in series, each enzymatic modification may be undertaken at a different pH though for convenience they may be undertaken at the same pH.

Buffers may be used to aid control of the pH. Suitable buffers and appropriate concentrations may be obtained from standard sources. Inorganic salt buffers are typically used, such as potassium phosphate, sodium phosphate, potassium acetate, sodium acetate, potassium citrate, sodium citrate and the like. A suitable buffer concentration may be 10 mM to 500 mM, especially 25 mM to 250 mM and in particular 50 mM to 100 mM. Buffer concentrations of about 50 mM, such as 50 mM or about 100 mM, such as 100 mM, may be used.

Any appropriate temperature may be used, though typically between 10 degC to 60 degC, especially 15 degC to 50 degC, in particular 15 degC to 45 degC, such as 20 degC to 42 degC.

An appropriate time such that the reaction proceeds sufficiently is usually up to 10 days, especially up to 5 days, in particular up to 3 days. Desirably the enzyme and reaction conditions are chosen such that the reaction proceeds sufficiently in a period of up to 2 days, especially up to 1 day, in particular up to 18 hrs, such as 12 hrs, for example up to 6 hrs.

The reaction will be undertaken in a suitable solvent, typically water or an aqueous solution with water miscible co-solvent(s) such as methanol, ethanol, n-propanol, i-propanol, tetrahydrofuran, ethylene glycol, glycerol,1,3-propanediol or acetonitrile. Any co-solvent(s) should be present in amounts which are not excessively deleterious to the reaction proceeding, such as 50% or less v/v, especially 20% or less, in particular 10% or less, such as 5% or less, for example 2% or less (in total).

The reaction may be homogeneous or heterogeneous, monophasic, bi-phasic or multiphasic with particulates, dispersed solids in suspension and/or colloidal micelles present. Desirably the reaction will be monophasic.

The starting saponins may be present at a concentration of 0.001 to 100 g per litre, especially 0.005 to 75 g per litre, in particular 0.01 to 50 g per litre, such as 0.1 to 25 g per litre, for example 1 to 10 g per litre.

The reaction may be carried out in various modes of operation such as batch mode, fed batch mode or continuous mode.

The reaction is typically performed at a scale which can provide commercial quantities of product saponin. A batch reaction volume may be at least 10 ml, especially at least 100 ml, in particular at least 1 L. A batch reaction volume may be 500 ml to 2000 L, especially 1 L to 1000 L, in particular 10 L to 500 L, such as 25 L to 200 L.

### Completion and Mass Balance

Enzymes are desirably adequately selective for the conversion of a starting saponin into a product saponin rather than other conversions of the starting saponin. As used herein, the term selectivity means at least 25% (mole basis) of converted starting saponin results in the intended product saponin, in particular at least 50%, especially at least 75%, such as at least 90% (e.g. at least 95%).

The concept of selectivity may also be applied in the context of the conversion of a plurality of starting saponins into a plurality of product saponins such that at least 25% of converted starting saponins (mole basis) result in the intended product saponins, in particular at least 50%, especially at least 75%, such as at least 90% (e.g. at least 95%).

Desirably conversion of a starting saponin into a product saponin is complete. However, rate of conversion, specificity of conversion (including rate of non-specific conversion(s)), product inhibition, starting saponin stability under reaction conditions, product saponin stability under reaction conditions and the like mean that conversions may not be complete or that it is desirable (e.g. for maximum yield or to obtain a balance between yield and process time) for a conversion to be stopped prior to completion.

### Removal of Enzymes

At the point the reaction has progressed to the desired extent it may be stopped by denaturing or otherwise removing the enzyme. For example, the pH of reaction mixture may be adjusted to about pH to 3.5 to 4, especially pH 3.5 to 4, in particular pH 3.8 and/or the addition of sufficient quantities of anti-solvents or denaturing solvents such as acetonitrile. Precipitated enzyme may be removed by filtration.

### Other Definitions

By the term ‘Preceding peak’ is meant the peak immediately preceding the QS-21 main peak in the HPLC-UV methods described herein (see FIG. 2).

By the term ‘m/z’ is meant the mass to charge ratio of the monoisotope peak. Unless otherwise specified, ‘m/z’ is determined by negative ion electrospray mass spectrometry.

By the term ‘ion abundance’ is meant the amount of a specified m/z measured in the sample, or in a given peak as required by the context. The mass chromatogram for the specified m/z may be extracted from the MS total ion chromatogram in the UPLC-UV/MS methods described herein. The mass chromatogram plots the signal intensity versus time. Ion abundance is measured as the area of the integrated peak. The area for a specified m/z/area for a relative reference m/z=relative abundance.

By the term ‘UV absorbance at 214 nm’ is meant the area of an integrated peak in the UV absorbance chromatogram. The (area for a specified peak)/(area of all integrated peaks in the chromatogram)×100=percentage area for the specified peak.

By the term ‘UV absorbance at 214 nm and relative ion abundance’ is meant an estimate for the percentage of a given m/z for co-eluting species. (Percentage area for given UV peak)×(relative ion abundance for m/z of interest in given peak)/(sum of all relative ion abundance for given peak)=percentage of m/z of interest in the given UV peak, assumes relative ion abundance included for all coeluting species.

By the term ‘wherein the monoisotope of the most abundant species is 1988 m/z’ is meant the monoisotope of the most abundant species, first peak in the isotopic group with highest response per m/z is m/z 1987.9. The most abundant species may be determined by creating a combined spectrum across the entire total ion chromatogram using the UPLC-UV/MS method (negative ion electrospray) as described herein.

By the term ‘dried’ is meant that substantially all solvent has been removed. A dried extract will typically contain less than 5% solvent w/w, especially less than 2.5% (such as less than 5% water w/w, especially less than 2.5%). Suitably the dried extract will contain 100 ppm or less acetonitrile (w/w).

Further, there is provided a method for the manufacture of a saponin composition comprising the steps of:
(i) treating a crude aqueous extract of Quillaja saponaria by polyvinylpolypyrrolidone (PVPP) adsorption;
(ii) enzymatically modifying the treated extract with a glucosidase and/or a rhamnosidase;
(iii) purifying the modified extract by reverse phase chromatography using a polystyrene resin; and
(iv) purifying the modified extract by reverse phase chromatography using a phenyl resin to provide a saponin composition.

There is also provided a method for the manufacture of a saponin composition comprising the steps of:
(i) treating a crude aqueous extract of Quillaja saponaria by polyvinylpolypyrrolidone adsorption;
(ii) enzymatically modifying the treated extract with an engineered glucosidase polypeptide;
(iii) purifying the modified extract by reverse phase chromatography using a polystyrene resin; and
(iv) purifying the modified extract by reverse phase chromatography using a phenyl resin to provide a saponin composition.

Additionally provided a method for the manufacture of a saponin composition comprising the steps of:
(i) treating a crude aqueous extract of Quillaja saponaria by polyvinylpolypyrrolidone adsorption;
(ii) enzymatically modifying the treated extract with an engineered rhamnosidase polypeptide;
(iii) purifying the modified extract by reverse phase chromatography using a polystyrene resin; and
(iv) purifying the modified extract by reverse phase chromatography using a phenyl resin to provide a saponin composition.

Also provided is a method for the manufacture of a saponin composition comprising the steps of:
(i) treating a crude aqueous extract of Quillaja saponaria by polyvinylpolypyrrolidone adsorption;
(ii) enzymatically modifying the treated extract with an engineered glucosidase and an engineered rhamnosidase;
(iii) purifying the modified extract by reverse phase chromatography using a polystyrene resin; and
(iv) purifying the modified extract by reverse phase chromatography using a phenyl resin to provide a saponin composition.

Typically, the crude aqueous extract is a bark extract. Suitably the QS-21 main peak content in an aqueous solution of crude aqueous extract of Quillaja saponaria is at least 1 g/L, such as at least 2 g/L, especially at least 2.5 g/L and in particular at least 2.8 g/L (e.g. as determined by UV absorbance relative to a control sample of known concentration).

The step of purifying the extract by polyvinylpolypyrrolidone adsorption involves treatment of the extract with polyvinylpolypyrrolidone adsorbant e.g. resin. Typically, the extract is agitated with the polyvinylpolypyrrolidone resin. The extract may subsequently be separated from the polyvinylpolypyrrolidone resin with adsorbed impurities by filtration. This step of the process generally removes polyphenolic impurities such as tannins.

The step of purifying the extract by reverse phase chromatography using a polystyrene resin typically uses acetonitrile and water as solvent, usually acidified with a suitable acid such as acetic acid. An example of a suitable resin is Amberchrom XT20. Chromatography may be undertaken using isocratic conditions, though is typically operated under a solvent gradient (continuous, such as linear, or stepped), such as those provided in the Examples. This step of the process generally removes non-saponin material and enriches the desired saponins. Each polystyrene chromatography run is typically at a scale of between 25-200 g of QS-21, such as between 50-150 g and in particular between 70-110 g (amounts being based on QS-21 main peak content in the material by UV).

Purifying the extract by reverse phase chromatography using a phenyl resin typically uses acetonitrile and water as solvent, usually acidified with a suitable acid such as acetic acid. Chromatography may be undertaken using a solvent gradient (continuous, such as linear, or stepped), though is typically operated under isocratic conditions. This step of the process provides the final purification of the desired saponins. Selected fractions may be pooled to maximise yield of material matching the required criteria. Each phenyl chromatography run is typically at a scale of between 4-40 g of QS-21, such as between 10-30 g and in particular between 13-21 g (amounts being based on QS-21 main peak content in the material by UV).

The method may comprise the further step of removing solvent to provide a dried saponin extract. Consequently, the invention provides a method for the manufacture of a saponin composition comprising the steps of:
(i) treating a crude aqueous extract of Quillaja saponaria by polyvinylpolypyrrolidone adsorption;
(ii) enzymatically modifying the treated extract with a glucosidase and/or a rhamnosidase;
(iii) purifying the modified extract by reverse phase chromatography using a polystyrene resin; and
(iv) purifying the modified extract by reverse phase chromatography using a phenyl resin; and
(v) removing solvent to provide a dried saponin composition.

The invention also provides a method for the manufacture of a saponin composition comprising the steps of:
(i) treating a crude aqueous extract of Quillaja saponaria by polyvinylpolypyrrolidone adsorption;
(ii) enzymatically modifying the treated extract with an engineered glucosidase polypeptide;
(iii) purifying the modified extract by reverse phase chromatography using a polystyrene resin; and
(iv) purifying the modified extract by reverse phase chromatography using a phenyl resin; and
(v) removing solvent to provide a dried saponin composition.

Further provided is a method for the manufacture of a saponin composition comprising the steps of:
(i) treating a crude aqueous extract of Quillaja saponaria by polyvinylpolypyrrolidone adsorption;
(ii) enzymatically modifying the treated extract with an engineered rhamnosidase polypeptide;
(iii) purifying the modified extract by reverse phase chromatography using a polystyrene resin; and
(iv) purifying the modified extract by reverse phase chromatography using a phenyl resin; and
(v) removing solvent to provide a dried saponin composition.

Additionally provided is a method for the manufacture of a saponin composition comprising the steps of:
(i) treating a crude aqueous extract of Quillaja saponaria by polyvinylpolypyrrolidone adsorption;
(ii) enzymatically modifying the treated extract with an engineered glucosidase polypeptide and an engineered rhamnosidase polypeptide;
(iii) purifying the modified extract by reverse phase chromatography using a polystyrene resin; and
(iv) purifying the modified extract by reverse phase chromatography using a phenyl resin; and
(v) removing solvent to provide a dried saponin composition.

In order to improve drying efficiency, it may be desirable to undertake further steps of concentrating the extract, such as by capture and release using an appropriate technique, for example reverse phase chromatography (e.g. using a C8 resin), and/or exchanging the solvent in advance of the drying step.

Also provided is a method for the manufacture of a saponin composition comprising the steps:
(i) treating a crude aqueous extract of Quillaja saponaria by polyvinylpolypyrrolidone adsorption;
(ii) enzymatically modifying the treated extract with a glucosidase and/or a rhamnosidase;
(iii) purifying the modified extract by reverse phase chromatography using a polystyrene resin; and
(iv) purifying the modified extract by reverse phase chromatography using a phenyl resin;
(v) optionally concentrating the modified extract;
(vi) optionally exchanging the solvent; and
(vii) removing the remaining solvent to provide a dried saponin composition;
   wherein steps (v) and (vi) may be optionally be in reverse order or undertaken concurrently, though are typically in the order shown.

Further provided is a method for the manufacture of a saponin composition comprising the steps:
(i) treating a crude aqueous extract of Quillaja saponaria by polyvinylpolypyrrolidone adsorption;
(ii) enzymatically modifying the treated extract with an engineered glucosidase polypeptide;
(iii) purifying the modified extract by reverse phase chromatography using a polystyrene resin; and
(iv) purifying the modified extract by reverse phase chromatography using a phenyl resin;
(v) optionally concentrating the modified extract;
(vi) optionally exchanging the solvent; and
(vii) removing the remaining solvent to provide a dried saponin composition;
   wherein steps (v) and (vi) may be optionally be in reverse order or undertaken concurrently, though are typically in the order shown.

Additionally provided is a method for the manufacture of a saponin composition comprising the steps:
(i) treating a crude aqueous extract of Quillaja saponaria by polyvinylpolypyrrolidone adsorption;
(ii) enzymatically modifying the treated extract with an engineered rhamnosidase polypeptide;
(iii) purifying the modified extract by reverse phase chromatography using a polystyrene resin; and
(iv) purifying the modified extract by reverse phase chromatography using a phenyl resin;
(v) optionally concentrating the modified extract;
(vi) optionally exchanging the solvent; and
(vii) removing the remaining solvent to provide a dried saponin composition;
   wherein steps (v) and (vi) may be optionally be in reverse order or undertaken concurrently, though are typically in the order shown.

The invention provides a method for the manufacture of a saponin composition comprising the steps:
(i) treating a crude aqueous extract of Quillaja saponaria by polyvinylpolypyrrolidone adsorption;
(ii) enzymatically modifying the treated extract with an engineered glucosidase polypeptide and an engineered rhamnosidase polypeptide;
(iii) purifying the modified extract by reverse phase chromatography using a polystyrene resin; and
(iv) purifying the modified extract by reverse phase chromatography using a phenyl resin;
(v) optionally concentrating the modified extract;
(vi) optionally exchanging the solvent; and
(vii) removing the remaining solvent to provide a dried saponin composition;
   wherein steps (v) and (vi) may be optionally be in reverse order or undertaken concurrently, though are typically in the order shown.

Also provided is a method for the manufacture of a saponin composition comprising the steps:
(i) treating a crude aqueous extract of Quillaja saponaria by polyvinylpolypyrrolidone adsorption;
(ii) enzymatically modifying the treated extract with a glucosidase and/or a rhamnosidase;
(iii) purifying the modified extract by diafiltration, ultrafiltration or dialysis;
(iv) purifying the modified extract by reverse phase chromatography using a polystyrene resin;
(v) purifying the modified extract by reverse phase chromatography using a phenyl resin;
(vi) concentrating the modified extract by reverse phase chromatography using a C8 resin;
(vii) exchanging the solvent; and
(viii) removing the remaining solvent to provide a dried saponin composition.

Further provided is a method for the manufacture of a saponin composition comprising the steps:
(i) treating a crude aqueous extract of Quillaja saponaria by polyvinylpolypyrrolidone adsorption;
(ii) enzymatically modifying the treated extract with an engineered glucosidase polypeptide;
(iii) purifying the modified extract by diafiltration, ultrafiltration or dialysis;
(iv) purifying the modified extract by reverse phase chromatography using a polystyrene resin;
(v) purifying the modified extract by reverse phase chromatography using a phenyl resin;
(vi) concentrating the modified extract by reverse phase chromatography using a C8 resin;
(vii) exchanging the solvent; and
(viii) removing the remaining solvent to provide a dried saponin composition.

Additionally provided is a method for the manufacture of a saponin composition comprising the steps:
(i) treating a crude aqueous extract of Quillaja saponaria by polyvinylpolypyrrolidone adsorption;
(ii) enzymatically modifying the treated extract with an engineered rhamnosidase polypeptide;
(iii) purifying the modified extract by diafiltration, ultrafiltration or dialysis;
(iv) purifying the modified extract by reverse phase chromatography using a polystyrene resin;
(v) purifying the modified extract by reverse phase chromatography using a phenyl resin;
(vi) concentrating the modified extract by reverse phase chromatography using a C8 resin;
(vii) exchanging the solvent; and
(viii) removing the remaining solvent to provide a dried saponin composition.

The invention provides a method for the manufacture of a saponin composition comprising the steps:
(i) treating a crude aqueous extract of Quillaja saponaria by polyvinylpolypyrrolidone adsorption;
(ii) enzymatically modifying the treated extract with an engineered glucosidase polypeptide and an engineered rhamnosidase polypeptide;
(iii) purifying the modified extract by diafiltration, ultrafiltration or dialysis;
(iv) purifying the modified extract by reverse phase chromatography using a polystyrene resin;
(v) purifying the modified extract by reverse phase chromatography using a phenyl resin;
(vi) concentrating the modified extract by reverse phase chromatography using a C8 resin;
(vii) exchanging the solvent; and
(viii) removing the remaining solvent to provide a dried saponin composition.

The step of purifying the extract by diafiltration, ultrafiltration or dialysis, is suitably purification by diafiltration. typically using tangential flow. An appropriate example of a membrane is a 30 kDa cut-off. This step of the process generally removes salts, sugars and other low molecular weight materials.

Concentration of the extract may be performed using any suitable technique. For example, concentration may be performed using a capture and release methodology, such as reverse phase chromatography, in particular using a C8 resin. The reverse phase chromatography typically uses acetonitrile and water as solvent, usually acidified with a suitable acid such as acetic acid. Chromatography is typically operated under a solvent gradient, with the saponin extract captured in low organic solvent and eluted in high organic solvent, in particular, a stepped solvent gradient.

Exchanging the solvent may be performed using any suitable technique, in particular diafiltration, ultrafiltration or dialysis, especially diafiltration. Solvent exchange may be useful, for example, in reducing the acetonitrile content such as described in WO2014016374. A suitable membrane may be selected to allow solvent exchange while retaining the saponin extract, such as a 1 kDa membrane.

Drying, by removing the solvent, may be undertaken by any suitable means, in particular by lyophilisation. During drying, degradation of the saponin extract can occur, leading to the formation of Iyo impurity. Consequently, it is desirable to dry under conditions which limit formation of Iyo impurity, such as by limiting the drying temperature and/or drying time. Suitably removal of solvent is undertaken by a single lyophilisation process. The extent of drying required will depend on the nature of the solvent, for example non-pharmaceutically acceptable solvents will desirably be removed to a high degree, whereas some pharmaceutically acceptable solvents (such as water) may be removed to a lesser degree.

Suitably the methods of the present invention are undertaken at a scale of between 25-1000 g of QS-21, such as between 50-500 g and in particular between 100-500 g (amounts being based on QS-21 main peak content in the material by UV).

Provided is a product saponin prepared according to the present invention. There is provided the use of a product saponin prepared according to the present invention in the manufacture of a medicament. Additionally, provided is a product saponin prepared according to the present invention for use as a medicament, in particular as an adjuvant. Also provided is an adjuvant composition comprising a product saponin prepared according to the present invention.

There is provided a crude extract (such as from Quillaja species, especially Quillaja saponaria), such as water and/or lower alcohol extract, especially aqueous extract which has been treated by a glucosidase. There is also provided a crude extract (such as from Quillaja species, especially Quillaja saponaria), such as water and/or lower alcohol extract, especially aqueous extract which has been treated by a rhamnosidase. Also provided is a crude extract (such as from Quillaja species, especially Quillaja saponaria), such as water and/or lower alcohol extract, especially aqueous extract which has been treated by a glucosidase and a rhamnosidase.

There is provided a crude bark extract (such as from Quillaja species, especially Quillaja saponaria), such as water and/or lower alcohol extract, especially aqueous extract which has been treated by a glucosidase. There is also provided a crude bark extract (such as from Quillaja species, especially Quillaja saponaria), such as water and/or lower alcohol extract, especially aqueous extract which has been treated by a rhamnosidase. Also provided is a crude saponin extract (such as from Quillaja species, especially Quillaja saponaria), such as water and/or lower alcohol extract, especially aqueous extract which has been treated by a glucosidase and a rhamnosidase.

There is provided a PVPP treated extract (such as from Quillaja species, especially Quillaja saponaria), such as water and/or lower alcohol extract, especially aqueous extract which has been treated by a glucosidase. There is also provided a PVPP treated extract (such as from Quillaja species, especially Quillaja saponaria), such as water and/or lower alcohol extract, especially aqueous extract which has been treated by a rhamnosidase. Also provided is a PVPP treated extract (such as from Quillaja species, especially Quillaja saponaria), such as water and/or lower alcohol extract, especially aqueous extract which has been treated by a glucosidase and a rhamnosidase.

There is provided a PVPP treated bark extract (such as from Quillaja species, especially Quillaja saponaria), such as water and/or lower alcohol extract, especially aqueous extract which has been treated by a glucosidase. There is also provided a PVPP treated bark extract (such as from Quillaja species, especially Quillaja saponaria), such as water and/or lower alcohol extract, especially aqueous extract which has been treated by a rhamnosidase. Also provided is a PVPP treated saponin extract (such as from Quillaja species, especially Quillaja saponaria), such as water and/or lower alcohol extract, especially aqueous extract which has been treated by a glucosidase and a rhamnosidase.

Also provided is a saponin composition containing at least 93% QS-21 main peak and <0.25% 2018 component by UV absorbance at 214 nm. Suitably wherein the monoisotope of the most abundant species is 1987.9 m/z. Desirably, the saponin composition contains at least 98% QS-21 group by UV absorbance at 214 nm. Desirably, the extract contains 1% or less of Iyo impurity by UV absorbance at 214 nm. Desirably, the extract contains 1% or less of largest peak outside the QS-21 group by UV absorbance at 214 nm.

Also provided is a saponin composition containing at least 98% QS-21 group, at least 93% QS-21 main peak, <0.25% 2018 component, 1% or less of largest peak outside the QS-21 group by UV absorbance at 214 nm and wherein the monoisotope of the most abundant species is 1987.9 m/z. Suitably the saponin composition contains <0.23% 2018 component, especially <0.21% 2018 component, in particular <0.21% 2018 component, such as 0.2% or less 2018 component.

The saponin compositions desirably comprise at least 40%, such as at least 50%, suitably at least 60%, especially at least 70% and desirably at least 80%, for example at least 90% (as determined by UV absorbance at 214 nm and by relative ion abundance) QS-21 1988 A component, QS-21 1856 A component and/or QS-21 2002 A component. In certain embodiments, the saponin composition comprises at least 40%, such as at least 50%, in particular at least 60%, especially at least 65%, such as at least 70%, QS-21 1988 A component as determined by UV absorbance at 214 nm and by relative ion abundance. In certain embodiments the saponin composition contain 90% or less, such as 85% or less, or 80% or less, QS-21 1988 A component as determined by UV absorbance at 214 nm and by relative ion abundance. In certain embodiments, the saponin composition contain from 40% to 90% QS-21 1988 A component, such as 50% to 85% QS-21 1988 A component, especially 70% to 80% QS-21 1988 A component as determined by UV absorbance at 214 nm and by relative ion abundance. In certain embodiments, the saponin compositions contain 30% or less, such as 25% or less, QS-21 1856 A as determined by UV absorbance at 214 nm and by relative ion abundance. In certain embodiments the saponin composition contain at least 5%, such as at least 10% QS-21 1856 A by UV absorbance at 214 nm and by relative ion abundance. In certain embodiments, the saponin compositions contain from 5% to 30% QS-21 1856 A, such as 10% to 25% QS-21 1856 A as determined by UV absorbance at 214 nm and by relative ion abundance. In certain embodiments, the saponin composition contains 40% or less, such as 30% or less, in particular 20% or less, especially 10% or less QS-21 2002 A component by UV absorbance at 214 nm and by relative ion abundance. In certain embodiments, the saponin composition contain at least 0.5%, such as at least 1%, QS-21 2002 A component by UV absorbance at 214 nm and by relative ion abundance. In certain embodiments, the saponin composition contain from 0.5% to 40% QS-21 2002 A component, such as 1% to 10% QS-21 2002 A component as determined by UV absorbance at 214 nm and by relative ion abundance.

By the term ‘Iyo impurity’ is meant the triterpenoid glycosides identified as ‘Lyophilization Peak’ in FIG. 6. Suitably the Iyo impurity in the UPLC-UV/MS methods described herein has a retention time of approximately 4.7 min and the primary component of the peak having a monoisotopic molecular weight of 1855.9. The terms 2018 component, QS-21 main peak, QS-21 group may be understood such as by reference to the examples herein.

The saponin compositions of the present invention (i.e. a composition comprising a product saponin prepared according to the present invention) may be combined with further adjuvants, such as a TLR4 agonist, in particular lipopolysaccharide TLR4 agonists, such as lipid A derivatives, especially a monophosphoryl lipid A e.g. 3-de-O-acylated monophosphoryl lipid A (3D-MPL). 3D-MPL is sold under the name ‘MPL’ by GlaxoSmithKline Biologicals N.A. and is referred throughout the document as 3D-MPL. See, for example, U.S. Pat. Nos. 4,436,727; 4,877,611; 4,866,034 and 4,912,094. 3D-MPL can be produced according to the methods described in GB 2 220 211 A. Chemically it is a mixture of 3-deacylated monophosphoryl lipid A with 4, 5 or 6 acylated chains.

Other TLR4 agonists which may be of use in the present invention include Glucopyranosyl Lipid Adjuvant (GLA) such as described in WO2008/153541 or WO2009/143457 or the literature articles Coler R N et al. (2011) Development and Characterization of Synthetic Glucopyranosyl Lipid Adjuvant System as a Vaccine Adjuvant. PLoS ONE 6(1): e16333. doi:10.1371/journal.pone.0016333 and Arias M A et al. (2012) Glucopyranosyl Lipid Adjuvant (GLA), a Synthetic TLR4 Agonist, Promotes Potent Systemic and Mucosal Responses to Intranasal Immunization with HIVgp140. PLoS ONE 7(7): e41144. doi:10.1371/journal.pone.0041144. WO2008/153541 or WO2009/143457 are incorporated herein by reference for the purpose of defining TLR4 agonists which may be of use in the present invention.

A particular alkyl glucosaminide phosphate (AGP) of interest is set forth as follows:

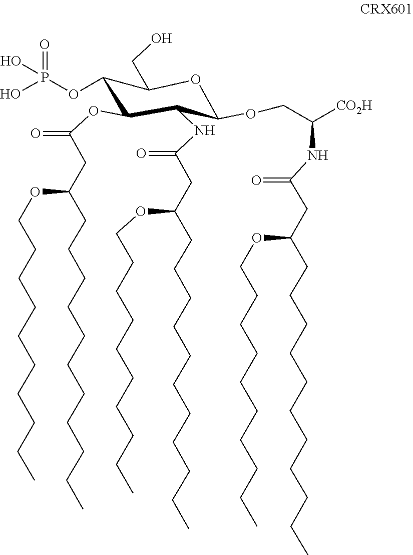

TLR4 agonists of interest include:

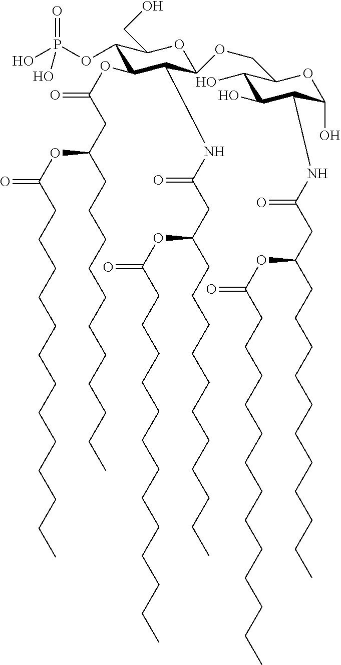

3-deacyl monophosphoryl hexa-acyl lipid A.

Another TLR4 agonist of interest is:

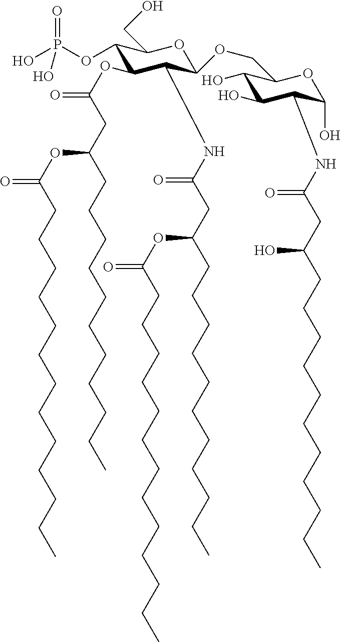

3-deacyl monophosphoryl lipid A.

A TLR4 agonist of interest is dLOS (as described in Han, 2014):

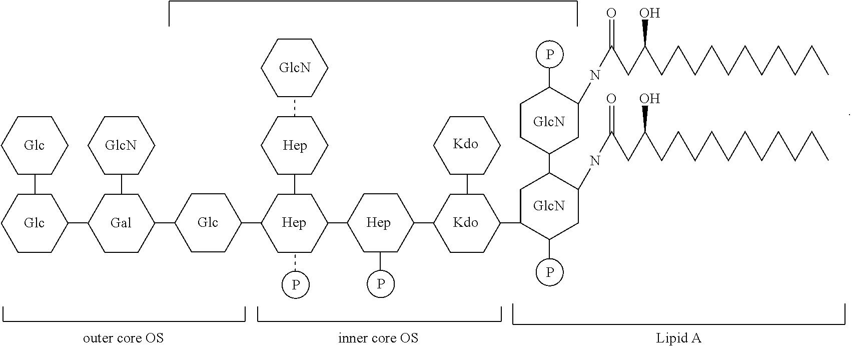

A typical adult human dose of adjuvant will comprise a saponin composition, such as a Q-21 composition, at amounts between 1 and 100 ug per human dose. The saponin extract may be used at a level of about 50 ug. Examples of suitable ranges are 40-60 ug, suitably 45-ug or 49-51 ug, such as 50 ug. In a further embodiment, the human dose comprises saponin composition, such as a Q-21 composition, at a level of about 25 ug. Examples of lower ranges include 20-30 ug, suitably 22-28 ug or 24-26 ug, such as 25 ug. Human doses intended for children may be reduced compared to those intended for an adult (e.g. reduction by 50%).

The TLR4 agonists, such as a lipopolysaccharide, such as 3D-MPL, can be used at amounts between 1 and 100 ug per human dose. 3D-MPL may be used at a level of about 50 ug. Examples of suitable ranges are 40-60 ug, suitably 45-55 ug or 49-51 ug, such as 50 ug. In a further embodiment, the human dose comprises 3D-MPL at a level of about 25 ug. Examples of lower ranges include 20-30 ug, suitably 22-28 ug or 24-26 ug, such as 25 ug. Human doses intended for children may be reduced compared to those intended for an adult (e.g. reduction by 50%).

When both a TLR4 agonist and a saponin composition, such as a Q-21 composition, are present in the adjuvant, then the weight ratio of TLR4 agonist to saponin is suitably between 1:5 to 5:1, suitably 1:1. For example, where 3D-MPL is present at an amount of 50 ug or 25 ug, then suitably QS-21 may also be present at an amount of 50 ug or 25 ug per human dose.

Adjuvants may also comprise a suitable carrier, such as an emulsion (e.g. an oil in water emulsion, such as a squalene containing oil in water emulsion) or liposomes.

The present invention provides an adjuvant composition comprising a saponin composition according to the present invention. Suitably the adjuvant composition further comprises a TLR4 agonist.

### Liposomes

The term ‘liposome’ is well known in the art and defines a general category of vesicles which comprise one or more lipid bilayers surrounding an aqueous space. Liposomes thus consist of one or more lipid and/or phospholipid bilayers and can contain other molecules, such as proteins or carbohydrates, in their structure. Because both lipid and aqueous phases are present, liposomes can encapsulate or entrap water-soluble material, lipid-soluble material, and/or amphiphilic compounds.

Liposome size may vary from 30 nm to several um depending on the phospholipid composition and the method used for their preparation.

The liposomes of use in the present invention suitably contain DOPC, or, consist essentially of DOPC and sterol (with saponin and optionally TLR4 agonist).

In the present invention, the liposome size will be in the range of 50 nm to 200 nm, especially 60 nm to 180 nm, such as 70-165 nm. Optimally, the liposomes should be stable and have a diameter of ˜100 nm to allow convenient sterilization by filtration.

Structural integrity of the liposomes may be assessed by methods such as dynamic light scattering (DLS) measuring the size (Z-average diameter, Zav) and polydispersity of the liposomes, or, by electron microscopy for analysis of the structure of the liposomes. In one embodiment the average particle size is between 95 and 120 nm, and/or, the polydispersity (Pdl) index is not more than 0.3 (such as not more than 0.2).

### Further Excipients

In a further embodiment, a buffer is added to an adjuvant composition. The pH of a liquid preparation is adjusted in view of the components of the composition and necessary suitability for administration to the subject. Suitably, the pH of a liquid mixture is at least 4, at least 5, at least 5.5, at least 5.8, at least 6. The pH of the liquid mixture may be less than 9, less than 8, less than 7.5 or less than 7. In other embodiments, pH of the liquid mixture is between 4 and 9, between 5 and 8, such as between 5.5 and 8. Consequently, the pH will suitably be between 6-9, such as 6.5-8.5. In a particularly preferred embodiment the pH is between 5.8 and 6.4. An appropriate buffer may be selected from acetate, citrate, histidine, maleate, phosphate, succinate, tartrate and TRIS. In one embodiment, the buffer is a phosphate buffer such as Na/Na₂PO₄, Na/K₂PO₄ or K/K₂PO₄.

The buffer can be present in the liquid mixture in an amount of at least 6 mM, at least 10 mM or at least 40 mM. The buffer can be present in the liquid mixture in an amount of less than 100 mM, less than 60 mM or less than 40 mM.

It is well known that for parenteral administration solutions should have a pharmaceutically acceptable osmolality to avoid cell distortion or lysis. A pharmaceutically acceptable osmolality will generally mean that solutions will have an osmolality which is approximately isotonic or mildly hypertonic. Suitably the compositions (when reconstituted, if presented in dried form) will have an osmolality in the range of 250 to 750 mOsm/kg, for example, the osmolality may be in the range of 250 to 550 mOsm/kg, such as in the range of 280 to 500 mOsm/kg. In a particularly preferred embodiment the osmolality may be in the range of 280 to 310 mOsm/kg. Osmolality may be measured according to techniques known in the art, such as by the use of a commercially available osmometer, for example the Advanced® Model 2020 available from Advanced Instruments Inc. (USA).

An “isotonicity agent” is a compound that is physiologically tolerated and imparts a suitable tonicity to a formulation to prevent the net flow of water across cell membranes that are in contact with the formulation. In some embodiments, the isotonicity agent used for the composition is a salt (or mixtures of salts), conveniently the salt is sodium chloride, suitably at a concentration of approximately 150 nM. In other embodiments, however, the composition comprises a non-ionic isotonicity agent and the concentration of sodium chloride in the composition is less than 100 mM, such as less than 80 mM, e.g. less than 50 mM, such as less mM, less than 30 mM and especially less than 20 mM. The ionic strength in the composition may be less than 100 mM, such as less than 80 mM, e.g. less than 50 mM, such as less 40 mM or less than 30 mM.

In a particular embodiment, the non-ionic isotonicity agent is a polyol, such as sucrose and/or sorbitol. The concentration of sorbitol may e.g. between about 3% and about 15% (w/v), such as between about 4% and about 10% (w/v). Adjuvants comprising an immunologically active saponin fraction and a TLR4 agonist wherein the isotonicity agent is salt or a polyol have been described in WO2012/080369.

Suitably, a human dose volume of between 0.05 ml and 1 ml, such as between 0.1 and ml, in particular a dose volume of about 0.5 ml, or 0.7 ml. The volumes of the compositions used may depend on the delivery route and location, with smaller doses being given by the intradermal route. A unit dose container may contain an overage to allow for proper manipulation of materials during administration of the unit dose.

The ratio of saponin:DOPC will typically be in the order of 1:50 to 1:10 (w/w), suitably between 1:25 to 1:15 (w/w), and preferably 1:22 to 1:18 (w/w), such as 1:20 (w/w).

Suitably the saponin is presented in a less reactogenic composition where it is quenched with an exogenous sterol, such as cholesterol. Cholesterol is disclosed in the Merck Index, 13th Edn., page 381, as a naturally occurring sterol found in animal fat. Cholesterol has the formula (C₂₇H₄₆O) and is also known as (3β)-cholest-5-en-3-ol.

The ratio of saponin:sterol will typically be in the order of 1:100 to 1:1 (w/w), suitably between 1:10 to 1:1 (w/w), and preferably 1:5 to 1:1 (w/w). Suitably excess sterol is present, the ratio of saponin:sterol being at least 1:2 (w/w). In one embodiment, the ratio of saponin:sterol is 1:5 (w/w). In one embodiment, the sterol is cholesterol.

The amount of liposome (weight of lipid and sterol) will typically be in the range of 0.1 mg to 10 mg per human dose of a composition, in particular 0.5 mg to 2 mg per human dose of a composition.

In a particularly suitable embodiment, liposomes used in the invention comprise DOPC and a sterol, in particular cholesterol. Thus, in a particular embodiment, a composition used in the invention comprises saponin extract in the form of a liposome, wherein said liposome comprises DOPC and a sterol, in particular cholesterol.

A particular adjuvant of interest features liposomes comprising DOPC and cholesterol, with TLR4 agonist and a saponin prepared according to the present invention, especially 3D-MPL and a saponin prepared according to the present invention.

Another adjuvant of interest features liposomes comprising DOTAP and DMPC, with TLR4 agonist and a saponin prepared according to the present invention, especially dLOS and a saponin prepared according to the present invention.

### Antigens

The adjuvants prepared according to the present invention may be utilised in conjunction with an immunogen or antigen. In some embodiments a polynucleotide encoding the immunogen or antigen is provided.

The adjuvant may be administered to a subject separately from an immunogen or antigen, or the adjuvant may be combined, either during manufacturing or extemporaneously, with an immunogen or antigen to provide an immunogenic composition for combined administration.

As used herein, a subject is a mammalian animal, such as a rodent, non-human primate, or human.

Consequently, there is provided a method for the preparation of an immunogenic composition comprising an immunogen or antigen, or a polynucleotide encoding the immunogen or antigen, said method comprising the steps of:
(i) preparing an adjuvant composition comprising a saponin prepared according to the present invention;
(ii) mixing the adjuvant with an immunogen or antigen, or a polynucleotide encoding the immunogen or antigen.

There is also provided the use of an adjuvant comprising a saponin prepared according to the present invention in the manufacture of a medicament. Suitably the medicament comprises an immunogen or antigen, or a polynucleotide encoding the immunogen or antigen. Further provided is an adjuvant comprising a saponin prepared according to the present invention for use as a medicament. Suitably the medicament comprises an immunogen or antigen, or a polynucleotide encoding the immunogen or antigen.

By the term immunogen is meant a polypeptide which is capable of eliciting an immune response. Suitably the immunogen is an antigen which comprises at least one B or T cell epitope. The elicited immune response may be an antigen specific B cell response, which produces neutralizing antibodies. The elicited immune response may be an antigen specific T cell response, which may be a systemic and/or a local response. The antigen specific T cell response may comprise a CD4+ T cell response, such as a response involving CD4+ T cells expressing a plurality of cytokines, e.g. IFNgamma, TNFalpha and/or IL2. Alternatively, or additionally, the antigen specific T cell response comprises a CD8+ T cell response, such as a response involving CD8+ T cells expressing a plurality of cytokines, e.g., IFNgamma, TNFalpha and/or IL2.

The antigen may be derived (such as obtained from) from a human or non-human pathogen including, e.g., bacteria, fungi, parasitic microorganisms or multicellular parasites which infect human and non-human vertebrates, or from a cancer cell or tumor cell.

In one embodiment the antigen is a recombinant protein, such as a recombinant prokaryotic protein.

A plurality of antigens may be provided. For example, a plurality of antigens may be provided to strengthen the elicited immune response (e.g. to ensure strong protection), a plurality of antigens may be provided to broaden the immune response (e.g. to ensure protection against a range of pathogen strains or in a large proportion of a subject population) or a plurality of antigens may be provided to currently elicit immune responses in respect of a number of disorders (thereby simplifying administration protocols). Where a plurality of antigens are provided, these may be as distinct proteins or may be in the form of one or more fusion proteins.

Antigen may be provided in an amount of 0.1 to 100 ug per human dose. The present invention may be applied for use in the treatment or prophylaxis of a disease or disorder associated with one or more antigens described above. In one embodiment the disease or disorder is selected from malaria, tuberculosis, COPD, HIV and herpes.

The adjuvant may be administered separately from an immunogen or antigen, or may be combined, either during manufacturing or extemporaneously), with an immunogen or antigen to provide an immunogenic composition for combined administration.

### Sterilisation

For parenteral administration in particular, compositions should be sterile. Sterilisation can be performed by various methods although is conveniently undertaken by filtration through a sterile grade filter. Sterilisation may be performed a number of times during preparation of an adjuvant or immunogenic composition, but is typically performed at least at the end of manufacture.

By “sterile grade filter” it is meant a filter that produces a sterile effluent after being challenged by microorganisms at a challenge level of greater than or equal to 1×10⁷/cm² of effective filtration area. Sterile grade filters are well known to the person skilled in the art of the invention for the purpose of the present invention, sterile grade filters have a pore size between and 0.25 um, suitably 0.18-0.22 um, such as 0.2 or 0.22 um.

The membranes of the sterile grade filter can be made from any suitable material known to the skilled person, for example, but not limited to cellulose acetate, polyethersulfone (PES), polyvinylidene fluoride (PVDF), polytetrafluoroethylene (PTFE). In a particular embodiment of the invention one or more or all of the filter membranes of the present invention comprise polyethersulfone (PES), in particular hydrophilic polyethersulfone. In a particular embodiment of the invention, the filters used in the processes described herein are a double layer filter, in particular a sterile filter with built-in prefilter having larger pore size than the pore size of the end filter. In one embodiment the sterilizing filter is a double layer filter wherein the pre-filter membrane layer has a pore size between 0.3 and 0.5 nm, such as 0.35 or 0.45 nm. According to further embodiments, filters comprise asymmetric filter membrane(s), such as asymmetric hydrophilic PES filter membrane(s). Alternatively, the sterilizing filter layer may be made of PVDF, e.g. in combination with an asymmetric hydrophilic PES pre-filter membrane layer. In light of the intended medical uses, materials should be of pharmaceutical grade (such as parenteral grade).

### Clauses of the Invention

The invention is illustrated by the following clauses:
Clause 1. A method for making a product saponin, said method comprising the step of enzymatically converting a starting saponin to the product saponin.
Clause 2. A method for increasing the amount of a product saponin in a composition, said method comprising the step of enzymatically converting a starting saponin to the product saponin.
Clause 3. A method for reducing the amount of a starting saponin in a composition, said method comprising the step of enzymatically converting the starting saponin to a product saponin.
Clause 4. The method according to any one of clauses 1 to 3, wherein the starting saponin is a naturally occurring saponin.
Clause 5. The method according to any one of clauses 1 to 3, wherein the starting saponin is an artificial saponin.
Clause 6. The method according to any one of clauses 1 to 5, wherein the starting saponin is a steroid glycoside.
Clause 7. The method according to any one of clauses 1 to 6, wherein the starting saponin is a terpenoid glycoside.
Clause 8. The method according to clause 7, wherein the starting saponin is a triterpenoid glycoside.
Clause 9. The method according to clause 8, wherein the starting saponin is a quillaic acid glycoside.
Clause 10. The method according to any one of clauses 1 to 4 or 6 to 9, wherein the starting saponin is obtainable from plants of the genera Gypsophilia, Saponaria or Quillaja.
Clause 11. The method according to clause 10, wherein the starting saponin is obtainable from Quillaja species.
Clause 12. The method according to clause 11, wherein the starting saponin is obtainable from Quillaja brasiliensis.
Clause 13. The method according to clause 12, wherein the starting saponin is obtained from Quillaja brasiliensis.
Clause 14. The method according to clause 11, wherein the starting saponin is obtainable from Quillaja saponaria.
Clause 15. The method according to clause 14, wherein the starting saponin is obtained from Quillaja saponaria.
Clause 16. The method according to either clause 14 or 15, wherein the starting saponin is a QS-18 family component.
Clause 17. The method according to clause 16, wherein the starting saponin is a QS-18 2150 A component.
Clause 18. The method according to clause 17, wherein the starting saponin is QS-18 2150 A V1.
Clause 19. The method according to clause 17, wherein the starting saponin is QS-18 2150 A V2.
Clause 20. The method according to clause 16, wherein the starting saponin is QS-18 2018 A component.
Clause 21. The method according to clause 16, wherein the starting saponin is a QS-18 2164 A component.
Clause 22. The method according to clause 21, wherein the starting saponin is QS-18 2164 A V1.
Clause 23. The method according to clause 21, wherein the starting saponin is QS-18 2164 A V2.
Clause 24. The method according to clause 16, wherein the starting saponin is a QS-18 2150 B component.
Clause 26. The method according to clause 24, wherein the starting saponin is QS-18 2150 B V1.
Clause 26. The method according to clause 24, wherein the starting saponin is QS-18 2150 B V2.
Clause 27. The method according to clause 16, wherein the starting saponin is QS-18 2018 B component.
Clause 28. The method according to clause 16, wherein the starting saponin is a QS-18 2164 B component.
Clause 29. The method according to clause 28, wherein the starting saponin is QS-18 2164 B V1.
Clause 30. The method according to clause 28, wherein the starting saponin is QS-18 2164 B V2.
Clause 31. The method according to clause 15, wherein the starting saponin is a desglucosyl-QS-17 family component.
Clause 32. The method according to clause 31, wherein the starting saponin is a desglucosyl-QS-17 2134 A component.
Clause 33. The method according to clause 32, wherein the starting saponin is desglucosyl-QS-17 2134 A V1.
Clause 34. The method according to clause 32, wherein the starting saponin is desglucosyl-QS-17 2134 A V2.
Clause 35. The method according to clause 31, wherein the starting saponin is desglucosyl-QS-17 2002 A component.
Clause 36. The method according to clause 31, wherein the starting saponin is a desglucosyl-QS-17 2148 A component.
Clause 37. The method according to clause 36, wherein the starting saponin is desglucosyl-QS-17 2148 A V1.
Clause 38. The method according to clause 36, wherein the starting saponin is desglucosyl-QS-17 2148 A V2.
Clause 39. The method according to clause 31, wherein the starting saponin is a desglucosyl-QS-17 2134 B component.
Clause 40. The method according to clause 39, wherein the starting saponin is desglucosyl-QS-17 2134 B V1.
Clause 41. The method according to clause 39, wherein the starting saponin is desglucosyl-QS-17 2134 B V2.
Clause 42. The method according to clause 31, wherein the starting saponin is desglucosyl-QS-17 2002 B component.
Clause 43. The method according to clause 31, wherein the starting saponin is a desglucosyl-QS-17 2148 B component.
Clause 44. The method according to clause 43, wherein the starting saponin is desglucosyl-QS-17 2148 B V1.
Clause 45. The method according to clause 43, wherein the starting saponin is desglucosyl-QS-17 2148 B V2.
Clause 46. The method according to clause 15, wherein the starting saponin is a QS-17 family component.
Clause 47. The method according to clause 46, wherein the starting saponin is a QS-17 2296 A component.
Clause 48. The method according to clause 47, wherein the starting saponin is QS-17 2296 A V1.
Clause 49. The method according to clause 48, wherein the starting saponin is QS-17 2296 A V2.
Clause 50. The method according to clause 46, wherein the starting saponin is QS-17 2164 A component.
Clause 51. The method according to clause 46, wherein the starting saponin is a QS-17 2310 A component.
Clause 52. The method according to clause 51, wherein the starting saponin is QS-17 2310 A V1.
Clause 53. The method according to clause 51, wherein the starting saponin is QS-17 2310 A V2.
Clause 54. The method according to clause 46, wherein the starting saponin is a QS-17 2296 B component.
Clause 55. The method according to clause 54, wherein the starting saponin is QS-17 2296 B V1.
Clause 56. The method according to clause 54, wherein the starting saponin is QS-17 2296 B V2.
Clause 57. The method according to clause 46, wherein the starting saponin is QS-17 2164 B component.
Clause 58. The method according to clause 46, wherein the starting saponin is a QS-17 2310 B component.
Clause 59. The method according to clause 58, wherein the starting saponin is QS-17 2310 B V1
Clause 60. The method according to clause 58, wherein the starting saponin is QS-17 2310 B V2.
Clause 61. The method according to clause 15, wherein the starting saponin is a desarabinofuranosyl-QS-18 family component.
Clause 62. The method according to clause 61, wherein the starting saponin is a desarabinofuranosyl-QS-18 2018 A component.
Clause 63. The method according to clause 62, wherein the starting saponin is desarabinofuranosyl-QS-18 2018 A V1.
Clause 64. The method according to clause 62, wherein the starting saponin is desarabinofuranosyl-QS-18 2018 A V2.
Clause 65. The method according to clause 61, wherein the starting saponin is desarabinofuranosyl-QS-18 1886 A component.
Clause 66. The method according to clause 61, wherein the starting saponin is a desarabinofuranosyl-QS-18 2032 A component.
Clause 67. The method according to clause 65, wherein the starting saponin is desarabinofuranosyl-QS-18 2032 A V1.
Clause 68. The method according to clause 65, wherein the starting saponin is desarabinofuranosyl-QS-18 2032 A V2.
Clause 69. The method according to clause 61, wherein the starting saponin is a desarabinofuranosyl-QS-18 2018 B component.
Clause 70. The method according to clause 69, wherein the starting saponin is desarabinofuranosyl-QS-18 2018 B V1.
Clause 71. The method according to clause 69, wherein the starting saponin is desarabinofuranosyl-QS-18 2018 B V2.
Clause 72. The method according to clause 61, wherein the starting saponin is desarabinofuranosyl-QS-18 1886 B component.
Clause 73. The method according to clause 61, wherein the starting saponin is a desarabinofuranosyl-QS-18 2032 B component.
Clause 74. The method according to clause 73, wherein the starting saponin is desarabinofuranosyl-QS-18 2032 B V1.
Clause 75. The method according to clause 73, wherein the starting saponin is desarabinofuranosyl-QS-18 2032 B V2.
Clause 76. The method according to clause 15, wherein the starting saponin is an acetylated desglucosyl-QS-17 family component.
Clause 77. The method according to clause 76, wherein the starting saponin is an acetylated desglucosyl-QS-17 2176 A component.
Clause 78. The method according to clause 77, wherein the starting saponin is acetylated desglucosyl-QS-17 2176 A V1.
Clause 79. The method according to clause 77, wherein the starting saponin is acetylated desglucosyl-QS-17 2176 A V2.
Clause 80. The method according to clause 76, wherein the starting saponin is acetylated desglucosyl-QS-17 2044 A component.
Clause 81. The method according to clause 76, wherein the starting saponin is an acetylated desglucosyl-QS-17 2190 A component.
Clause 82. The method according to clause 81, wherein the starting saponin is acetylated desglucosyl-QS-17 2190 A V1.
Clause 83. The method according to clause 81, wherein the starting saponin is acetylated desglucosyl-QS-17 2190 A V2.
Clause 84. The method according to any one of clauses 1 to 83, wherein the product saponin is a naturally occurring saponin.
Clause 85. The method according to any one of clauses 1 to 83, wherein the product saponin is an artificial saponin.
Clause 86. The method according to any one of clauses 1 to 85, wherein the product saponin is a steroid glycoside.
Clause 87. The method according to any one of clauses 1 to 85, wherein the product saponin is a terpenoid glycoside.
Clause 88. The method according to clause 87, wherein the product saponin is a triterpenoid glycoside.
Clause 89. The method according to clause 88, wherein the product saponin is a quillaic acid glycoside.
Clause 90. The method according to any one of clauses 1 to 84 or 86 to 89, wherein the product saponin is obtainable from plants of the genera Gypsophilia, Saponaria or Quillaja.
Clause 91. The method according to clause 90, wherein the product saponin is obtainable Quillaja species.
Clause 92. The method according to clause 91, wherein the product saponin is obtainable from Quillaja brasiliensis.
Clause 93. The method according to clause 92, wherein the product saponin is obtainable from Quillaja saponaria.
Clause 94. The method according to any one of clauses 16, 31 or 93, wherein the product saponin is a QS-21 family component.
Clause 95. The method according to any one of clauses 17, 32 or 94, wherein the product saponin is a QS-21 1988 A component.
Clause 96. The method according to any one of clauses 18, 33 or 95, wherein the product saponin is QS-21 1988 A V1.
Clause 97. The method according to any one of clauses 19, 34 or 95, wherein the product saponin is QS-21 1988 A V2.
Clause 98. The method according to any one of clauses 20, 35 or 94, wherein the product saponin is QS-21 1856 A component.
Clause 99. The method according to any one of clauses 21, 36 or 94, wherein the product saponin is a QS-21 2002 A component.
Clause 100. The method according to any one of clauses 22, 37 or 99, wherein the product saponin is QS-21 2002 A V1.
Clause 101. The method according to any one of clauses 23, 38 or 99, wherein the product saponin is QS-21 2002 A V2.
Clause 102. The method according to any one of clauses 24, 39 or 94, wherein the product saponin is a QS-21 1988 B component.
Clause 103. The method according to any one of clauses 25, 40 or 102, wherein the product saponin is QS-21 1988 B V1.
Clause 104. The method according to any one of clauses 26, 41 or 102, wherein the product saponin is QS-21 1988 B V2.
Clause 105. The method according to any one of clauses 27, 42 or 94, wherein the product saponin is QS-21 1856 B component.
Clause 106. The method according to any one of clauses 28, 43 or 94, wherein the product saponin is a QS-21 2002 B component.
Clause 107. The method according to any one of clauses 29, 44 or 106, wherein the product saponin is QS-21 2002 B V1.
Clause 108. The method according to any one of clauses 30, 45 or 106, wherein the product saponin is QS-21 2002 B V2.
Clause 109. The method according to either clause 46 or 93, wherein the product saponin is a QS-18 family component.
Clause 110. The method according to either clause 47 or 109, wherein the product saponin is a QS-18 2150 A component.
Clause 111. The method according to either clause 48 or 110, wherein the product saponin is QS-18 2150 A V1.
Clause 112. The method according to either clause 49 or 110, wherein the product saponin is QS-18 2150 A V2.
Clause 113. The method according to either clause 50 or 109, wherein the product saponin is QS-18 2018 A component.
Clause 114. The method according to either clause 51 or 109, wherein the product saponin is a QS-18 2164 A component.
Clause 115. The method according to either clause 52 or 114, wherein the product saponin is QS-18 2164 A V1.
Clause 116. The method according to either clause 53 or 114, wherein the product saponin is QS-18 2164 A V2.
Clause 117. The method according to either clause 54 or 109, wherein the product saponin is a QS-18 2150 B component.
Clause 118. The method according to either clause 55 or 117, wherein the product saponin is QS-18 2150 B V1.
Clause 119. The method according to either clause 56 or 117, wherein the product saponin is QS-18 2150 B V2.
Clause 120. The method according to either clause 57 or 109, wherein the product saponin is QS-18 2018 B component.
Clause 121. The method according to either clause 58 or 120, wherein the product saponin is a QS-18 2164 B component.
Clause 122. The method according to either clause 59 or 121, wherein the product saponin is QS-18 2164 B V1.
Clause 123. The method according to either clause 60 or 121, wherein the product saponin is QS-18 2164 B V2.
Clause 124. The method according to either clause 46 or 93, wherein the product saponin is a desglucosyl-QS-17 family component.
Clause 125. The method according to either clause 47 or 124, wherein the product saponin is a desglucosyl-QS-17 2134 A component.
Clause 126. The method according to either clause 48 or 125, wherein the product saponin is desglucosyl-QS-17 2134 A V1.
Clause 127. The method according to either clause 49 or 125, wherein the product saponin is desglucosyl-QS-17 2134 A V2.
Clause 128. The method according to either clause 50 or 124, wherein the product saponin is desglucosyl-QS-17 2002 A component.
Clause 129. The method according to either clause 51 or 124, wherein the product saponin is a desglucosyl-QS-17 2148 A component.
Clause 130. The method according to either clause 52 or 129, wherein the product saponin is desglucosyl-QS-17 2148 A V1.
Clause 131. The method according to either clause 53 or 129, wherein the product saponin is desglucosyl-QS-17 2148 A V2.
Clause 132. The method according to either clause 54 or 124, wherein the product saponin is a desglucosyl-QS-17 2134 B component.
Clause 133. The method according to either clause 55 or 132, wherein the product saponin is desglucosyl-QS-17 2134B V1.
Clause 134. The method according to either clause 56 or 132, wherein the product saponin is desglucosyl-QS-17 2134 B V2.
Clause 135. The method according to either clause 57 or 124, wherein the product saponin is desglucosyl-QS-17 2002 B component.
Clause 136. The method according to either clause 58 or 124, wherein the product saponin is a desglucosyl-QS-17 2148 B component.
Clause 137. The method according to either clause 59 or 136, wherein the product saponin is desglucosyl-QS-17 2148 B V1.
Clause 138. The method according to either clause 60 or 136, wherein the product saponin is desglucosyl-QS-17 2148 B V2.
Clause 140. The method according to either clause 61 or 93, wherein the product saponin is a desarabinofuranosyl-QS-21 family component.
Clause 140. The method according to either clause 62 or 139, wherein the product saponin is a desarabinofuranosyl-QS-21 1856 A component.
Clause 141. The method according to either clause 63 or 140, wherein the product saponin is desarabinofuranosyl-QS-21 1856 A V1.
Clause 142. The method according to either clause 64 or 140, wherein the product saponin is desarabinofuranosyl-QS-21 1856 A V2.
Clause 143. The method according to either clause 65 or 139, wherein the product saponin is desarabinofuranosyl-QS-21 1712 A component.
Clause 144. The method according to either clause 66 or 139, wherein the product saponin is a desarabinofuranosyl-QS-21 1870 A component.
Clause 145. The method according to either clause 67 or 144, wherein the product saponin is desarabinofuranosyl-QS-21 1870 A V1.
Clause 146. The method according to either clause 68 or 144, wherein the product saponin is desarabinofuranosyl-QS-21 1870 A V2.
Clause 147. The method according to either clause 69 or 139, wherein the product saponin is a desarabinofuranosyl-QS-21 1856 B component.
Clause 148. The method according to either clause 70 or 147, wherein the product saponin is desarabinofuranosyl-QS-21 1856 B V1.
Clause 149. The method according to either clause 71 or 147, wherein the product saponin is desarabinofuranosyl-QS-21 1856 B V2.
Clause 150. The method according to either clause 72 or 139, wherein the product saponin is desarabinofuranosyl-QS-21 1712 B component.
Clause 151. The method according to either clause 73 or 139, wherein the product saponin is a desarabinofuranosyl-QS-21 1870 B component.
Clause 152. The method according to either clause 74 or 151, wherein the product saponin is desarabinofuranosyl-QS-21 1870 B V1.
Clause 153. The method according to either clause 75 or 151, wherein the product saponin is desarabinofuranosyl-QS-21 1870 B V2.
Clause 154. The method according to either clause 76 or 93, wherein the product saponin is an acetylated QS-21 family component.
Clause 155. The method according to either clause 77 or 154, wherein the product saponin is an acetylated QS-21 2030 A component.
Clause 156. The method according to either clause 78 or 155, wherein the product saponin is acetylated QS-21 2030 A V1.
Clause 157. The method according to either clause 79 or 155, wherein the product saponin is acetylated QS-21 2030 A V2.
Clause 158. The method according to either clause 80 or 154, wherein the product saponin is acetylated QS-21 1898 A component.
Clause 159. The method according to either clause 81 or 155, wherein the product saponin is an acetylated QS-21 2044 A component.
Clause 160. The method according to either clause 82 or 159, wherein the product saponin is acetylated QS-21 2044 A V1.
Clause 161. The method according to either clause 83 or 159, wherein the product saponin is acetylated QS-21 2044 A V2.
Clause 162. The method according to any one of clauses 1 to 161, wherein a single starting saponin is converted to a single product saponin.
Clause 163. The method according to any one of clauses 1 to 161, wherein a plurality of starting saponins are converted to a plurality of product saponins.
Clause 164. The method according to clause 163, wherein the plurality of starting saponins comprises QS-18 family components, such as described in any one of clauses 18 to 31.
Clause 165. The method according to either clause 163 or 164, wherein the plurality of starting saponins comprises desglucosyl-QS-17 family components, such as described in any one of clauses 33 to 46.
Clause 166. The method according to any one of clauses 163 to 165, wherein the plurality of starting saponins comprises QS-17 family components, such as described in any one of clauses 48 to 61.
Clause 167. The method according to any one of clauses 163 to 166, wherein the plurality of starting saponins comprises desarabinofuranosyl-QS-18 family components, such as described in any one of clauses 63 to 76.
Clause 169. The method according to any one of clauses 163 to 167, wherein the plurality of starting saponins comprises acetylated desglucosyl-QS-17 family components, such as described in any one of clauses 78 to 84.
Clause 169. The method according to any one of clauses 1 to 4 or 6 to 168, wherein the starting saponin is obtained by extraction from starting material.
Clause 170. The method according to clause 169, wherein the starting saponin is obtained by extraction from plant material.
Clause 171. The method according to clause 170, wherein the starting saponin is obtained by extraction from plant material from plants of the genera Gypsophilia, Saponaria or Quillaja.
Clause 172. The method according to clause 171, wherein the starting saponin is obtained by extraction from plant material obtained from Quillaja species.
Clause 173. The method according to clause 172, wherein the starting saponin is obtained by extraction from plant material obtained from Quillaja brasiliensis.
Clause 174. The method according to clause 172, wherein the starting saponin is obtained by extraction from plant material obtained from Quillaja saponaria.
Clause 175. The method according to any one of clauses 169 to 174, wherein the starting saponin is obtained by extraction from complete plants.
Clause 176. The method according to any one of clauses 169 to 174, wherein the starting saponin is obtained by extraction from selected plant tissues.
Clause 177. The method according to clause 176, wherein the starting saponin is obtained by extraction from plant material including wood or bark, such as plant material which is wood or bark.
Clause 178. The method according to clause 177, wherein the starting saponin is obtained by extraction from plant material including bark, such as plant material which is bark.
Clause 179. The method according to any one of clauses 169 to 188, wherein the starting saponin is obtained by extraction from plant material obtained from an adult plant.
Clause 180. The method according to any one of clauses 169 to 179, wherein the starting saponin is obtained by extraction from plant material obtained from a young plant.
Clause 181. The method according to any one of clauses 169 to 180, wherein the starting saponin is obtained by extraction using water and/or lower alcohols.
Clause 182. The method according to clause 181, wherein the starting saponin is obtained by aqueous extraction.
Clause 183. The method according to any one of clauses 1 to 182, wherein the starting saponin is a minor component in a saponin containing composition, such as a minor component in a plant material extract.
Clause 184. The method according to any one of clauses 1 to 182, wherein the starting saponin is a major component in a saponin containing composition, such as a major component in a plant material extract.
Clause 185. The method according to any one of clauses 169 to 184, wherein the starting saponin is partially purified.
Clause 186. The method according to any one of clauses 169 to 184, wherein the starting saponin is substantially purified.
Clause 187. The method according to any one of clauses 169 to 184, wherein the starting saponin is in the form of a component of Quil A.
Clause 188. The method according to any one of clauses 169 to 184, wherein the starting saponin is in the form of a component of Fraction A.
Clause 189. The method according to any one of clauses 169 to 184, wherein the starting saponin is in the form of a component of Fraction B.
Clause 190. The method according to any one of clauses 169 to 184, wherein the starting saponin is in the form of a component of Fraction C.
Clause 191. The method according to any one of clauses 169 to 184, wherein the starting saponin is in the form of a component of QS-7.
Clause 192. The method according to any one of clauses 169 to 184, wherein the starting saponin is in the form of a component of QS-17.
Clause 193. The method according to any one of clauses 169 to 184, wherein the starting saponin is in the form of a component of QS-18.
Clause 194. The method according to any one of clauses 169 to 184, wherein the starting saponin is in the form of a component of QS-21.
Clause 195. The method according to any one of clauses 169 to 184, wherein the starting saponin is in the form of a component of a crude bark extract.
Clause 196. The method according to any one of clauses 169 to 195, further comprising the step of extracting the starting saponin from the starting material.
Clause 197. The method according to any one of clauses 1 to 194 or 196, further comprising the step of purifying the starting saponin from extracted material.
Clause 198. The method according to any one of clauses 1 to 197, wherein the enzymatic conversion involves the removal of a single sugar residue.
Clause 199. The method according to clause 198, wherein the enzymatic conversion involves the removal of a glucose residue.
Clause 200. The method according to clause 199, wherein the enzymatic conversion involves the removal of a beta-glucose residue.
Clause 201. The method according to clause 200, wherein the enzymatic conversion involves the removal of the beta-glucose residue:
Clause 202. The method according to clause 198, wherein the enzymatic conversion involves the removal of a rhamnose residue.
Clause 203. The method according to clause 202, wherein the enzymatic conversion involves the removal of a alpha-rhamnose residue.
Clause 204. The method according to clause 203, wherein the enzymatic conversion involves the removal of the alpha-rhamnose residue:
Clause 205. The method according to any one of clauses 1 to 197, wherein the enzymatic conversion involves the removal of a plurality of sugar residues.
Clause 206. The method according to any one of clauses 1 to 205, wherein the method involves multiple enzymatic conversions.
Clause 207. The method according to clause 206, wherein the multiple enzymatic conversions are undertaken in series.
Clause 208. The method according to clause 206, wherein the multiple enzymatic conversions are undertaken in parallel.
Clause 209. The method according to any one of clauses 206 to 208, wherein the multiple enzymatic conversions comprise, such as consist of, the removal of glucose and rhamnose.
Clause 210. The method according to clause 209, wherein the multiple enzymatic conversions comprise, such as consist of, the removal of a beta-glucose residue and an alpha-rhamnose residue.
Clause 211. The method according to clause 210, wherein the multiple enzymatic conversions comprise, such as consist of, the removal of:
Clause 212. The method according to any one of clauses 1 to 30, 46 to 75, 84 to 108, 124 to 153 or 162 to 197 wherein the enzymatic conversion is carried out by an enzyme demonstrating beta exo glucosidase activity.
Clause 213. The method according to any one of clauses 1 to 15, 31 to 45, 76 to 123 or 154 to 197 wherein the enzymatic conversion is carried out by an enzyme demonstrating alpha exo rhamnosidase activity.
Clause 214. The method according to any one of clauses 1 to 213, wherein the glycosidase is of external origin.
Clause 215. The method according to any one of clauses 1 to 214, wherein the enzymatic conversion occurs in an extra cellular environment.
Clause 216. The method according to any one of clauses 1 to 215, wherein the glycosidase is recombinantly produced.
Clause 217. The method according to any one of clauses 1 to 216, wherein the glycosidase is provided in the form of a lysate, such as a clarified lysate and in particular an E. coli lysate or clarified lysate.
Clause 218. The method according to any one of clauses 1 to 217, further comprising the step of purifying the product saponin.
Clause 219. The method according to any one of clauses 1 to 218, wherein the enzymatic conversion is undertaken at pH 4 to 9, especially pH 5 to 8, and in particular pH to 7.5 such as pH 5.5 to 6.5.
Clause 220. The method according to any one of clauses 1 to 219, wherein the enzymatic conversion is undertaken at 10 degC to 60 degC, especially 15 degC to 50 degC, in particular 15 degC to 45 degC, such as 20 degC to 42 degC.
Clause 221. The method according to any one of clauses 1 to 220, wherein the enzymatic conversion occurs over a period of up to 2 days, especially up to 1 day, in particular up to 18 hrs, such as 12 hrs, for example up to 6 hrs.
Clause 222. The method according to any one of clauses 1 to 221, wherein the enzymatic conversion occurs in water or an aqueous solution with water miscible co-solvent(s).
Clause 223. The method according to any one of clauses 1 to 222, wherein the starting saponins are present at a concentration of 0.001 to 100 g per litre, especially to 75 g per litre, in particular 0.01 to 50 g per litre, such as 0.1 to 25 g per litre, for example 1 to 10 g per litre.
Clause 224. The method according to any one of clauses 1 to 223, wherein the enzymatic conversion occurs in a batch reaction volume of 500 ml to 2000 L, especially 1 L to 1000 L, in particular 10 L to 500 L, such as 25 L to 200 L.
Clause 225. The method according to any one of clauses 1 to 224, wherein the weight of each glycosidase present is in the range of 0.0001 mg to 25 mg per ml, especially 0.0001 mg to 5 mg per ml, in particular 0.0001 mg to 1 mg per ml, such as 0.001 mg to 0.5 mg per ml.
Clause 226. The method according to any one of clauses 1 to 225, wherein the weight of each glycosidase present is in the range of 0.01 mg to 100 mg of dried clarified lysate per ml, especially 0.01 mg to 30 mg per ml, in particular 0.01 mg to 5 mg per ml, such as 0.01 mg to 1 mg per ml.
Clause 227. Use of a glycosidase for enzymatically converting a starting saponin to the product saponin, such as in a method of any one of clauses 1 to 226.
Clause 228. The method or use according to any one of clauses 212 or 214 to 227, wherein the glucosidase comprises, such as consists of, an amino acid sequence according to SEQ ID No. 262, 208, 63, 229, 250, 5, 101, 207, 169, 247, 302, 324, 319, 9, 240, 325, 338, 850, 879, 868, 826, 804, 888, 881, 891, 816, 827, 857, 853, 842, 814, 886, 885, 838, 829, 808, 828, 870, 873, 844, 882, 874, 825, 824, 823, 810, 894, 849, 803, 890, 841, 832, 830, 845, 871, 837, 883 or 809 or functional variants thereof.
Clause 229. A method for identifying a candidate enzyme having beta exo glucosidase activity, comprising selecting an enzyme comprising, such as consisting of, an amino acid sequence according to SEQ ID No. 262, 208, 63, 229, 250, 5, 101, 207, 169, 247, 302, 324, 319, 9, 240, 325, 338, 850, 879, 868, 826, 804, 888, 881, 891, 816, 827, 857, 853, 842, 814, 886, 885, 838, 829, 808, 828, 870, 873, 844, 882, 874, 825, 824, 823, 810, 894, 849, 803, 890, 841, 832, 830, 845, 871, 837, 883 or 809 or functional variants thereof.
Clause 230. A method for identifying an enzyme having beta exo glucosidase activity, comprising testing a glycosidase, such as an enzyme comprising, such as consisting of, an amino acid sequence according to SEQ ID No. 262, 208, 63, 229, 250, 5, 101, 207, 169, 247, 302, 324, 319, 9, 240, 325, 338, 850, 879, 868, 826, 804, 888, 881, 891, 816, 827, 857, 853, 842, 814, 886, 885, 838, 829, 808, 828, 870, 873, 844, 882, 874, 825, 824, 823, 810, 894, 849, 803, 890, 841, 832, 830, 845, 871, 837, 883 or 809 or functional variants thereof, for ability to remove glucose from a saponin.
Clause 231. The use or method according to any one of clauses 227 to 230, wherein the enzyme comprises, such as consists of, an amino acid sequence according to SEQ ID No. 262, 208, 63, 229, 250, 5, 101, 207, 169, 247, 302, 324, 319, 9, 240, 325 or 338, or functional variants thereof.
Clause 232. The use or method according to clause 231, wherein the enzyme comprises, such as consists of, an amino acid sequence according to SEQ ID No. 262, 208, 63, 229, 250, 5, 101, 207, 169, 247, 302, 324 or 319, or functional variants thereof.
Clause 233. The use or method according to clause 232, wherein the enzyme comprises, such as consists of, an amino acid sequence according to SEQ ID No. 262, 208, 63, 229, 250, 5, 101 or 207, or functional variants thereof.
Clause 234. The use or method according to clause 233, wherein the enzyme comprises, such as consists of, an amino acid sequence according to SEQ ID No. 262, or functional variants thereof.
Clause 235. The use or method according to clause 234, wherein the enzyme is an engineered glucosidase polypeptide according to any one of clauses 260 to 355.
Clause 236. The use or method according to any one of clauses 227 to 230, wherein the enzyme comprises, such as consists of, an amino acid sequence according to SEQ ID No. 850, 879, 868, 826, 804, 888, 881, 891, 816, 827, 857, 853, 842, 814, 886, 885, 838, 829, 808, 828, 870, 873, 844, 882, 874, 825, 824, 823, 810, 894, 849, 803, 890, 841, 832, 830, 845, 871, 837, 883 or 809, or functional variants thereof.
Clause 237. The use or method according to clause 236, wherein the enzyme comprises, such as consists of, an amino acid sequence according to SEQ ID No. 850, 879, 868, 826, 804, 888, 881, 891, 816, 827, 857, 853, 842, 814, 886, 885, 838, 829, 808, 828, 870, 873, 844, 882, 874, 825, 824, 823, 810, 894, 849, 803, 890 or 841, or functional variants thereof.
Clause 238. The use or method according to clause 237, wherein the enzyme comprises, such as consists of, an amino acid sequence according to SEQ ID No. 850, 879, 868, 826, 804, 888, 881, 891, 816, 827, 857, 853, 842, 814, 886, 885, 838, 829, 808, 828, 870, 873, 844, 882, 874, 825, 824, 823, 810 or 894, or functional variants thereof.
Clause 239. The method or use according to any one of clauses 213 to 227, wherein the rhamnosidase comprises, such as consists of, an amino acid sequence according to SEQ ID No. 992, 1003, 1052, 1073, 1017, 1055, 1075, 1001, 1007, 1061, 1079, 1027, 1039, 1041, 989, 1053, 1018, 1066, 1082, 1076, 993, 1077, 1046, 1015, 1063, 1054, 1074, 1067 or 1033, or functional variants thereof.
Clause 240. A method for identifying a candidate enzyme having alpha exo rhamnosidase activity, comprising selecting an enzyme comprising, such as consisting of, an amino acid sequence according to SEQ ID No. 992, 1003, 1052, 1073, 1017, 1055, 1075, 1001, 1007, 1061, 1079, 1027, 1039, 1041, 989, 1053, 1018, 1066, 1082, 1076, 993, 1077, 1046, 1015, 1063, 1054, 1074, 1067 or 1033, or functional variants thereof.
Clause 241. A method for identifying an enzyme having alpha exo rhamnosidase activity, comprising testing a glycosidase, such as enzyme comprising, such as consisting of, an amino acid sequence according to SEQ ID No. 992, 1003, 1052, 1073, 1017, 1055, 1075, 1001, 1007, 1061, 1079, 1027, 1039, 1041, 989, 1053, 1018, 1066, 1082, 1076, 993, 1077, 1046, 1015, 1063, 1054, 1074, 1067 or 1033, or functional variants thereof for ability to remove rhamnose from a saponin.
Clause 242. The use or method according to any one of clauses 239 to 241, wherein the enzyme comprises, such as consists of, an amino acid sequence according to SEQ ID No. 992, 1003, 1052, 1073, 1017, 1055, 1075, 1001, 1007, 1061, 1079, 1027, 1039, 1041, 989, 1053, 1018, 1066, 1082, 1076, 993 or 1077, or functional variants thereof.
Clause 243. The use or method according to clause 242, wherein the enzyme comprises, such as consists of, an amino acid sequence according to SEQ ID No. 992, 1003, 1052, 1073, 1017, 1055, 1075, 1001, 1007, 1061, 1079, 1027, 1039, 1041 or 989, or functional variants thereof.
Clause 244. The use or method according to clause 243, wherein the enzyme comprises, such as consists of, an amino acid sequence according to SEQ ID No. 1017, or functional variants thereof.
Clause 245. The use or method according to clause 243, wherein the enzyme is an engineered rhamnosidase polypeptide according to any one of clauses 376 to 463.
Clause 246. The use or method according to any one of clauses 228 to 245, wherein the functional variant comprises a sequence having at least 80% identity to the reference sequence, especially at least 90%, in particular at least 95%, such as at least 98%, for example at least 99% identity.
Clause 247. The use or method according to any one of clauses 228 to 245, wherein the functional variant comprises a sequence having a fragment of at least at least 100, especially at least 200, particularly at least 300, such as at least 400, for example at least 500 contiguous amino acids of the reference sequence.
Clause 248. The use or method according to any one of clauses 228 to 245, wherein the functional variant comprises a sequence having 1 to 20 additions, deletions and/or substitutions relative to the reference sequence, especially 1 to 15 additions, deletions and/or substitutions, particularly 1 to 10 additions, deletions and/or substitutions, such as 1 to 5 additions, deletions and/or substitutions.
Clause 249. The use or method according to any one of clauses 228 to 245, wherein the enzyme comprises, such as consists of, a reference sequence.
Clause 250. A saponin prepared by the method of any one of clauses 1 to 226, 228, 231 to 239 or 242 to 249.
Clause 251. A saponin containing composition comprising a product saponin prepared by the method of any one of clauses 1 to 226, 228, 231 to 239 or 242 to 249.
Clause 252. The saponin containing composition according to clause 251, comprising QS-21 family components.
Clause 253. An adjuvant composition comprising a saponin or saponin containing composition according to any one of clauses 250 to 252.
Clause 254. An adjuvant composition prepared using a saponin or saponin containing composition according to any one of clauses 250 to 252.
Clause 255. Use of a saponin or saponin containing composition according to any one of clauses 250 to 252 in the manufacture of an adjuvant composition.
Clause 256. An immunogenic composition comprising a saponin or saponin containing composition according to any one of clauses 250 to 252 and an antigen.
Clause 257. An immunogenic composition comprising a saponin or saponin containing composition according to any one of clauses 250 to 252 and a polynucleotide encoding an antigen.
Clause 258. A kit of parts comprising:
   (i) a saponin or saponin containing composition according to any one of clauses 250 to 252
   (ii) an antigen.
Clause 259. A kit of parts comprising:
   (i) a saponin or saponin composition according to any one of clauses 250 to 252
   (ii) a polynucleotide encoding an antigen.
Clause 260. An engineered glucosidase polypeptide comprising an amino acid sequence that is at least 80% identical to the amino acid sequence of SEQ ID No. 262, or a functional fragment thereof, wherein the engineered glucosidase polypeptide includes at least one residue substitution from:
   F44Y;
   V60L;
   G117A;
   F170N;
   V263G or V263L;
   N351H or N351Q;
   A355H, A355I, A355L, A355M, A355R, A355T or A355W;
   A356P;
   R357A, R357C, R357K, R357M or R357Q;
   G362C;
   T365A, T365N or T365S;
   L367C;
   V394R;
   V395Y;
   Q396E, Q396G, Q396N, Q396P, Q396R, Q396S or Q396Y;
   F430W;
   R435F;
   V438T;
   V440F;
   F442M or F442Q;
   G444T;
   A473F or A473R;
   L474C, L474I or L474V;
   I475F;
   L492C, L492G, L492H, L492I, L492N, L492Q, L492V, L492W or L492Y;
   Q493F or Q493H;
   P494H or P494I;
   S495I, S495K or S495Q;
   G496P or G496W;
   D498A, D498E, D498F, D498I, D498K, D498L, D498N, D498P, D498R, D498S, D498T or D498V;
   A502R;
   M504G or M504R;
   L507A or L507R;
   T508M;
   L529M;
   F535P;
   A536D or A536E;
   A537R;
   F541A, F541I, F541L, F541M or F541V;
   L542I;
   Q543G or Q543L;
   E547L; and
   Y585W.
Clause 261. The polypeptide according to clause 260, having one of the substitutions.
Clause 262. The polypeptide according to clause 260, having two of the substitutions.
Clause 263. The polypeptide according to clause 260, having three of the substitutions.
Clause 264. The polypeptide according to clause 260, having four of the substitutions.
Clause 265. The polypeptide according to clause 260, having five of the substitutions.
Clause 266. The polypeptide according to clause 260, having six of the substitutions.
Clause 267. The polypeptide according to clause 260, having seven of the substitutions.
Clause 268. The polypeptide according to clause 260, having eight of the substitutions.
Clause 269. The polypeptide according to clause 260, having nine of the substitutions.
Clause 270. The polypeptide according to clause 260, having ten of the substitutions.
Clause 271. The polypeptide according to clause 260, having eleven of the substitutions.
Clause 272. The polypeptide according to clause 260, having twelve of the substitutions.
Clause 273. The polypeptide according to clause 260, having thirteen of the substitutions.
Clause 274. The polypeptide according to clause 260, having fourteen of the substitutions.
Clause 275. The polypeptide according to clause 260, having fifteen of the substitutions.
Clause 276. The polypeptide according to clause 260, having sixteen of the substitutions.
Clause 277. The polypeptide according to clause 260, having seventeen of the substitutions.
Clause 277. The polypeptide according to clause 260, having eighteen of the substitutions.
Clause 278. The polypeptide according to clause 260, having nineteen of the substitutions.
Clause 279. The polypeptide according to clause 260, having twenty of the substitutions.
Clause 280. The polypeptide according to clause 260, having twenty-one of the substitutions.
Clause 281. The polypeptide according to clause 260, having twenty-two of the substitutions.
Clause 282. The polypeptide according to clause 260, having twenty-three of the substitutions.
Clause 283. The polypeptide according to clause 260, having twenty-four of the substitutions.
Clause 284. The polypeptide according to clause 260, having twenty-five of the substitutions.
Clause 285. The polypeptide according to clause 260, having twenty-six to thirty of the substitutions.
Clause 286. The polypeptide according to clause 260, having thirty-one to forty-three substitutions of the substitutions.
Clause 287. The polypeptide according to any one of clauses 260 to 286, comprising F44Y.
Clause 288. The polypeptide according to any one of clauses 260 to 287, comprising V60L.
Clause 289. The polypeptide according to any one of clauses 260 to 288, comprising G117A.
Clause 290. The polypeptide according to any one of clauses 260 to 289, comprising F170N.
Clause 291. The polypeptide according to any one of clauses 260 to 290, comprising V263G or V263L.
Clause 292. The polypeptide according to clause 291, comprising V263L.
Clause 293. The polypeptide according to any one of clauses 260 to 292, comprising N351H or N351Q.
Clause 294. The polypeptide according to clause 293, comprising N351H.
Clause 295. The polypeptide according to any one of clauses 260 to 294, comprising A355H, A355I, A355L, A355M, A355R, A355T or A355W.
Clause 296. The polypeptide according to clause 295, comprising A355H.
Clause 297. The polypeptide according to clause 295, comprising A355I.
Clause 298. The polypeptide according to clause 295, comprising A355L.
Clause 299. The polypeptide according to clause 295, comprising A355M.
Clause 300. The polypeptide according to clause 295, comprising A355R.
Clause 301. The polypeptide according to clause 295, comprising A355T.
Clause 302. The polypeptide according to clause 295, comprising A355W.
Clause 303. The polypeptide according to any one of clauses 260 to 302, comprising A356P.
Clause 304. The polypeptide according to any one of clauses 260 to 303, comprising R357A, R357C, R357K, R357M or R357Q.
Clause 305. The polypeptide according to clause 304, comprising R357M.
Clause 306. The polypeptide according to any one of clauses 260 to 305, comprising G362C.
Clause 307. The polypeptide according to any one of clauses 260 to 306, comprising T365A, T365N or T365S.
Clause 308. The polypeptide according to clause 307, comprising T365N.
Clause 309. The polypeptide according to any one of clauses 260 to 308, comprising L367C.
Clause 310. The polypeptide according to any one of clauses 260 to 309, comprising V394R.
Clause 311. The polypeptide according to any one of clauses 260 to 310, comprising V395Y.
Clause 312. The polypeptide according to any one of clauses 260 to 311, comprising Q396E, Q396G, Q396N, Q396P, Q396R, Q396S or Q396Y.
Clause 313. The polypeptide according to clause 312, comprising Q396R.
Clause 314. The polypeptide according to any one of clauses 260 to 313, comprising F430W.
Clause 315. The polypeptide according to any one of clauses 260 to 314, comprising R435F.
Clause 316. The polypeptide according to any one of clauses 260 to 315, comprising V438T.
Clause 317. The polypeptide according to any one of clauses 260 to 316, comprising V440F.
Clause 318. The polypeptide according to any one of clauses 260 to 317, comprising F442M or F442Q.
Clause 319. The polypeptide according to clause 318, comprising F442Q.
Clause 320. The polypeptide according to any one of clauses 260 to 319, comprising G444T.
Clause 321. The polypeptide according to any one of clauses 260 to 320, comprising A473F or A473R.
Clause 322. The polypeptide according to clause 321, comprising A473F.
Clause 323. The polypeptide according to any one of clauses 260 to 322, comprising L474C, L474I or L474V.
Clause 324. The polypeptide according to clause 323, comprising L474C.
Clause 325. The polypeptide according to any one of clauses 260 to 324, comprising I475F.
Clause 326. The polypeptide according to any one of clauses 260 to 325, comprising L492C, L492G, L492H, L492I, L492N, L492Q, L492V, L492W or L492Y.
Clause 327. The polypeptide according to clause 326, comprising L492H.
Clause 328. The polypeptide according to clause 326, comprising L492N.
Clause 329. The polypeptide according to clause 326, comprising L492V.
Clause 330. The polypeptide according to any one of clauses 260 to 329, comprising Q493F or Q493H.
Clause 331. The polypeptide according to any one of clauses 260 to 330, comprising P494I.
Clause 332. The polypeptide according to any one of clauses 260 to 331, comprising S495I, S495K or S495Q.
Clause 333. The polypeptide according to any one of clauses 260 to 332, comprising G496P or G496W.
Clause 334. The polypeptide according to any one of clauses 260 to 333, comprising G496P.
Clause 335. The polypeptide according to any one of clauses 260 to 334, comprising D498A, D498E, D498F, D498I, D498K, D498L, D498N, D498P, D498R, D498S, D498T or D498V.
Clause 336. The polypeptide according to clause 335, comprising D498P.
Clause 337. The polypeptide according to any one of clauses 260 to 336, comprising A502R;
Clause 338. The polypeptide according to any one of clauses 260 to 337, comprising M504G or M504R.
Clause 339. The polypeptide according to any one of clauses 260 to 338, comprising M504R.
Clause 340. The polypeptide according to any one of clauses 260 to 339, comprising L507A or L507R.
Clause 341. The polypeptide according to clause 340, comprising L507R.
Clause 342. The polypeptide according to any one of clauses 260 to 341, comprising T508M.
Clause 343. The polypeptide according to any one of clauses 260 to 342, comprising L529M.
Clause 344. The polypeptide according to any one of clauses 260 to 343, comprising F535P.
Clause 345. The polypeptide according to any one of clauses 260 to 344, comprising A536D or A536E.
Clause 346. The polypeptide according to any one of clauses 260 to 345, comprising A537R.
Clause 347. The polypeptide according to any one of clauses 260 to 346, comprising F541A, F541I, F541L, F541M or F541V.
Clause 348. The polypeptide according to clause 347, comprising F541I.
Clause 349. The polypeptide according to any one of clauses 260 to 348, comprising L542I.
Clause 350. The polypeptide according to any one of clauses 260 to 349, comprising Q543G or Q543L.
Clause 351. The polypeptide according to any one of clauses 260 to 350, comprising E547L.
Clause 352. The polypeptide according to any one of clauses 260 to 351, comprising Y585W.
Clause 353. The polypeptide according to clause 260, comprising one mutation which is T365N.
Clause 354. The polypeptide according to clause 260 or 265 to 353, comprising R357M, T365N, A473F, L474C and I475F.
Clause 355. The polypeptide according to clause 260 or 267 to 353, comprising F44Y, R357M, T365N, F442Q, A473F, L474C and I475F.
Clause 356. The polypeptide according to clause 260 or 269 to 353, comprising F44Y, V263L, R357M, T365N, F442Q, A473F, L474C, I475F and F541I.
Clause 357. The polypeptide according to clause 260 to 353, comprising at least one residue substitution from F44Y, V263L, A355W, R357M, T365N, L367C, Q396R, F442Q, L474C, I475F and F541I,
Clause 358. The polypeptide according to clause 357, comprising F44Y, V263L, A355W, R357M, T365N, L367C, Q396R, F442Q, L474C, 1475F and F541I,
Clause 359. The polypeptide according to any one of clauses 260 to 358, comprising an amino acid sequence that is at least 80% identical to the amino acid sequence of SEQ ID No. 262, or a functional fragment thereof, wherein the engineered glucosidase polypeptide includes at least one residue substitution from:
   F44Y;
   V263L;
   N351H;
   A355H, A355M or A355W;
   R357M;
   T365N;
   L367C;
   Q396R;
   V438T;
   F442Q;
   L474C;
   I475F;
   L492V, L492N or L492H,
   M504R;
   L507R; and
   F541I.
Clause 359. The polypeptide according to any one of clauses 260 to 358 comprising an amino acid sequence that is at least 85% identical to the amino acid sequence of SEQ ID No. 262.
Clause 360. The polypeptide according to any one of clauses 260 to 358 comprising an amino acid sequence that is at least 90% identical to the amino acid sequence of SEQ ID No. 262.
Clause 361. The polypeptide according to any one of clauses 260 to 358 comprising an amino acid sequence that is at least 95% identical to the amino acid sequence of SEQ ID No. 262.
Clause 362. The polypeptide according to any one of clauses 260 to 358 comprising an amino acid sequence that is at least 98% identical to the amino acid sequence of SEQ ID No. 262.
Clause 363. The polypeptide according to any one of clauses 260 to 358, wherein the functional variant comprises a sequence having a fragment of at least 100, especially at least 200, particularly at least 300, such as at least 400, for example at least 500 contiguous amino acids of SEQ ID No. 262.
Clause 364. The polypeptide according to clause 260, comprising the amino acid sequence of SEQ ID No. 1179.
Clause 365. The polypeptide according to clause 260, comprising the amino acid sequence of SEQ ID No. 1180.
Clause 366. The polypeptide according to clause 260, comprising the amino acid sequence of SEQ ID No. 1181.
Clause 367. The polypeptide according to clause 260, comprising the amino acid sequence of SEQ ID No. 1182.
Clause 368. The polypeptide according to clause 260, comprising the amino acid sequence of SEQ ID No. 1183.
Clause 369. The polypeptide according to any one of clauses 260 to 368, further comprising an affinity tag.
Clause 370. The polypeptide according to clause 369, wherein the affinity tag is a poly-his tag, such as a hexa-his tag.
Clause 371. The polypeptide according to either clause 369 or 370, wherein the affinity tag is N-terminally located.
Clause 372. The polypeptide according to clause 371, comprising an amino acid sequence of SEQ ID No. 1177.
Clause 373. The polypeptide according to either clause 369 or 370, wherein the affinity tag is C-terminally located.
Clause 374. The polypeptide according to clause 373, comprising an amino acid sequence of SEQ ID No. 1178.
Clause 375. The polypeptide according to any one of clauses 260 to 374, comprising 1000 residues or fewer, especially 950 residues or fewer, in particular 900 residues or fewer, such as 850 residues or fewer.
Clause 376. An engineered rhamnosidase polypeptide comprising an amino acid sequence that is at least 80% identical to the amino acid sequence of SEQ ID No. 1017, or a functional fragment thereof, wherein the engineered rhamnosidase polypeptide includes at least one residue substitution from:
   (i) A56C
   (ii) A143P
   (iii) Q181H, Q181R or Q181S
   (v) G215S
   (vi) F216M
   (vii) G218D or G218N
   (viii) K219G
   (ix) A238M
   (x) T252Y
   (xi) T311W
   (xii) V326C
   (xiii) G357C
   (xiv) S369C, S369I, S369K or S369M
   (xv) I487M, I487Q or I487V
   (xvi) K492N
   (xvii) V499T
   (xviii) G508S
   (xix) R543C
   (xx) L557Y
   (xxi) G634A
   (xxii) S635N
   (xxiii) A690C and
   (xxiv) Q921H.
Clause 377. A polypeptide comprising an amino acid sequence of sequence of SEQ ID No. 1017 with one to twenty-four mutations selected from the list consisting of:
   (i) A56C
   (ii) A143P
   (iii) Q181H, Q181R or Q181S
   (iv) L214M
   (v) G215S
   (vi) F216M
   (vii) G218D or G218N
   (viii) K219G
   (ix) A238M
   (x) T252Y
   (xi) T311W
   (xii) V326C
   (xiii) G357C
   (xiv) S369C, S369I, S369K or S369M
   (xv) I487M, I487Q or I487V
   (xvi) K492N
   (xvii) V499T
   (xviii) G508S
   (xix) R543C
   (xx) L557Y
   (xxi) G634A
   (xxii) S635N
   (xxiii) A690C and
   (xxiv) Q921H.
Clause 378. The polypeptide according to either clause 376 or 377, having one of the substitutions.
Clause 379. The polypeptide according to either clause 376 or 377, having two of the substitutions.
Clause 380. The polypeptide according to either clause 376 or 377, having three of the substitutions.
Clause 381. The polypeptide according to either clause 376 or 377, having four of the substitutions.
Clause 382. The polypeptide according to either clause 376 or 377, having five of the substitutions.
Clause 383. The polypeptide according to either clause 376 or 377, having six of the substitutions.
Clause 384. The polypeptide according to either clause 376 or 377, having seven of the substitutions.
Clause 385. The polypeptide according to either clause 376 or 377, having eight of the substitutions.
Clause 386. The polypeptide according to either clause 376 or 377, having nine of the substitutions.
Clause 387. The polypeptide according to either clause 376 or 377, having ten of the substitutions.
Clause 388. The polypeptide according to either clause 376 or 377, having eleven of the substitutions.
Clause 389. The polypeptide according to either clause 376 or 377, having twelve of the substitutions.
Clause 390. The polypeptide according to either clause 376 or 377, having thirteen of the substitutions.
Clause 391. The polypeptide according to either clause 376 or 377, having fourteen of the substitutions.
Clause 392. The polypeptide according to either clause 376 or 377, having fifteen of the substitutions.
Clause 393. The polypeptide according to either clause 376 or 377, having sixteen of the substitutions.
Clause 394. The polypeptide according to either clause 376 or 377, having seventeen of the substitutions.
Clause 395. The polypeptide according to either clause 376 or 377, having eighteen of the substitutions.
Clause 396. The polypeptide according to either clause 376 or 377, having nineteen of the substitutions.
Clause 397. The polypeptide according to either clause 376 or 377, having twenty of the substitutions.
Clause 398. The polypeptide according to either clause 376 or 377, having twenty-one of the substitutions.
Clause 399. The polypeptide according to either clause 376 or 377, having twenty-two of the substitutions.
Clause 400. The polypeptide according to either clause 376 or 377, having twenty-three of the substitutions.
Clause 401. The polypeptide according to either clause 376 or 377, having twenty-four of the substitutions.
Clause 402. The polypeptide according to any one of clauses 376 to 401, comprising A56C.
Clause 403. The polypeptide according to any one of clauses 376 to 402, comprising A143P.
Clause 404. The polypeptide according to any one of clauses 376 to 403, comprising Q181H, Q181R or Q181S.
Clause 405. The polypeptide according to clause 404, comprising Q181H.
Clause 406. The polypeptide according to clause 404, comprising Q181R.
Clause 407. The polypeptide according to clause 404, comprising Q181S.
Clause 408. The polypeptide according to any one of clauses 376 to 407, comprising L214M.
Clause 409. The polypeptide according to any one of clauses 376 to 408, comprising G215S.
Clause 410. The polypeptide according to any one of clauses 376 to 409, comprising F216M.
Clause 411. The polypeptide according to any one of clauses 376 to 410, comprising G218D or G218N.
Clause 412. The polypeptide according to clause 411, comprising G218D.
Clause 413. The polypeptide according to clause 411, comprising G218N.
Clause 414. The polypeptide according to any one of clauses 376 to 413, comprising K219G.
Clause 415. The polypeptide according to any one of clauses 376 to 414, comprising A238M.
Clause 416. The polypeptide according to any one of clauses 376 to 415, comprising T252Y.
Clause 417. The polypeptide according to any one of clauses 376 to 416, comprising T311W.
Clause 418. The polypeptide according to any one of clauses 376 to 417, comprising V326C.
Clause 419. The polypeptide according to any one of clauses 376 to 418, comprising G357C.
Clause 420. The polypeptide according to any one of clauses 376 to 419, comprising S369C, S369I, S369K or S369M.
Clause 421. The polypeptide according to clause 420, comprising S369C.
Clause 422. The polypeptide according to clause 420, comprising S369I.
Clause 423. The polypeptide according to clause 420, comprising S369K.
Clause 424. The polypeptide according to clause 420, comprising S369M.
Clause 425. The polypeptide according to any one of clauses 376 to 424, comprising I487M, I487Q or I487V.
Clause 426. The polypeptide according to clause 424, comprising I487M.
Clause 427. The polypeptide according to clause 424, comprising I487Q.
Clause 428. The polypeptide according to clause 424, comprising I487V.
Clause 429. The polypeptide according to any one of clauses 376 to 428, comprising K492N.
Clause 430. The polypeptide according to any one of clauses 376 to 429, comprising V499T.
Clause 431. The polypeptide according to any one of clauses 376 to 430, comprising G508S.
Clause 432. The polypeptide according to any one of clauses 376 to 431, comprising R543C.
Clause 433. The polypeptide according to any one of clauses 376 to 432, comprising L557Y.
Clause 434. The polypeptide according to any one of clauses 376 to 433, comprising G634A.
Clause 435. The polypeptide according to any one of clauses 376 to 434, comprising S635N.
Clause 436. The polypeptide according to any one of clauses 376 to 435, comprising A690C.
Clause 437. The polypeptide according to any one of clauses 376 to 436, comprising Q921H.
Clause 438. The polypeptide according to either clause 376 or 377, comprising one mutation which is K219G.
Clause 439. The polypeptide according to either clause 376, 377 or 381 to 401, comprising A143P, L214M, K219G and Q921H.
Clause 440. The polypeptide according to clause 376, 377 or 382 to 401, comprising A143P, L214M, K219G, G357C and Q921H.
Clause 441. The polypeptide according to clause 376, 377 or 386 to 401, comprising A143P, L214M, G215S, G218N, K219G, G357C, G508S, G634A and Q921H.
Clause 442. The polypeptide according to clause 376 to 401, comprising at least one residue substitution from A143P, L214M, G215S, G218D, K219G, G357C, G508S, G634A, A690C and Q921H.
Clause 443. The polypeptide according to clause 442, comprising A143P, L214M, G215S, G218D, K219G, G357C, G508S, G634A, A690C and Q921H.
Clause 444. The polypeptide according to clause 376, 377 or 385 to 401 comprising A143P, L214M, G215S, K219G, G357C, G508S, G634A and Q921H and one to sixteen mutations selected from the list consisting of:
   (i) A56C
   (iii) Q181H, Q181R or Q181S
   (vi) F216M
   (vii) G218D or G218N
   (ix) A238M
   (x) T252Y
   (xi) T311W
   (xii) V326C
   (xiv) S369C, S369I, S369K or S369M
   (xv) I487M, I487Q or I487V
   (xvi) K492N
   (xvii) V499T
   (xix) R543C
   (xx) L557Y
   (xxii) S635N and
   (xxiii) A690C.
Clause 445. The polypeptide according to clause 444, comprising G218D or G218N.
Clause 446. The polypeptide according to any one of clauses 376 to 445 comprising an amino acid sequence that is at least 85% identical to the amino acid sequence of SEQ ID No. 1017.
Clause 447. The polypeptide according to any one of clauses 376 to 445 comprising an amino acid sequence that is at least 90% identical to the amino acid sequence of SEQ ID No. 1017.
Clause 448. The polypeptide according to any one of clauses 376 to 445 comprising an amino acid sequence that is at least 95% identical to the amino acid sequence of SEQ ID No. 1017.
Clause 449. The polypeptide according to any one of clauses 376 to 445 comprising an amino acid sequence that is at least 98% identical to the amino acid sequence of SEQ ID No. 1017.
Clause 450. The polypeptide according to any one of clauses 376 to 445, wherein the functional variant comprises a sequence having a fragment of at least 100, especially at least 200, particularly at least 300, such as at least 400, for example at least 500 contiguous amino acids of SEQ ID No. 1017.
Clause 451. The polypeptide according to clause 376, comprising the amino acid sequence of SEQ ID No. 1189.
Clause 452. The polypeptide according to clause 376, comprising the amino acid sequence of SEQ ID No. 1190.
Clause 453. The polypeptide according to clause 376, comprising the amino acid sequence of SEQ ID No. 1191.
Clause 454. The polypeptide according to clause 376, comprising the amino acid sequence of SEQ ID No. 1192.
Clause 455. The polypeptide according to clause 376, comprising the amino acid sequence of SEQ ID No. 1193.
Clause 456. The polypeptide according to any one of clauses 376 to 455, further comprising an affinity tag.
Clause 457. The polypeptide according to clause 456, wherein the affinity tag is a poly-his tag, such as a hexa-his tag.
Clause 458. The polypeptide according to either clause 456 or 457, wherein the affinity tag is N-terminally located.
Clause 459. The polypeptide according to clause 458, comprising an amino acid sequence of SEQ ID No. 1177.
Clause 460. The polypeptide according to either clause 456 or 457, wherein the affinity tag is C-terminally located.
Clause 461. The polypeptide according to clause 460, comprising an amino acid sequence of SEQ ID No. 1178.
Clause 462. The polypeptide according to any one of clauses 376 to 461, comprising 1100 residues or fewer, especially 1050 residues or fewer, in particular 1000 residues or fewer, such as 950 residues or fewer.
Clause 463. The polypeptide according to any one of clauses 376 to 462, consisting of an amino acid sequence of SEQ ID No. 1 with one to twenty-four mutations selected from the list consisting of: A56C
   (ii) A143P
   (iii) Q181H, Q181R or Q181S
   (iv) L214M
   (v) G215S
   (vi) F216M
   (vii) G218D or G218N
   (viii) K219G
   (ix) A238M
   (x) T252Y
   (xi) T311W
   (xii) V326C
   (xiii) G357C
   (xiv) S369C, S369I, S369K or S369M
   (xv) I487M, I487Q or I487V
   (xvi) K492N
   (xvii) V499T
   (xviii) G508S
   (xix) R543C
   (xx) L557Y
   (xxi) G634A
   (xxii) S635N
   (xxiii) A690C and
   (xxiv) Q921H.
Clause 464. A polynucleotide comprising a sequence encoding an engineered glucosidase or engineered rhamnosidase polypeptide according to any one of clauses 260 to 463.

The teaching of all references in the present application, including patent applications and granted patents, are herein fully incorporated by reference to the fullest extent possible. A composition or method or process defined as “comprising” certain elements is understood to encompass a composition, method or process (respectively) consisting of those elements. As used herein, ‘consisting essentially of’ means additional components may be present provided they do not alter the overall properties or function.

In respect of numerical values, the terms ‘approximately’, ‘around’ or ‘about’ will typically mean a value within plus or minus 10 percent of the stated value, especially within plus or minus 5 percent of the stated value and in particular the stated value.

Throughout the specification, including the claims, where the context permits, the term “comprising” and variants thereof such as “comprises” are to be interpreted as including the stated element (e.g., integer) or elements (e.g., integers) without necessarily excluding any other elements (e.g., integers). Thus a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

As used herein, the singular forms “a,” “an” and “the” include plural references unless the content clearly dictates otherwise.

As used herein, ng refers to nanograms, ug or μg refers to micrograms, mg refers to milligrams, mL or ml refers to milliliter, and mM refers to millimolar. Similar terms, such as um, are to be construed accordingly.

Unless specifically stated, a process comprising a step of mixing two or more components does not require any specific order of mixing. Thus components can be mixed in any order. Where there are three components then two components can be combined with each other, and then the combination may be combined with the third component, etc.

The invention will be further described by reference to the following, non-limiting, examples:

### EXAMPLES

### Example 1—HPLC of a Crude Aqueous Extract of Quillaja saponaria

Crude bark extract was separated by reverse phase HPLC using a C4 column and gradient elution: mobile phase A—water/acetonitrile, 7/3 v/v with 0.15% trifluoroacetic acid; mobile phase B—acetonitrile with 0.15% trifluoroacetic acid. UV detection was at 214 nm.

Crude bark extract samples are diluted as necessary with purified water. Polyvinylpolypyrrolidone (PVPP; 60 mg/mL) was added, the mixture stirred for approximately 30 minutes, and then centrifuged to separate the PVPP resin from the supernatant.

The supernatant was then analysed to provide an HPLC UV chromatogram.

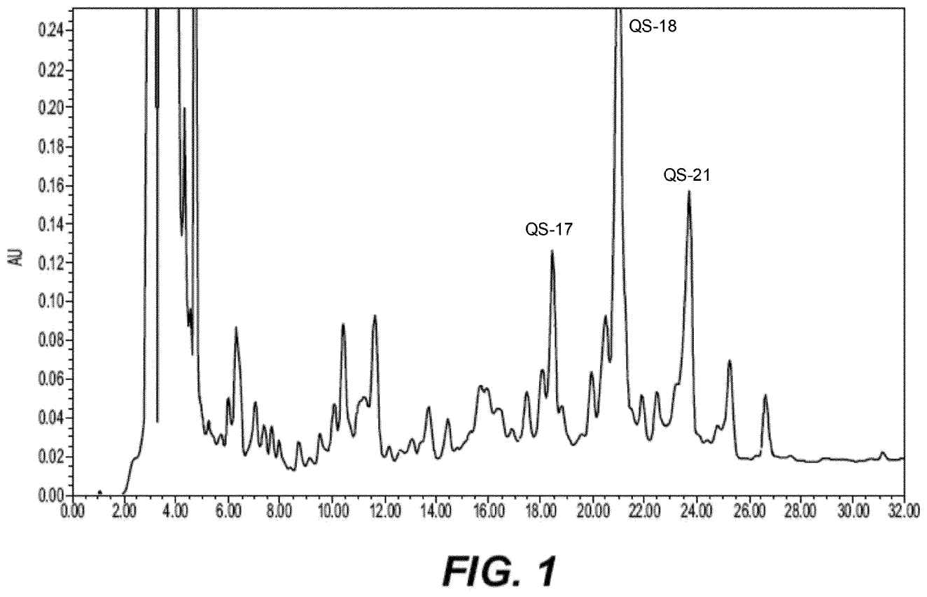

FIG. 1 provides a representative example of an HPLC UV chromatogram. The peak corresponding to the QS-21 fraction is indicated.

### Example 2—Analytical Methods

### HPLC-UV

### Equipment

Waters Alliance 2690/2695 separations module
Waters 2487 UV Detector or 2996 PDA Detector
Vydac Protein C4 4.6×250 mm 5 um column
Mobile Phase A (MPA)—0.15% trifluoroacetic acid in water/acetonitrile (70:30 v/v)
Mobile Phase B (MPB)—0.15% trifluoroacetic acid in acetonitrile

TABLE 1
HPLC-UV Linear gradient conditions

| Time | Flow rate (ml/min) | % MPA | % MPB |
|---|---|---|---|
| 0 | 1 | 100 | 0 |
| 30 | 1 | 78.6 | 21.4 |
| 33 | 1 | 14.3 | 85.7 |

40 ul of sample is injected. UV detection is set at 214 nM.

Using a blank injection for reference, integration of peaks in the chromatogram provides a total absorbance. Peak of interest (e.g. QS-21 main peak) is compared to total absorbance to determine peak content as a percentage.

The HPLC-UV method is also conveniently used to determine QS-21 main peak content and Preceding peak to QS-21 main peak ratio.

### UPLC-UV

### Equipment

Waters Acquity UPLC
Waters Acquity Tunable UV Detector
Waters Acquity BEH C18 2.1×100 mm 1.7 um column
Mobile Phase A (MPA)—0.025% acetic acid in water/acetonitrile (70:30 v/v)
Mobile Phase B (MPB)—0.025% trifluoroacetic acid in water/acetonitrile (30:70 v/v)

TABLE 2
UPLC-UV Linear gradient conditions

| Time | Flow rate (ml/min) | % MPA | % MPB |
|---|---|---|---|
| 0 | 0.5 | 88 | 12 |
| 10.2 | 0.5 | 65.7 | 34.3 |
| 11.2 | 0.5 | 10 | 90 |
| 13.2 | 0.5 | 10 | 90 |

Column temperature 28 degrees C. 10 ul of sample is injected. UV detection is set at 214 nM.

Using a blank injection for reference, integration of peaks in the chromatogram provides a total absorbance. Peak of interest (e.g. QS-21 main peak) is compared to total absorbance to determine peak content as a percentage.

The UPLC-UV method is also conveniently used to determine 2018/QS-21 Ratio.

### UPLC-UV/MS

### Equipment

Waters Acquity UPLC
Waters Acquity Tunable UV Detector
Waters Single-Quadrupole Mass Detector SQD1 (scanning range 1400 to 2040 M/Z)
Waters Acquity BEH C18 2.1×100 mm 1.7 um column
Mobile Phase A (MPA)—0.025% trifluoroacetic acid in water/acetonitrile/isopropyl alcohol (75:20:5 v/v)
Mobile Phase B (MPB)—0.025% trifluoroacetic acid in water/acetonitrile/isopropyl alcohol (10:72:18 v/v)

TABLE 3
UPLC-UV/MS Linear gradient conditions

| Time | Flow rate (ml/min) | % MPA | % MPB |
|---|---|---|---|
| 0 | 0.6 | 100 | 0 |
| 6.23 | 0.6 | 23 | 77 |

Test sample is prepared in 0.2% acetic acid in water/acetonitrile (70:30 v/v). Column temperature 55 degrees C. 10 ul of sample is injected. UV detection is set at 214 nM.

The term ‘QS-21 group’ is meant the triterpenoid glycosides identified from the B-isomer to the peak preceding the Iyo impurity in the UPLC-UV/MS methods described herein. Although retention times vary slightly between runs, the QS-21 group is located at approximately 3.8 min (QS-21 B-isomer) to approximately 4.5 minutes (prior to Iyo impurity peak, containing desarabinofuranosyl-QS-21 1856 A component).

Using a blank injection for reference, integration of peaks in the chromatogram that elute after the solvent front between 0.5 and around 5.50 minutes and do not appear in the blank is undertaken.

The monoisotope of the most abundant species is identified by combining TIC over the entire chromatogram to create a combined spectrum.

Ratio of QS-21 2002 A component to QS-21 1988 A component is calculated by comparing the ion current associated with the QS-21 2002 A component with the ion current associated with the QS-21 1988 A component within the QS-21 main peak.

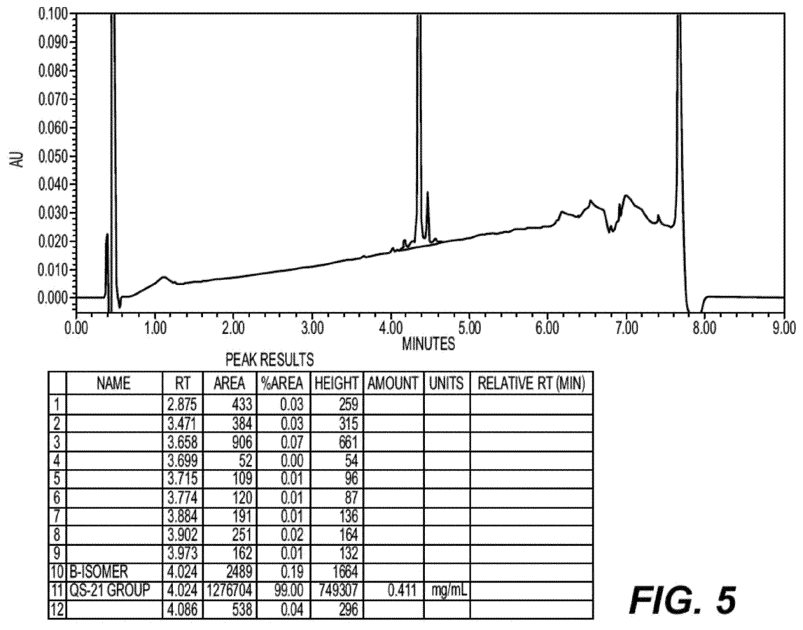

FIG. 5 provides a chromatogram of an exemplary saponin extract. FIG. 6 shows expanded detail of the region including the QS-21 group and impurity peak.

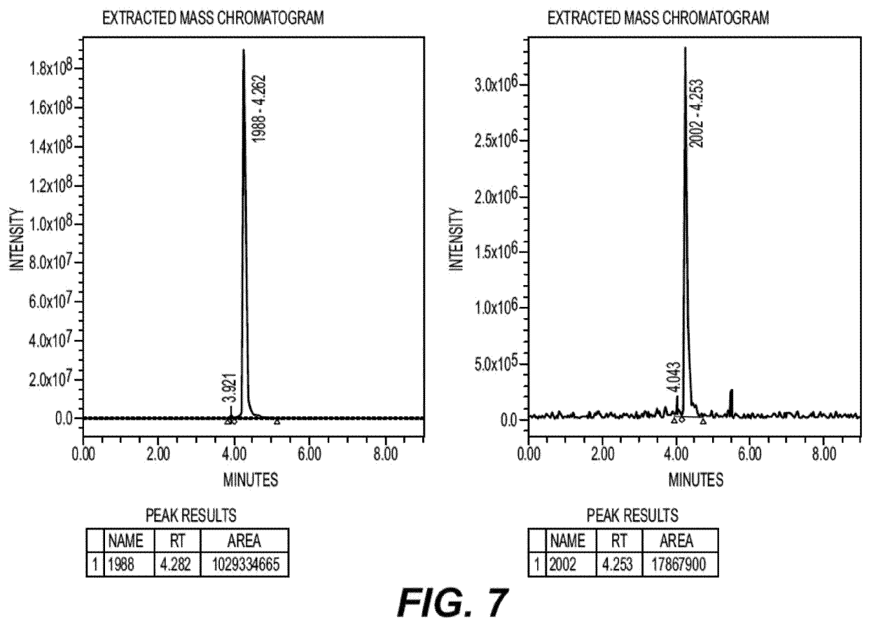

FIGS. 7A and 7B provide extracted mass chromatograms for QS-21 1988 A (FIG. 7A) and QS-21 2002 A (FIG. 7B) molecular weight ions of an exemplary purified Quillaja saponaria saponin extract.

### Example 3—Purification of a Crude Aqueous Extract of Quillaja saponaria

Crude aqueous extract of Quillaja saponaria material having a 2018 component to QS-21 main peak ratio of 0.064 or lower and a Preceding peak to QS-21 main peak ratio of 0.4 or lower, was treated with PVPP (1 kg PVPP per litre of crude aqueous extract). After adsorption the mixture was filtered to separate the PVPP and bound impurities from the liquor.

FIG. 2 provides an example HPLC-UV chromatogram for crude aqueous extract of Quillaja saponaria (used for Preceding peak to QS-21 main peak ratio determination and QS-21 main peak content).

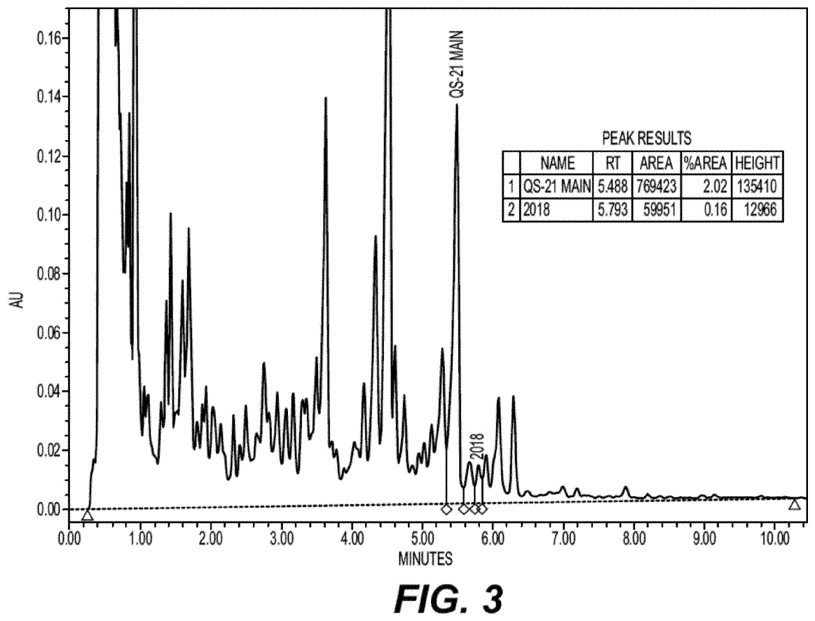

FIG. 3 provides an example UPLC-UV chromatogram for crude aqueous extract of Quillaja saponaria (used for 2018 component to QS-21 main peak ratio determination).

Filtered liquor was concentrated and further purified by ultrafiltration/diafiltration using water and a 30 kD Hellicon membrane.

Resulting permeate was purified by reverse phase chromatography using a polystyrene resin (Amberchrom XT20).

TABLE 4
Reverse phase chromatography polystyrene resin gradient conditions

| | | Initial | | Final | |
|---|---|---|---|---|---|
| | Duration | % Eluent | % Eluent | % Eluent | % Eluent |
| Step | (min) | A | B | A | B |
| Injection + Rinse | 11.3 | 100% | 0% | 100% | 0% |
| Gradient Elution 1 | 3.0 | 100% | 0% | 71% | 29% |
| Gradient Elution 2 | 50.0 | 71% | 29% | 53% | 47% |
| Gradient Elution 3 | 3.0 | 53% | 47% | 0% | 100% |
| Regeneration | 10 | 0% | 100% | 0% | 100% |
| Gradient | 3.0 | 0% | 100% | 100% | 0% |
| Equilibration | 13.0 | 100% | 0% | 100% | 0% |

Eluent A: 5% Acetonitrile and 0.25% acetic acid
Eluent B: 90% Acetonitrile and 0.25% acetic acid
Column: 30 cm ID, approximately 17.7 to 20.5 L volume
Loading: 50-110 g per injection

Fractions were pooled to provide polystyrene purified saponin extract with a composition:

% QS-21 main peak≥18% (by HPLC)

and

2018 component/QS-21 main peak ratio≤0.054 (by UPLC-UV).

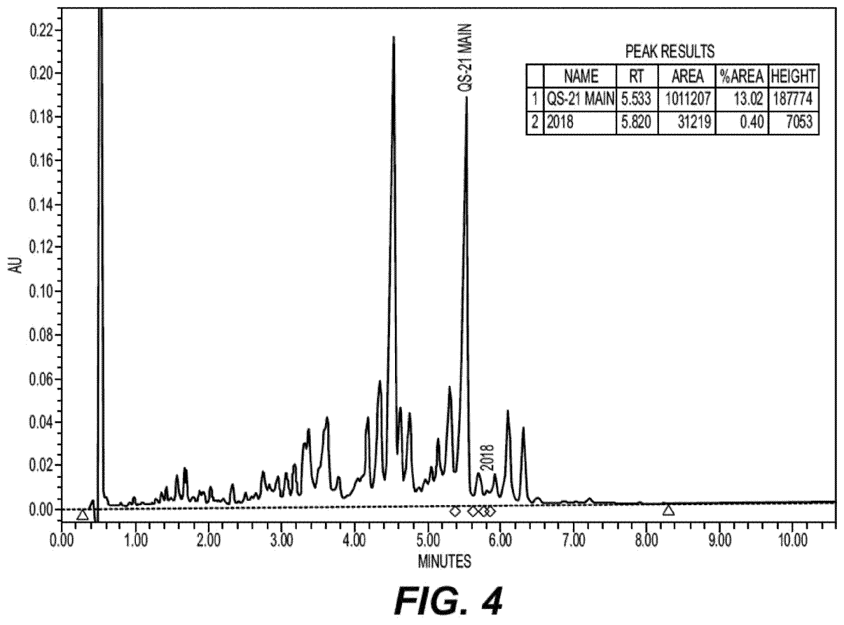

FIG. 4 provides an example UPLC-UV chromatogram for a polystyrene purified saponin extract pool.

The combined polystyrene purified fraction pool was further purified by reverse phase chromatography using a phenyl resin (EPDM).

TABLE 5
Reverse phase chromatography phenyl resin gradient conditions

| | Duration | | |
|---|---|---|---|
| Step | (min) | % Eluent C | % Eluent B |
| Injection + Rinse | 2.0 | 100% | 0% |
| Isocratic Elution | 58.0 | 100% | 0% |
| Regeneration | 5.0 | 0% | 100% |
| Equilibration | 10.0 | 100% | 0% |

Eluent B: 90% Acetonitrile and 0.25% acetic acid
Eluent C: 35.2% acetonitrile and 0.25% acetic acid
Column: 45 cm ID, approximately 39.8 to 42.9 L volume
Loading: 13-21 g per injection

QS-21 containing fractions were pooled to provide phenyl purified saponin extract with a composition:

% QS-21 group≥98.5%

QS-21 main peak≥94.5%

2018 component≤2.7%

Main peak outside of the QS-21 group≤1% (by UPLC-UV/MS).

The combined phenyl purified saponin extract was concentrated by capture and release with reverse phase chromatography using a C8 resin (Lichroprep RP8) and the following conditions:
Loaded to column conditioned at 24% acetonitrile and 0.20% acetic acid.
Eluted with 60% acetonitrile and 0.20% acetic acid.
11 cm column, approximately 0.87 to 0.97 L volume
Load: 50-142 g per injection

The C8 concentrated saponin extract was subjected to solvent exchange using ultrafiltration/diafiltration and a Pellicon 1 kDa membrane to reduce acetonitrile content below 21%.

The resulting solvent exchanged saponin extract was then lyophilised in a single step to provide a final purified saponin extract product.

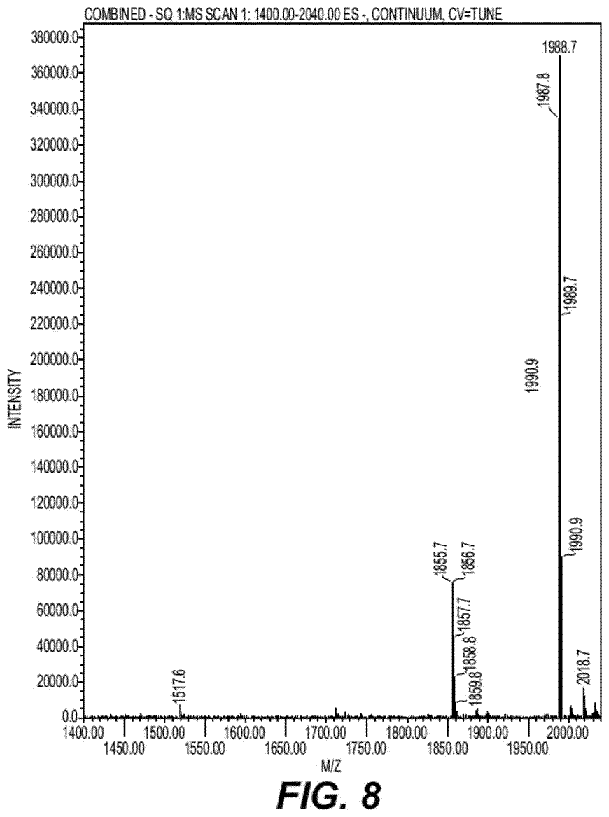

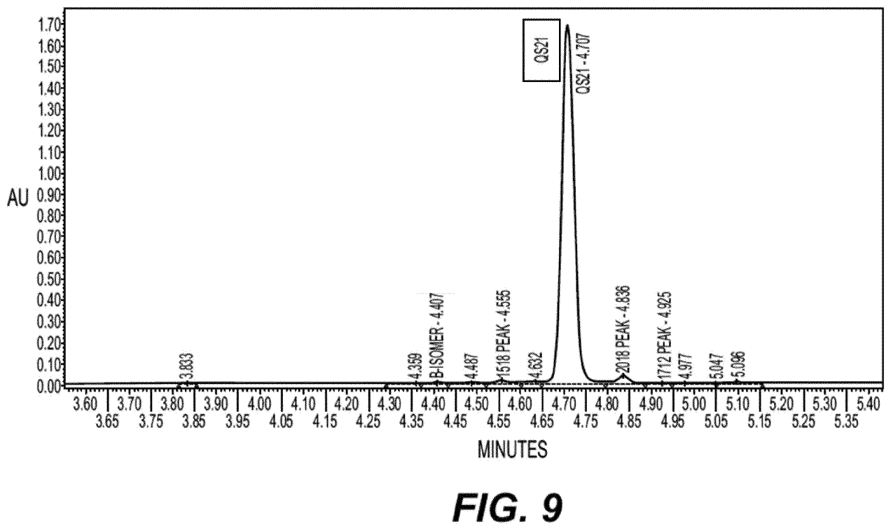

The use of the process as described in Example 3 can consistently provide a purified saponin extract of Quillaja saponaria having a defined content in terms of QS-21 main peak and 2018 component, presenting a chromatographic profile comparable to the chromatograms shown in FIGS. 5 to 9.

### Example 4—Screening of Glucosidases for Deglucosylation of QS-18 to QS-21

### Method

### Enzyme Selection

The enzyme family of hydrolases (E.C.3.2.1.-) that act on glycosidic bonds (‘glycoside hydrolases’ (GH) or ‘glycosidases’), contains at present approximately one million members having wide ranging activities across molecules containing glycans and polysaccharides. A typical QS-18 family molecule contains a number of such glycosidic bonds, with the presence of the 1-3 bond between the alpha-L-rhamnose on the linear tetrasaccharide and the branched terminal beta-D-glucose differentiating the QS-18 family from the QS-21 family. The specific hydrolysis of this bond by a beta-glucosidase, i.e. an enzyme with exo-beta-1,3-glucosidase activity (E.C. 3.2.1.21 and E.C. 3.2.1.58) will therefore convert QS-18 family components to QS-21 family components. Specific members of the glycoside hydrolase family having exo-beta-1,3-glucosidase activity were initially identified using the CAZy (Carbohydrate Active enZyme) database (www.cazy.com), with GH families 1, 3 and 5 purported to have enzyme members with the desired exo-beta-1,3 activity. All sequences annotated by CAZy from GH families 1,3 and 5 were obtained, and separate curated hidden Markov model profiles constructed for each which were then used to identify additional familial enzymes by searching the 209 million protein member Uniprot (www.uniprot.org) knowledgebase with the software HMMER (Eddy, 1998). In total, 22,594 sequences: 12,049, 9,278 and 1,267 representatives from GH families 1,3 and 5, respectively, were identified using this method. MMSeqs2 (Hauser, 2016) was then used to cluster each group of enzyme sequences using the default clustering workflow and parameters with a minimum sequence identity and coverage of 30% and 80%, respectively. In cases where the initial clustering yielded clusters with more than 1000 members, a second sub-clustering was performed at a higher 50% or 70% identity to ensure diverse exemplars from these larger clusters were represented more prominently. All clusters were then examined, and exemplars selected from each with preferences for annotation quality, known experimental activity, existing three dimensional structures from the Protein Data Bank (www.wwpdb.org) or known extremophile organisms as annotated by Uniprot. A final set of 400 diverse candidate enzymes was selected. Polynucleotide sequences encoding each selected enzyme linked to an N-terminal 6×His tag and Tev-cleavage site were prepared (amino acid sequence for His-tag linker, inserted N-terminally of normal start methionine, is provided in SEQ ID No. 1177) using a proprietary genetic-algorithm based codon optimization code.

Details of the candidate enzyme and polynucleotide sequences are summarised below in Table 6.

TABLE 6
Glucosidase candidate sequences

| | | | AA | DNA |
|---|---|---|---|---|
| UniParc Ref @1 | Uniprot ref | Organism | Seq ID | Seq ID |
| UPI00049B1A8C | A0A061B3J2 | Cyberlindnera fabianii | 1 | 401 |
| UPI0004E3EF7B | A0A085EII0 | Flavobacterium gilvum | 2 | 402 |
| UPI00050EE490 | A0A090X649 | Algibacter lectus | 3 | 403 |
| UPI0005ECB51E | A0A0F0LB94 | Microbacterium azadirachtae | 4 | 404 |
| UPI0006588DAD | A0A0J0UT37 | Actinobacteria bacterium | 5 | 405 |
| UPI0007968552 | A0A136KWB3 | Chloroflexi bacterium | 6 | 406 |
| UPI0002080410 | A0A181C809 | Komagataeibacter rhaeticus | 7 | 407 |
| UPI0008211BFC | A0A1C5WEL8 | Bacteroides sp. | 8 | 408 |
| UPI00085A2BD0 | A0A1D8FZW3 | Streptomyces rubrolavendulae | 9 | 409 |
| UPI00098C60F6 | A0A1S8KYM5 | Clostridium roseum | 10 | 410 |
| UPI0009CE0D4C | A0A1V5M6V6 | uncultured bacterium | 11 | 411 |
| UPI0009D4127D | A0A1V6AN95 | Firmicutes bacterium | 12 | 412 |
| UPI00045601AB | A0A061H1Z3 | Anthracocystis flocculosa | 13 | 413 |
| UPI0004FF77C7 | A0A086ZKU2 | Bifidobacterium boum | 14 | 414 |
| UPI00051EDBDE | A0A098LTR2 | Jejuia pallidilutea | 15 | 415 |
| UPI00062105AB | A0A0F8B2B0 | Ceratocystis fimbriata | 16 | 416 |
| UPI0006583AB1 | A0A0J0UVW7 | Actinobacteria bacterium | 17 | 417 |
| UPI0007AABFAD | A0A143QAX3 | Rhodococcus sp. | 18 | 418 |
| UPI0007F2D02D | A0A194VF47 | Valsa mali | 19 | 419 |
| UPI000821004D | A0A1C5WSI4 | uncultured Bacteroides sp. | 20 | 420 |
| UPI00084089B2 | A0A1E3ALT2 | Eisenbergiella tayi | 21 | 421 |
| UPI000978E914 | A0A1V2MY14 | Streptomyces sp. | 22 | 422 |
| UPI0009CBF21C | A0A1V5MH90 | Firmicutes bacterium | 23 | 423 |
| UPI0009D5B1F0 | A0A1V6BAK3 | Tenericutes bacterium | 24 | 424 |
| UPI0004A87350 | A0A067Z479 | Gluconobacter oxydans | 25 | 425 |
| UPI00050787A2 | A0A087B8Q8 | Bifidobacterium catenulatum | 26 | 426 |
| UPI0005965863 | A0A0B7K538 | Bionectria ochroleuca | 27 | 427 |
| UPI0006377CA4 | A0A0G0GD78 | Parcubacteria sp. | 28 | 428 |
| UPI0006228575 | A0A0M2H276 | Microbacterium ketosireducens | 29 | 429 |
| UPI0006C454B4 | A0A173R3W4 | Roseburia faecis | 30 | 430 |
| UPI0007F1D695 | A0A1A6A050 | Kwoniella dejecticola | 31 | 431 |
| UPI0008232A70 | A0A1C6C862 | uncultured Clostridium sp. | 32 | 432 |
| UPI000866CB30 | A0A1E4S2F8 | Cyberlindnera jadinii | 33 | 433 |
| UPI0009D3483D | A0A1V5G4W6 | Bacteroidetes bacterium | 34 | 434 |
| UPI0009CC9AEA | A0A1V5Q4R4 | Verrucomicrobia bacterium | 35 | 435 |
| UPI0009C6DE73 | A0A1V6BV25 | Bacteroidetes bacterium | 36 | 436 |
| UPI0004E051A9 | A0A077WUK7 | Lichtheimia ramosa | 37 | 437 |
| UPI0005060F52 | A0A087BWT7 | Bifidobacterium mongoliense | 38 | 438 |
| UPI000591CED9 | A0A0B8NZY1 | Vibrio ishigakensis | 39 | 439 |
| UPI00063B706F | A0A0G2HEV5 | Phaeomoniella chlamydospora | 40 | 440 |
| UPI0006C04F59 | A0A0M8K5H7 | Ardenticatena maritima | 41 | 441 |
| UPI000197E031 | A0A173WPC4 | Coprococcus comes | 42 | 442 |
| UPI0007DDBAB3 | A0A1A9GNJ0 | Nocardioides dokdonensis | 43 | 443 |
| UPI000822F7EB | A0A1C6EBC1 | uncultured Clostridium sp. | 44 | 444 |
| UPI000878EB40 | A0A1F2PFB4 | Acetobacterium wieringae | 45 | 445 |
| UPI0009D0A2C1 | A0A1V5HNJ3 | Tenericutes bacterium | 46 | 446 |
| UPI0009CB8A73 | A0A1V5UFI1 | Tenericutes bacterium | 47 | 447 |
| UPI0009CC90AF | A0A1V6CDT2 | uncultured bacterium | 48 | 448 |
| UPI0004D8E473 | A0A078SYD0 | Parabacteroides distasonis | 49 | 449 |
| UPI0005006C6A | A0A087CJB0 | Bifidobacterium psychraerophilum | 50 | 450 |
| UPI00059A3BA8 | A0A0C2YJF2 | Hebeloma cylindrosporum | 51 | 451 |
| UPI0006579FEA | A0A0G4JRR8 | Brenneria goodwinii | 52 | 452 |
| UPI00073C9E60 | A0A0U5GN20 | Aspergillus calidoustus | 53 | 453 |
| UPI0006C6E0C3 | A0A174BEZ2 | Bacteroides finegoldii | 54 | 454 |
| UPI0008153D6D | A0A1B2A943 | Altererythrobacter dongtanensis | 55 | 455 |
| UPI0008206E7B | A0A1C6FWD9 | uncultured Anaerotruncus sp. | 56 | 456 |
| UPI0008AC0E17 | A0A1F5UJR6 | Candidatus firestone | 57 | 457 |
| UPI0009C6C8E4 | A0A1V5HS22 | Tenericutes bacterium | 58 | 458 |
| UPI0009CEE1AB | A0A1V5VBL2 | Lentisphaerae bacterium | 59 | 459 |
| UPI0009CAAB0A | A0A1V6FZ47 | Planctomycetes bacterium | 60 | 460 |
| UPI0004DD62AC | A0A084G332 | Pseudallescheria apiosperma | 61 | 46 |
| UPI000507F00A | A0A090Q4N8 | Nonlabens sediminis | 62 | 462 |
| UPI0005CC42CA | A0A0C5VDU3 | Gynuella sunshinyii | 63 | 463 |
| UPI00063E4005 | A0A0G4N9Q7 | Verticillium longisporum | 64 | 464 |
| UPI00073C6CD3 | A0A0V8TAB5 | Cellulomonas sp. | 65 | 465 |
| UPI0006C024BB | A0A174FBX7 | Hungatella hathewayi | 66 | 466 |
| UPI000688E74D | A0A1C2DG64 | Mesorhizobium sp. | 67 | 467 |
| UPI000822FAB7 | A0A1C6GRT5 | Clostridium sp. | 68 | 468 |
| UPI0009284E74 | A0A1M3CSY6 | Chlamydiales bacterium | 69 | 469 |
| UPI0009CB461D | A0A1V5HUX3 | Spirochaetes bacterium | 70 | 470 |
| UPI0009C92D6A | A0A1V5VHB9 | bacterium | 71 | 471 |
| UPI0009D12B86 | A0A1V6H2W5 | Thermotogae bacterium | 72 | 472 |
| UPI0004DCBED3 | A0A084GGE2 | Pseudallescheria apiosperma | 73 | 473 |
| UPI00050E0BE3 | A0A090VF17 | Algibacter lectus | 74 | 474 |
| UPI0005B075C8 | A0A0C9TWP5 | Paxillus involutus | 75 | 475 |
| UPI00063DF006 | A0A0G4NA55 | Verticillium longisporum | 76 | 476 |
| UPI00076F8EA4 | A0A110B1H1 | Mucilaginibacter gotjawali | 77 | 477 |
| UPI0006C0FFEF | A0A174IWW4 | Bacteroides uniformis | 78 | 478 |
| UPI0008222B77 | A0A1C5W0N6 | Coprococcus sp. | 79 | 479 |
| UPI0006C36823 | A0A1C6K2X5 | Blautia sp. | 80 | 480 |
| UPI00092B3CD4 | A0A1M3ELH4 | Cellulomonas sp. | 81 | 481 |
| UPI0009C85428 | A0A1V5J984 | bacterium | 82 | 482 |
| UPI0009CA1D5C | A0A1V5WHQ7 | Spirochaetes bacterium | 83 | 483 |
| UPI0009C7041F | A0A1V6IJ57 | Tenericutes bacterium | 84 | 484 |
| UPI0004E2A41F | A0A085EG29 | Flavobacterium gilvum | 85 | 485 |
| UPI0005102470 | A0A090WWZ1 | Algibacter lectus | 86 | 486 |
| UPI0005B0F0AE | A0A0C9WDY0 | Hydnomerulius pinastri | 87 | 487 |
| UPI00065C2666 | A0A0H5NWN2 | Nocardia farcinica | 88 | 488 |
| UPI000760375B | A0A125MG18 | Bacteroides cellulosilyticus | 89 | 489 |
| UPI0007DF4250 | A0A178ZBN2 | Fonsecaea erecta | 90 | 490 |
| UPI0008209852 | A0A1C5W9N9 | Bacteroides sp. | 91 | 491 |
| UPI00086C34C1 | A0A1D3UGH8 | Tannerella forsythia | 92 | 492 |
| UPI00097E83BB | A0A1R4KI92 | Microbacterium esteraromaticum | 93 | 493 |
| UPI0009D255E5 | A0A1V5LJK9 | bacterium | 94 | 494 |
| UPI0009C5A3CF | A0A1V5Z2L2 | Candidatus hydrogenedentes | 95 | 495 |
| UPI0009C55799 | A0A1V6J4J8 | Bacteroidetes bacterium | 96 | 496 |
| UPI0009D4067F | A0A1V6RQ41 | Penicillium solitum | 97 | 497 |
| UPI0008737AA2 | A0A288Q812 | Weissella soli | 98 | 498 |
| UPI000CAC57D4 | A0A2K4ZN91 | Acetatifactor muris | 99 | 499 |
| UPI000D237A4A | A0A2T2NYD4 | Corynespora cassiicola | 100 | 500 |
| UPI000D77C91D | A0A316V6M3 | Meira miltonrushii | 101 | 501 |
| UPI00004E1F76 | A0A380YVC7 | Bacteroides fragilis | 102 | 502 |
| UPI000F0C30E8 | A0A3G2SB79 | Malassezia restricta | 103 | 503 |
| UPI000FFFEFB2 | A0A430LYA2 | Fusarium euwallaceae | 104 | 504 |
| UPI0010251887 | A0A4Q2E070 | Psathyrella aberdarensis | 105 | 505 |
| UPI0000E69509 | A0KLP6 | Aeromonas hydrophila | 106 | 506 |
| UPI00000B86CB | A4F7P9 | Saccharopolyspora erythraea | 107 | 507 |
| UPI000180240E | B5I181 | Streptomyces sviceus | 108 | 508 |
| UPI000A250F78 | A0A1Y2AWB7 | Naematelia encephala | 109 | 509 |
| UPI000C0221F1 | A0A2C9D612 | Hartmannibacter diazotrophicus | 110 | 510 |
| UPI000CEB5AB1 | A0A2L2BPE2 | Pontimonas salivibrio | 111 | 511 |
| UPI000D5B9C38 | A0A2V1CH24 | Cadophora sp. | 112 | 512 |
| UPI000D779558 | A0A316V8S0 | Meira miltonrushii | 113 | 513 |
| UPI000DC42E3D | A0A395IJW4 | Monilinia fructigena | 114 | 514 |
| UPI000F3E476C | A0A3M6XGS0 | Hortaea werneckii | 115 | 515 |
| UPI00101460D7 | A0A445N7U0 | Streptomyces netropsis | 116 | 516 |
| UPI001139C6A8 | A0A4S9IFI0 | Aureobasidium pullulans | 117 | 517 |
| UPI0000EA5CFF | A1CTN9 | Aspergillus clavatus | 118 | 518 |
| UPI0001523037 | A5CT94 | Clavibacter michiganensis | 119 | 519 |
| UPI0001831CF5 | B6H7R5 | Penicillium rubens | 120 | 520 |
| UPI000B365547 | A0A1Y4NTL9 | Lachnoclostridium sp. | 121 | 521 |
| UPI000C09BF88 | A0A2D5IXB9 | Rhodobacteraceae bacterium | 122 | 522 |
| UPI0004B5EEF2 | A0A2M9UUC4 | Bacteroides fragilis | 123 | 523 |
| UPI000D7FE1ED | A0A2V5IY78 | Aspergillus indologenus | 124 | 524 |
| UPI000D802B25 | A0A316YR39 | Acaromyces ingoldii | 125 | 525 |
| UPI000DC60823 | A0A395J1U5 | Monilinia fructigena | 126 | 526 |
| UPI000F52D773 | A0A3N6CA02 | Paenibacillus xylanexedens | 127 | 527 |
| UPI000F6DAAAE | A0A448HIG0 | Actinomyces howellii | 128 | 528 |
| UPI00113D19DF | A0A4V5N914 | Friedmanniomyces endolithicus | 129 | 529 |
| UPI0000EA8672 | A1DNS0 | Neosartorya fischeri | 130 | 530 |
| UPI0000D7314B | A6V4K6 | Pseudomonas aeruginosa | 131 | 531 |
| UPI00018E7266 | B8MF24 | Talaromyces stipitatus | 132 | 532 |
| UPI000A1A5FD7 | A0A1Y5RVF8 | Aquimixticola soesokkakensis | 133 | 533 |
| UPI000C98D37C | A0A2D9YGV1 | Rhodobacterales bacterium | 134 | 534 |
| UPI0006F9793E | A0A2N9AS40 | Methylorubrum extorquens | 135 | 535 |
| UPI00000CF7C8 | A0A2X2YBP0 | Clostridium perfringens | 136 | 536 |
| UPI0008AEBEA3 | A0A348NID6 | Acholeplasmatales bacterium | 137 | 537 |
| UPI000EC3C979 | A0A3B8VHE8 | Bacteroidetes bacterium | 138 | 538 |
| UPI000F637E38 | A0A3P6K8E8 | Clostridium carnis | 139 | 539 |
| UPI000B93B5C9 | A0A448HNB5 | Mycolicibacterium flavescens | 140 | 540 |
| UPI000F6EFA71 | A0A4V6LJ94 | Streptococcus gallolyticus | 141 | 541 |
| UPI0000EB54CC | A1JNB7 | Yersinia enterocolitica | 142 | 542 |
| UPI0000ED8A80 | A7HFG4 | Anaeromyxobacter sp. | 143 | 543 |
| UPI00018E7D70 | B8MK55 | Talaromyces stipitatus | 144 | 544 |
| UPI000A2E3FAA | A0A1Z5SL14 | Hortaea werneckii | 145 | 545 |
| UPI000C529025 | A0A2E2Q8X2 | Micavibrio sp. | 146 | 546 |
| UPI000CE6B996 | A0A2N9MBS0 | Acidobacteriia bacterium | 147 | 547 |
| UPI000DA3A972 | A0A2X4T570 | Corynebacterium jeikeium | 148 | 548 |
| UPI000E8D37A1 | A0A353PZH8 | Clostridiaceae bacterium | 149 | 549 |
| UPI000748C096 | A0A3B9PA35 | Anaerolineaceae bacterium | 150 | 550 |
| UPI000FF41956 | A0A409WSY0 | Gymnopilus dilepis | 151 | 551 |
| UPI000F7105D4 | A0A448R8N0 | Kocuria rosea | 152 | 552 |
| UPI0011696FAB | A0A509DWZ3 | Teredinibacter sp. | 153 | 553 |
| UPI0000EFB564 | A2QS42 | Aspergillus niger | 154 | 554 |
| UPI000164423D | B0D734 | Laccaria bicolor | 155 | 555 |
| UPI00017357F6 | B9XH33 | Pedosphaera parvula | 156 | 556 |
| UPI00042469F6 | A0A239TGH2 | Megamonas hypermegale | 157 | 557 |
| UPI000BC209C3 | A0A2H3E300 | Armillaria gallica | 158 | 558 |
| UPI000D2EAE87 | A0A2P8AV03 | Micromonospora sp. | 159 | 559 |
| UPI000DA286EE | A0A2X5CJC5 | Klebsiella oxytoca | 160 | 560 |
| UPI000DFAEF6C | A0A367ZIH8 | Candidatus ozemobacter | 161 | 561 |
| UPI000E399EE8 | A0A3D8R2C2 | Coleophoma crateriformis | 162 | 562 |
| UPI000FBC01E3 | A0A427XHS2 | Apiotrichum porosum | 163 | 563 |
| UPI00068E4E50 | A0A449BJ27 | Acholeplasma hippikon | 164 | 564 |
| UPI001185F074 | A0A516RGT1 | Streptomyces spectabilis | 165 | 565 |
| UPI0000EFCED2 | A2R8G2 | Aspergillus niger | 166 | 566 |
| UPI00000D8BFA | B0RYA0 | Xanthomonas campestris | 167 | 567 |
| UPI00019C9CD7 | C2FDL2 | Lactobacillus paracasei | 168 | 568 |
| UPI000BC490A3 | A0A261XUH4 | Bifiguratus adelaidae | 169 | 569 |
| UPI000CC31AE2 | A0A2H5YYA1 | bacterium | 170 | 570 |
| UPI000D26C9C6 | A0A2P9IY35 | Actinomadura parvosata | 171 | 571 |
| UPI00024F22B8 | A0A2Z5Y4P3 | Melissococcus plutonius | 172 | 572 |
| UPI000E020871 | A0A377KJS3 | Enterococcus durans | 173 | 573 |
| UPI000DD17A7D | A0A3G2S2J6 | Malassezia restricta | 174 | 574 |
| UPI000FA2DE87 | A0A427XZQ0 | Apiotrichum porosum | 175 | 575 |
| UPI0010355193 | A0A4P6PWP5 | Streptomonospora sp. | 176 | 576 |
| UPI00119640A1 | A0A558LH47 | Lactobacillus gasseri | 177 | 577 |
| UPI0000EFD0AA | A2RAJ1 | Aspergillus niger | 178 | 578 |
| UPI000170BF91 | B0XXG1 | Neosartorya fumigata | 179 | 579 |
| UPI0001A5B234 | C4Z6T5 | Eubacterium eligens | 180 | 580 |
| UPI000BC64A75 | A0A261XVM8 | Bifiguratus adelaidae | 181 | 581 |
| UPI000CAA854E | A0A2H6EX57 | bacterium | 182 | 582 |
| UPI000D24EC2C | A0A2T2N5H9 | Corynespora cassiicola | 183 | 583 |
| UPI000D77A0B7 | A0A316UK83 | Pseudomicrostroma glucosiphilum | 184 | 584 |
| UPI0002DAAFCC | A0A380HD31 | Staphylococcus saprophyticus | 185 | 585 |
| UPI000F0C6B8E | A0A3G2S932 | Malassezia restricta | 186 | 586 |
| UPI000FBA70E2 | A0A427YCL1 | Saitozyma podzolica | 187 | 587 |
| UPI00102822B4 | A0A4Q1BRC6 | Tremella mesenterica | 188 | 588 |
| UPI0000527506 | A0JZ86 | Arthrobacter sp. | 189 | 589 |
| UPI000157388C | A3LRB0 | Scheffersomyces stipitis | 190 | 590 |
| UPI0001712E02 | B1XZK8 | Leptothrix cholodnii | 191 | 591 |
| UPI000166883C | C4ZLL7 | Thauera sp. | 192 | 592 |
| UPI00018494AB | C5CDW5 | Kosmotoga olearia | 193 | 593 |
| UPI0001CD671F | D4L3Y2 | Roseburia intestinalis | 194 | 594 |
| UPI0001E0DC00 | E0PDF8 | Streptococcus equinus | 195 | 595 |
| UPI0001F80C4B | E8JUK5 | Streptococcus cristatus | 196 | 596 |
| UPI0001D2DBBC | F2JLH3 | Cellulosilyticum lentocellum | 197 | 597 |
| UPI0001C48657 | F5WY15 | Streptococcus gallolyticus | 198 | 598 |
| UPI00021D443A | F9Y8Z7 | Ketogulonicigenium vulgare | 199 | 599 |
| UPI000228255D | G3AGX1 | Spathaspora passalidarum | 200 | 600 |
| UPI00023F6F5F | G8T9J3 | Niastella koreensis | 201 | 601 |
| UPI000260108C | I3IDC0 | Cellvibrio sp. | 202 | 602 |
| UPI000272D1E0 | J1ACAb | Flavobacterium sp. | 203 | 603 |
| UPI00028E7FE1 | K2S5D3 | Macrophomina phaseolina | 204 | 604 |
| UPI00018483A2 | C5CHI5 | Kosmotoga olearia | 205 | 605 |
| UPI0001CD5918 | D4LRF6 | Blautia obeum | 206 | 606 |
| UPI0001E18CDA | E0Q541 | Bifidobacterium dentium | 207 | 607 |
| UPI0001F55F8F | E8N5R8 | Anaerolinea thermophila | 208 | 608 |
| UPI0002050DA2 | F2N7E4 | Coriobacterium glomerans | 209 | 609 |
| UPI000210C886 | F5XJQ3 | Microlunatus phosphovorus | 210 | 610 |
| UPI0001C18877 | G0Q1U8 | Streptomyces sp. | 211 | 611 |
| UPI0002282B61 | G3AIV6 | Spathaspora passalidarum | 212 | 612 |
| UPI0002402F4A | H0ET34 | Glarea lozoyensis | 213 | 613 |
| UPI0002633B36 | I3UJK0 | Glaciozyma antarctica | 214 | 614 |
| UPI00027DFD35 | J7LN00 | Arthrobacter sp. | 215 | 615 |
| UPI00028D698C | K3VMA9 | Fusarium pseudograminearum | 216 | 616 |
| UPI000006AA61 | C6GGC9 | Hypocrea rufa | 217 | 617 |
| UPI0001CDA964 | D4M6W6 | Ruminococcus torques | 218 | 618 |
| UPI0001E17370 | E0Q9Z7 | Bifidobacterium dentium | 219 | 619 |
| UPI0001FC71F6 | F0R2D7 | Bacteroides salanitronis | 220 | 620 |
| UPI00020E6D19 | F3ZQ40 | Bacteroides coprosuis | 221 | 621 |
| UPI00020D4CC6 | F6CWF6 | Marinomonas posidonica | 222 | 622 |
| UPI000227E8ED | G0SE64 | Chaetomium thermophilum | 223 | 623 |
| UPI00022B9A15 | G4L5K1 | Tetragenococcus halophilus | 224 | 624 |
| UPI00024F0867 | H6CCZ8 | Paenibacillus sp. | 225 | 625 |
| UPI000265AA4B | I4B8U7 | Turneriella parva | 226 | 626 |
| UPI00027DFDB2 | J7LQK9 | Arthrobacter sp. | 227 | 627 |
| UPI00029074B1 | K5W7V1 | Agaricus bisporus | 228 | 628 |
| UPI0001B67634 | C7YIP3 | Nectria haematococca | 229 | 629 |
| UPI0001A25287 | D4VSZ0 | Bacteroides xylanisolvens | 230 | 630 |
| UPI0001DD9790 | E1R331 | Sediminispirochaeta smaragdinae | 231 | 631 |
| UPI0001FC42E9 | F0RPV2 | Deinococcus proteolyticus | 232 | 632 |
| UPI0002033A0A | F4C226 | Sphingobacterium sp. | 233 | 633 |
| UPI0001E54133 | F6ET40 | Sphingobium chlorophenolicum | 234 | 634 |
| UPI00021CACC4 | G0V3V5 | Caloramator australicus | 235 | 635 |
| UPI000230E3BF | G6F370 | Commensalibacter intestini | 236 | 636 |
| UPI00024F07AC | H6CIT2 | Paenibacillus sp. | 237 | 637 |
| UPI0002638AF3 | I4EIA9 | Nitrolancea hollandica | 238 | 638 |
| UPI000392C3ED | J9VVK7 | Cryptococcus neoformans | 239 | 639 |
| UPI0002988588 | K7S596 | Acidipropionibacterium acidipropionici | 240 | 640 |
| UPI0001B93465 | C9PT75 | Prevotella sp. | 241 | 641 |
| UPI0001D08095 | D5ALU0 | Rhodobacter capsulatus | 242 | 642 |
| UPI0001E74370 | E3FJ05 | Stigmatella aurantiaca | 243 | 643 |
| UPI0002010060 | F0RVK3 | Sphaerochaeta globosa | 244 | 644 |
| UPI000207D78C | F4GH96 | Sphaerochaeta coccoides | 245 | 645 |
| UPI00020EFBCB | F6ICQ5 | Novosphingobium sp. | 246 | 646 |
| UPI000225331F | G1XH86 | Arthrobotrys oligospora | 247 | 647 |
| UPI00000C6907 | G6FFS4 | Lactococcus lactis | 248 | 648 |
| UPI000255313A | H8FXH7 | Phaeospirillum molischianum | 249 | 649 |
| UPI0002609C68 | I4EW72 | Modestobacter marinus | 250 | 650 |
| UPI00028AC337 | K0K125 | Saccharothrix espanaensis | 251 | 651 |
| UPI00029BA293 | K8DAK0 | Cronobacter sakazakii | 252 | 652 |
| UPI0001BBDF1E | C9SVX1 | Verticillium alfalfae | 253 | 653 |
| UPI0001CCFD36 | D6CY10 | Bacteroides xylanisolvens | 254 | 654 |
| UPI0001EBD98A | E4RUR0 | Leadbetterella byssophila | 255 | 655 |
| UPI00020102A5 | F0RYB6 | Sphaerochaeta globosa | 256 | 656 |
| UPI000207D6A0 | F4GLH6 | Sphaerochaeta coccoides | 257 | 657 |
| UPI000212252C | F7Q0Y2 | Haloplasma contractile | 258 | 658 |
| UPI0002265447 | G1Y4C7 | Nitrospirillum amazonense | 259 | 659 |
| UPI00023420B8 | G8AWD9 | Azospirillum brasilense | 260 | 660 |
| UPI000255314F | H8FXJ8 | Phaeospirillum molischianum | 261 | 661 |
| UPI000260A2FA | I4EYD5 | Modestobacter marinus | 262 | 662 |
| UPI000283EB8D | K0KVJ2 | Wickerhamomyces ciferrii | 263 | 663 |
| UPI0002A5D085 | K9XKL8 | Gloeocapsa sp. | 264 | 664 |
| UPI0001A3BCB6 | D1C7U8 | Sphaerobacter thermophilus | 265 | 665 |
| UPI0001CCEF1F | D6D4V2 | Bacteroides xylanisolvens | 266 | 666 |
| UPI0001F149E8 | E6K4W5 | Prevotella buccae | 267 | 667 |
| UPI0001FF1101 | F0XBR0 | Grosmannia clavigera | 268 | 668 |
| UPI00020F9774 | F4R4W2 | Melampsora larici-populina | 269 | 669 |
| UPI0002138E9C | F8N7G1 | Prevotella multisaccharivorax | 270 | 670 |
| UPI0002255A63 | G2G8K4 | Streptomyces zinciresistens | 271 | 671 |
| UPI0001D9FC40 | G8NY42 | Granulicella mallensis | 272 | 672 |
| UPI00021F1FEE | I1RH94 | Gibberella zeae | 273 | 673 |
| UPI00026090A2 | I4EYK6 | Modestobacter marinus | 274 | 674 |
| UPI0002869F55 | K0NRS8 | Lactobacillus equicursoris | 275 | 675 |
| UPI0002A93280 | L2F9W0 | Colletotrichum fructicola | 276 | 676 |
| UPI0001BF8AF6 | D2B261 | Streptosporangium roseum | 277 | 677 |
| UPI00019F252A | D7UX19 | Listeria grayi | 278 | 678 |
| UPI0001F11EFF | E6LF07 | Enterococcus italicus | 279 | 679 |
| UPI000203D9EA | F2IIT6 | Fluviicola taffensis | 280 | 680 |
| UPI00020CA641 | F5N4W9 | Shigella flexneri | 281 | 681 |
| UPI0002189080 | F9EFW5 | Actinomyces sp. | 282 | 682 |
| UPI00022EBE5A | G2X5V6 | Verticillium dahliae | 283 | 683 |
| UPI00023ED5A8 | G8S0M0 | Actinoplanes sp. | 284 | 684 |
| UPI000023EA29 | I1S320 | Gibberella zeae | 285 | 685 |
| UPI00027CE685 | J0WV06 | Auricularia subglabra | 286 | 686 |
| UPI00028E86D6 | K2N268 | Nitratireductor indicus | 287 | 687 |
| UPI0002AD999E | L7VH66 | Thermoclostridium stercorarium | 288 | 688 |
| UPI0002AF2DE3 | L8A1S2 | Geobacillus sp. | 289 | 689 |
| UPI000327C3CC | M9ZC55 | uncultured bacterium | 290 | 690 |
| UPI000059ACE1 | Q0BAK2 | Burkholderia ambifaria | 291 | 691 |
| UPI0000676B8D | Q2UIR4 | Aspergillus oryzae | 292 | 692 |
| UPI00004269B5 | Q66DJ0 | Yersinia pseudotuberculosis | 293 | 693 |
| UPI000331CC98 | R9AF64 | Wallemia ichthyophaga | 294 | 694 |
| UPI0003522404 | S3DIM6 | Glarea lozoyensis | 295 | 695 |
| UPI0003BF753A | V2YU31 | Moniliophthora roreri | 296 | 696 |
| UPI0003DBCB4F | W2ULL3 | Zhouia amylolytica | 297 | 697 |
| UPI0003F362FF | W9NQQ3 | Fusarium oxysporum | 298 | 698 |
| UPI00006EA168 | A5FAA5 | Flavobacterium johnsoniae | 299 | 699 |
| UPI00002394F5 | Q75193 | Oryza sativa | 300 | 700 |
| UPI0002B65681 | M1MCD1 | Clostridium saccharoperbutylacetonicum | 301 | 701 |
| UPI0002D21DB1 | N1MBN6 | Rhodococcus sp. | 302 | 702 |
| UPI000059C0CD | Q0BCV8 | Burkholderia ambifaria | 303 | 703 |
| UPI00005CE9E7 | Q3BVH7 | Xanthomonas campestris | 304 | 704 |
| UPI00000C7604 | Q9A6F8 | Caulobacter vibrioides | 305 | 705 |
| UPI000338772A | R9GRA7 | Arcticibacter svalbardensis | 306 | 706 |
| UPI00035A4604 | S7VQ28 | Winogradskyella psychrotolerans | 307 | 707 |
| UPI0003C4ED6A | V5DXT8 | Methyloglobulus morosus | 308 | 708 |
| UPI0003E05C04 | W3WWV37 | Pestalotiopsis fici | 309 | 709 |
| UPI000434E698 | W9YNR7 | Capronia coronata | 310 | 710 |
| UPI00001268FD | P48825 | Aspergillus aculeatus | 311 | 711 |
| UPI0000DD5996 | Q0GC07 | Thermotoga neapolitana | 312 | 712 |
| UPI0002B666E8 | M1MJF4 | Clostridium saccharoperbutylacetonicum | 313 | 713 |
| UPI000006B001 | O74799 | Schizosaccharomyces pombe | 314 | 714 |
| UPI000161BD2B | Q0TXF6 | Phaeosphaeria nodorum | 315 | 715 |
| UPI00003806C8 | Q3RGJ3 | Xylella fastidiosa Dixon | 316 | 716 |
| UPI000006A330 | Q9P6J6 | Schizosaccharomyces pombe | 317 | 717 |
| UPI000337E887 | R9GWD6 | Arcticibacter svalbardensis | 318 | 718 |
| UPI0003885717 | T0KJI7 | Colletotrichum gloeosporioides | 319 | 719 |
| UPI0003C9E340 | V5R1E8 | uncultured bacterium | 320 | 720 |
| UPI0003E06A61 | W4S715 | Xanthomonas arboricola | 321 | 721 |
| UPI00021EC697 | X0A8X8 | Fusarium oxysporum | 322 | 722 |
| UPI00001108D0 | P22073 | Paenibacillus polymyxa | 323 | 723 |
| UPI0001BE5ADA | D1GCC6 | Kluyveromyces marxianus | 324 | 724 |
| UPI0002C04A25 | M5A594 | Ilumatobacter coccineus | 325 | 725 |
| UPI0000126912 | P12614 | Agrobacterium sp. | 326 | 726 |
| UPI000038ECF7 | Q11P53 | Cytophaga hutchinsonii | 327 | 727 |
| UPI00003C5CA4 | Q47PF5 | Thermobifida fusca | 328 | 728 |
| UPI0000069E5E | Q9UVJ6 | Botryotinia fuckeliana | 329 | 729 |
| UPI00033994D2 | R9PTK2 | Agarivorans albus | 330 | 730 |
| UPI00038B7639 | T0UDU2 | Enterococcus sp. | 331 | 731 |
| UPI0003D8A7DC | V5WKT4 | Salinispira pacifica | 332 | 732 |
| UPI0003ECF15E | W6P696 | Bacteroides xylanisolvens | 333 | 733 |
| UPI000430029A | X0AB46 | Fusarium oxysporum | 334 | 734 |
| UPI0000126906 | Q08638B | Thermotoga maritima | 335 | 735 |
| UPI0000083EA8 | Q8T0W7 | Neotermes koshunensis | 336 | 736 |
| UPI0002BF34B3 | M5CHG9 | Thanatephorus cucumeris | 337 | 737 |
| UPI000053581D | P14002 | Hungateiclostridium thermocellum | 338 | 738 |
| UPI0000D76A70 | Q1IJ89 | Koribacter versatilis | 339 | 739 |
| UPI00005203D8 | Q4WLX5 | Neosartorya fumigata | 340 | 740 |
| UPI00032DB8C1 | R1FKF0 | Amycolatopsis vancoresmycina | 341 | 741 |
| UPI0003519C76 | S0EKU1 | Gibberella fujikuroi | 342 | 742 |
| UPI000390F839 | T2I2H5 | Bifidobacterium longum | 343 | 743 |
| UPI0003DFF0DA | W0FLD1 | uncultured bacterium | 344 | 744 |
| UPI0002D3D994 | W6P9J9 | Bacteroides xylanisolvens | 345 | 745 |
| UPI00042F8016 | X0HA35 | Fusarium oxysporum | 346 | 746 |
| UPI00000A7F40 | Q41290 | Sorghum bicolor | 347 | 747 |
| UPI000050B701 | Q53EH2 | Clostridium cellulovorans | 348 | 748 |
| UPI0002C29EAA | M5G1U5 | Dacryopinax primogenitus | 349 | 749 |
| UPI0000126913 | P27034 | Rhizobium radiobacter | 350 | 750 |
| UPI00006E0CC7 | Q25BW5A | Phanerochaete chrysosporium | 351 | 751 |
| UPI0000126905 | Q59437 | Enterobacter agglomerans | 352 | 752 |
| UPI00033056C4 | R4YWU4 | Candidatus microthrix | 353 | 753 |
| UPI0003427F29 | S0ET37 | Chthonomonas calidirosea | 354 | 754 |
| UPI0003B04208 | U4KMR7 | Acholeplasma brassicae | 355 | 755 |
| UPI0003E13BD3 | W0LJ28 | Chania multitudinisentens | 356 | 756 |
| UPI0003ECCF80 | W7MW98 | Gibberella moniliformis | 357 | 757 |
| UPI0004305856 | X0I2U8 | Fusarium oxysporum | 358 | 758 |
| UPI00006E0CC7 | Q25BW5B | Phanerochaete chrysosporium | 359 | 759 |
| UPI00000A7EFC | Q9FYS3 | Secale cereale | 360 | 760 |
| UPI0002C0273E | M5QUM2 | Anoxybacillus gonensis | 361 | 761 |
| UPI0000126906 | Q08638A | Thermotoga maritima | 362 | 762 |
| UPI00003C2ACC | Q2RP51 | Rhodospirillum rubrum | 363 | 763 |
| UPI00000B9013 | Q60038 | Thermotoga neapolitana | 364 | 764 |
| UPI00032F466E | R4Z6M6 | Candidatus microthrix | 365 | 765 |
| UPI00032874EC | S0FPI8 | Ruminiclostridium cellobioparum | 366 | 766 |
| UPI0003B0490C | U4KMV4 | Acholeplasma brassicae | 367 | 767 |
| UPI0003DB6EFD | W1BBP5 | Klebsiella pneumoniae | 368 | 768 |
| UPI00045BB507 | X8JI88 | Rhizoctonia solani | 370 | 770 |
| UPI0000072C73 | Q9H227 | Homo sapiens | 371 | 771 |
| UPI0000DD5304 | Q0GMU3 | uncultured bacterium | 372 | 772 |
| UPI0002CA9E4F | M8CQD9 | Thermoanaerobacter thermohydrosulfuricus | 373 | 773 |
| UPI0000E297D8 | Q091M8 | Stigmatella aurantiaca | 374 | 774 |
| UPI00006676B1 | Q2T7C9 | Burkholderia thailandensis | 375 | 775 |
| UPI0000426E8F | Q665S2 | Yersinia pseudotuberculosis | 376 | 776 |
| UPI00032BF71F | R8BQM8 | Togninia minima | 377 | 777 |
| UPI00035213C2 | S3BVJ8 | Ophiostoma piceae | 378 | 778 |
| UPI0003B117B7 | U4LIL3 | Pyronema omphalodes | 379 | 779 |
| UPI0001F76D1F | W1QIW0 | Ogataea parapolymorpha | 380 | 780 |
| UPI0003F2059F | W8F6U0 | Hymenobacter swuensis | 381 | 781 |
| UPI00006E8E1B | A5FEF5 | Flavobacterium johnsoniae | 382 | 782 |
| UPI000012690B | P22505 | Paenibacillus polymyxa | 383 | 783 |
| UPI000009D014 | Q8L7J2 | Oryza sativa | 384 | 784 |
| UPI0000E580F2 | B8AVF0 | Oryza sativa | 385 | 785 |
| UPI000EA7F16F | A0A452CSM4 | Nannochloris | 386 | 786 |
| UPI00006AE508 | B8CYA8 | Halothermothrix orenii | 387 | 787 |
| UPI000018B2B4 | Q7RWP2 | Neurospora crassa | 388 | 788 |
| UPI000192BB5F | B9V8P5 | Micrococcus antarcticus | 389 | 789 |
| UPI000285E79E | K0A8J9 | Exiguobacterium antarcticum | 390 | 790 |
| UPI00000BEB61 | Q9RA61 | Thermus thermophilus | 391 | 791 |
| UPI00078BF747 | A0A2T4AR08 | Trichoderma harzianum | 392 | 792 |
| UPI000006AA61 | Q12715 | Hypocrea jecorina | 393 | 793 |
| UPI00000B411B | Q59976 | Streptomyces sp. | 394 | 794 |
| UPI00000C7E56 | Q99YP9 | Streptococcus pyogenes | 395 | 795 |
| UPI000012691B | P26205 | Trifolium repens | 396 | 796 |
| UPI000006C8FF | Q8TGI8 | Talaromyces emersonii | 397 | 797 |
| UPI0000126903 | P26208 | Hungateiclostridium thermocellum | 398 | 798 |
| UPI000219FE3E | F9ULH8 | Lactobacillus plantarum | 399 | 799 |
| UPI0003F2033A | A0A2I4PGZ0 | Agrobacterium tumefaciens | 400 | 800 |

Experiment 4-1—Screening of Glucosidases for Deglycosylation with Purified QS-18 (0.04 mg/ml) at pH 7.5 and Room Temperature

Nucleotide sequences were sub-cloned into pET24b+ for expression. 10 uL of E. coli cells (One Shot® BL21(DE3) chemically competent E. coli) were transferred to each well of a 96 well PCR plate (prechilled on ice). 10 uL autoclaved water was added to the DNA, resuspended by pipetting, and 1 uL of plasmid DNA (15-30 ng) was transferred to the competent cells. Immediately after addition, the resulting mixture was mixed by pipetting. Cells were heat shocked by placing the plate in a thermal cycler at 42° C. for 30 seconds then transferred directly to an ice bath for 2 min. 100 uL sterile Lysogeny Broth (LB) medium was added to each well containing transformed cells. The content of each plate was transferred into a 96-deep well plate pre-aliquoted with 400 uL LB and the plate was incubated at 37° C. with shaking and 85% humidity for 1 hour. After outgrowth, 500 uL of sterile LB containing 100 ug/mL kanamycin was added to the plates containing cells and plates incubated at 37° C. with shaking overnight (18 hours) with humidity control (85%).

1000 mLs of Overnight Express Media was supplemented with 1 mL kanamycin 50 mg/mL and 20 mL of 50% v/v glycerol added (50 ug/mL kanamycin final and 1% glycerol). 96-Well Assay Block 2 mL plates were aliquoted with 380 uL of media per well. Pre-inoculum (20 uL) from transformation plates was added. The plates were sealed appropriately and incubated at 37° C. at 300 with shaking for 2 h. After 2 h the temperature was lowered to 20° C. and incubation continued for 20 h.

The liquid cultures were centrifuged for 10 minutes at 4° C. The supernatant was discarded, plates blotted on an absorbent material to remove residue and the plates frozen at −80° C.

Lysis buffer was prepared according to the following protocol:
1. Polymyxin B sulphate (0.5 mg/mL) was suspended in 100 mM potassium phosphate pH 7.5
2. The mixture was sonicated until polymyxin B sulphate had dissolved
3. Lysozyme (1 mg/mL) and benzonase (0.1 uL/mL lysis buffer) were added

2 copies of each cell pellet plate were removed from −80 degC freezer and allowed to thaw. 200 uL of lysis buffer was added to each well of one copy of the cell pellet plates. Plates were shaken at room temperature for 10 mins. 190 uL of cell pellet/lysis buffer was transferred to a corresponding fresh cell pellet plate. These plates were incubated at room temperature with shaking for 2 hours. Lysate was clarified by centrifugation (10 min, 4 degC).

QS-18 was obtained by analogous methods to Example 3, collecting a QS-18 containing phenyl fraction following phenyl treatment (presence of m/z corresponding to key components was confirmed by MS and the phenyl fraction then used without further treatment). QS-18 solution was prepared by diluting aqueous QS-18 (ca 1 mg/mL) with 100 mM potassium phosphate pH 7.5 to 0.2 mg/ml. 40 uL clarified lysate was transferred into fresh 96 well PCR plates. 10 uL QS-18 solution added to each well of lysate to a final concentration of 0.04 mg/ml. Incubated at room temperature with shaking for 20 h. Quenched with 50 uL MeCN and shaken at room temperature for 10 mins. Samples were analysed by LC-MS/MS using a Waters Acquity H class coupled to a Waters Xevo Tandem Quadrupole (TQD) Mass Spectrometer.

### LCMS/MS 3 Min Method

UPLC Conditions

| Column | Waters BEH C18 1.7 um 2.1 × 50 mm | | |
|---|---|---|---|
| Temperature | 40° C. ± 3° C. | | |
| Flow Rate | 0.5 mL/min | | |
| Gradient Curve | 6 | | |
| Injection Volume | 1 μL | | |
| Autosampler Temperature | 10° C. ± 3° C. | | |
| Run Time | 3 mins | | |
| Post-Inject Wash | 50% v/v methanol | | |
| Mobile Phase A | 0.1% v/v Formic Acid in Water | | |
| Mobile Phase B | 0.1% v/v Formic Acid in Acetonitrile | | |
| Syringe Wash Solvent | 50% v/v Methanol | | |
| Seal Wash | 50% v/v Methanol | | |
| Purge Solvent | 50% v/v Acetonitrile | | |
| Gradient | Time | %A | %B |
| | 0 | 55 | 45 |
| | 0.75 | 55 | 45 |
| | 2.00 | 5 | 95 |
| | 2.1 | 5 | 95 |
| | 2.2 | 55 | 45 |
| | 3.0 | 55 | 45 |

Mass Spec Conditions (or similar to give maximum sensitivity)

| Source | | Analyser | | Extended Tab | |
|---|---|---|---|---|---|
| Capillary Voltage | 3.9 kV | LM Resolution 1 | 11.6 | Source Temp | 150° C. |
| Cone | 36 V | HM Resolution 1 | 14.9 | Extractor | 3 V |
| Desolvation | 500° C. | Ion Energy 1 | 0.4 | RF Lens | 2.5 V |
| Temperature | | | | | |
| Source Temperature | 150° C. | LM Resolution 2 | 12.2 | Entrance | 30 |
| Source Gas Flow | | HM Resolution 2 | 14.8 | Exit | 30 |
| Desolvation Gas Flow | 1000 L/Hr | Ion Energy 2 | 0.8 | Gain | 1 |

| | | | |
|---|---|---|---|
| Cone (L/Hr) | 0 | Collision Energy | 3 |

| | |
|---|---|
| Collision Gas | Argon |

| | | Dwell | | Cone | Collision |
|---|---|---|---|---|---|
| | | Time | | Voltage | Energy |
| Component | Mode | (secs) | MRM Transitions | (V) | (v) |
| QS-18 2150 | ESI - | Auto | 1074.50 > 836.4 | 34 | 16 |
| QS-21 1988 | ESI - | Auto | 993.46 > 755.5 | 34 | 16 |

Enzyme activity was calculated as:

$$\% conversion = \frac{100 \times QS - 21 peak area}{( QS - 21 peak area + QS - 18 peak area )}$$

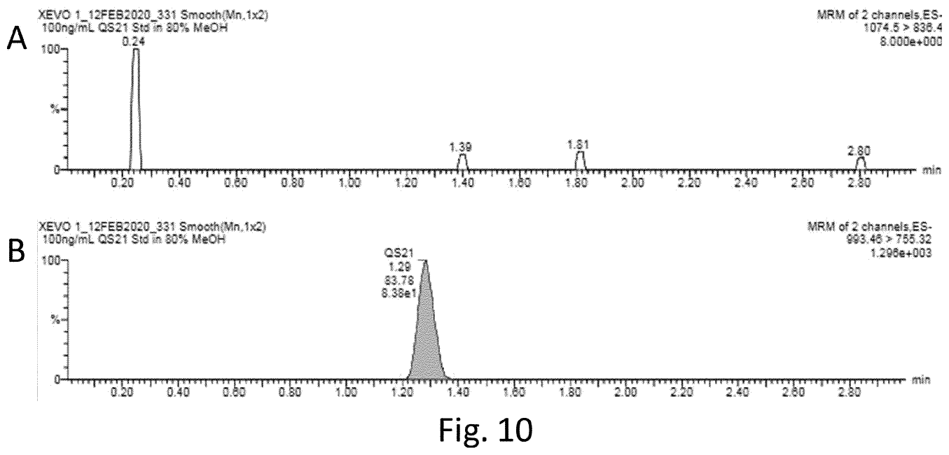

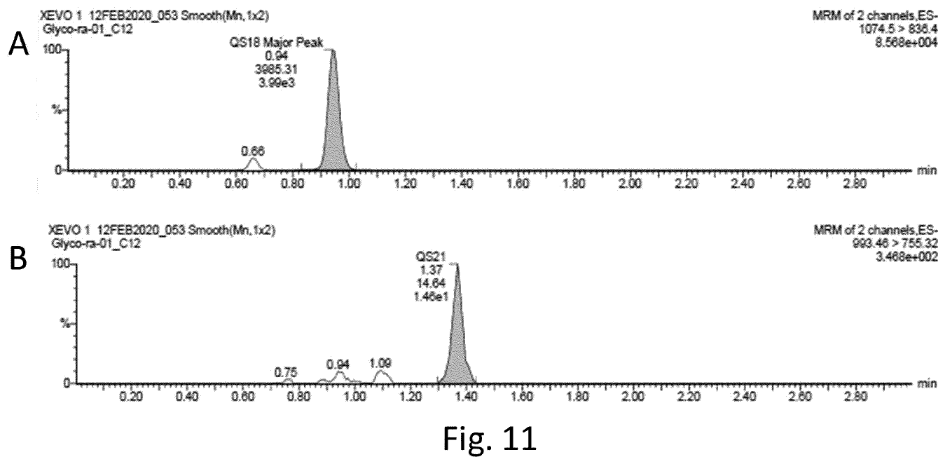

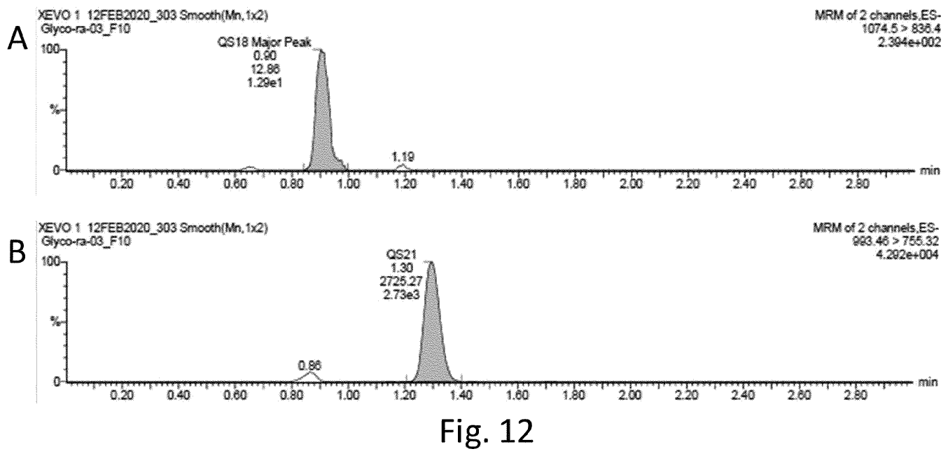

The negative control reactions, which utilised a plasmid expressing an unrelated protein, had an average % conversion of 0.42% with a standard deviation (S.D.) of 0.10%. Candidate enzymes with % conversion >0.72%, i.e. >3 S.D. above negative control, were considered to be positive hits and are listed below in Table 7. Sample results are shown in FIG. 10 for a QS-21 standard, FIG. 11 for negative control and FIG. 12 for treatment with the glucosidase of SEQ ID No. 262.

Experiment 4-2—Screening of Glucosidases for Deglycosylation with Purified QS-18 (1 mg/ml) at pH 6 and 30 Deg C.

Lysates were prepared in an identical manner to Experiment 4-1 above, except the lysis buffer was prepared in 100 mM potassium phosphate buffer pH 6. QS-18 solution was prepared by dissolving QS-18 in 100 mM potassium phosphate buffer pH 6 (2 mg/mL).

12 uL clarified lysate was transferred into fresh 96 well PCR plates. 12 uL QS-18 solution was added (1 mg/ml final concentration), plates sealed and incubated overnight (30° C.) for 18 hrs. After quenching with 25 uL MeCN and shaking for 10 mins (RT), samples were analysed using the LC-MS/MS protocol described in Experiment 4-1 and enzyme activity determined in an analogous manner.

The negative control reactions had an average % conversion of 0.38% with a standard deviation (S.D.) of 0.06%. Sequences with % conversion >0.56% i.e. >3 S.D. above negative control are listed in Table 7.

Experiment 4-3—Screening of Glucosidases for Deglycosylation with QS-18 in Crude Bark Extract (1 in 2000 Dilution) at pH 7 and 30 Deg C.

Lysates were prepared according to the following procedure.

50 uL of 50% v/v glycerol was transferred to each well of a flat bottom 96 well plate. 50 uL from each well of the overnight culture plate (in LB) from Experiment 4-1 was transferred and mixed by pipette aspiration. The plate was then covered with a foil seal and frozen at −80° C. as a glycerol stock of the transformants. Glycerol stock plates were removed from −80° C. freezer and allowed to thaw. Overnight cultures were prepared by pipetting 5 mL LB into 50 mL tubes with Kanamycin as a selection marker at a final concentration of 50 μg/mL. Cultures were inoculated with 10 μL of glycerol stock and incubated overnight at 37° C. with shaking.

Flask cultures were prepared by pipetting 25 mL Terrific Broth (TB) into 250 mL conical flasks with Kanamycin as selection marker at a final concentration of 50 μg/mL. Overnight cultures OD₆₀₀ was measured using a spectrophotometer and initial inoculum volume calculated for a starting OD˜0.1. Cultures were inoculated and incubated at 37° C. with shaking up to OD˜0.6.

Cultures were induced with 1 mM IPTG and temperature was reduced to 20° C. with shaking. Cultures were then incubated overnight. Cultures were harvested in individual 1 mL aliquots (in 2 mL tubes). 1 mL aliquots were centrifuged at 13000 g for 3 min and supernatant discarded. Pellets were frozen at −20° C.

Lysis buffer was prepared according to the following protocol:
Polymyxin B sulphate (0.5 mg/mL) and Lysozyme (1 mg/mL) were suspended in 50 mM phosphate buffer, 0.3 M NaCl, pH 8.
Benzonase (20 U/mL) and 0.05% Tween-20 were added.

1 mL of Lysis buffer was added to a pellet from 1 mL culture aliquot. Lysed samples were incubated at room temperature with shaking for 2 hours. Lysate was clarified by centrifugation at 13000 g, 5 min, 4° C.

Crude bark extract (CBE) obtained by aqueous extraction of Quillaja saponaria and containing at least 2.80 mg/ml QS-21 (by HPLC-UV) was diluted 1 in 400 in 50 mM potassium phosphate buffer at pH 7. 100 ul diluted CBE was added to 400 ul of each lysate to give a final dilution of 1 in 2000.

As diluted CBE was added to the lysate, the solution was vortexed for ˜5 seconds and then a 80 ul sample taken and quenched with 160 ul methanol (MeOH). This was used as a time 0 sample. The reaction solutions and controls were then left to shake at 30° C. Samples were taken after 1 h in the same way as the time 0 sample. Samples were analysed by LCMS/MS using the protocol described in Experiment 4-1 and enzyme activity determined in an analogous manner.

Enzyme activity is calculated as the % conversion of the QS-18 present in the crude bark extract:

$$\% conversion = \frac{100 \times ( \% QS - {18}_{t = 0} - \% QS - {18}_{t = 1 hr} )}{\% QS - {18}_{t = 0}}$$

$$where$$

$$\% QS - 18 = \frac{100 \times QS - 18 peak area}{( QS - 21 peak area + QS - 18 peak area )}$$

### Experiment 4-4—Screening of Glucosidases for Deglycosylation of QS-18 in Crude Bark Extract (1 in 20 Dilution) to QS-21

Lysates were prepared as in Experiment 4-2.

Crude bark extract (CBE) was adjusted to pH 6 by dropwise addition of 2M NaOH with stirring. 25 uL of clarified lysate was transferred to a reaction plate, 22.5 ul of 100 mM potassium phosphate buffer pH 6 was added, 2.5 ul CBE at pH 6 was added. Reaction plates were sealed, incubated at 25 degC with shaking for 18 hours, then quenched by addition of 50 uL acetonitrile (MeCN). Quenched reaction plates were re-sealed and incubated at 20 degC with shaking for 10 min. The reaction plates were centrifuged (10 min, 4 degC) and analysed by UV HPLC with the method below:

### UV Method EM2020N435545v1_2

| Column details | ACQUITY UPLC BEH C18 Column, | | |
|---|---|---|---|
| | 130 Å, 1.7 μm, 2.1 mm × 50 mm | | |
| Column temperature | 40° C. | | |
| Mobile phase A | 0.05% v/v TFA in Water HPLC | | |
| Mobile phase B | 0.05% v/v TFA in Acetonitrile HPLC | | |
| Flow rate | 1 mL per minute | | |
| | Time | % Mobile | % Mobile |
| | (mins) | Phase A | Phase B |
| Gradient profile | 0.0 | 97 | 3 |
| 5.5 min gradient | 3.7 | 5 | 95 |
| | 4.0 | 5 | 95 |
| | 4.1 | 97 | 3 |
| | 5.5 | 97 | 3 |

| | |
|---|---|
| Detector type | UV detector: DAD or VWD |
| Detector wavelength | 214 nm |
| Agilent DAD 1260 | 4.0 nm (default setting) |
| and 1290 Bandwidth | |
| Agilent 1290 and 1260 | Off |
| DAD reference wavelength | |
| Injection volume | Default injection volume used is 1 ul |

Three key peaks of interest are apparent using this chromatography: Left Peak (retention time approximately 2.30-2.35 min) comprising mainly QS-17 family components; Middle Peak (retention time approximately 2.37-2.42 min) comprising mainly QS-18 family components and desglucosyl-QS-17 family components; and Right Peak (retention time approximately 2.44-2.50 min) comprising mainly QS-21 family components. Peak identity was supported by MS/MS.

Enzyme activity is calculated as the % conversion of the Middle Peak present in the crude bark extract:

$$\% conversion = \frac{100 \times ( \% Middle {Peak}_{t = 0} - \% Middle {Peak}_{t = 1 hr} )}{\% Middle {Peak}_{t = 0}}$$

$$where$$

$$\% Middle = \frac{100 \times Middle Peak area}{( Right Peak area + Middle Peak area )}$$

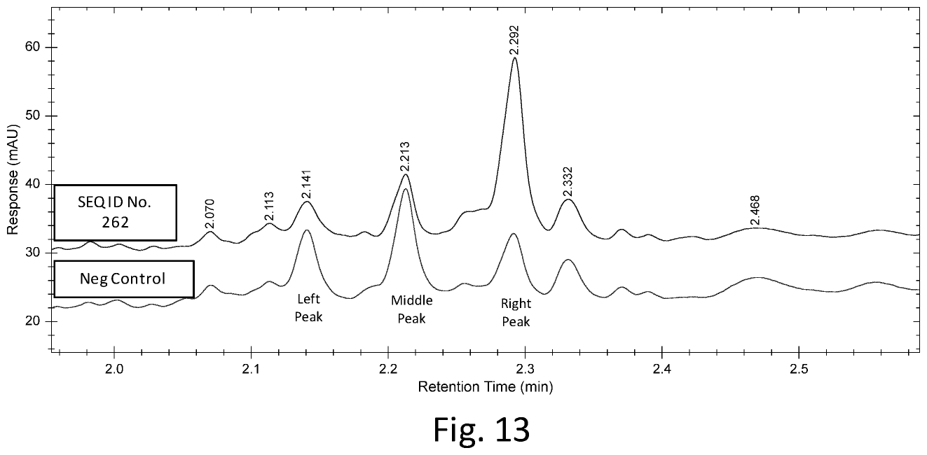

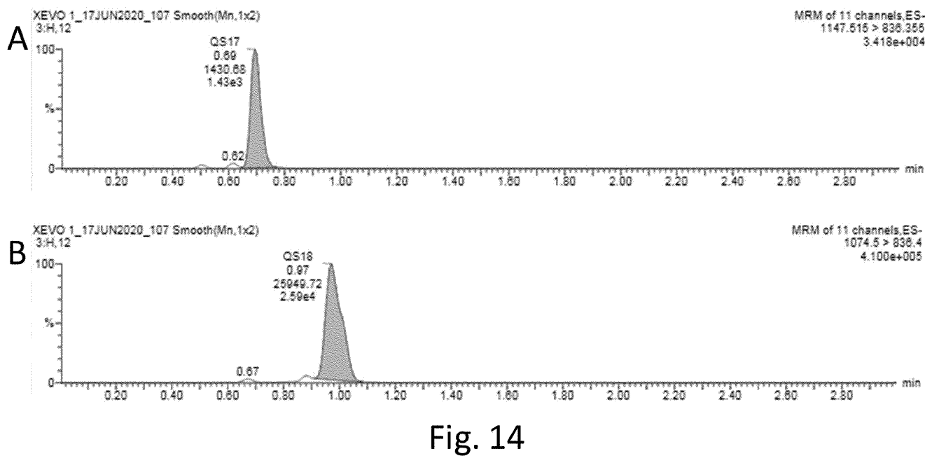

FIG. 13 provides exemplary chromatograms following glucosidase SEQ ID No. 262 treatment and for the negative control.

### Results

TABLE 7

| | | Expt 4-1 | Expt 4-2 | Expt 4-3 | Expt 4-4 |
|---|---|---|---|---|---|
| AA Seq ID | DNA Seq ID | % conv | % conv | % conv | % conv |
| 262 | 662 | 99.5 | 97.9 | 99.7 | 68.6 |
| 208 | 608 | 100.0 | 94.0 | 26.0 | <3 |
| 63 | 463 | 95.6 | 54.2 | N.T. | <3 |
| 229 | 629 | 91.1 | 14.2 | N.T. | <3 |
| 250 | 650 | 88.5 | 36.0 | 2.6 | <3 |
| 5 | 405 | 70.8 | 16.6 | 1.4 | <3 |
| 101 | 501 | 28.2 | 1.0 | 1.8 | <3 |
| 207 | 607 | 27.4 | 4.4 | 0.2 | <3 |
| 169 | 569 | 19.8 | 2.1 | 2.4 | <3 |
| 247 | 647 | 18.0 | 2.0 | 2.7 | <3 |
| 302 | 702 | 17.2 | 2.1 | N.T. | <3 |
| 324 | 724 | 13.6 | 0.9 | N.T. | <3 |
| 319 | 719 | 10.2 | 0.4 | N.T. | <3 |
| 9 | 409 | 9.2 | 0.9 | N.T. | <3 |
| 240 | 640 | 9.0 | 0.5 | N.T. | <3 |
| 325 | 725 | 6.3 | 0.9 | N.T. | <3 |
| 338 | 738 | 6.3 | 0.5 | N.T. | <3 |
| 116 | 516 | 4.7 | 0.5 | N.T. | <3 |
| 41 | 441 | 4.0 | 0.7 | N.T. | N.T. |
| 133 | 533 | 3.7 | 0.4 | N.T. | <3 |
| 350 | 750 | 2.6 | 0.7 | N.T. | <3 |
| 234 | 634 | 2.5 | 0.7 | N.T. | <3 |
| 380 | 780 | 2.4 | 0.5 | N.T. | <3 |
| 32 | 432 | 2.2 | 0.6 | N.T. | N.T. |
| 312 | 712 | 1.6 | 0.4 | N.T. | N.T. |
| 222 | 622 | 1.6 | 0.4 | N.T. | <3 |
| 354 | 754 | 1.6 | 0.5 | N.T. | <3 |
| 339 | 739 | 1.4 | 0.5 | N.T. | <3 |
| 365 | 765 | 1.3 | 0.4 | N.T. | <3 |
| 35 | 435 | 1.2 | 0.4 | N.T. | N.T. |
| 176 | 576 | 1.2 | 0.4 | N.T. | <3 |
| 370 | 770 | 1.1 | 0.5 | N.T. | N.T. |
| 152 | 552 | 1.1 | 0.5 | N.T. | N.T. |
| 274 | 674 | 0.9 | 0.3 | N.T. | N.T. |
| 84 | 484 | 0.8 | 0.4 | N.T. | N.T. |
| 102 | 502 | 0.7 | 0.4 | N.T. | N.T. |
| 178 | 578 | 0.5 | 0.8 | N.T. | N.T. |
| 4 | 404 | 0.4 | 0.6 | N.T. | N.T. |
| Negative control | | 0.42 | 0.38 | 0 | <3 |
| | | S.D. = 0.10 | S.D. = 0.06 | | |

N.T. indicates not tested

Based on detection of QS-18 2150 and QS-21 1988 components by LCMS/MS (Examples 4-1, 4-2 and 4-3) or UV HPLC quantification of Middle Peak (mainly QS-18 family and desglucosyl-QS-17 family) and Right Peak (mainly QS-21 family) (Example 4-4), Example 4 shows that a number of suitable glucosidases could be identified by screening a set of candidate enzymes (38 from 400, 9.5%). Glucosidases were capable of converting QS-18 family components to QS-21 family components at a range of pHs, concentrations of starting materials and purity of starting materials.

Although certain candidate enzymes did not demonstrate notable conversion under the conditions tested, this may be due to issues with enzyme expression, suitability of conditions (i.e. enzymes may function under other conditions) or a fundamental lack of required enzyme activity.

### Example 5—Screening of Additional Glucosidases for Deglycosylation of QS-18 to QS-21

### Method

### Enzyme Selection

Additional candidate glucosidases were selected based on amino acid similarity to an active site model based on positive hits from Example 4.

A final set of 94 additional candidate enzymes was selected. Codon optimized polynucleotide sequences encoding each selected enzyme linked to an N-terminal 6×His tag were prepared. Details of the additional candidate enzyme and polynucleotide sequences are summarised below in Table 8.

TABLE 8
Additional glucosidase candidate sequences

| | | | AA | DNA |
|---|---|---|---|---|
| | | | Seq | Seq |
| UniParc | Uniprot | Organism | ID | ID |
| UPI000503B26C | A0A086YYS8 | Bifidobacterium actinocoloniiforme | 801 | 895 |
| UPI000502B461 | A0A087CD28 | Bifidobacterium psychraerophilum | 802 | 896 |
| UPI00052B8681 | A0A0A2K704 | Penicillium italicum | 803 | 897 |
| UPI0005ED0AE6 | A0A0M2HDB3 | Microbacterium trichothecenolyticum | 804 | 898 |
| UPI0008206F38 | A0A1C616A3 | uncultured Clostridium sp. | 805 | 899 |
| UPI000BC57319 | A0A261Y7Q8 | Bifiguratus adelaidae | 806 | 900 |
| UPI000B3B73CD | A0A378ZIK3 | Paenibacillus thiaminolyticus | 807 | 901 |
| UPI000F8FB9BA | A0A3S9WE68 | Microbacterium lemovicicum | 808 | 902 |
| UPI00019923DC | C1A1N4 | Rhodococcus erythropolis | 809 | 903 |
| UPI00022C19B5 | G4CZU8 | Cutibacterium avidum | 810 | 904 |
| UPI0002C5A938 | M5BD10 | Clavibacter michiganensis | 811 | 905 |
| UPI0003DE3509 | W0ZC23 | Microbacterium sp. | 812 | 906 |
| UPI000507F38A | A0A086YZL4 | Bifidobacterium actinocoloniiforme | 813 | 907 |
| UPI000506119A | A0A087CR26 | Bifidobacterium reuteri | 814 | 908 |
| UPI0005A5CAC2 | A0A0A8RX48 | Propionibacterium freudenreichii | 815 | 909 |
| UPI0005EC18F9 | A0A0M2HRU0 | Microbacterium hydrocarbonoxydans | 816 | 910 |
| UPI00094B6D3B | A0A1Q8KSV8 | Pseudonocardia sp. | 817 | 911 |
| UPI000BC13DF0 | A0A292PKV5 | Tuber aestivum | 818 | 912 |
| UPI000E5B4EA5 | A0A383S7A9 | Propionibacterium australiense | 819 | 913 |
| UPI001004B2C9 | A0A428T6E2 | Fusarium sp. | 820 | 914 |
| UPI0001B69B5C | C7ZBV0 | Nectria haematococca | 821 | 915 |
| UPI00023EBB15 | G8S3E7 | Actinoplanes sp. | 822 | 916 |
| UPI0003289BC6 | N0CMW2 | Streptomyces fulvissimus | 823 | 917 |
| UPI0003E02BF1 | W3WXF1 | Pestalotiopsis fici | 824 | 918 |
| UPI0005025F2E | A0A086ZGP0 | Bifidobacterium bohemicum | 825 | 919 |
| UPI0005084E52 | A0A087D0Q2 | Bifidobacterium saeculare | 826 | 920 |
| UPI00059674D6 | A0A0B7JW24 | Bionectria ochroleuca | 827 | 921 |
| UPI0006C6D6F9 | A0A174AU04 | Bifidobacterium pseudocatenulatum | 828 | 922 |
| UPI00094ABAB3 | A0A1Q8LPB4 | Pseudonocardia sp. | 829 | 923 |
| UPI000CB49A0B | A0A2H5Z8Q4 | bacterium | 830 | 924 |
| UPI000E38A995 | A0A3D8Q771 | Coleophoma crateriformis | 831 | 925 |
| UPI001102DFA3 | A0A4Z0Y5Y8 | Arthrobotrys oligospora | 832 | 926 |
| UPI0001ECDCBD | E3RFS2 | Pyrenophora teres | 833 | 927 |
| UPI00024F2A26 | H6MTQ7 | Gordonia polyisoprenivorans | 834 | 928 |
| UPI0000E28E5D | Q08S21 | Stigmatella aurantiaca | 835 | 929 |
| UPI0003E03A52 | W3WZ03 | Pestalotiopsis fici | 836 | 930 |
| UPI0003B7B6EE | A0A087BEN9 | Bifidobacterium magnum | 837 | 931 |
| UPI000503F283 | A0A087DFL8 | Bifidobacterium stellenboschense | 838 | 932 |
| UPI00059673EE | A0A0B7K316 | Bionectria ochroleuca | 839 | 933 |
| UPI0006C069F3 | A0A174LVE3 | Hungatella hathewayi | 840 | 934 |
| UPI00097EB800 | A0A1R4J2F9 | Mycetocola reblochoni | 841 | 935 |
| UPI0009ABD7B3 | A0A2P9IX34 | Nonomuraea sp. | 842 | 936 |
| UPI000E391DF6 | A0A3D8T9C2 | Coleophoma crateriformis | 843 | 937 |
| UPI0000EC83AE | A1R2K1 | Paenarthrobacter aurescens | 844 | 938 |
| UPI0001F21F08 | E4N4F6 | Kitasatospora setae | 845 | 939 |
| UPI00027E251E | J7L3Z8 | Nocardiopsis alba | 846 | 940 |
| UPI0000DBA338 | Q0SCI4 | Rhodococcus jostii | 847 | 941 |
| UPI0003EC942D | W5X324 | uncultured microorganism | 848 | 942 |
| UPI0005084B63 | A0A087BJ88 | Bifidobacterium merycicum | 849 | 943 |
| UPI0005018B5B | A0A087DGT3 | Bifidobacterium scardovii | 850 | 944 |
| UPI00021EC81E | A0A0C4DJL4 | Fusarium oxysporum | 851 | 945 |
| UPI0007CE199C | A0A177BXH1 | Paraphaeosphaeria sporulosa | 852 | 946 |
| UPI0009A50F8D | A0A1V4IY77 | Clostridium oryzae | 853 | 947 |
| UPI000D22F5EB | A0A2T2N4T6 | Corynespora cassiicola | 854 | 948 |
| UPI000F7332F8 | A0A3N4J9R6 | Choiromyces venosus | 855 | 949 |
| UPI0000F550EC | A4F982 | Saccharopolyspora erythraea | 856 | 950 |
| UPI000204906E | F2R0Y2 | Streptomyces venezuelae | 857 | 951 |
| UPI00027DFDB0 | J7LVB1 | Arthrobacter sp. | 858 | 952 |
| UPI00033420CB | R7RMQ6 | Thermobrachium celere | 859 | 953 |
| UPI0003EA65B9 | W7HT93 | Drechslerella stenobrocha | 860 | 954 |
| UPI0003B38542 | A0A087BMR5 | Bifidobacterium minimum | 861 | 955 |
| UPI000409825E | A0A087E3K8 | Bifidobacterium thermacidophilum | 862 | 956 |
| UPI0005C0229F | A0A0D1X9G0 | Verruconis gallopava | 863 | 957 |
| UPI0007CECAD5 | A0A178AER7 | Stagonospora sp. | 864 | 958 |
| UPI0009D58C49 | A0A1V6ALF9 | Firmicutes bacterium | 865 | 959 |
| UPI000D50EED1 | A0A2T6ZNU7 | Tuber borchii | 866 | 960 |
| UPI000F728167 | A0A3N4KUR3 | Morchella conica | 867 | 961 |
| UPI00018152EB | B5YC96 | Dictyoglomus thermophilum | 868 | 962 |
| UPI000210C01C | F5XL24 | Microlunatus phosphovorus | 869 | 963 |
| UPI00028BB1B6 | K4IPD2 | Bifidobacterium asteroides | 870 | 964 |
| UPI00035AE576 | S8BQ60 | Dactylellina haptotyla | 871 | 965 |
| UPI0003F2D7DD | W9JF54 | Fusarium oxysporum | 872 | 966 |
| UPI0005067FF9 | A0A087BRY2 | Bifidobacterium longum | 873 | 967 |
| UPI0004105550 | A0A087EK01 | Bifidobacterium tsurumiense | 874 | 968 |
| UPI0005BF9DF9 | A0A0D1YG50 | Exophiala spinifera | 875 | 969 |
| UPI0007CE7DBA | A0A178E511 | Pyrenochaeta sp. | 876 | 970 |
| UPI0009CDA3F1 | A0A1V6FLK2 | Firmicutes bacterium | 877 | 971 |
| UPI000D5A9E68 | A0A2V1B599 | Cadophora sp. | 878 | 972 |
| UPI000F7330CD | A0A3N4L4M3 | Morchella conica | 879 | 973 |
| UPI000189C68F | B8DV42 | Bifidobacterium animalis | 880 | 974 |
| UPI00020ED2C9 | F5YGD5 | Treponema azotonutricium | 881 | 975 |
| UPI0002988429 | K7S0E5 | Acidipropionibacterium acidipropionici | 882 | 976 |
| UPI0003D93613 | V5WNB6 | Salinispira pacifica | 883 | 977 |
| UPI000503588E | A0A087C760 | Bifidobacterium mongoliense | 884 | 978 |
| UPI000499F5D1 | A0A087VUW4 | Bifidobacterium indicum | 885 | 979 |
| UPI000659257F | A0A0G4K5C2 | Brachyspira suanatina | 886 | 980 |
| UPI0007F2FD44 | A0A194X8Q9 | Phialocephala scopiformis | 887 | 981 |
| UPI000A2BB4A5 | A0A1Y5P895 | uncultured Microbacterium sp. | 888 | 982 |
| UPI000D5BC9BD | A0A2V1E673 | Periconia macrospinosa | 889 | 983 |
| UPI000F3DC30B | A0A3P5WTE2 | Arthrobacter ulcerisalmonis | 890 | 984 |
| UPI0001664880 | B8H9A2 | Pseudarthrobacter chlorophenolicus | 891 | 985 |
| UPI0002120C63 | F7PUF3 | Haloplasma contractile | 892 | 986 |
| UPI0002B73341 | M2ULB2 | Cochliobolus heterostrophus | 893 | 987 |
| UPI0003DE58EA | W0Z818 | Microbacterium sp. | 894 | 988 |

Experiment 5-1—Screening of Additional Glucosidases for Deglycosylation with Purified QS-18 (0.04 mg/ml) at pH 7.5 and 30 Deg C.

The 94 additional genes, together with positive control (DNA encoding SEQ ID No. 262) and negative control, were transformed, expressed, lysed and reacted in the same manner as described above for Experiment 4-1, except the reaction was incubated at 30 degrees C. for 18 hours.

Samples were analysed by LCMS/MS according to the procedure in Experiment 4-1. The results for all enzymes demonstrating a % conversion of at least 3 are shown in Table 9.

Experiment 5-2—Screening of Additional Glucosidases for Deglycosylation with QS-18 in Crude Bark Extract (80%) at pH 6 and 35 Deg C.

A plate was lysed at pH 6 as described in Experiment 4-2. 40 uL of clarified lysate was transferred to a reaction plate

The pH of CBE was adjusted to pH 6 by dropwise addition of 2M NaOH with stirring. 160 uL of pH 6 CBE was added to each well of the reaction plate. The reaction plate was sealed and incubated at 35 deg C. with shaking for 18 hours.

The reaction plate was quenched by adding 200 uL of MeCN (2% acetic acid (AcOH), 1 mg/mL hexanophenone) to each well of the plates. The quenched reaction plate was re-sealed and incubated at 20 deg C. with shaking for 10 min. The reaction plate was then centrifuged (10 min, 4 degC).

200 uL was transferred from each well of the quenched plate to the corresponding wells of a fresh 96 well plate and sealed. The plate was analysed by UV HPLC with the method of Experiment 4-4.

Experiment 5-3—Screening of Additional Glucosidases for Deglycosylation with QS-18 in Treated Bark Extract (80%) at pH 6 and 35 Deg C.

Experiment 5-1 was repeated, replacing CBE with Treated Bark Extract (TBE) at pH 6. TBE was prepared from CBE by PVPP treatment and concentration, to provide TBE with a QS-21 concentration of approximately 4 g/L. TBE was adjusted to pH 6 by dropwise addition of 2M NaOH with stirring.

TABLE 9

| AA | DNA | Experiment 5-1 | Experiment 5-2 | Experiment 5-3 |
|---|---|---|---|---|
| Seq ID | Seq ID | % conv | % conv | % conv |
| 262 | 662 | 98.7 | 5.5 | 8.5 |
| 850 | 944 | 95.3 | <0.5 | <0.5 |
| 879 | 973 | 90.1 | <0.5 | <0.5 |
| 868 | 962 | 89.9 | <0.5 | <0.5 |
| 826 | 920 | 86.9 | 0.8 | 1.4 |
| 804 | 898 | 83.2 | <0.5 | <0.5 |
| 888 | 982 | 78.0 | <0.5 | <0.5 |
| 881 | 975 | 77.5 | <0.5 | <0.5 |
| 891 | 985 | 74.7 | <0.5 | <0.5 |
| 816 | 910 | 73.5 | <0.5 | <0.5 |
| 827 | 921 | 69.2 | <0.5 | <0.5 |
| 857 | 951 | 69.0 | <0.5 | <0.5 |
| 853 | 947 | 67.7 | <0.5 | 2.5 |
| 842 | 936 | 64.0 | <0.5 | <0.5 |
| 814 | 908 | 58.4 | <0.5 | <0.5 |
| 886 | 980 | 55.5 | <0.5 | <0.5 |
| 885 | 979 | 54.8 | <0.5 | <0.5 |
| 838 | 932 | 53.4 | <0.5 | <0.5 |
| 829 | 923 | 52.3 | <0.5 | <0.5 |
| 808 | 902 | 52.0 | <0.5 | <0.5 |
| 828 | 922 | 50.7 | <0.5 | <0.5 |
| 870 | 964 | 49.7 | <0.5 | <0.5 |
| 873 | 967 | 43.9 | <0.5 | <0.5 |
| 844 | 938 | 43.6 | <0.5 | <0.5 |
| 882 | 976 | 31.4 | <0.5 | <0.5 |
| 874 | 968 | 29.4 | <0.5 | <0.5 |
| 825 | 919 | 24.5 | <0.5 | <0.5 |
| 824 | 918 | 23.5 | <0.5 | <0.5 |
| 823 | 917 | 23.1 | <0.5 | <0.5 |
| 810 | 904 | 22.7 | <0.5 | <0.5 |
| 894 | 988 | 21.3 | <0.5 | <0.5 |
| 849 | 943 | 17.8 | <0.5 | <0.5 |
| 803 | 897 | 13.1 | <0.5 | <0.5 |
| 890 | 984 | 12.6 | <0.5 | <0.5 |
| 841 | 935 | 12.3 | <0.5 | <0.5 |
| 832 | 926 | 9.8 | <0.5 | <0.5 |
| 830 | 924 | 9.3 | <0.5 | <0.5 |
| 845 | 939 | 9.2 | <0.5 | <0.5 |
| 871 | 965 | 7.8 | <0.5 | <0.5 |
| 837 | 931 | 7.6 | <0.5 | <0.5 |
| 883 | 977 | 7.0 | <0.5 | <0.5 |
| 809 | 903 | 6.3 | <0.5 | <0.5 |
| 875 | 969 | 4.7 | <0.5 | <0.5 |
| 862 | 956 | 4.2 | <0.5 | <0.5 |
| 864 | 958 | 3.9 | <0.5 | <0.5 |
| 848 | 942 | 3.8 | <0.5 | <0.5 |
| 805 | 899 | 3.8 | <0.5 | <0.5 |
| 854 | 948 | 3.7 | <0.5 | <0.5 |
| 840 | 934 | 3.5 | <0.5 | <0.5 |
| 817 | 911 | 3.5 | <0.5 | <0.5 |
| 819 | 913 | 3.2 | <0.5 | <0.5 |
| 876 | 970 | 3.1 | <0.5 | <0.5 |

| | | | |
|---|---|---|---|
| Negative control | 0.8 | <0.5 | <0.5 |

Based on detection of QS-18 2150 and QS-21 1988 components by LCMS/MS (Example 5-1) or UV HPLC quantification of Middle Peak (mainly QS-18 family and desglucosyl-QS-17 family) and Right Peak (mainly QS-21 family) (Examples 5-2 and 5-3), Example 5 shows that a number of suitable glucosidases could be identified by screening a set of candidate enzymes, and also that candidate enzymes demonstrating similarity to previously identified suitable glucosidases were more likely to also be suitable glucosides (51 from 94, 54%). Glucosidases were capable of converting QS-18 family components to QS-21 family components at a range of pHs, concentrations of starting materials and purity of starting materials.

Again, although certain candidate enzymes did not demonstrate notable conversion under the conditions tested, this may be due to issues with enzyme expression, suitability of conditions (i.e. enzymes may function under other conditions) or a fundamental lack of required enzyme activity.

### Example 6—Screening of Rhamnosidases for Derhamnosylation of QS-17 to QS-18

### Method

### Enzyme Selection

Conversion of QS-17 family components to QS-18 family components involves hydrolysis of the 1,2 glycosidic bond between the alpha-L-arabinofuranose and alpha-L-rhamnose found at the terminus of the acyl chain portion of the molecules. Glycoside hydrolases from families 78 and 106 exhibit the exo-alpha-1,2 rhamnosidase activity (E.C. 3.2.1.40) necessary to cleave this bond as annotated by the CAZy (Carbohydrate Active enZyme) database (www.cazy.com). All sequences annotated by CAZy from GH families 78 and 106 were obtained, and separate curated hidden Markov model profiles constructed for each which were then used to identify additional familial enzymes by searching the 209 million protein member Uniprot (www.uniprot.org) knowledgebase with the software HMMER (Eddy, 1998). In total, 11,749 sequences were identified: 10,653, and 1096 representatives from GH families 78 and 106, respectively. MMSeqs2 (Hauser, 2016) was then used to cluster each group of enzyme sequences using the default clustering workflow and parameters with a minimum sequence identity and coverage of 30% and 80%, respectively. In cases where the initial clustering yielded clusters with more than 1000 members, a second sub-clustering was performed at a higher 50% or 70% identity to ensure diverse exemplars from these larger clusters were represented more prominently. All clusters were then examined, and exemplars selected from each with preferences for annotation quality, known experimental activity, existing three dimensional structures from the Protein Data Bank (www.wwpdb.org) or known extremophile organisms as annotated by Uniprot. A final set of 94 diverse candidate enzymes was selected. Polynucleotide sequences encoding each selected enzyme linked to a C-terminal 6×His tag and Tev-cleavage site (amino acid sequence for linker His-tag, inserted N-terminally of stop codon, is provided in SEQ ID No. 1178) were prepared using a proprietary genetic-algorithm based codon optimization code.

Details of the candidate enzyme and polynucleotide sequences are summarised below in Table 10.

TABLE 10
Rhamnosidase candidate sequences

| | | | AA | DNA |
|---|---|---|---|---|
| | | | Seq | Seq |
| UniParc | Uniprot | Organism | ID | ID |
| UPI0001A31108 | D0MFR0 | Rhodothermus marinus | 989 | 1083 |
| UPI0001D90BFE | D7C463 | Streptomyces bingchenggensis | 990 | 1084 |
| UPI0001A3AEAC | D2QL60 | Spirosoma linguale | 991 | 1085 |
| UPI0001CD6D48 | D4L2K8 | Roseburia intestinalis | 992 | 1086 |
| UPI000442EF22 | X5DG83 | Draconibacterium orientale | 993 | 1087 |
| UPI00019E052B | C7QC24 | Catenulispora acidiphila | 994 | 1088 |
| UPI000005ADE1 | Q8A916 | Bacteroides thetaiotaomicron | 995 | 1089 |
| UPI000172B2E3 | B1ZRE4 | Opitutus terrae | 996 | 1090 |
| UPI00015FE0BE | A9KJP8 | Lachnoclostridium phytofermentans | 997 | 1091 |
| UPI00022DA3ED | M4NH01 | Rhodanobacter denitrificans | 998 | 1092 |
| UPI0001D07633 | D5ETD9 | Prevotella ruminicola | 999 | 1093 |
| UPI000259E388 | I0AZ41 | Aspergillus terreus | 1000 | 1094 |
| UPI0001A461B6 | C7MA58 | Brachybacterium faecium | 1001 | 1095 |
| UPI00006E4A0D | A5FCH3 | Flavobacterium johnsoniae | 1002 | 1096 |
| UPI000245C507 | H2IYR2 | Rahnella aquatilis | 1003 | 1097 |
| UPI0003E4E711 | W4N6H0 | Bifidobacterium moukalabense | 1004 | 1098 |
| UPI000353BD9F | S4BBS9 | Enterococcus casseliflavus | 1005 | 1099 |
| UPI0001789C0D | D3EED1 | Geobacillus sp. | 1006 | 1100 |
| UPI000260A2FE | 14EYD9 | Modestobacter marinus | 1007 | 1101 |
| UPI00019EF0EE | C6XYM6 | Pedobacter heparinus | 1008 | 1102 |
| UPI00019B5915 | C6VZL3 | Dyadobacter fermentans | 1009 | 1103 |
| UPI000212C156 | F8FQQ3 | Paenibacillus mucilaginosus | 1010 | 1104 |
| UPI0003E2544D | W4D866 | Paenibacillus sp. | 1011 | 1105 |
| UPI000005ADD2 | A0A0P0FM19 | Bacteroides thetaiotaomicron | 1012 | 1106 |
| UPI00005BA60B | A9WDK5 | Chloroflexus aurantiacus | 1013 | 1107 |
| UPI00000B098C | Q9S3L0 | Thermoclostridium stercorarium | 1014 | 1108 |
| UPI0003E5C314 | W4N612 | Bifidobacterium moukalabense | 1015 | 1109 |
| UPI0003AE032C | U2USP4 | Olsenella profusa | 1016 | 1110 |
| UPI00019BDB13 | D2PMT5 | Kribbella flavida | 1017 | 1111 |
| UPI00000C7226 | Q9A9K2 | Caulobacter vibrioides | 1018 | 1112 |
| UPI000005BA09 | Q8A076 | Bacteroides thetaiotaomicron | 1019 | 1113 |
| UPI0003745394 | U5BUY4 | Rhodonellum psychrophilum | 1020 | 1114 |
| UPI0004F6D660 | A0A089M3T2 | Paenibacillus sp. | 1021 | 1115 |
| UPI0003ED860D | W7QMH5 | Catenovulum agarivorans | 1022 | 1116 |
| UPI000217D8B1 | G0L382 | Zobellia galactanivorans | 1023 | 1117 |
| UPI000005B845 | Q8A1H5 | Bacteroides thetaiotaomicron | 1024 | 1118 |
| UPI0001CD02E9 | D6CYE5 | Bacteroides xylanisolvens | 1025 | 1119 |
| UPI00018E07C6 | B8HAH3 | Pseudarthrobacter chlorophenolicus | 1026 | 1120 |
| UPI0001815896 | B5YC64 | Dictyoglomus thermophilum | 1027 | 1121 |
| UPI00039231C1 | T2KPL4 | Formosa agariphila | 1028 | 1122 |
| UPI0000DBA6EB | Q0S9T4 | Rhodococcus jostii | 1029 | 1123 |
| UPI0001D10896 | D5GZ45 | Lactobacillus crispatus | 1030 | 1124 |
| UPI0001B17DE6 | C6XVU2 | Pedobacter heparinus | 1031 | 1125 |
| UPI0001A3C989 | D2QUA5 | Spirosoma linguale | 1032 | 1126 |
| UPI00019EE3EB | C6XU05 | Pedobacter heparinus | 1033 | 1127 |
| UPI0003432C11 | R9ULQ4 | Paenibacillus mucilaginosus | 1034 | 1128 |
| UPI0001BC0C05 | D5VGD9 | Caulobacter segnis | 1035 | 1129 |
| UPI0001969377 | E2N9B1 | Bacteroides cellulosilyticus | 1036 | 1130 |
| UPI0001B17C60 | C6Y153 | Pedobacter heparinus | 1037 | 1131 |
| UPI000571C0C2 | T2KNB2 | Formosa agariphila | 1038 | 1132 |
| UPI00004C6D41 | Q5FJ31 | Lactobacillus acidophilus | 1039 | 1133 |
| UPI00001AC07D | Q7UYD5 | Rhodopirellula baltica | 1040 | 1134 |
| UPI0001BF9A6C | E3IY10 | Frankia inefficax | 1041 | 1135 |
| UPI0001B7FF91 | C9Z376 | Streptomyces scabiei | 1042 | 1136 |
| UPI00006E5F74 | A5FC22 | Flavobacterium johnsoniae | 1043 | 1137 |
| UPI00034E666D | S2YWB5 | Streptomyces sp. | 1044 | 1138 |
| UPI000198DF25 | C1F149 | Acidobacterium capsulatum | 1045 | 1139 |
| UPI0003ED82D3 | W7QYP5 | Catenovulum agarivorans | 1046 | 1140 |
| UPI0001A461B7 | C7MA59 | Brachybacterium faecium | 1047 | 1141 |
| UPI000243A177 | A0A0J9X262 | Klebsiella oxytoca | 1048 | 1142 |
| UPI0001B24769 | C7PA70 | Chitinophaga pinensis | 1049 | 1143 |
| UPI0002BCAF6B | M3FYL9 | Streptomyces bottropensis | 1050 | 1144 |
| UPI0001966B28 | D1PKC7 | Subdoligranulum variabile | 1051 | 1145 |
| UPI0001F8A51D | E8NDD8 | Microbacterium testaceum | 1052 | 1146 |
| UPI000053767B | Q01V09 | Solibacter usitatus | 1053 | 1147 |
| UPI0001A3EFE6 | D2B240 | Streptosporangium roseum | 1054 | 1148 |
| UPI0006CE1E82 | A0A0N1BME3 | alpha proteobacterium | 1055 | 1149 |
| UPI000247229E | H8KPI7 | Solitalea canadensis | 1056 | 1150 |
| UPI0002CB9583 | S0GSF0 | Parabacteroides goldsteinii | 1057 | 1151 |
| UPI00021B9B33 | G0J630 | Cyclobacterium marinum | 1058 | 1152 |
| UPI0000533669 | Q01TX2 | Solibacter usitatus | 1059 | 1153 |
| UPI00032D8F6D | R7ZW70 | Lunatimonas lonarensis | 1060 | 1154 |
| UPI0000D713F2 | Q1M7P3 | Rhizobium leguminosarum | 1061 | 1155 |
| UPI0001A3EBEB | D2AYU9 | Streptosporangium roseum | 1062 | 1156 |
| UPI000156F115 | A6LBL4 | Parabacteroides distasonis | 1063 | 1157 |
| UPI0003375A10 | R9K6L6 | Lachnospiraceae bacterium | 1064 | 1158 |
| UPI0001A2F0FA | C7P9Y8 | Chitinophaga pinensis | 1065 | 1159 |
| UPI0001BC0C15 | D5VGC3 | Caulobacter segnis | 1066 | 1160 |
| UPI00019EF6E1 | C6Y145 | Pedobacter heparinus | 1067 | 1161 |
| UPI00019EE1A9 | C6Y2X3 | Pedobacter heparinus | 1068 | 1162 |
| UPI000C8D4928 | A0A2D5SK32 | Deltaproteobacteria bacterium | 1069 | 1163 |
| UPI00019BFDCE | D1CHL4 | Thermobaculum terrenum | 1070 | 1164 |
| UPI000172B62A | B1ZY35 | Opitutus terrae | 1071 | 1165 |
| UPI00019BFABB | D2PXQ4 | Kribbella flavida | 1072 | 1166 |
| UPI0001B80091 | C9Z391 | Streptomyces scabiei | 1073 | 1167 |
| UPI00023EC5D1 | G8S540 | Actinoplanes sp. | 1074 | 1168 |
| UPI0003C3CD2B | V4NSJ1 | Asticcacaulis sp. | 1075 | 1169 |
| UPI00019BF65D | D2PT74 | Kribbella flavida | 1076 | 1170 |
| UPI00000BC760 | Q93RE7 | Bacillus sp. | 1077 | 1171 |
| UPI00006E5FAB | A5FCG3 | Flavobacterium johnsoniae | 1078 | 1172 |
| UPI00032EEB9C | R7ZS84 | Lunatimonas lonarensis | 1079 | 1173 |
| UPI0004B2D794 | A0A3E3IGR6 | Eisenbergiella massiliensis | 1080 | 1174 |
| UPI0003ED7515 | W7QF25 | Catenovulum agarivorans | 1081 | 1175 |
| UPI0000184198 | Q82PP4 | Streptomyces avermitilis | 1082 | 1176 |

### Experiment 6-1—Screening of Rhamnosidases for Derhamnosylation of Saponins in Treated Bark Extract (2%)

Synthetic nucleotide sequences corresponding to SEQ ID 989 to 1082 were subcloned, transformed, expressed and lysed in an identical manner to Experiment 4-1 with the exception that a single cell pellet plate was lysed with 200 ul lysis buffer.

Treated bark extract (TBE) solution was prepared by diluting 1 volume with 9 volumes of 100 mM potassium phosphate pH 7.5. 40 uL clarified lysate was transferred into fresh 96 well PCR plates. 10 uL 10× diluted TBE solution added to each well of lysate to a final concentration of 2% ( 1/50). Plates were incubated at 30 degC with shaking for 18 h. Quenched with 50 uL MeCN+2% AcOH and shaken at room temperature for 10 mins prior to centrifugation (10 min, 4 degC) to remove particulates. Samples were analysed by LCMS/MS using method of Experiment 4-1. The following MS-MS transitions were monitored to observe loss of rhamnose from starting saponins to product derhamnosylated saponins.

| | | Dwell | | Cone | Collision |
|---|---|---|---|---|---|
| | | Time | MRM | Voltage | Energy |
| Component | Mode | (secs) | Transitions | (V) | (v) |
| QS-17 2296 | ESI - | Auto | 1147.52 > 836.40 | 34 | 16 |
| QS-18 2150 | ESI - | Auto | 1074.50 > 836.40 | 34 | 16 |
| desglucosyl- | ESI - | Auto | 1066.49 > 755.33 | 34 | 16 |
| QS-17 2134 | | | | | |
| QS-21 1988 | ESI - | Auto | 993.46 > 755.5 | 34 | 16 |
| QS-17 2310 | ESI - | Auto | 1154.53 > 843.37 | 34 | 16 |
| QS-18 2164 | ESI - | Auto | 1081.50 > 843.37 | 34 | 16 |

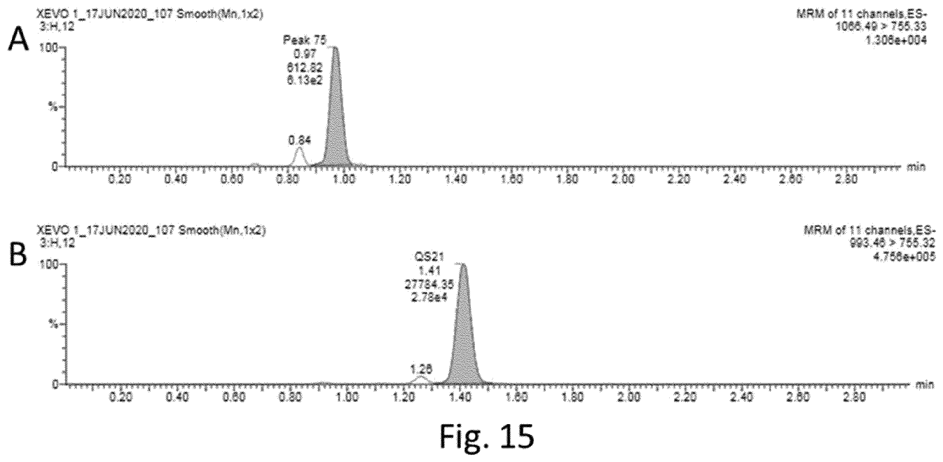

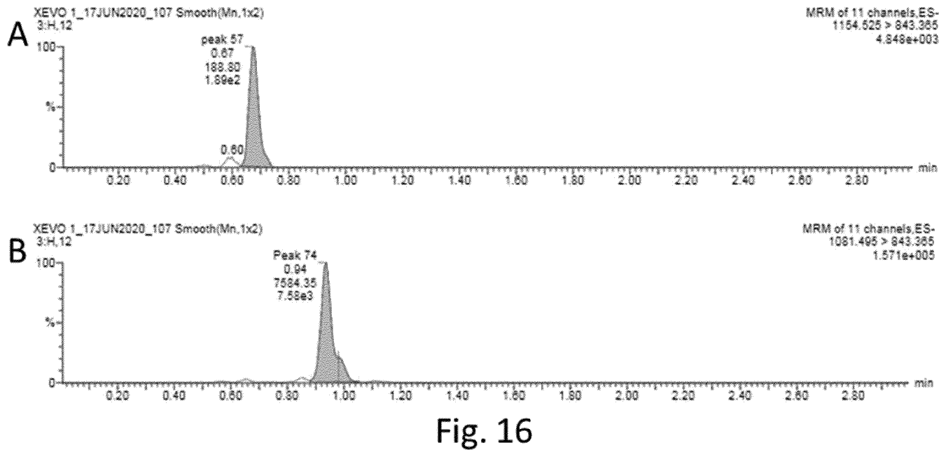

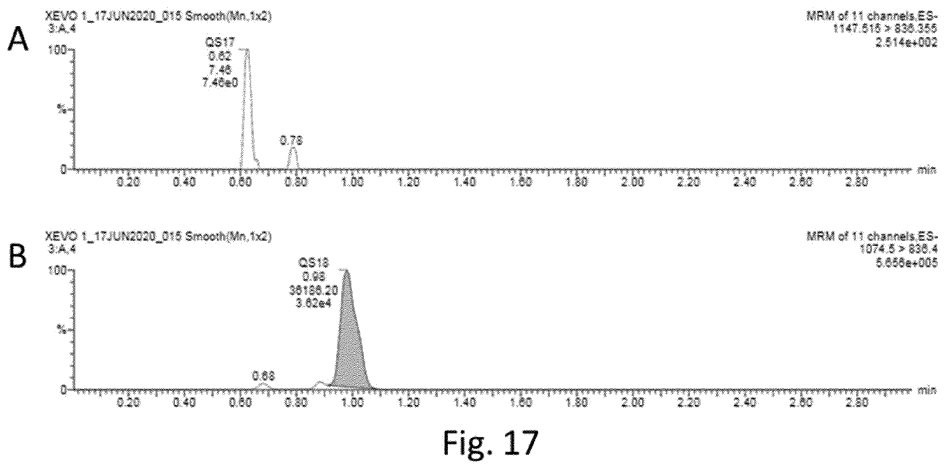

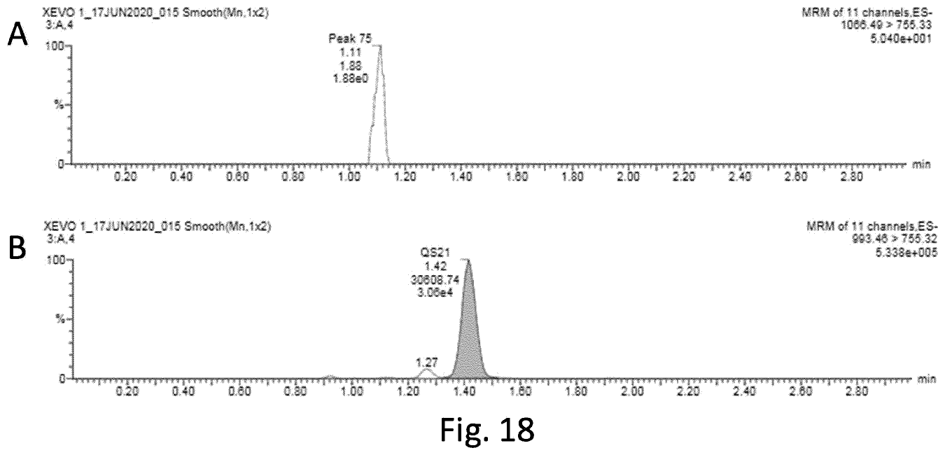

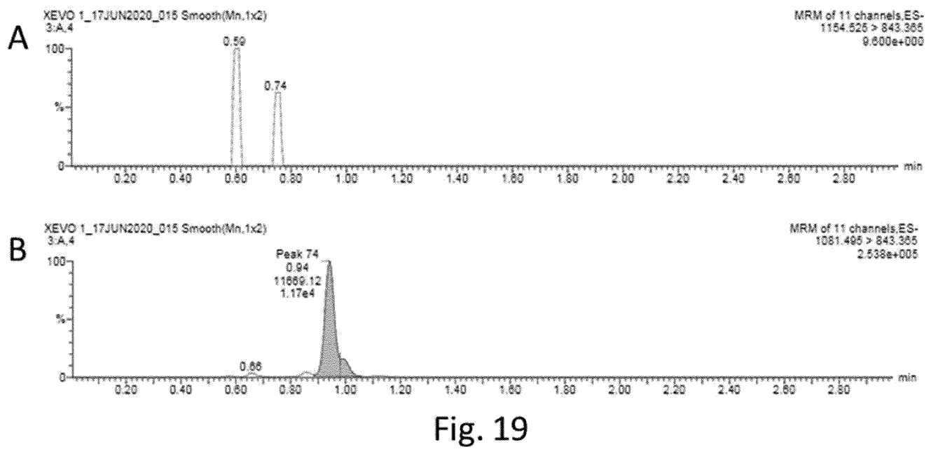

FIGS. 14 to 19 provide exemplary chromatograms following negative control treatment and rhamnosidase SEQ ID No. 1017 treatment.

Data is expressed as TIC peak area ratio percent (PAR %) for rhamnosylated starting saponin to derhamnosylated product:

$$PAR \% = \frac{100 \times rhamnosylated starting saponin}{\begin{matrix}( rhamnosylated starting saponin + \\ derhamnosylated product saponin )\end{matrix}}$$

Activity was measured for the removal of the alpha-O-rhamnosylation at the C2 position of the arabinofuranose moiety of QS-17 2296 component to produce QS-18 2150 component; desglucosyl-QS-17 2134 to produce QS-21 1988 component and QS-17 2310 component to produce QS-18 2164.

No activity was detected for the removal of the C3 saccharide rhamnose as demonstrated by no effect on QS-18 2164 component (QS-18 2164/QS-21 1988 ratio is unchanged). Additionally no endo cleavage of the C28 saccharide rhamnose attached to the C2 position of the fucose was observed.

TABLE 11

| AA Seq | DNA | QS-17 | Peak75 | Peak57 | ratio |
|---|---|---|---|---|---|
| ID | Seq ID | (PAR %) | (PAR %) | (PAR %) | PP/QS-21 |
| 992 | 1086 | 0.000 | 0.000 | 0.000 | 0.20 |
| 1003 | 1097 | 0.000 | 0.000 | 0.000 | 0.19 |
| 1052 | 1146 | 0.000 | 0.000 | 0.000 | 0.16 |
| 1073 | 1167 | 0.007 | 0.000 | 0.000 | 0.18 |
| 1017 | 1111 | 0.017 | 0.000 | 0.004 | 0.19 |
| 1055 | 1149 | 0.019 | 0.000 | 0.000 | 0.19 |
| 1075 | 1169 | 0.043 | 0.000 | 0.000 | 0.18 |
| 1001 | 1095 | 0.075 | 0.137 | 0.000 | 0.19 |
| 1007 | 1101 | 0.104 | 0.043 | 0.009 | 0.19 |
| 1061 | 1155 | 0.245 | 0.230 | 0.029 | 0.19 |
| 1079 | 1173 | 0.315 | 0.247 | 0.000 | 0.18 |
| 1027 | 1121 | 0.324 | 0.094 | 0.053 | 0.19 |
| 1039 | 1133 | 0.492 | 0.683 | 0.000 | 0.18 |
| 1041 | 1135 | 0.507 | 0.000 | 0.000 | 0.16 |
| 989 | 1083 | 0.516 | 0.478 | 0.305 | 0.18 |
| 1053 | 1147 | 1.127 | 0.944 | 0.550 | 0.18 |
| 1018 | 1112 | 1.512 | 2.651 | 0.547 | 0.17 |
| 1066 | 1160 | 2.194 | 1.220 | 0.896 | 0.18 |
| 1082 | 1176 | 3.122 | 1.614 | 0.000 | 0.21 |
| 1076 | 1170 | 3.766 | 1.961 | 1.295 | 0.19 |
| 993 | 1087 | 3.836 | 2.211 | 1.129 | 0.18 |
| 1077 | 1171 | 3.964 | 2.138 | 2.532 | 0.18 |
| 1046 | 1140 | 4.051 | 2.446 | 2.480 | 0.13 |
| 1015 | 1109 | 4.132 | 2.969 | 3.048 | 0.15 |
| 1063 | 1157 | 4.201 | 2.240 | 2.473 | 0.17 |
| 1054 | 1148 | 4.348 | 2.160 | 2.487 | 0.17 |
| 1074 | 1168 | 4.443 | 2.312 | 1.681 | 0.18 |
| 1067 | 1161 | 4.455 | 2.231 | 2.852 | 0.17 |
| 1033 | 1127 | 4.485 | 2.521 | 2.927 | 0.16 |
| 1071 | 1165 | 4.587 | 2.574 | 2.650 | 0.17 |
| 1059 | 1153 | 4.613 | 2.534 | 2.956 | 0.16 |
| 1064 | 1158 | 4.625 | 2.476 | 3.009 | 0.18 |
| 1019 | 1113 | 4.626 | 2.982 | 2.390 | 0.15 |
| 1047 | 1141 | 4.669 | 2.456 | 3.332 | 0.17 |
| 1057 | 1151 | 4.670 | 2.498 | 3.213 | 0.15 |
| 1038 | 1132 | 4.725 | 2.512 | 2.913 | 0.18 |
| 1032 | 1126 | 4.730 | 2.411 | 3.044 | 0.17 |
| 1045 | 1139 | 4.741 | 2.442 | 2.865 | 0.17 |
| 997 | 1091 | 4.754 | 2.115 | 2.906 | 0.17 |
| 1056 | 1150 | 4.774 | 2.416 | 2.796 | 0.17 |
| 996 | 1090 | 4.778 | 2.221 | 3.190 | 0.16 |
| 1069 | 1163 | 4.786 | 2.213 | 2.596 | 0.17 |
| 1080 | 1174 | 4.792 | 2.434 | 2.882 | 0.17 |
| 1010 | 1104 | 4.796 | 2.559 | 2.845 | 0.18 |
| 1035 | 1129 | 4.825 | 2.427 | 2.987 | 0.17 |
| 1034 | 1128 | 4.827 | 2.389 | 3.034 | 0.17 |
| 1036 | 1130 | 4.834 | 2.444 | 2.852 | 0.16 |
| 1008 | 1102 | 4.834 | 2.538 | 2.658 | 0.17 |
| 1042 | 1136 | 4.871 | 2.599 | 3.383 | 0.18 |
| 1016 | 1110 | 4.872 | 2.498 | 3.326 | 0.18 |
| 1023 | 1117 | 4.916 | 2.120 | 2.889 | 0.17 |
| 1062 | 1156 | 4.916 | 2.410 | 2.941 | 0.18 |
| 1002 | 1096 | 4.922 | 2.538 | 2.843 | 0.16 |
| 1029 | 1123 | 4.926 | 2.069 | 3.334 | 0.16 |
| 1037 | 1131 | 4.972 | 2.424 | 2.764 | 0.17 |
| 1048 | 1142 | 5.007 | 2.441 | 2.465 | 0.17 |
| 1000 | 1094 | 5.010 | 2.788 | 3.119 | 0.16 |
| 1065 | 1159 | 5.015 | 2.458 | 3.194 | 0.17 |
| 1020 | 1114 | 5.058 | 2.360 | 3.288 | 0.17 |
| 1012 | 1106 | 5.068 | 2.610 | 3.249 | 0.16 |
| 1004 | 1098 | 5.076 | 2.553 | 2.942 | 0.16 |
| 1028 | 1122 | 5.081 | 2.885 | 1.993 | 0.15 |
| 991 | 1085 | 5.084 | 2.453 | 3.443 | 0.17 |
| 1011 | 1105 | 5.089 | 2.854 | 3.124 | 0.19 |
| 1043 | 1137 | 5.098 | 2.791 | 3.119 | 0.15 |
| 998 | 1092 | 5.111 | 2.402 | 3.326 | 0.15 |
| 1058 | 1152 | 5.121 | 2.398 | 3.144 | 0.17 |
| 990 | 1084 | 5.144 | 2.151 | 0.188 | 0.21 |
| 1024 | 1118 | 5.145 | 2.806 | 2.924 | 0.17 |
| 1060 | 1154 | 5.159 | 2.382 | 2.888 | 0.15 |
| 1005 | 1099 | 5.166 | 2.767 | 2.799 | 0.17 |
| 1070 | 1164 | 5.168 | 2.275 | 3.311 | 0.18 |
| 994 | 1088 | 5.177 | 2.147 | 3.300 | 0.16 |
| 1022 | 1116 | 5.190 | 2.368 | 2.882 | 0.15 |
| 1049 | 1143 | 5.211 | 2.417 | 2.271 | 0.18 |
| 1031 | 1125 | 5.218 | 2.367 | 3.096 | 0.17 |
| 1078 | 1172 | 5.218 | 2.153 | 2.788 | 0.16 |
| 995 | 1089 | 5.223 | 2.545 | 3.002 | 0.17 |
| 1040 | 1134 | 5.244 | 2.372 | 3.084 | 0.18 |
| 1050 | 1144 | 5.251 | 2.528 | 2.305 | 0.17 |
| 1026 | 1120 | 5.271 | 2.501 | 2.755 | 0.17 |
| 1013 | 1107 | 5.272 | 2.388 | 2.576 | 0.17 |
| 1051 | 1145 | 5.289 | 2.426 | 2.918 | 0.16 |
| 1014 | 1108 | 5.309 | 2.741 | 3.359 | 0.18 |
| 1030 | 1124 | 5.315 | 2.127 | 3.621 | 0.17 |
| 1006 | 1100 | 5.339 | 2.645 | 3.237 | 0.18 |
| 1081 | 1175 | 5.348 | 2.473 | 2.624 | 0.17 |
| 1044 | 1138 | 5.370 | 2.274 | 3.156 | 0.16 |
| 1009 | 1103 | 5.408 | 2.379 | 3.762 | 0.16 |
| 1072 | 1166 | 5.457 | 2.462 | 3.245 | 0.17 |
| 1021 | 1115 | 5.476 | 2.296 | 3.148 | 0.16 |
| 1025 | 1119 | 5.495 | 2.330 | 2.933 | 0.18 |
| 1068 | 1162 | 5.521 | 2.370 | 3.156 | 0.17 |
| 999 | 1093 | 5.614 | 2.571 | 2.714 | 0.16 |

| | | | | |
|---|---|---|---|---|
| Negative Control 1 | 4.876 | 2.719 | 3.061 | 0.18 |
| Negative Control 2 | 5.225 | 2.158 | 2.429 | 0.17 |

### Experiment 6-2 Screening of Rhamnosidases for Derhamnosylation of Saponins in Treated Bark Extract (25%)

A subset of rhamnosidases were expressed and lysed as in the method of experiment 6-1.

Treated bark extract (TBE) solution was adjusted to pH 7.4 by addition of NaOH (2M). 75 uL clarified lysate was transferred into fresh 96 well PCR plates. 25 uL TBE solution (pH 7.4) was added to each well of lysate to a final concentration of 25%. Plates were incubated at degC with shaking for 19.5 h.

Quenched with 100 uL MeCN+2% AcOH and shaken at room temperature for 10 mins prior to centrifugation (10 min, 4 degC) to remove particulates. Samples were analysed by UV HPLC using method of Experiment 4-4. Three key peaks of interest are apparent using this chromatography: Left Peak (retention time approximately 2.30-2.35 min) comprising mainly QS-17 family components; Middle Peak (retention time approximately 2.37-2.42 min) comprising mainly QS-18 family components and desglucosyl-QS-17 family components; and Right Peak (retention time approximately 2.44-2.50 min) comprising mainly QS-21 family components. Peak identity was supported by MS/MS.

Enzyme mediated hydrolysis of the alpha-O-rhamnosylation at the C2 position of the arabinofuranose moiety of QS-17 family components leads to a decrease in Left Peak and an increase in Middle Peak due to formation of QS-18 family components. Enzyme mediated hydrolysis of the alpha-O-rhamnosylation at the C2 position of the arabinofuranose moiety of desglucosyl-QS-17 family components leads to a decrease in Middle Peak and an increase in Right Peak due to formation of QS-21 family components.

The relative percentage of each peak was determined. A decrease in Left Peak and a concomitant increase in Middle Peak and Right Peak is observed for enzymes active under these conditions. Results for the tested subset of rhamnosidases are provided below in Table 12.

TABLE 12

| AA Seq | DNA | Left | Middle | Right |
|---|---|---|---|---|
| ID | Seq ID | Peak | Peak | Peak |
| 1017 | 1111 | 6% | 63% | 31% |
| 1073 | 1167 | 6% | 62% | 31% |
| 1003 | 1097 | 7% | 63% | 31% |
| 1055 | 1149 | 7% | 62% | 31% |
| 992 | 1086 | 7% | 62% | 31% |
| 1007 | 1101 | 8% | 62% | 30% |
| 1052 | 1146 | 9% | 62% | 30% |
| 1075 | 1169 | 9% | 61% | 30% |
| 1061 | 1155 | 13% | 59% | 28% |
| 1001 | 1095 | 14% | 59% | 27% |
| 1079 | 1173 | 14% | 58% | 28% |
| 1041 | 1135 | 15% | 55% | 30% |
| 1039 | 1133 | 16% | 57% | 27% |
| 993 | 1087 | 18% | 56% | 27% |
| 1027 | 1121 | 18% | 55% | 27% |
| 1082 | 1176 | 19% | 55% | 26% |
| 989 | 1083 | 19% | 54% | 26% |
| 1053 | 1147 | 20% | 54% | 26% |
| 1066 | 1160 | 20% | 53% | 27% |
| 1018 | 1112 | 22% | 52% | 26% |
| 1076 | 1170 | 22% | 52% | 26% |
| 990 | 1084 | 23% | 51% | 26% |
| 1077 | 1171 | 23% | 51% | 26% |

| | | | |
|---|---|---|---|
| Negative control | 24% | 50% | 26% |

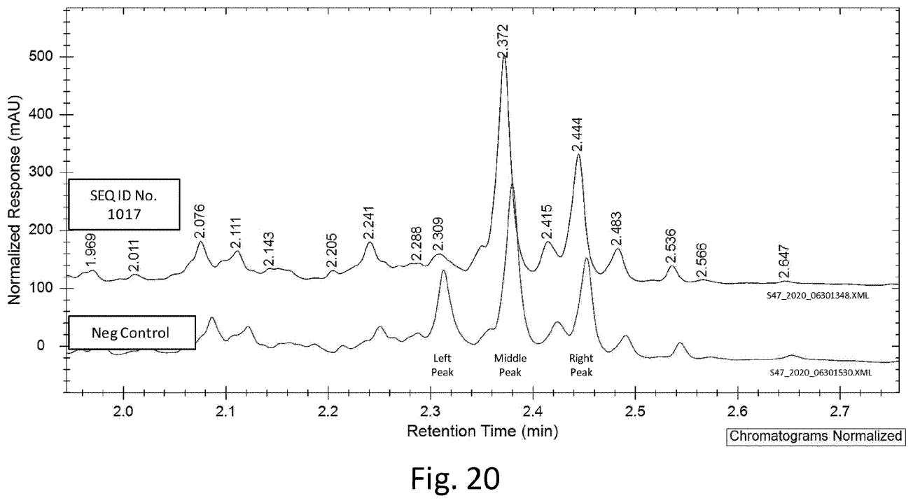

Example UV HPLC chromatograms are shown in FIG. 20 for SEQ ID No. 1017 treatment and negative control.

### Experiment 6-3 Screening of Rhamnosidases for Derhamnosylation of Saponins in Crude Bark Extract (80%)

Selected variants were expressed and lysed as in the method of experiment 6-1.

Crude bark extract (CBE) solution was adjusted to pH 7.4 by addition of NaOH (2M). 20 uL clarified lysate was transferred into fresh 96 well PCR plates. 80 uL CBE solution (pH 7.4) was added to each well of lysate to a final concentration of 80%. Plates were incubated at 30 degC with shaking for 19.5 h.

Quenched with 100 uL MeCN+2% AcOH and shaken at room temperature for 10 mins prior to centrifugation (10 min, 4 degC) to remove particulates. Samples were analysed by UV HPLC using method of Experiment 4-4.

Three key peaks of interest are apparent using this chromatography: Left Peak (retention time approximately 2.30-2.35 min) comprising mainly QS-17 family components; Middle Peak (retention time approximately 2.37-2.42 min) comprising mainly QS-18 family components and desglucosyl-QS-17 family components; and Right Peak (retention time approximately 2.44-2.50 min) comprising mainly QS-21 family components. Peak identity was supported by MS/MS.

Enzyme mediated hydrolysis of the alpha-O-rhamnosylation at the C2 position of the arabinofuranose moiety of QS-17 family components leads to a decrease in Left Peak and an increase in Middle Peak due to formation of QS-18 family components. Enzyme mediated hydrolysis of the alpha-O-rhamnosylation at the C2 position of the arabinofuranose moiety of desglucosyl-QS-17 family components leads to a decrease in Middle Peak and an increase in Right Peak due to formation of QS-21 family components.

The relative percentage of each peak was determined. A decrease in Left Peak and a concomitant increase in Middle Peak and Right Peak is observed for enzymes active under these conditions. Results for the tested subset of rhamnosidases are provided below in Table 13:

TABLE 13

| AA Seq | DNA | Left | Middle | Right |
|---|---|---|---|---|
| ID | Seq ID | Peak | Peak | Peak |
| 1017 | 1111 | 17% | 56% | 27% |
| 1073 | 1167 | 20% | 52% | 28% |
| 992 | 1086 | 21% | 49% | 30% |
| 1007 | 1101 | 24% | 49% | 27% |
| 1052 | 1146 | 25% | 49% | 27% |
| 1079 | 1173 | 25% | 48% | 27% |
| 993 | 1087 | 25% | 49% | 26% |
| 1041 | 1135 | 25% | 48% | 27% |
| 1055 | 1149 | 25% | 48% | 27% |
| 1039 | 1133 | 25% | 48% | 27% |
| 990 | 1084 | 25% | 47% | 28% |
| 1061 | 1155 | 26% | 48% | 27% |
| 1053 | 1147 | 26% | 48% | 26% |
| 1027 | 1121 | 26% | 48% | 26% |
| 1076 | 1170 | 26% | 48% | 27% |
| 1066 | 1160 | 26% | 48% | 27% |
| 1018 | 1112 | 26% | 48% | 26% |
| 1075 | 1169 | 26% | 47% | 27% |
| 1077 | 1171 | 26% | 48% | 27% |
| 1082 | 1176 | 26% | 47% | 27% |
| 1003 | 1097 | 27% | 47% | 26% |
| 989 | 1083 | 29% | 43% | 27% |
| 1001 | 1095 | 31% | 45% | 24% |

| | | | |
|---|---|---|---|
| Negative control | 27% | 48% | 26% |

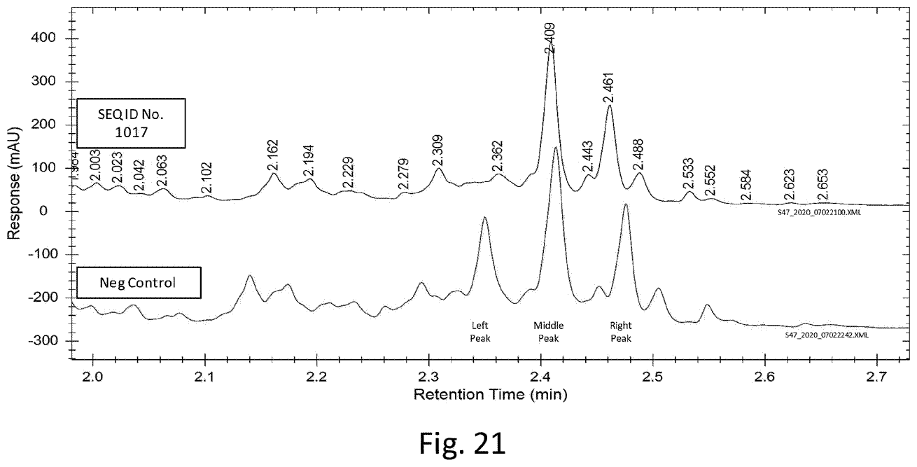

Example UV HPLC chromatograms are shown in FIG. 21 for SEQ ID No. 1017 treatment and negative control.

Based on detection of QS-17 2296, QS-17 2310, QS-18 2150, QS-18 2164, desglucosyl-QS-17 2134 and QS-21 1988 components by LCMS/MS (Example 6-1) or UV HPLC quantification of QS-17, QS-18 and QS-21 peaks (Examples 6-2 and 6-3), Example 6 shows that a number of rhamnosidases could be identified by screening a set of candidate enzymes (29 from 94, 31% achieving a QS-17 PAR % of 4.5 or less in Example 6-1). Rhamnosidases were capable of converting QS-17 family components to QS-18 family components and desglucosyl-QS-17 family components to QS-21 family components at a range of concentrations of starting materials and purity of starting materials.

Again, although certain candidate enzymes did not demonstrate notable conversion under the conditions tested, this may be due to issues with enzyme expression, suitability of conditions (i.e. enzymes may function under other conditions) or a fundamental lack of required enzyme activity.

### Example 7—Deglucosylation and Derhamnosylation of Saponins in Crude Bark Extract (50%)

### Method

E. coli cells expressing glucosidase SEQ ID No. 262 (as His-tagged enzyme, DNA SEQ ID No. 662) and separately rhamnosidase SEQ_ID No. 1017 (as His-tagged form, DNA SEQ ID No. 1111) were grown in a fermenter, isolated, lysed, clarified and the resulting lysate lyophilised to yield powder containing each of the expressed enzymes.

500 uL CBE was mixed with 500 uL volume sodium acetate buffer (50 mM, pH 6) containing 30 g/L lyophilised powder containing the glucosidase, and 3 g/L lyophilised powder containing the rhamnosidase, and incubated at 37 degC for 24 hours.

The reaction was quenched by the addition of an equal volume of MeOH and analysed by LC-MS/MS using the method of Experiment 4-1 monitoring the transitions in the table below

TABLE 14

| Component | MRM Transitions |
|---|---|
| QS-18 2150 | 1074.49 > 836.355 |
| QS-18 2164 | 1081.495 > 843.365 |
| QS-17 2296 | 1147.515 > 836.355 |
| QS-17 2310 | 1154.525 > 843.365 |
| QS-21 1988 | 993.46 > 755.33 |
| QS-21 2002 | 1000.47 > 762.34 |
| desarabinofuranosyl-QS-18 2018 | 1008.465 > 836.355 |
| desglucosyl-QS-17 2134 | 1066.49 > 755.33 |

### Results

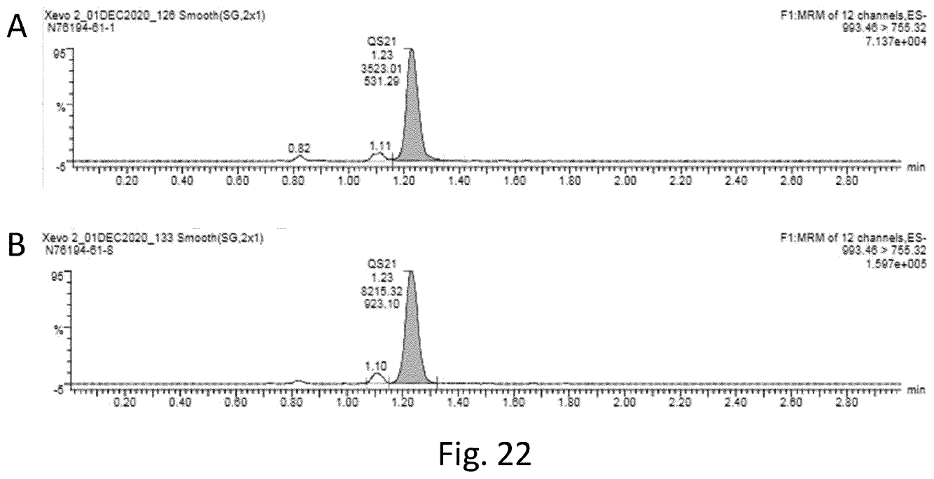

FIG. 22 provides exemplary LCMS/MS chromatograms for QS-21 1988 component content at TO (Panel A) and at 24 hrs (Panel B). Results for all components monitored are summarised below:

TABLE 15

| | Peak area | Percent change |
|---|---|---|
| Component | at 24 h | at 24 h |
| QS-18 2150 | 1621 | −80% |
| QS-18 2164 | 95 | −94% |
| QS-17 2296 | 0 | −100% |
| QS-17 2310 | 0 | −100% |
| QS-21 1988 | 8215 | 133% |
| QS-21 2002 | 1359 | 266% |
| desarabinofuranosyl-QS-18 2018 | 6 | −79% |
| desglucosyl-QS-17 2134 | 0 | −100% |

Components possessing alpha-O-rhamnosylation at the C2 position of the arabinofuranose moiety are reduced below the detection limit while components possessing a glucose moiety show >78% reduction after treatment for 24 h. The corresponding products of selective rhamnose and glucose hydrolysis show substantial increases.

### Example 8—Screening of Glucosidase Variants for Deglycosylation of Saponins in Crude Bark Extract

### Method

Libraries of genetic variants encoding mutations in the wild type Modestobacter marinus glucosidase (SEQ ID No. 262) were prepared using molecular biology techniques, enzymes were prepared linked to an N-terminally located His-tag. Single monoclonal colonies were grown in 400 ul of expression medium and protein expressed. Cell pellets were lysed in 200 ul of the relevant buffer (Table 16)

Lysate was diluted appropriately in the relevant buffer to allow a lysate loading of the indicated % loading (1% loading corresponds to use of 2 ul original lysate in a 200 ul reaction). In some experiments a rhamnosidase was also present during the screening reaction (and also in controls, negating any impact on results).

Crude bark extract (CBE) obtained by aqueous extraction of Quillaja saponaria and containing at least 2.80 mg/ml QS-21 (by HPLC-UV). The pH of CBE was adjusted to pH 6 by dropwise addition of 2M NaOH with stirring. The relevant concentration of the relevant glucosidase was added. The appropriate relative volume of pH 6 CBE (160 ul (for 80%) or 150 ul (for 75%)) was added to each well of the reaction plate. The reaction plate was sealed and incubated at the relevant temperature with shaking overnight for between 18 and 22 hours.

The reaction plate was quenched by adding 200 uL of MeCN (2% AcOH, 1 mg/mL hexanophenone) to each well of the plates. The quenched reaction plate was re-sealed and incubated at 20 deg C. with shaking for 10 min. The reaction plate was then centrifuged (10 min, 4 degC).

200 uL was transferred from each well of the quenched plate to the corresponding wells of a fresh 96 well plate and sealed. The plate was analysed by UV HPLC with the method below:

UV Method EM2020N435545v2_2

| Column details | ACQUITY UPLC BEH C18 Column, | | |
|---|---|---|---|
| | 130 Å, 1.7 μm, 2.1 mm × 50 mm | | |
| Column temperature | 40° C. | | |
| Mobile phase A | 0.05% v/v TFA in Water HPLC | | |
| Mobile phase B | 0.05% v/v TFA in Acetonitrile HPLC | | |
| Flow rate | 1 mL per minute | | |
| | Time | % Mobile | % Mobile |
| | (mins) | Phase A | Phase B |
| Gradient profile | 0.0 | 60 | 40 |
| 5.5 min gradient | 1.0 | 60 | 40 |
| | 2.1 | 5 | 95 |
| | 2.6 | 5 | 95 |
| | 2.7 | 60 | 40 |
| | 3.5 | 60 | 40 |

| | |
|---|---|
| Detector type | UV detector: DAD or VWD |
| Detector wavelength | 214 nm |
| Agilent DAD 1260 | 4.0 nm (default setting) |
| and 1290 Bandwidth | |
| Agilent 1290 and 1260 | Off |
| DAD reference wavelength | |
| Injection volume | Default injection volume used is 1 ul |

A negative control (a lysate not expressing test enzymes) and a positive control (expressing the parent comparator—wild type or previous variant as appropriate). Fold improvement over parent (FIOP) for the glucosidase (shorter method) is calculated as follows:

% right peak=100*right peak area/(right peak area+left peak area)

Average % right peak area is calculated for negative controls (per plate) and subtracted from all wells to give the increase in % right peak for each well above average negative control Average increase in % right peak is calculated for positive controls per plate

FIOP=observed increase in % right peak divided by average positive control increase

### Results

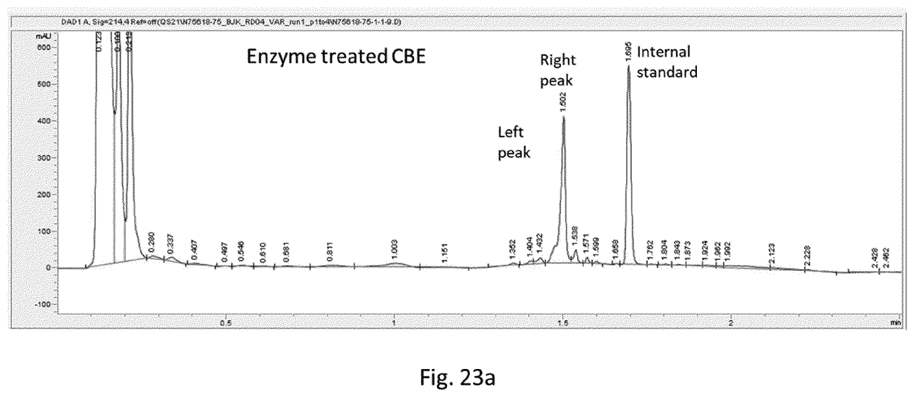

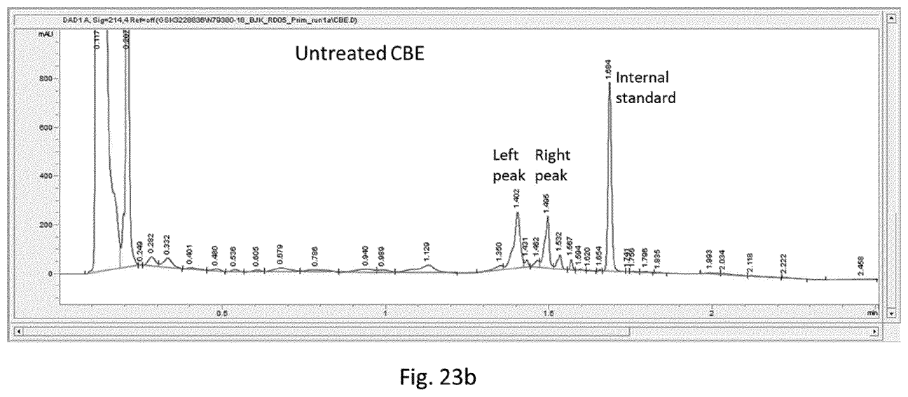

FIG. 23 provides illustrative chromatograms following treatment of CBE with enzymes and for a negative control.

TABLE 16

| | Mutations cf | Screen | | Lysate | CBE | | | Cumulative | |
|---|---|---|---|---|---|---|---|---|---|
| Enzyme | untagged WT | temp | pH | loading | loading | Buffer | FIOP | FIOP (cf WT) | Rham |
| WT | n/a | 35 | 6 | 20% | 80% | 100 mM | 1 | 1 | None |
| (SEQ ID | | | | | | Potassium | | | |
| No. 262) | | | | | | phosphate | | | |
| G1 | T365N | 35 | 6 | 20% | 80% | 100 mM | 5 | 5 | None |
| (SEQ ID | | | | | | Potassium | | | |
| No. 1179) | | | | | | phosphate | | | |
| G2 | T365N, | 20 | 6 | 10% | 80% | 100 mM | 10 | 50 | None |
| (SEQ ID | R357M, | | | | | Potassium | | | |
| No. 1180) | A473F, | | | | | phosphate | | | |
| | L474C, I475F | | | | | | | | |
| G3 | T365N, | 20 | 6 | 2.50% | 80% | 100 mM | 2.5 | 125 | Present |
| (SEQ ID | R357M, | | | | | Potassium | | | |
| No. 1181) | A473F, L474C, | | | | | phosphate | | | |
| | I475F, F44Y, | | | | | | | | |
| | F442Q | | | | | | | | |
| G4 | T365N, | 35 | 6 | 0.28% | 75% | 100 mM | 2.4 | 300 | Present |
| (SEQ ID | R357M, | | | | | Sodium | | | |
| No. 1182) | A473F, L474C, | | | | | acetate | | | |
| | I475F, F44Y, | | | | | | | | |
| | F442Q, | | | | | | | | |
| | V263L, F5411 | | | | | | | | |
| G5 | F44Y, V263L, | 35 | 6 | 0.14% | 75% | 100 mM | 3 | 900 | Present |
| (SEQ ID | R357M, | | | | | Sodium | | | |
| No. 1183) | T365N, | | | | | acetate | | | |
| | F442Q, | | | | | | | | |
| | L474C, I475F, | | | | | | | | |
| | F5411, A355W, | | | | | | | | |
| | L367C, Q396R | | | | | | | | |

TBD—to be determined
FIOP—fold improvement over parent (i.e. preceding enzyme), Cumulative FIOP is the product of preceding rounds and is nominally the fold improvement over the original wild-type (WT) starting point (however, since conditions change between rounds and WT isn't used as a control Cumulative FIOP is not a direct measure but an estimate).

The following mutations were associated with enzymes demonstrating improved activity in at least one instance:
F44Y;
V60L;
G117A;
F170N;
V263G or V263L;
N351H or N351Q;
A355H, A355I, A355L, A355M, A355R, A355T or A355W;
A356P;
R357A, R357C, R357K, R357M or R357Q;
G362C;
T365A, T365N or T365S;
L367C;
V394R;
V395Y;
Q396E, Q396G, Q396N, Q396P, Q396R, Q396S or Q396Y;
F430W;
R435F;
V438T;
V440F;
F442M or F442Q;
G444T;
A473F or A473R;
L474C, L474I or L474V;
I475F;
L492C, L492G, L492H, L492I, L492N, L492Q, L492V, L492W or L492Y;
Q493F or Q493H;
P494H or P494I;
S495I, S495K or S495Q;
G496P or G496W;
D498A, D498E, D498F, D498I, D498K, D498L, D498N, D498P, D498R, D498S, D498T or D498V;
A502R;
M504G or M504R;
L507A or L507R;
T508M;
L529M;
F535P;
A536D or A536E;
A537R;
F541A, F541I, F541L, F541M or F541V;
L542I;
Q543G or Q543L;
E547L; and
Y585W.

### Example 9—Screening of Rhamnosidase Variants for Derhamnosylation of Saponins in Crude Bark Extract

### Method

Libraries of genetic variants encoding mutations in the wild type Kribbella flavida rhamnosidase (SEQ ID No. 1017) were prepared using molecular biology techniques, enzymes were prepared linked to a C-terminally located His-tag. Single monoclonal colonies were grown in 400 ul of expression medium and protein expressed. Cell pellets were lysed in 200 ul of the relevant buffer (Table 17).

Lysate was diluted appropriately in the relevant buffer to allow a lysate loading of the indicated % loading (1% loading corresponds to use of 2 ul original lysate in a 200 ul reaction).

Crude bark extract (CBE) obtained by aqueous extraction of Quillaja saponaria and containing at least 2.80 mg/ml QS-21 (by HPLC-UV) was adjusted to pH 6 by dropwise addition of 2M NaOH with stirring. The relevant concentration of the relevant glucosidase was added. The appropriate relative volume of pH 6 CBE (160 ul (for 80%) or 150 ul (for 75%)) was added to each well of the reaction plate. The reaction plate was sealed and incubated at the relevant temperature and time.

The reaction plate was quenched by adding 200 uL of MeCN (2% AcOH, 1 mg/mL hexanophenone) to each well of the plates. The quenched reaction plate was re-sealed and incubated at 20 deg C. with shaking for 10 min. The reaction plate was then centrifuged (10 min, 4 degC).

200 uL was transferred from each well of the quenched plate to the corresponding wells of a fresh 96 well plate and sealed. The plate was analysed by UV HPLC with the method described in Example 4.

Three key peaks of interest are apparent using this chromatography: Left Peak (retention time approximately 2.30-2.35 min) comprising mainly QS-17 family components; Middle Peak (retention time approximately 2.37-2.42 min) comprising mainly QS-18 family components and desglucosyl-QS-17 family components; and Right Peak (retention time approximately 2.44-2.50 min) comprising mainly QS-21 family components. Peak identity was supported by MS/MS.

Enzyme activity is calculated as the % conversion of the Left Peak present in the crude bark extract:

$$\% conversion = 100 \times \frac{( \% Right Peak )}{( \% Left Peak + \% Right Peak )}$$

$$where$$

$$\% Right = \frac{100 \times Right Peak area}{( Right peak area + Left Peak area + Middle Peak area )}$$

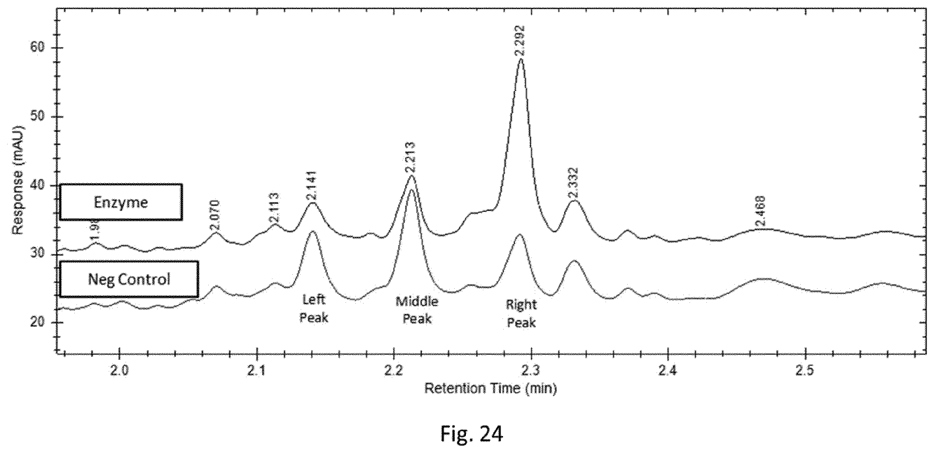

FIG. 24 provides illustrative chromatograms following enzyme treatment of CBE and for a negative control.

TABLE 17

| | | | Conc of | Diluted | | CBE | |
|---|---|---|---|---|---|---|---|
| | | | lysate in | Lysate | Conc CBE | solution | |
| | Temp | Time | reaction | volume | reaction | volume | |
| Enzyme | (deg C.) | (hours) | (% v/v) | (uL) | (% v/v) | (uL) | Buffer |
| R1 | 20 | 22 | 1.25 | 40 | 80 | 160 | 100 mM Potassium |
| (SEQ ID | | | | | | | phosphate pH 6 |
| No. 1189) | | | | | | | |
| R2 | 20 | 20 | 1.25 | 40 | 80 | 160 | 100 mM Potassium |
| (SEQ ID | | | | | | | phosphate pH 6 |
| No. 1190) | | | | | | | |
| R3 | 35 | 18 | 0.4 | 50 | 75 | 150 | 100 mM Sodium |
| (SEQ ID | | | | | | | acetate pH 5.8 |
| No. 1191) | | | | | | | |
| R4 | 35 | 18 | 0.125 | 50 | 75 | 150 | 100 mM Sodium |
| (SEQ ID | | | | | | | acetate pH 5.8 |
| No. 1192) | | | | | | | |
| R5 | 35 | 18 | 0.0625 | 50 | 75 | 150 | 100 mM Sodium |
| (SEQ ID | | | | | | | acetate pH 5.8 |
| No. 1193) | | | | | | | |

R1 (SEQ ID No. 1189)—K219G
R2 (SEQ ID No. 1190)—A143P, L214M, K219G, Q921H;
R3 (SEQ ID No. 1191)—A143P, L214M, K219G, G357C, Q921H
R4 (SEQ ID No. 1192)—A143P, L214M, G215S, G218N, K219G, G357C, G508S, G634A, Q921H
R5 (SEQ ID No. 1193)—A143P, L214M, G215S, G218D, K219G, G357C, G508S, G634A, A690C, Q921H

### Results

TABLE 18

| | | | | | | % conversion | FIOP (% | |
|---|---|---|---|---|---|---|---|---|
| | | | | | % conversion | (Right peak/ | area increase | Cumulative |
| | | Left | Middle | Right | (Right peak/ | Left Peak) | variant/pos | FIOP from |
| Enzyme | Treatment | Peak % | Peak % | Peak % | Left Peak) | (−neg control) | control) | WT |
| R1 | R1 | 27.04 | 45.89 | 27.08 | 50.04 | 2.74 | 1.7 | 1.7 |
| | Negative | 29.36 | 44.30 | 26.35 | 47.30 | 0.00 | | |
| | control | | | | | | | |
| | Positive | 27.94 | 45.30 | 26.76 | 48.92 | 1.62 | | |
| | control | | | | | | | |
| | (WT) | | | | | | | |
| R2 | R2 | 22.76 | 50.97 | 26.27 | 53.57 | 1.09 | 2.9 | 2.9 |
| | Negative | 23.63 | 50.27 | 26.10 | 52.49 | 0.00 | | |
| | control | | | | | | | |
| | Positive | 23.31 | 50.55 | 26.14 | 52.86 | 0.38 | | |
| | control | | | | | | | |
| | (WT) | | | | | | | |
| R3 | R3 | 18.85 | 51.70 | 29.45 | 60.97 | 13.06 | 3.3 | 9.4 |
| | Negative | 28.91 | 44.49 | 26.60 | 47.91 | 0.00 | | |
| | control | | | | | | | |
| | Positive | 25.57 | 46.84 | 27.59 | 51.90 | 3.99 | | |
| | control | | | | | | | |
| | (R2) | | | | | | | |
| R4 | R4 | 25.03 | 47.96 | 27.00 | 51.89 | 5.06 | 2.3 | 22.1 |
| | Negative | 29.62 | 44.29 | 26.09 | 46.84 | 0.00 | | |
| | control | | | | | | | |
| | Positive | 27.65 | 45.79 | 26.56 | 49.00 | 2.16 | | |
| | control | | | | | | | |
| | (R3) | | | | | | | |
| R5 | R5 | 26.59 | 46.48 | 26.93 | 50.32 | 2.62 | 1.3 | 28.7 |
| | Negative | 28.76 | 45.00 | 26.23 | 47.70 | 0.00 | | |
| | control | | | | | | | |
| | Positive | 27.09 | 46.12 | 26.79 | 49.72 | 2.02 | | |
| | control | | | | | | | |
| | (R4) | | | | | | | |

FIOP—fold improvement over parent (i.e. preceding enzyme), Cumulative FIOP is the product of preceding rounds and is nominally the fold improvement over the original WT starting point (however, since conditions change between rounds and WT isn't used as a control Cumulative FIOP is not a direct measure but an estimate).

The following mutations were associated with enzymes demonstrating improved activity in at least one instance:
(i) A56C
(ii) A143P
(iii) Q181H, Q181R or Q181S
(iv) L214M
(v) G215S
(vi) F216M
(vii) G218D or G218N
(viii) K219G
(ix) A238M
(x) T252Y
(xi) T311W
(xii) V326C
(xiii) G357C
(xiv) S369C, S369I, S369K or S369M
(xv) I487M, I487Q or I487V
(xvi) K492N
(xvii) V499T
(xviii) G508S
(xix) R543C
(xx) L557Y
(xxi) G634A
(xxii) S635N
(xxiii) A690C and
(xxiv) Q921H.

### Example 10—Deglucosylation and Derhamnosylation of Saponins in Crude Bark Extract (50%) Using Engineered Enzymes

### Method

Lyophilised powders from clarified cell lysate expressing glucosidases (from Example 8 WT glucosidase and engineered glucosidase polypeptides G1 to G5) and rhamnosidases (from Example 9 WT rhamnosidase and engineered rhamnosidase polypeptides R1 to R5) were dissolved in 200 mM sodium acetate aqueous solution at pH 5.8 to prepare the enzyme solutions at 4 fold the final reaction concentration as shown in Tables 19 and 20.

Each glucosidase solution was combined with an equal volume of 200 mM sodium acetate aqueous solution at pH 5.8 and separately with an equal volume of 200 mM sodium acetate aqueous solution at pH 5.8 containing 2 mg/ml rhamnosidase R5. This is a sufficient loading of rhamnosidase to effect complete hydrolysis of the relevant rhamnose moiety within 4 hours.

Each rhamnosidase solution was combined with an equal volume of 200 mM sodium acetate aqueous solution at pH 5.8 and separately with an equal volume of 200 mM sodium acetate aqueous solution at pH 5.8 containing 2 mg/ml glucosidase G5. This is a sufficient loading of glucosidase to effect complete hydrolysis of the relevant glucose moiety within 4 hours.

CBE was adjusted to pH of 6.0 to 6.2 with 2M sodium hydroxide and an equal volume added to the enzyme solution to prepare the reaction mix (i.e. 50% CBE concentration in reaction mix) and the concentration of glucosidase and/or rhamnosidase is shown in Tables 19 and 20. The reaction mix was heated to 35 degC for the time indicated in Tables 19 and 20, after which the reaction was quenched by addition of an equal volume of MeCN containing 2% acetic acid and shaken at room temperature for 10 mins prior to centrifugation (10 min, 4 degC) to remove particulates. Samples were analysed by UV HPLC using method of Experiment 4-4.

Results

The change in composition of the Left, Middle and Right peaks is shown in in Tables 19 and 20. The composition changes by the action of the enzymes depending on the presence or absence of the partner enzyme. The extent of reaction is proportional to the enzyme concentration and the reaction time under these conditions. The tables show data for enzyme concentrations and the reaction times providing for equivalent extents of reaction. The improvement resulting from the mutations introduced for each variant is equal to the fold change in enzyme concentration×time (i.e. fold improvement=(enzyme concentration×time) for preceding variant÷(enzyme concentration×time) for later variant). A cumulative fold improvement over the original enzyme variant is calculated by the product of the individual fold improvements. The glucosidase G5 shows approximately 800 fold improvement over WT glucosidase. The rhamnosidase R5 shows approximately 30 fold improvement over WT rhamnosidase.

Variants G5 and R5 were found to demonstrate activity across a range of reaction conditions from 25 degC to 40 degC, from pH 5 to 7 (maintaining >80% relative activity for pH 5.4 to 6.2, and >50% for pH 5.2 to 7), and with a range of CBE loadings to at least 150% (achieved by redissolving lyophilised CBE in a smaller volume).

TABLE 19

| | | | | | | | | | | | | |
|---|---|---|---|---|---|---|---|---|---|---|---|---|
| | | | | | | | | | % | | | Cumulative |
| | | Rham- | | | | | | % | change | % | Fold | Fold |
| | Glucosidase | nosidase | | | % | % | % | change | in | change | improvement in | improvement in |
| | conc | conc | Rham- | Time | Left | Middle | Right | in Left | Middle | in Right | primary enzyme | primary enzyme |
| Glucosidase | (mg/ml) | (mg/ml) | nosidase | (h) | Peak | Peak | Peak | Peak | Peak | Peak | loading × time | loading × time |

| | | | |
|---|---|---|---|
| Starting CBE (untreated) | 30 | 44 | 26 |

Glucosidase in the absence of Rhamnosidase

| | | | | | | | | | | | | |
|---|---|---|---|---|---|---|---|---|---|---|---|---|
| WT | 50 | 0 | — | 7 | 23 | 24 | 52 | −22% | −45% | 201% | 1 | 1 |
| G1 | 6.25 | 0 | — | 7 | 28 | 18 | 54 | −8% | −59% | 209% | 8.0 | 8 |
| G2 | 1.56 | 0 | — | 7 | 26 | 19 | 55 | −15% | −57% | 213% | 4.0 | 32 |
| G3 | 0.78 | 0 | — | 7 | 24 | 22 | 53 | −19% | −50% | 206% | 2.0 | 64 |
| G4 | 0.39 | 0 | — | 7 | 23 | 23 | 54 | −22% | −49% | 208% | 2.0 | 128 |
| G5 | 0.1 | 0 | — | 4 | 27 | 19 | 54 | −11% | −56% | 208% | 6.5 | 834 |

Glucosidase in the presence of Rhamnosidase

| | | | | | | | | | | | | |
|---|---|---|---|---|---|---|---|---|---|---|---|---|
| WT | 50 | 0.5 | R5 | 7 | 16 | 16 | 68 | −45% | −64% | 261% | 1 | 1 |
| G1 | 6.25 | 0.5 | R5 | 7 | 18 | 16 | 66 | −39% | −64% | 253% | 8.0 | 8 |
| G2 | 1.56 | 0.5 | R5 | 7 | 22 | 8 | 70 | −26% | −82% | 269% | 4.0 | 32 |
| G3 | 0.78 | 0.5 | R5 | 7 | 21 | 9 | 70 | −31% | −79% | 270% | 2.0 | 64 |
| G4 | 0.39 | 0.5 | R5 | 7 | 22 | 9 | 69 | −28% | −79% | 267% | 2.0 | 128 |
| G5 | 0.1 | 0.5 | R5 | 4 | 20 | 7 | 73 | −34% | −83% | 280% | 6.5 | 834 |

TABLE 20

| | | | | | | | | | | | | |
|---|---|---|---|---|---|---|---|---|---|---|---|---|
| | | | | | | | | | % | | | Cumulative |
| | | Rham- | | | | | | % | change | % | Fold | Fold |
| | Glucosidase | nosidase | | | % | % | % | change | in | change | improvement in | improvement in |
| | conc | conc | Rham- | Time | Left | Middle | Right | in Left | Middle | in Right | primary enzyme | primary enzyme |
| Glucosidase | (mg/ml) | (mg/ml) | nosidase | (h) | Peak | Peak | Peak | Peak | Peak | Peak | loading × time | loading × time |

| | | | |
|---|---|---|---|
| Starting CBE (untreated) | 30 | 44 | 26 |

Rhamnosidase in the absence of Glucosidase

| | | | | | | | | | | | | |
|---|---|---|---|---|---|---|---|---|---|---|---|---|
| — | 0 | 5 | WT | 7 | 18 | 52 | 30 | −40% | 17% | 117% | 1 | 1 |
| — | 0 | 2.5 | R1 | 7 | 19 | 51 | 29 | −35% | 17% | 112% | 2.0 | 2 |
| — | 0 | 1.25 | R2 | 7 | 18 | 52 | 29 | −39% | 19% | 113% | 2.0 | 4 |
| — | 0 | 0.63 | R3 | 7 | 16 | 53 | 30 | −45% | 21% | 116% | 2.0 | 8 |
| — | 0 | 0.31 | R4 | 7 | 16 | 54 | 30 | −45% | 22% | 115% | 2.0 | 16 |
| — | 0 | 0.16 | R5 | 7 | 17 | 56 | 28 | −45% | 27% | 106% | 1.9 | 31 |

Rhamnosidase in the presence of Glucosidase

| | | | | | | | | | | | | |
|---|---|---|---|---|---|---|---|---|---|---|---|---|
| G5 | 0.5 | 5 | WT | 7 | 18 | 3 | 80 | −41% | −94% | 306% | 1 | 1 |
| G5 | 0.5 | 2.5 | R1 | 7 | 18 | 2 | 80 | −40% | −96% | 309% | 2.0 | 2 |
| G5 | 0.5 | 1.25 | R2 | 7 | 18 | 2 | 80 | −40% | −96% | 309% | 2.0 | 4 |
| G5 | 0.5 | 0.63 | R3 | 7 | 19 | 0 | 81 | −38% | −99% | 311% | 2.0 | 8 |
| G5 | 0.5 | 0.31 | R4 | 7 | 17 | 3 | 79 | −42% | −93% | 306% | 2.0 | 16 |
| G5 | 0.5 | 0.31 | R5 | 4 | 20 | 3 | 77 | −34% | −93% | 296% | 1.7 | 27 |

### Example 11—Deglucosylation and Derhamnosylation of Saponins in Crude Bark Extract (50%) Using Engineered Enzymes

### Method

Lyophilised powders from clarified cell lysate expressing engineered glucosidase polypeptide G3 from Example 8 and engineered rhamnosidase polypeptide R2 were dissolved in 200 mM sodium acetate aqueous solution at pH 5.8 to a concentration of 3 g/L (glucosidase) and 2 g/L (rhamnosidase). For a 1 L reaction, sodium acetate buffer 200 mM (700 mL) was charged to a stirred reactor. Under constant agitation, glucosidase enzyme powder (2.1 g) and rhamnosidase enzyme powder (1.4 g) were added and agitated for 30 mins until all the enzyme powder was suspended. The resulting enzyme solution (700 mL) was depth filtered (nom. 3-9 μm) and then sterile filtered (0.2 μm).

CBE (700 mL) containing 4.1 g/L QS-21, with a Preceding Peak ratio of 0.25 and a 2018/QS-21 ratio of 0.054 to 0.057 was depth filtered and then sterile filtered (0.2 μm).

Filtered CBE (500 mL) was charged to a stirred reactor, heated to 37 degC and the pH adjusted to pH of 6.0 to 6.2 with 2M sodium hydroxide. Enzyme solution (500 mL) was then charged to the reactor and the solution stirred at 37 degC for 5 hours.

After 5 hours glacial acetic acid was charged into the reaction mixture gradually under moderate agitation to adjust the pH to ˜pH3.8 (target range pH3.5 to 4.0).

The enzyme treated CBE was then purified analogously to the processes provided in Example 3.

### Results

The purified saponin extract was determined to contain at least 98% QS-21 group, at least 93% QS-21 main peak, 0.2% 2018 component, 1% or less of largest peak outside the QS-21 group by UV absorbance at 214 nm and wherein the monoisotope of the most abundant species was 1987.9 m/z.

The increase of QS-21 by mass, based on the QS-21 concentration and the sample volumes, shows a 2.6-3.0× increase in the enzyme treated CBE. The increase of % QS-21 (as % of saponins) showed a 3.0-3.1× increase.

Due to the improved saponin profile of the enzyme treated material a greater recovery yield is obtained while remaining within desired specifications (notably during polystyrene and phenyl resin chromatography where a greater proportion of QS-21 can be recovered). Overall dual enzyme treatment was found to result in approx. 5.2- to 5.3-fold increase in yield compared to a conventional (non-enzyme treated) process.

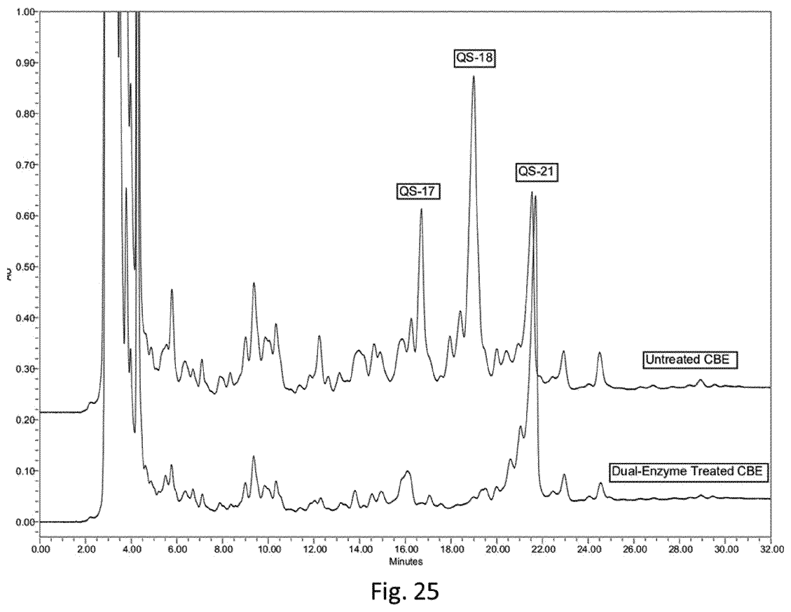

FIG. 25 provides an example HPLC-UV chromatogram of untreated and enzyme treated CBE.

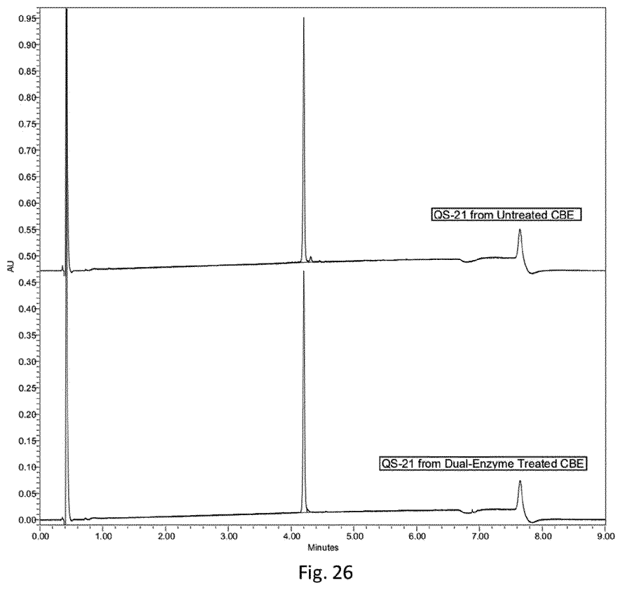

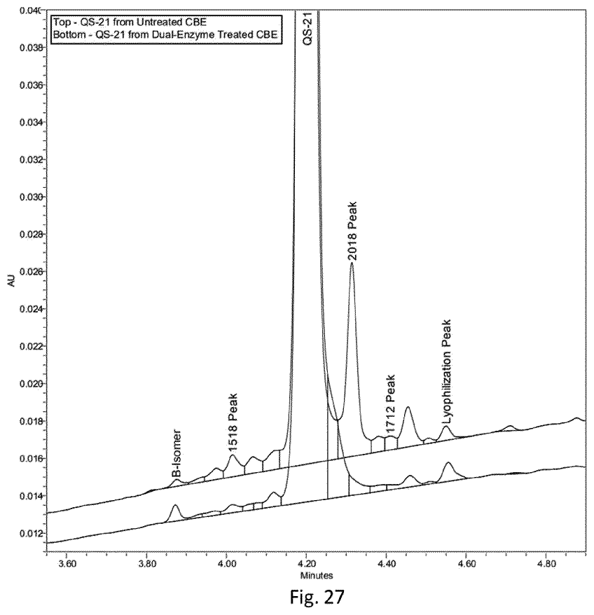

FIG. 26 (full acquisition) and FIG. 27 (zoom) provide example UPLC-UV chromatograms of purified material obtained from untreated and enzyme treated CBE.

### BIBLIOGRAPHY

Altschul S. et al. Basic local alignment search tool Journal of Molecular Biology 1990 215:403-410
Altschul S. et al. Gapped BLAST and PSI-BLAST: a new generation of protein database search programs Nucleic Acids Research 1997 3389-3402
Bomford R. et al. Adjuvanticity and ISCOM formation by structurally diverse saponins Vaccine 1992 10(9):572-577
Cibulski S. et al. Leaf saponins of Quillaja brasiliensis enhance long-term specific immune responses and promote dose-sparing effect in BVDV experimental vaccines Vaccine 2018
36:55-65

Cleland J. et al. Isomerization and Formulation Stability of the Vaccine Adjuvant QS-21 Journal of Pharmaceutical Sciences 1996 85(1):22-28
Dalsgaard K. et al. in 1974 (“Saponin adjuvants”, Archiv. für die gesamte Virusforschung, Vol. 44, Springer Verlag, Berlin, p243-254)
De Becker G. et al. The adjuvant monophosphoryl lipid A increases the function of antigen-presenting cells International Immunology 2000 12:807-815
Didierlaurent A. M. et al. Enhancement of Adaptive Immunity by the Human Vaccine Adjuvant AS01 Depends on Activated Dendritic Cells Journal of Immunology 2014 193(4):1920-1930
Didierlaurent A. M. et al. Adjuvant system AS01: helping to overcome the challenges of modern vaccines Expert Review of Vaccines 2017 16(1): 55-63
Eddy S. Profile Hidden Markov Models Bioinformatics 1988 14:755-763
Garcon N. and Van Mechelen M. Recent clinical experience with vaccines using MPL- and QS-21-containing adjuvant systems Expert Review of Vaccines 2011 10:471-486
Han et al 2014 Characterization of the Structure and Immunostimulatory Activity of a Vaccine Adjuvant, De-O-Acylated Lipooligosaccharide PLoS ONE 9(1):e85838 (and associated correction Han et al 2014 PLoS ONE 9(4):94517)
Hauser M et al. MMseqs software suite for fast and deep clustering and searching of large protein sequence sets Bioinformatics 2016 32(9):1323-1330
Henikoff S. et al. Amino acid substitution matrices from protein blocks Proc Natl Acad Sci USA 1989 89:10915-10919
Hyslop N. St. G. and Morrow A. W. The Influence of Aluminium Hydroxide Content, Dose Volume and the Inclusion of Saponin on the Efficacy of Inactivated Foot-and-Mouth Disease Vaccines Research in Veterinary Science 1969 10:109-120
Ismaili J. et al. Monophosphoryl lipid A activates both human dendritic cells and T cells. Journal of Immunology 2002 168:926-932
Kensil C. R. et al. Separation and characterization of saponins with adjuvant activity from Quillaja saponaria Molina cortex Journal of Immunology 1991 146:431-437
Kensil C. R. et al. (1995) Structural and Immunological Characterization of the Vaccine Adjuvant QS-21. In: Powell M. F., Newman M. J. (eds) Vaccine Design. Pharmaceutical Biotechnology, vol 6. Springer, Boston, MA. https://doi.org/10.1007/978-1-4615-1823-5_22
Kensil C. R. and Kammer R. QS-21: a water-soluble triterpene glycoside adjuvant. Expert Opinion on Investigational Drugs 1998 7:1475-1482
Kite G. C. et al. Metabolomic analysis of saponins in crude extracts of Quillaja saponaria by liquid chromatography/mass spectrometry for product authentication Rapid Communications in Mass Spectrometry 2004 18: 2859-2870
Lambrecht B. N. et al. Mechanism of action of clinically approved adjuvants. Current Opinion in Immunology 2009 21:23-29

Lecas L. et al. Off-line two-dimensional liquid chromatography separation for the quality control of saponins samples from Quillaja saponaria Journal of Separation Science 2021 44:3070-3079
Li H. et al. Cutting edge: inflammasome activation by alum and alum's adjuvant effect are mediated by NLRP3 Journal of Immunology 2008 181:17-21
Livingston P. O. et al. Phase 1 trial of immunological adjuvant QS-21 with a GM2 ganglioside-keyhole limpet haemocyanin conjugate vaccine in patients with malignant melanoma Vaccine 1994 12:1275-1280
Ragupathi G. et al. Natural and synthetic saponin adjuvant QS-21 for vaccines against cancer Expert Review of Vaccines 2011 10:463-470
Reichert C. L. et al. Quillaja Saponin Characteristics and Functional Properties Annual Review of Food Science and Technology 2019 10:43-73
Martin M. et al. Role of innate immune factors in the adjuvant activity of monophosphoryl lipid A Infection and Immunity 2003 71:2498-2507
Marty-Roix R. et al. Identification of QS-21 as an Inflammasome-activating Molecular Component of Saponin Adjuvants Journal of Biological Chemistry 2016 291:1123-1136
Mata-Haro V. et al. The vaccine adjuvant monophosphoryl lipid A as a TRIF-biased agonist of TLR4 Science 2007 316:1628-1632
Needleman S. et al. A General Method Applicable to the Search for Similarities in the Amino Acid Sequences of Two Proteins Journal of Molecular Biology 1970 48:443-453
Newman M. J. et al. Saponin adjuvant induction of ovalbumin-specific CD8+ cytotoxic T lymphocyte responses Journal of Immunology 1992 148:2357-2362
Nyberg N. et al. Separation and structural analysis of some saponins from Quillaja saponaria Molina Carbohydrate Research 2000 323:87-97
Nyberg N. et al. Solid-Phase Extraction NMR Studies of Chromatographic Fractions of Saponins from Quillaja saponaria Anal. Chem. 2003 75 2:268-274
Pearson W. et al. Improved tools for biological sequence comparison Proc. Natl. Acad. Sci. USA 85:2444-2448
Schlotterbeck T. et al. The Use of Leaves from Young Trees of Quillaja saponaria (Molina) Plantations as a New Source of Saponins Economic Botany 2015 69(3):262-272
Soltysik S. et al. Structure/function studies of QS-21 adjuvant: assessment of triterpene aldehyde and glucuronic acid roles in adjuvant function Vaccine 1995 13:1403-1410
Smith T. et al. Comparison of biosequences Advances in Applied Mathematics 1981 2:482-489
Wallace F. et al. Analysis of an immunoadjuvant saponin fraction from Quillaja brasiliensis leaves by electrospray ionization ion trap multiple-stage mass spectrometry Phytochemistry Letters 2017 20:228-233
Wallace F. et al. Structural characterisation of new immunoadjuvant saponins from leaves and the first study of saponins from the bark of Quillaja brasiliensis by liquid chromatography electrospray ionisation ion trap mass spectrometry Phytochemical Analysis 2019 30:644-652
Yendo A. et al. (2017) Purification of an Immunoadjuvant Saponin Fraction from Quillaja brasiliensis Leaves by Reversed-Phase Silica Gel Chromatography In: Christopher B. Fox (ed.), Vaccine Adjuvants: Methods and Protocols, Methods in Molecular Biology, vol. 1494, Springer, New York DOI 10.1007/978-1-4939-6445-1_6.
Re-evaluation of Quillaia extract (E 999) as a food additive and safety of the proposed extension of use European Food Safety Authority Journal 2019 17(3):5622
Current Protocols in Molecular Biology, F. M. Ausubel, et al., eds., Current Protocols, a joint venture between Greene Publishing Associates, Inc. and John Wiley & Sons, Inc., (1995 Supplement).

1. A method for making a product saponin, said method comprising the step of enzymatically converting a starting saponin to the product saponin.

2-3. (canceled)

4. The method according to claim 1, wherein the starting saponin is a quillaic acid glycoside.

5-6. (canceled)

7. The method according to claim 1, wherein the starting saponin is a QS-18 family component.

8. The method according to claim 1, wherein the starting saponin is a desglucosyl-QS-17 family component.

9. The method according to claim 1, wherein the starting saponin is a QS-17 family component.

10. The method according to claim 1, wherein the starting saponin is a desarabinofuranosyl-QS-18 family component.

11. The method according to claim 1, wherein the starting saponin is an acetylated desglucosyl-QS-17 family component.

12-17. (canceled)

18. The method according to claim 1, wherein a single starting saponin is converted to a single product saponin.

19. The method according to claim 1, wherein a plurality of starting saponins is converted to a plurality of product saponins.

20. The method according to claim 1, wherein the starting saponin is obtained by extraction from plant material.

21. (canceled)

22. The method according to claim 1, wherein the enzymatic conversion involves the removal of a beta-glucose residue by a glucosidase.

23. The method according to claim 22, wherein the glucosidase comprises an amino acid sequence according to SEQ ID No. 262, 208, 63, 229, 250, 5, 101, 207, 169, 247, 302, 324, 319, 9, 240, 325, 338, 850, 879, 868, 826, 804, 888, 881, 891, 816, 827, 857, 853, 842, 814, 886, 885, 838, 829, 808, 828, 870, 873, 844, 882, 874, 825, 824, 823, 810, 894, 849, 803, 890, 841, 832, 830, 845, 871, 837, 883 or 809 or functional variants thereof.

24. The method according to claim 1, wherein the step of enzymatic conversion involves the removal of an alpha-rhamnose residue by a rhamnosidase.

25. The method according to claim 24, wherein the rhamnosidase comprises an amino acid sequence according to SEQ ID No. 992, 1003, 1052, 1073, 1017, 1055, 1075, 1001, 1007, 1061, 1079, 1027, 1039, 1041, 989, 1053, 1018, 1066, 1082, 1076, 993, 1077, 1046, 1015, 1063, 1054, 1074, 1067 or 1033, or functional variants thereof.

26. (canceled)

27. A saponin prepared by the method of claim 1.

28. (canceled)

29. An adjuvant composition comprising the saponin according to claim 27.

30. An immunogenic composition comprising the saponin according to claim 27, and an antigen or a polynucleotide encoding an antigen.

31. (canceled)

32. An engineered glucosidase polypeptide comprising an amino acid sequence that is at least 80% identical to the amino acid sequence of SEQ ID No. 262, or a functional fragment thereof, wherein the engineered glucosidase polypeptide includes at least one residue substitution from:
F44Y;
V60L;
G117A;
F170N;
V263G or V263L;
N351H or N351Q;
A355H, A355I, A355L, A355M, A355R, A355T or A355W;
A356P;
R357A, R357C, R357K, R357M or R357Q;
G362C;
T365A, T365N or T365S;
L367C;
V394R;
V395Y;
Q396E, Q396G, Q396N, Q396P, Q396R, Q396S or Q396Y;
F430W;
R435F;
V438T;
V440F;
F442M or F442Q;
G444T;
A473F or A473R;
L474C, L474I or L474V;
I475F;
L492C, L492G, L492H, L492I, L492N, L492Q, L492V, L492W or L492Y;
Q493F or Q493H;
P494H or P494I;
S495I, S495K or S495Q;
G496P or G496W;
D498A, D498E, D498F, D498I, D498K, D498L, D498N, D498P, D498R, D498S, D498T or D498V;
A502R;
M504G or M504R;
L507A or L507R;
T508M;
L529M;
F535P;
A536D or A536E;
A537R;
F541A, F541I, F541L, F541M or F541V;
L542I;
Q543G or Q543L;
E547L; and
Y585W.

33. (canceled)

34. An engineered rhamnosidase polypeptide comprising an amino acid sequence that is at least 80% identical to the amino acid sequence of SEQ ID No. 1017, or a functional fragment thereof, wherein the engineered rhamnosidase polypeptide includes at least one residue substitution from:
(i) A56C
(ii) A143P
(iii) Q181H, Q181R or Q181S
(iv) L214M
(v) G215S
(vi) F216M
(vii) G218D or G218N
(viii) K219G
(ix) A23 8M
(x) T252Y
(xi) T311W
(xii) V326C
(xiii) G357C
(xiv) S369C, S369I, S369K or S369M
(xv) I487M, I487Q or I487V
(xvi) K492N
(xvii) V499T
(xviii) G508S
(xix) R543C
(xx) L557Y
(xxi) G634A
(xxii) S635N
(xxiii) A690C and
(xxiv) Q921H.

35. (canceled)

36. A polynucleotide comprising a sequence encoding an engineered glucosidase polypeptide according to claim 32.

37. A polynucleotide comprising a sequence encoding an engineered rhamnosidase polypeptide according to claim 34.
